(19) United States
(12) Patent Application Publication
(10) Pub. No.: US 2026/0217766 A1
(43) Pub. Date: Jul. 30, 2026

(54) CYCLOPEPTIDE PCSK9 INHIBITOR AND ITS MEDICAL USE

(71) Applicant: SHENZHEN SUNGENING BIOLOGICAL CO., LTD, Shenzhen (CN)

(72) Inventors: Fanglin LI, Shenzhen (CN); Shibin ZHUANG, Shenzhen (CN); Weijun ZHONG, Shenzhen (CN); Qiang LUO, Shenzhen (CN); Zonglian LIN, Shenzhen (CN); Jinhao XIE, Shenzhen (CN); Xingyi LIN, Shenzhen (CN); Hanlin WEI, Shenzhen (CN); Borui ZHAN, Shenzhen (CN); Shanjia LI, Shenzhen (CN); Xinyue SHEN, Shenzhen (CN); Dongmei HE, Shenzhen (CN); Renchong YAO, Shenzhen (CN); Shenghong HUANG, Shenzhen (CN); Yan CHEN, Shenzhen (CN); Chester Chenguang YUAN, Shenzhen (CN)

(21) Appl. No.: 19/084,240

(22) Filed: Mar. 19, 2025

(30) Foreign Application Priority Data
Jan. 25, 2025 (CN) 202510120478.6

(51) Int. Cl. C07K 7/56; A61K 38/00; A61P 3/06

(52) U.S. Cl. CPC C07K 7/56; A61P 3/06; A61K 38/00

(57) ABSTRACT

Disclosed herein is a series of cyclic peptide PCSK9 inhibitors, along with methods of using the same and therapeutic applications thereof in the treatment of cardiovascular diseases, hypercholesterolemia, and related disorders.

### TECHNICAL FIELD

The present disclosure relates to cyclic peptide inhibitors of proprotein convertase subtilisin/kexin type 9 (PCSK9) and their therapeutic applications.

### BACKGROUND

Cardiovascular diseases (CVDs) represent the leading cause of global mortality, with prevalence increasing with age. Atherosclerotic cardiovascular diseases (ASCVDs), including coronary heart disease, myocardial infarction, and stroke, account for significantly more deaths than cancers or other conditions. Elevated plasma levels of low-density lipoprotein cholesterol (LDL-C) are a primary driver of ASCVD pathogenesis.

Current clinical strategies to reduce LDL-C rely heavily on statins, which inhibit 3-hydroxy-3-methylglutaryl-coenzyme A reductase (HMG-CoA reductase), the rate-limiting enzyme in hepatic cholesterol biosynthesis. By upregulating low-density lipoprotein receptor (LDL-R) expression, statins enhance LDL-C clearance. However, limitations persist: patients with severe hyperlipidemia, familial hypercholesterolemia, or statin intolerance often fail to achieve target LDL-C levels. Long-term statin use is also associated with adverse effects, including myopathy, hepatotoxicity, and elevated diabetes risk. Thus, safer, more effective therapies with improved patient compliance remain an unmet need.

The discovery of PCSK9 as a therapeutic target has revolutionized lipid-lowering strategies. Identified in 2003, PCSK9 is a hepatic serine protease that binds LDL-R, promoting its lysosomal degradation and reducing LDL-R surface density, thereby elevating plasma LDL-C. Inhibition of PCSK9 restores LDL-R recycling, significantly lowering LDL-C.

Currently marketed PCSK9 inhibitors include monoclonal antibodies (mAbs) and small interfering RNA (siRNA) agents. The first mAbs, evolocumab (Amgen/Astellas) and alirocumab (Sanofi/Regeneron), approved in 2015, are administered subcutaneously every two weeks. Domestic mAbs, including tafolecimab (Innovent Biologics), ebronucimab (Akeso Biopharma), and ongericimab (Junshi Biosciences), have recently entered the market. Despite efficacy, long-term subcutaneous administration poses challenges: poor patient compliance, injection-site reactions, allergic responses (e.g., rash, angioedema), and rare anaphylaxis. Additionally, mild hyperglycemia and flu-like symptoms are reported.

Inclisiran (Leqvio®), an siRNA therapeutic (Alnylam/Novartis, approved 2020), enables sustained LDL-C reduction with biannual dosing after initial loading. While improving convenience, siRNA therapies face limitations: high cost, cold-chain storage requirements, and theoretical risks of off-target effects due to codon degeneracy.

Recent analyses of >2.14 million patients reveal a U-shaped correlation between LDL-C and all-cause mortality in coronary artery disease (CAD) patients. Both extremes—LDL-C<50 mg/dL and ≥130 mg/dL—are associated with increased mortality. Notably, very low LDL-C (<70 mg/dL) may elevate hemorrhagic stroke and diabetes risks. For long-acting agents like inclisiran, prolonged LDL-C suppression without dose titration raises safety concerns.

Despite clinical success, injectable PCSK9 inhibitors remain unsuitable for needle-averse populations. Oral alternatives, particularly small-molecule inhibitors, have faced developmental hurdles; none have progressed to Phase III trials. Recently, Merck's cyclic peptide MK-0616 demonstrated robust Phase II efficacy, prompting rapid advancement to Phase III studies, reigniting hope for oral PCSK9 inhibition.

### SUMMARY

Through structural analysis, design, synthesis, and screening of PCSK9-LDL-R interaction complexes, the present disclosure identifies a series of highly active cyclic peptide PCSK9 inhibitors with superior in vivo efficacy.

In one aspect, the disclosure provides a compound of Formula (I) or a pharmaceutically acceptable salt thereof:

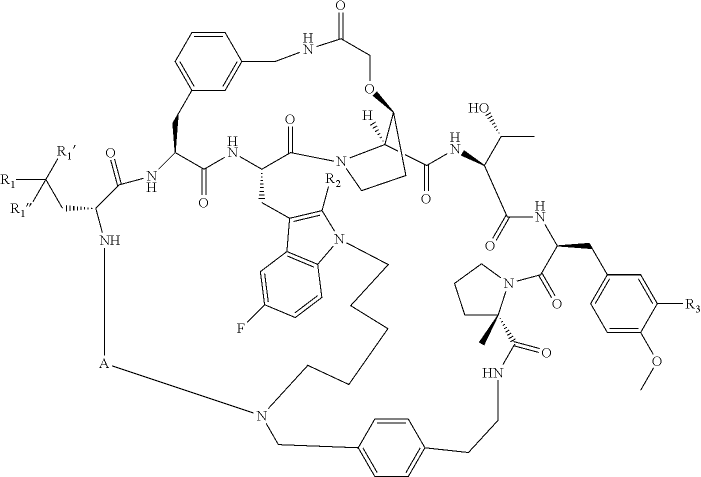

wherein, R₁ is selected from —CH₃, —C(O)—(CH₂)n₁-N⁺(CH₃)₃, or —C(O)—(CH₂CH₂O)n₂-CH₂CH₂N⁺(CH₃)₃, where n₁ is selected from an integer of 1 to 14, and n₂ is selected from an integer of 1 to 5.

Wherein, R₁′ and R₁″ are each independently selected from absence, hydrogen, or CH₃.

Wherein, R₂ is selected from hydrogen, halogen, —SCH₃, —SCH₂X, or —SCHX₂.

Wherein, R₃ is selected from hydrogen, or halogen.

Wherein, A is selected from

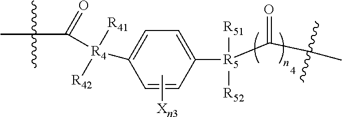

Wherein, R₄ and R₅ are each independently selected from a bond, a linear C₁-C₅ alkyl, phenyl, provided that R₄ and R₅ are not a bond or phenyl at the same time.

Wherein, R₄₁ and R₄₂ are each independently selected from absence, hydrogen, deuterium, substituted or unsubstituted C₁-C₃ alkyl, halogen, or, when R₄ is selected from a linear alkyl, R₄₁ and R₄₂ together with one carbon atom to which they are connected, form a C₃-C₅ cycloalkyl.

Wherein, R₅₁ and R₅₂ are each independently selected from absence, hydrogen, deuterium, substituted or unsubstituted C₁-C₃ alkyl, halogen, or, when R₅ is selected from a linear C₁-C₅ alkyl, R₅₁ and R₅₂ together with one carbon atom to which they are connected, form a C₃-C₅ cycloalkyl.

Wherein, n₃ is selected from an integer of 0 or 1-4; and n₄ is selected from 0 or 1.

Wherein, among the substituted C₁-C₃ alkyl, the substituent is selected from deuterium, or halogen.

Further, when R₁ is not —CH₃, either of R₁′ and R₁″ is selected from absence, the other one is selected from hydrogen; or, when R₁ is —CH₃, R₁′ and R₁″ are —CH₃ at the same time.

Further, R₁ is selected from —C(O)—(CH₂)n₁-N⁺(CH₃)₃, or —C(O)—(CH₂CH₂O)n₂-CH₂CH₂N⁺(CH₃)₃. That is, the present disclosure may be selected from the following structures or a pharmaceutically acceptable salt thereof:

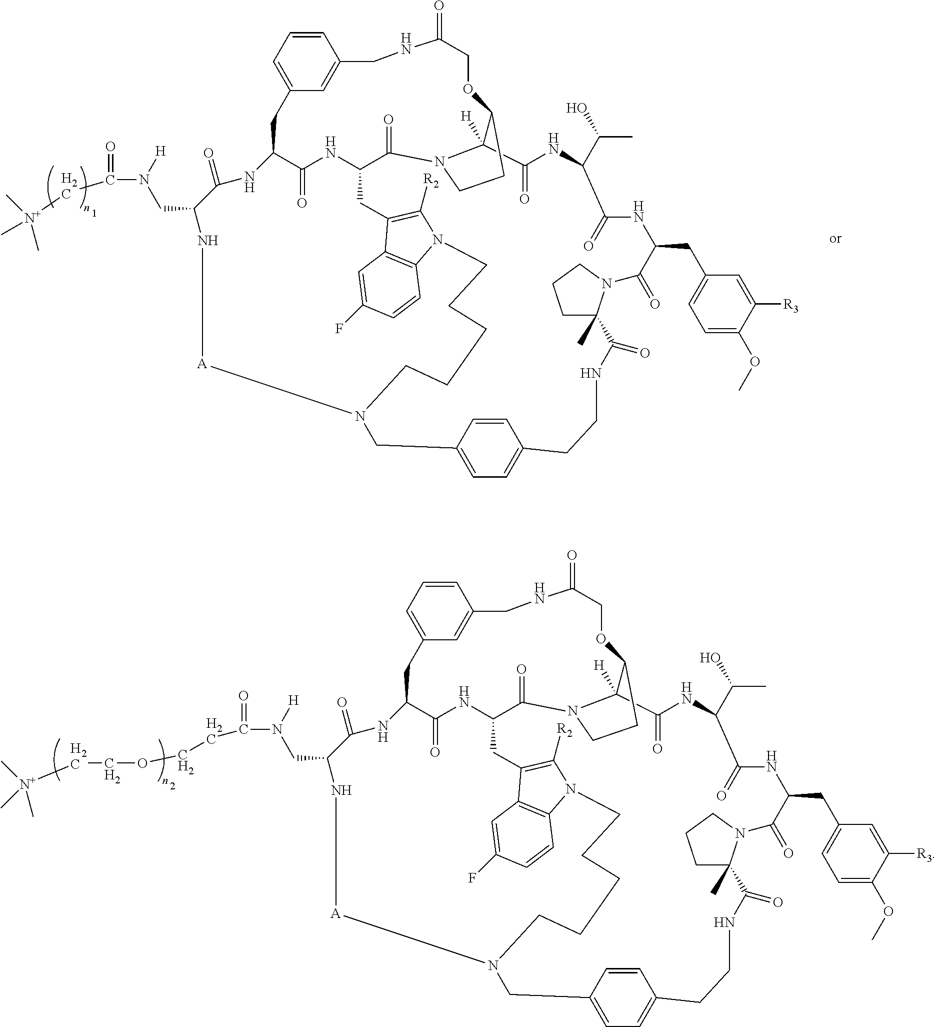

More further, R₁ is selected from —C(O)—(CH₂)n₁-N⁺(CH₃)₃.

Further, R₃ is selected from hydrogen, that is, the present disclosure may be selected from the following structures or a pharmaceutically acceptable salt thereof:

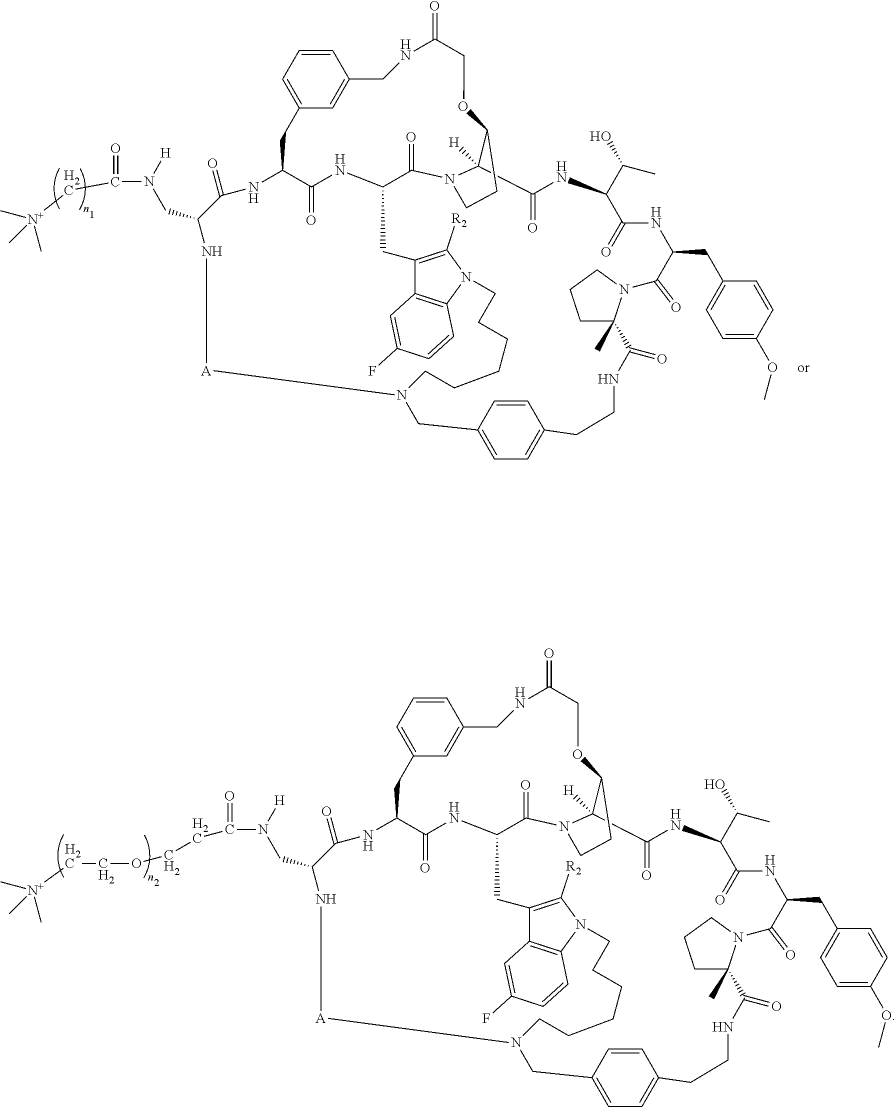

Further, R₂ is selected from hydrogen, —SCHX₂, or —SCX₃, that is, the present disclosure may be selected from the following structures or a pharmaceutically acceptable salt thereof:

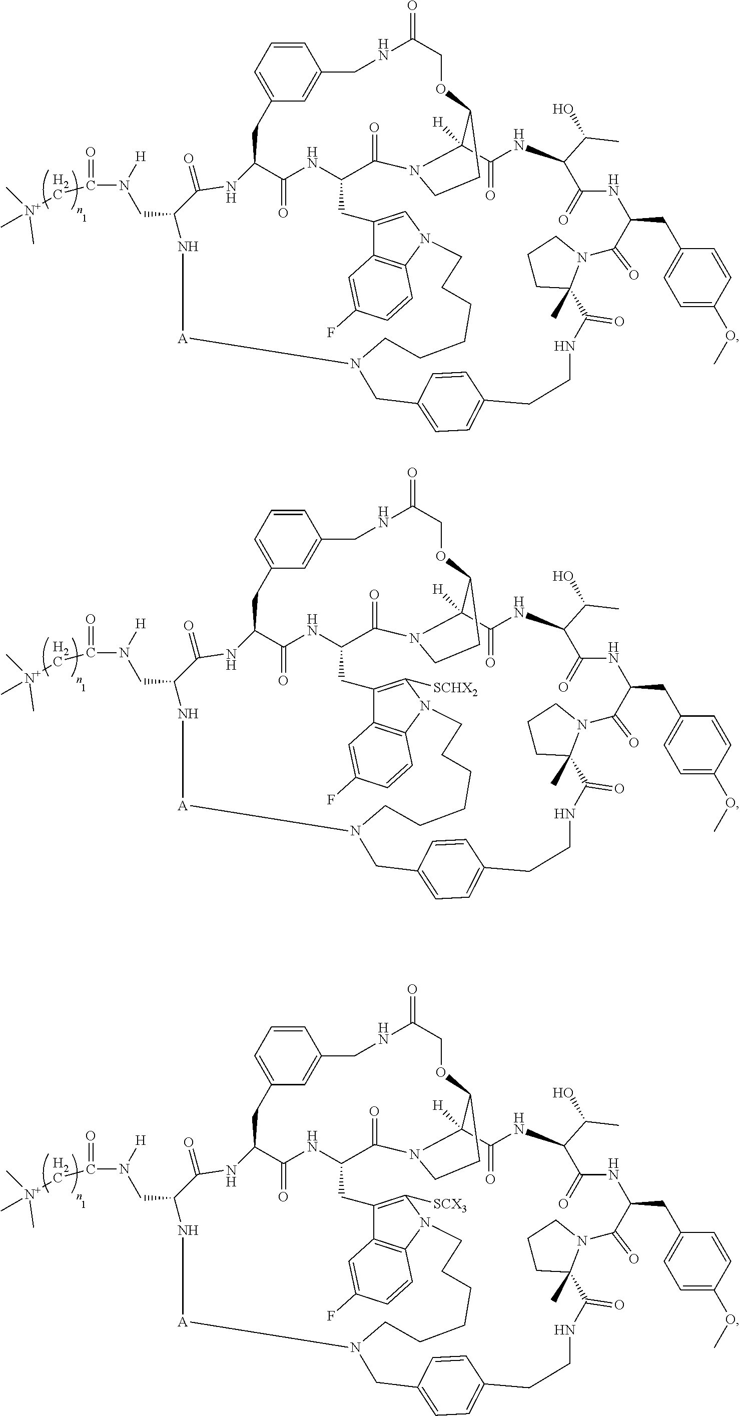

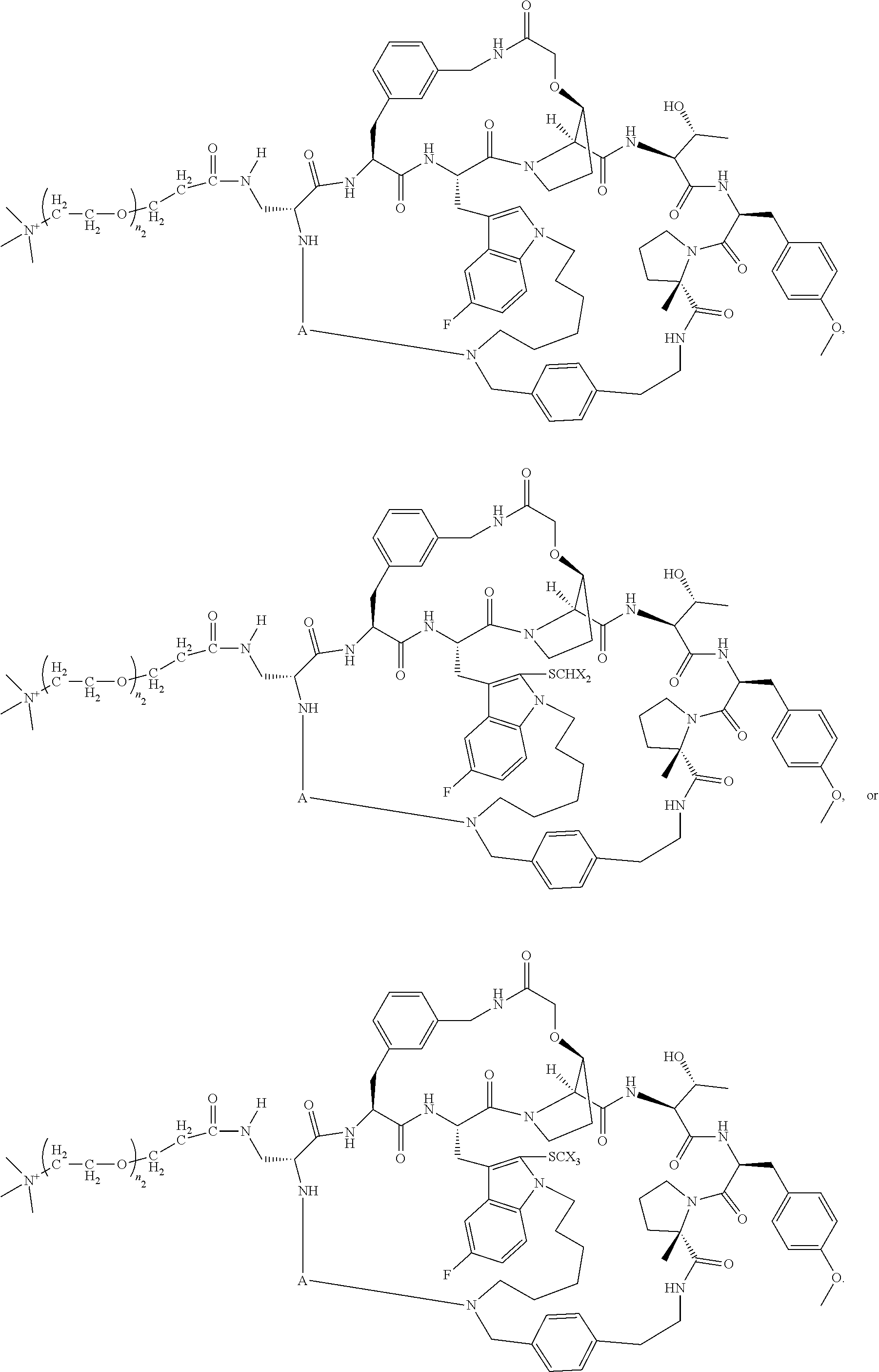

The structures or groups shown in A may exist in the above structural formulas.

Further, when R₄ is a bond, R₄₁ and R₄₂ are absent; or, when R₅ is a bond, R₅₁ and R₅₂ are absent.

Further, when R₄ is a linear alkyl, R₄₁ and R₄₂ are hydrogen, deuterium, substituted or unsubstituted C₁-C₃ alkyl, halogen, or R₄₁ and R₄₂ are connected to the same carbon atom, and together with the carbon atom to which they are connected form a C₃-C₅ cycloalkyl; or, when R₅ is a linear alkyl, R₅₁ and R₅₂ are hydrogen, deuterium, substituted or unsubstituted C₁-C₃ alkyl, halogen, or R₅₁ and R₅₂ are connected to the same carbon atom, and together with the carbon atom to which they are connected form a C₃-C₅ cycloalkyl.

Further, when R₄ is phenyl, R₄₁ and R₄₂ are hydrogen, deuterium, substituted or unsubstituted C₁-C₃ alkyl, or halogen; or, when R₅ is phenyl, R₅₁ and R₅₂ are hydrogen, deuterium, substituted or unsubstituted C₁-C₃ alkyl, or halogen.

Further, R₄ and R₅ are each independently selected from a bond, a linear C₁-C₂ alkyl, phenyl, R₄ and R₅ are not a bond or phenyl at the same time.

Further, R₄₁ and R₄₂ are each independently selected from absence, H, D, —CH₃, halogen, —CH₂X, —CHX₂, —CX₃, —CH₂D, —CHD₂, or —CD₃;

Further, R₅₁ and R₅₂ are each independently selected from absence, H, D, —CH₃, halogen, —CH₂X, —CHX₂, —CX₃, —CH₂D, —CHD₂, or —CD₃.

More further, A is selected from the following structures:

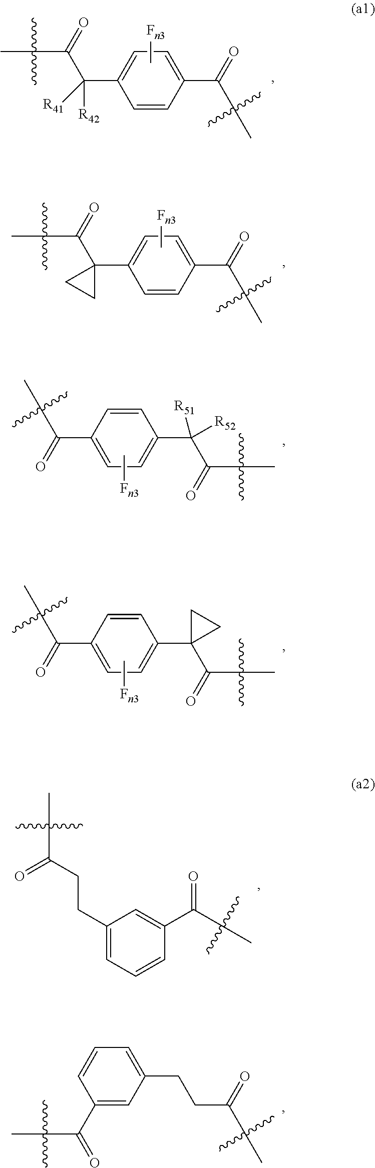

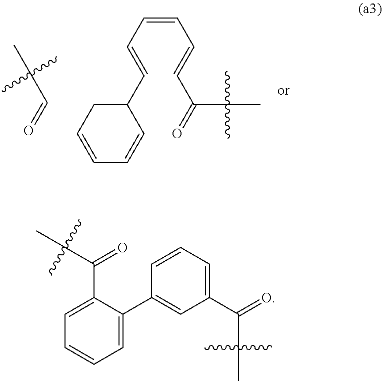

More further, R₄₁ and R₄₂ are each independently selected from hydrogen, methyl or halogen.

More further, R₅₁ and R₅₂ are each independently selected from hydrogen.

Wherein, the above halogen is preferably F.

Wherein, when n₄ is selected from 0, n₃ is selected from an integer of 0 or 1-4. For example, n₄ is selected from 0, n₃ is selected from 0; n₄ is selected from 0, n₃ is selected from 1; n₄ is selected from 0, n₃ is selected from 2; n₄ is selected from 0, n₃ is selected from 3; and n₄ is selected from 0, n₃ is selected from 4.

Wherein, when n₄ is selected from 1, n₃ is selected from an integer of 0 or 1-4. For example, n₄ is selected from 1, n₃ is selected from 0; n₄ is selected from 1, n₃ is selected from 1; n₄ is selected from 1, n₃ is selected from 2; n₄ is selected from 1, n₃ is selected from 3; and n₄ is selected from 1, n₃ is selected from 4.

Wherein, n₃ is further selected from 0, 1 or 2.

Further, the structural formulas of the compounds are selected from the following:

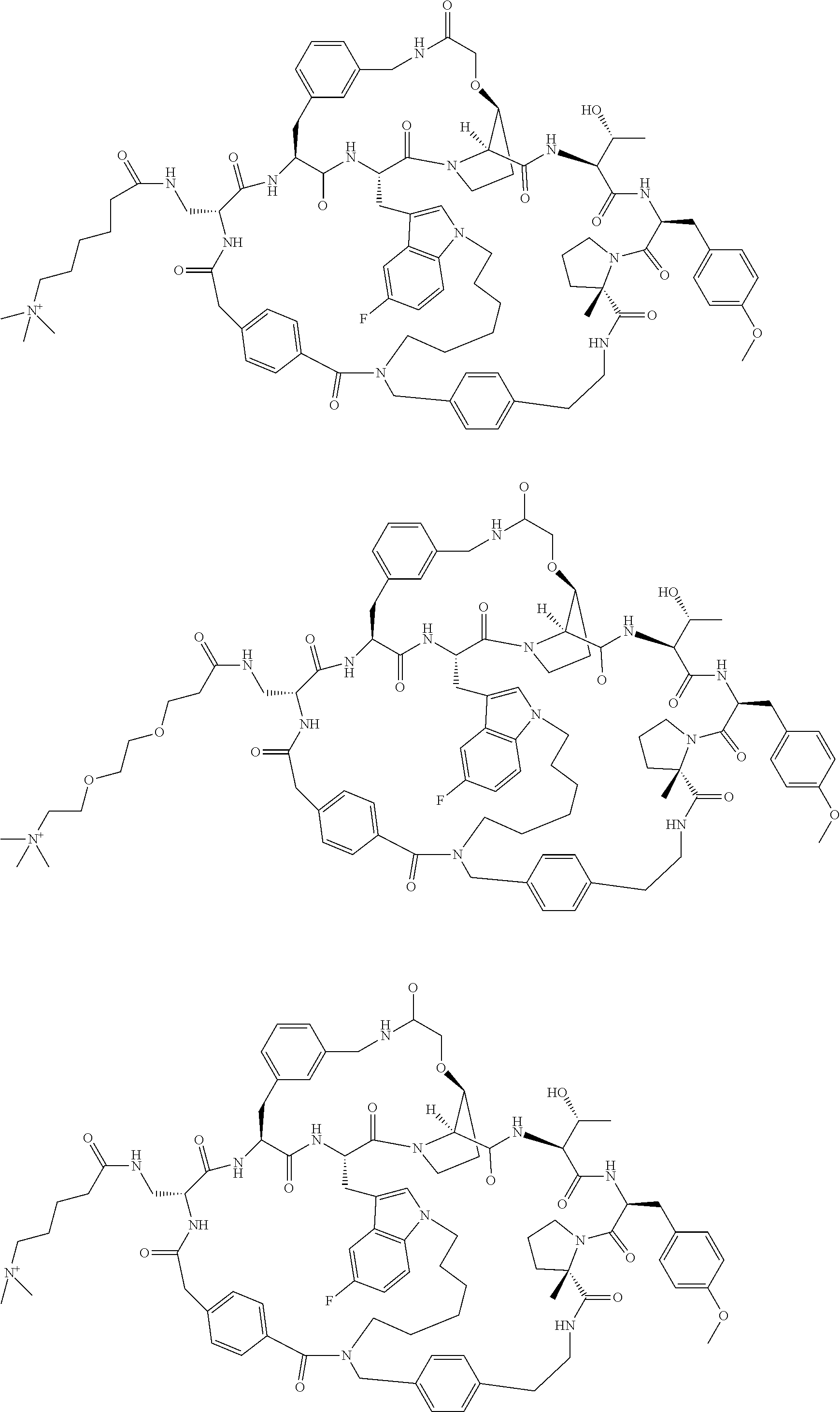

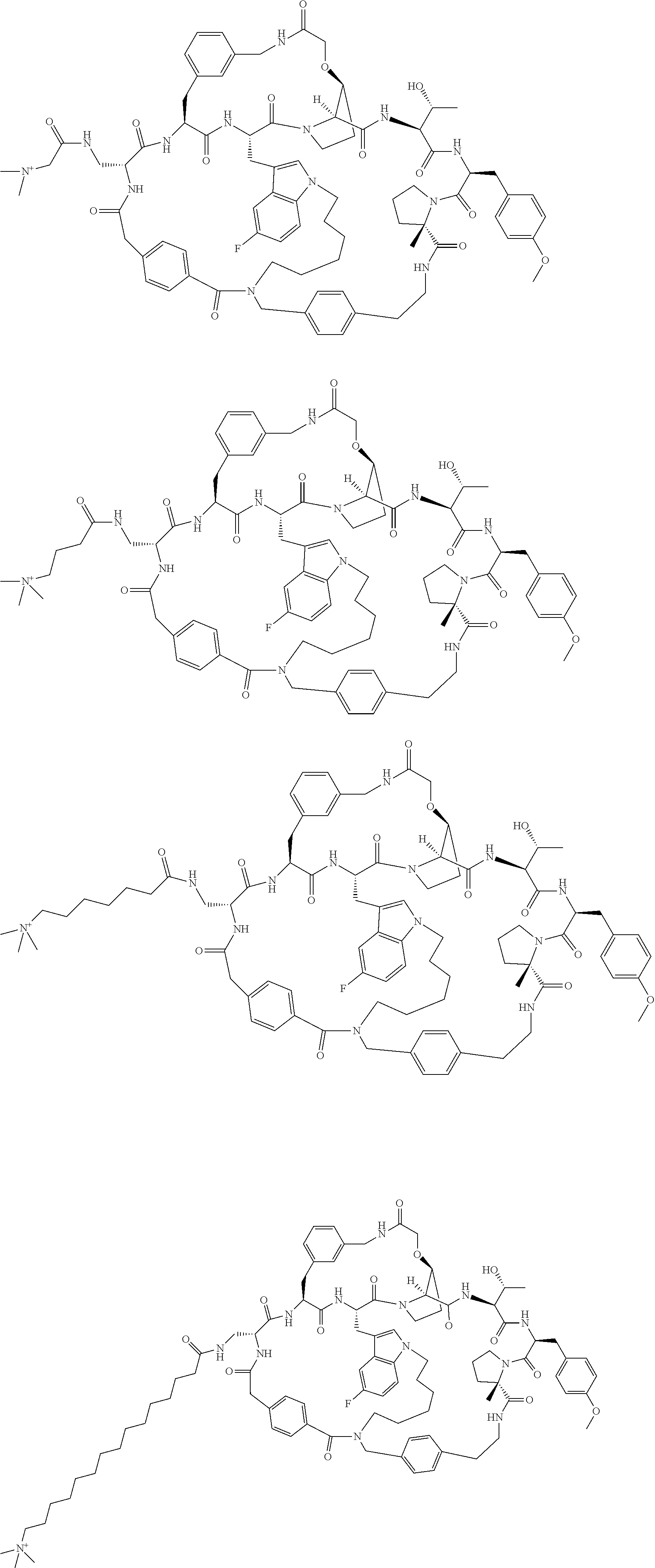

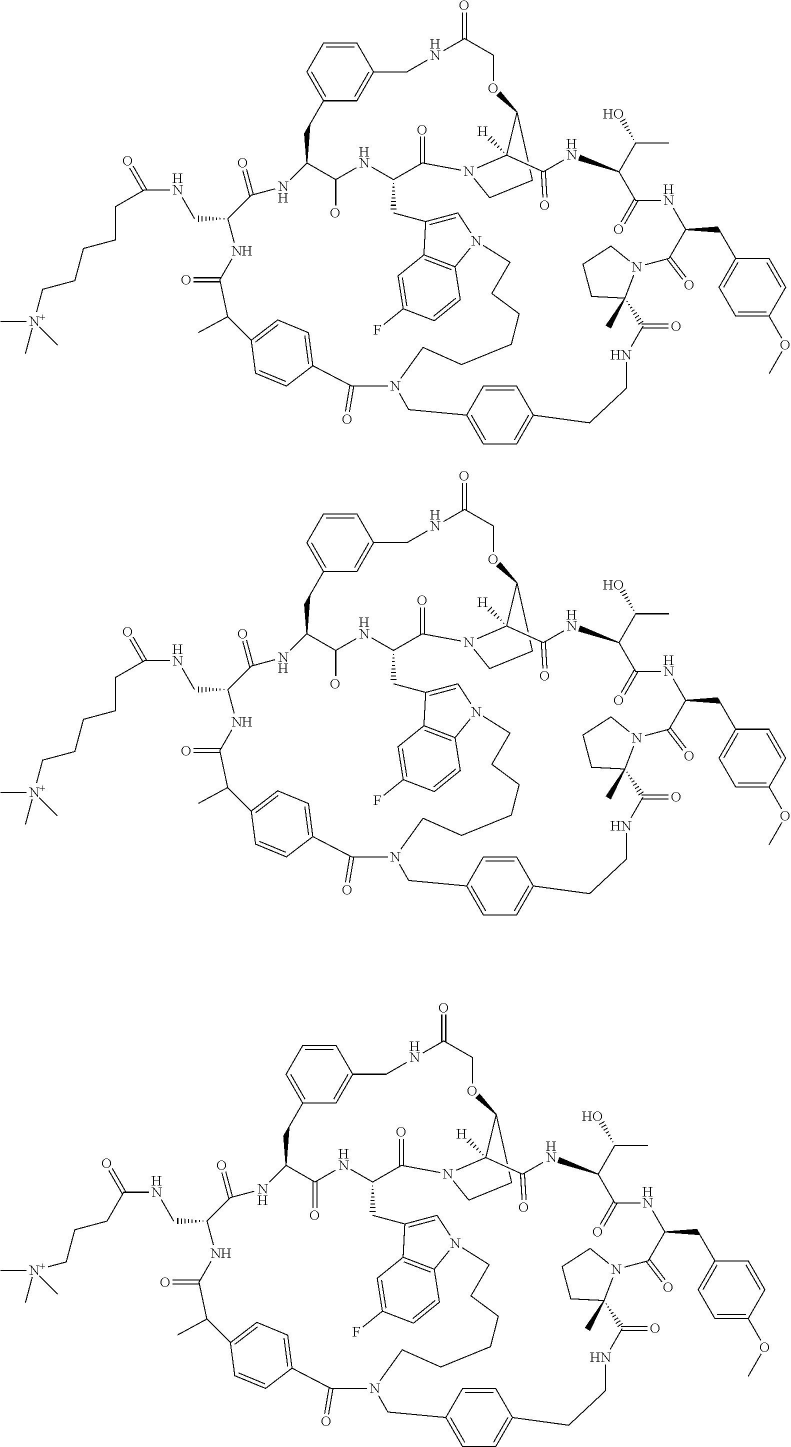

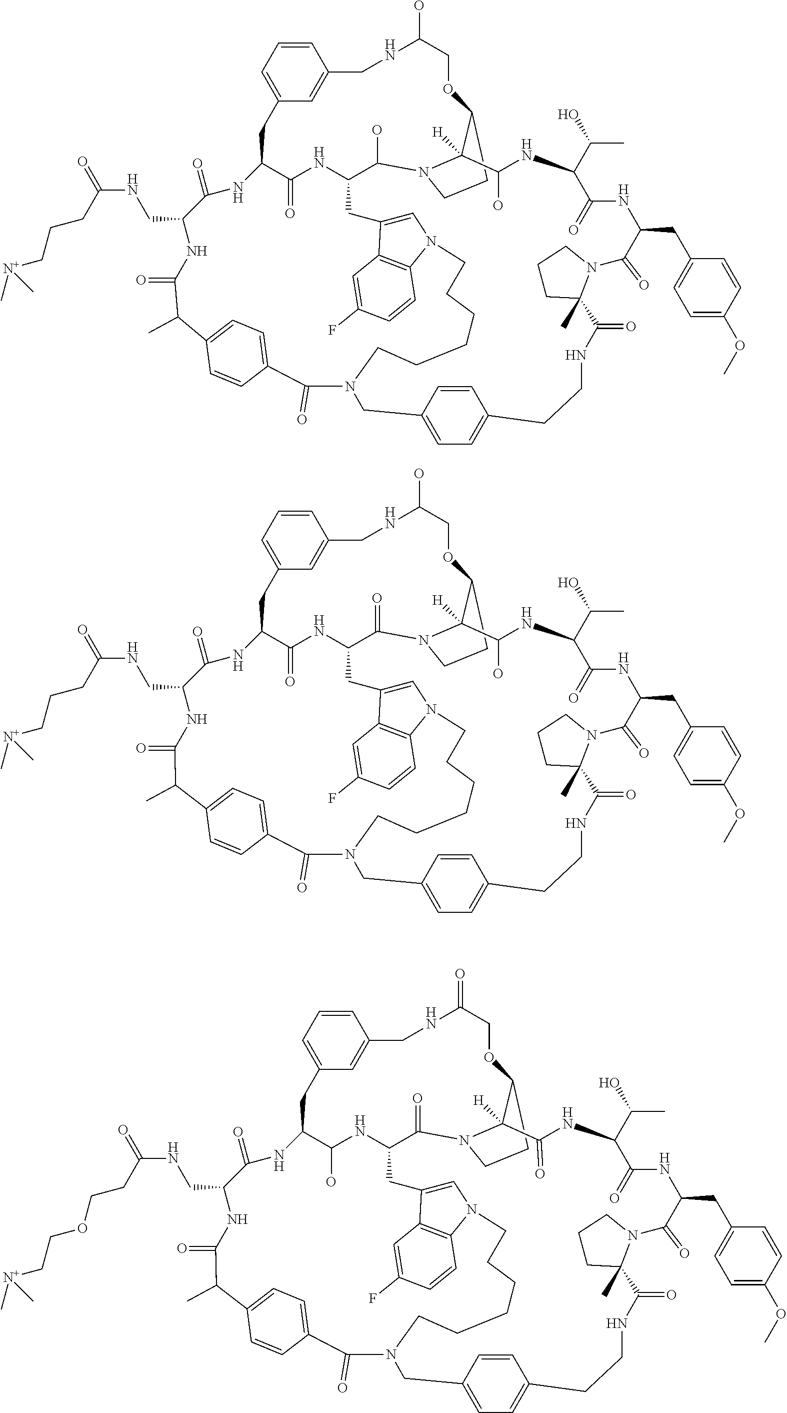

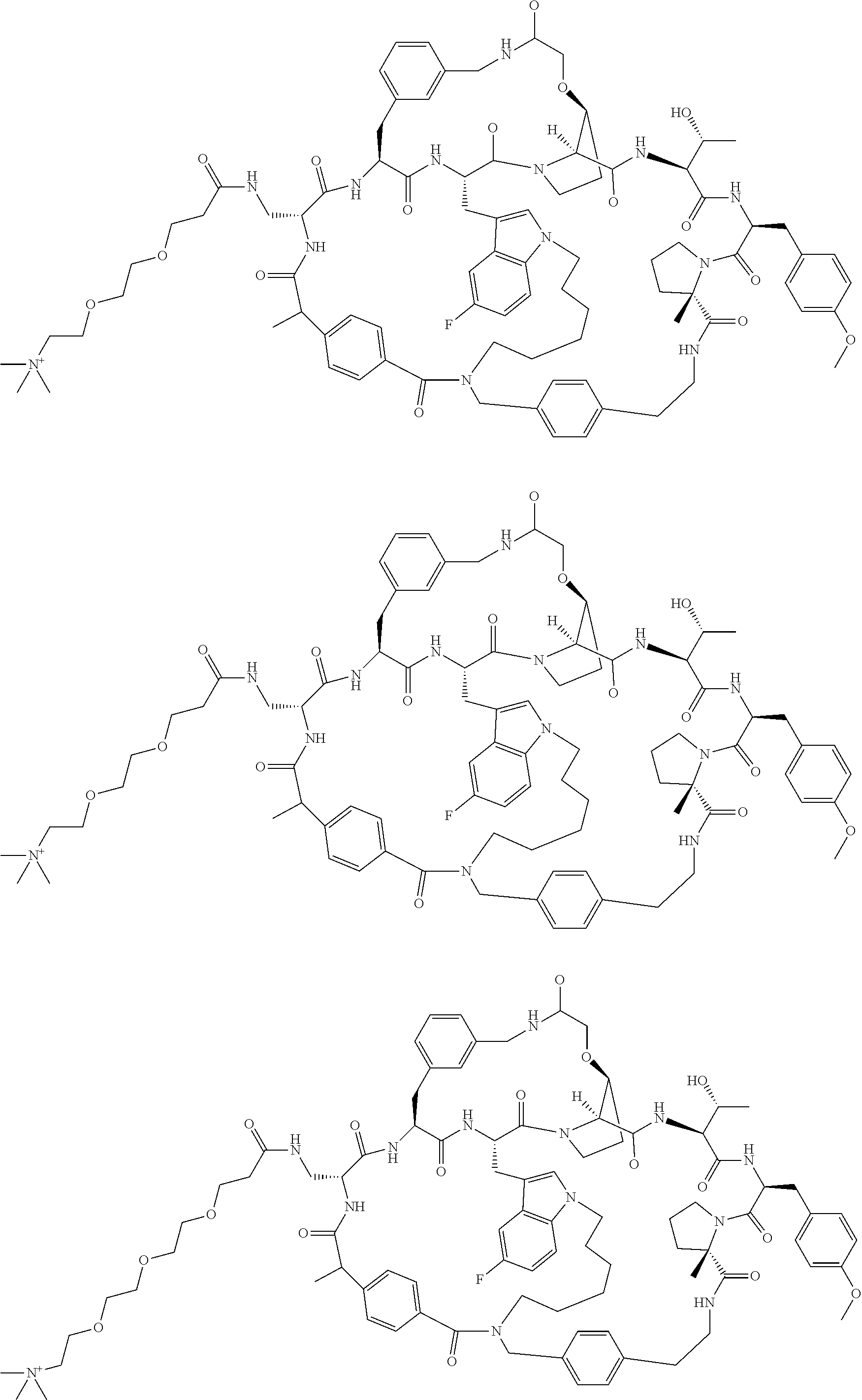

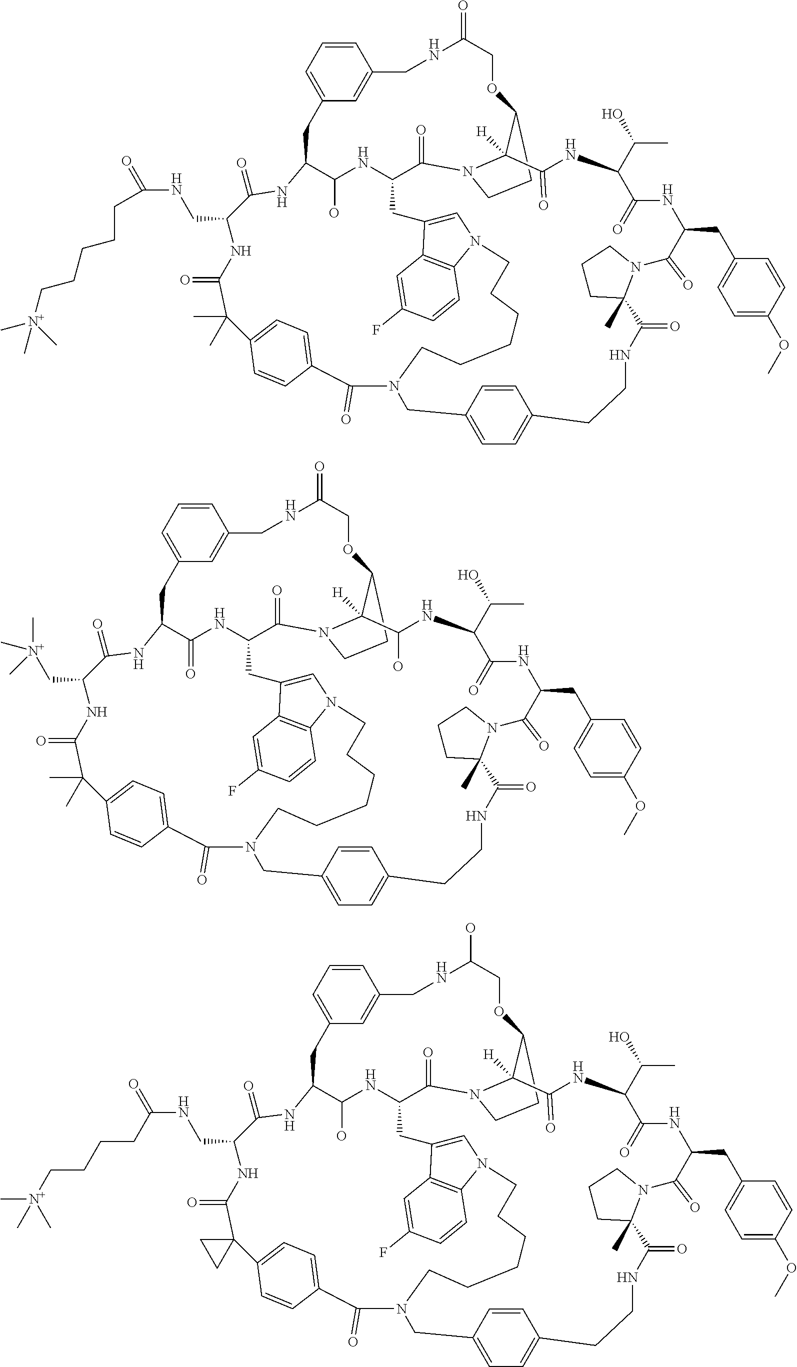

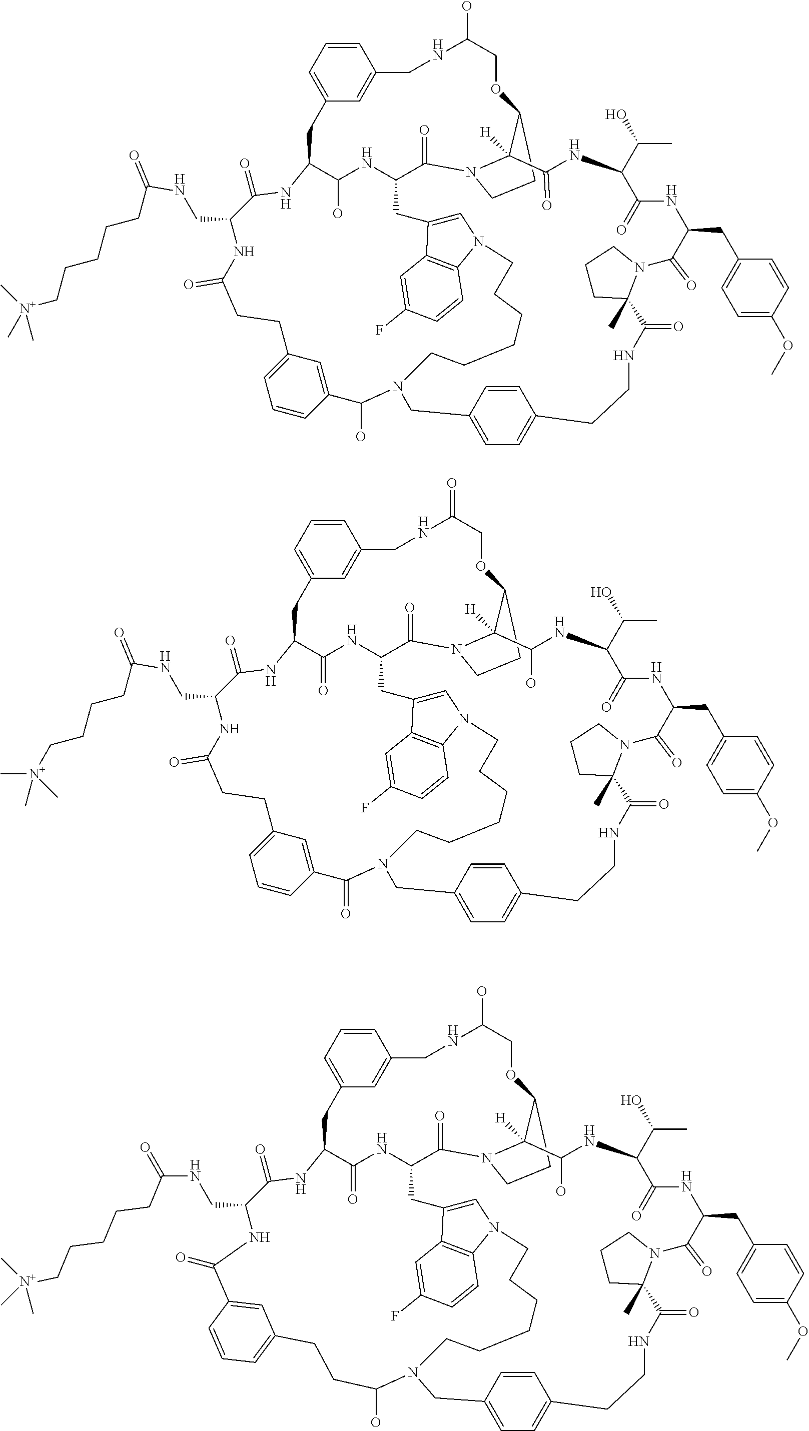

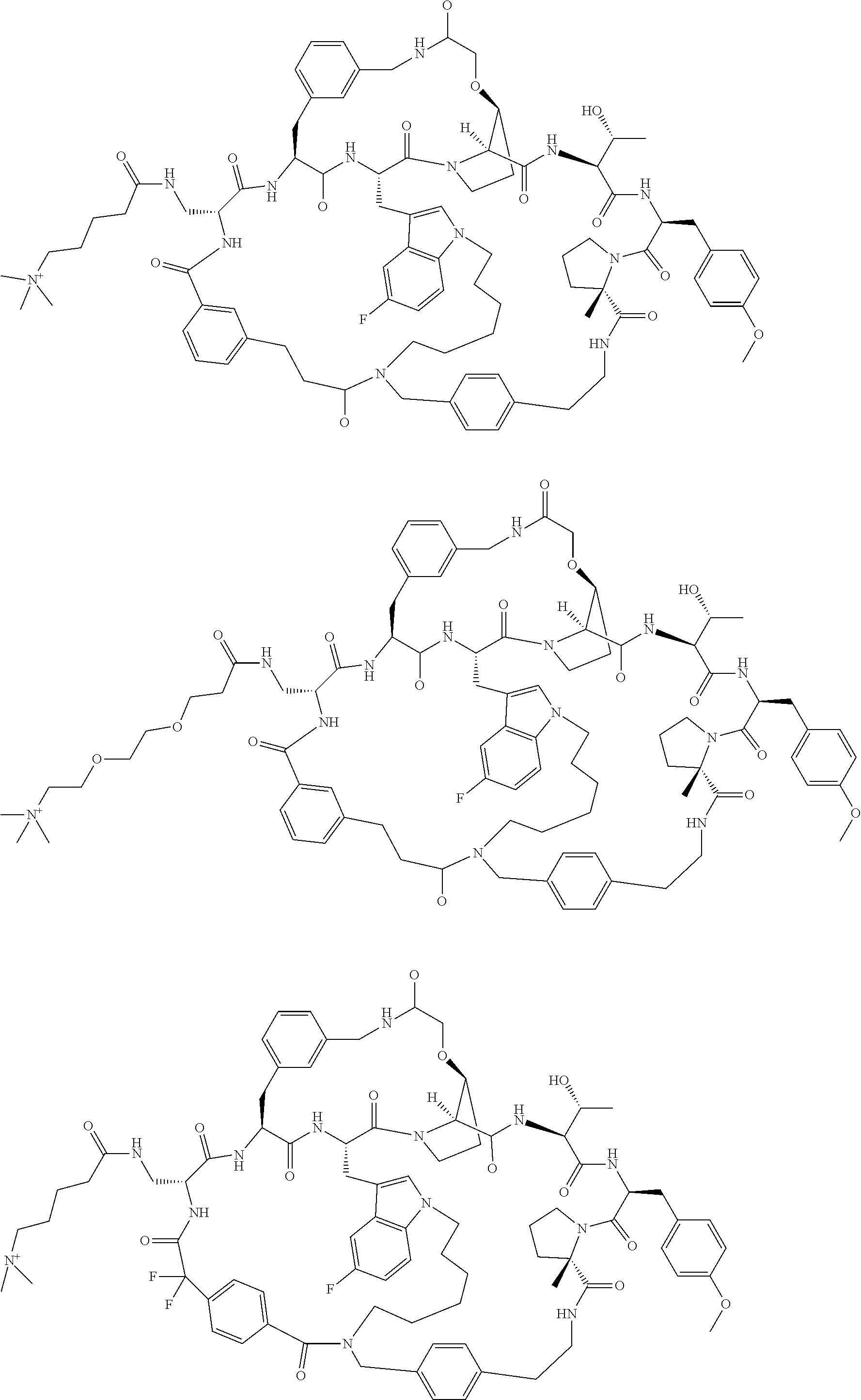

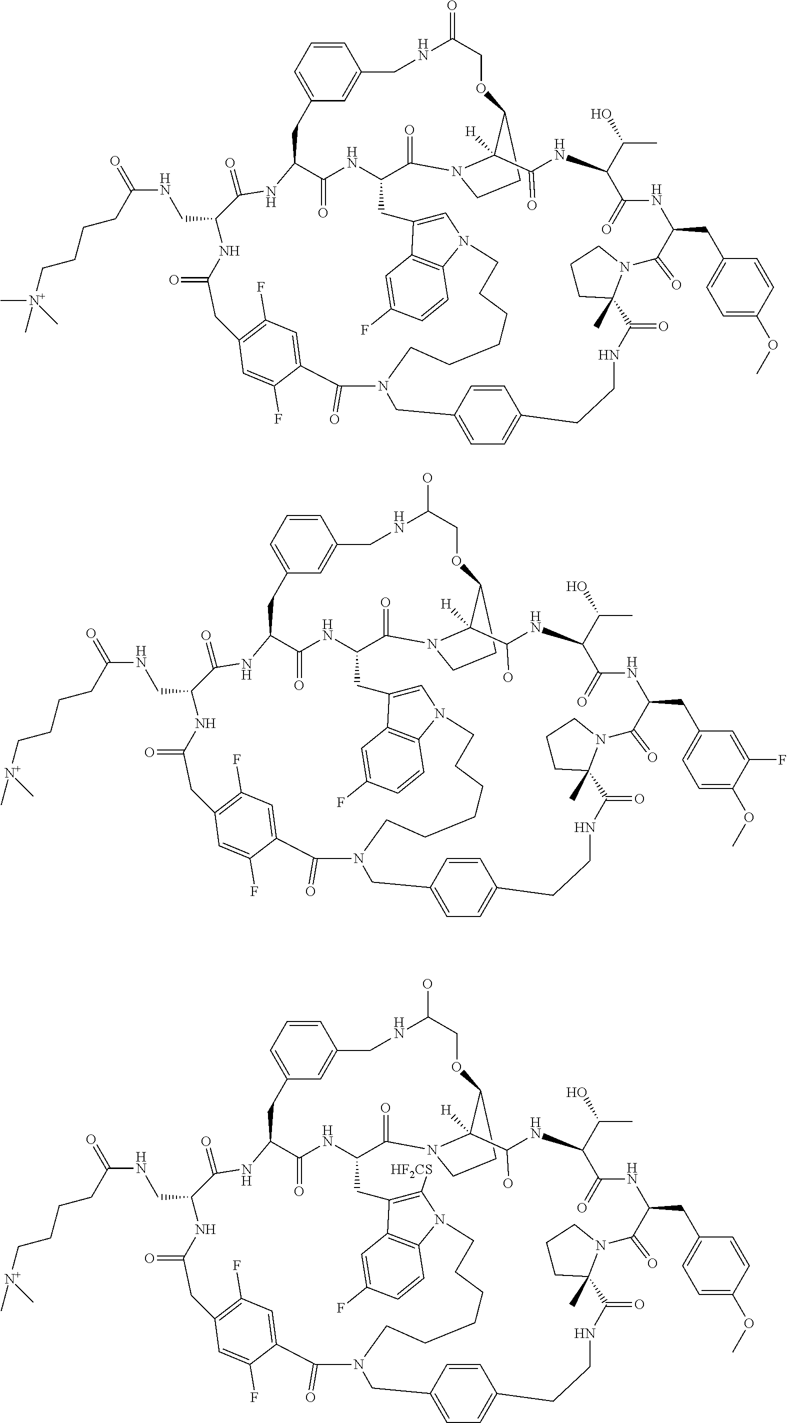

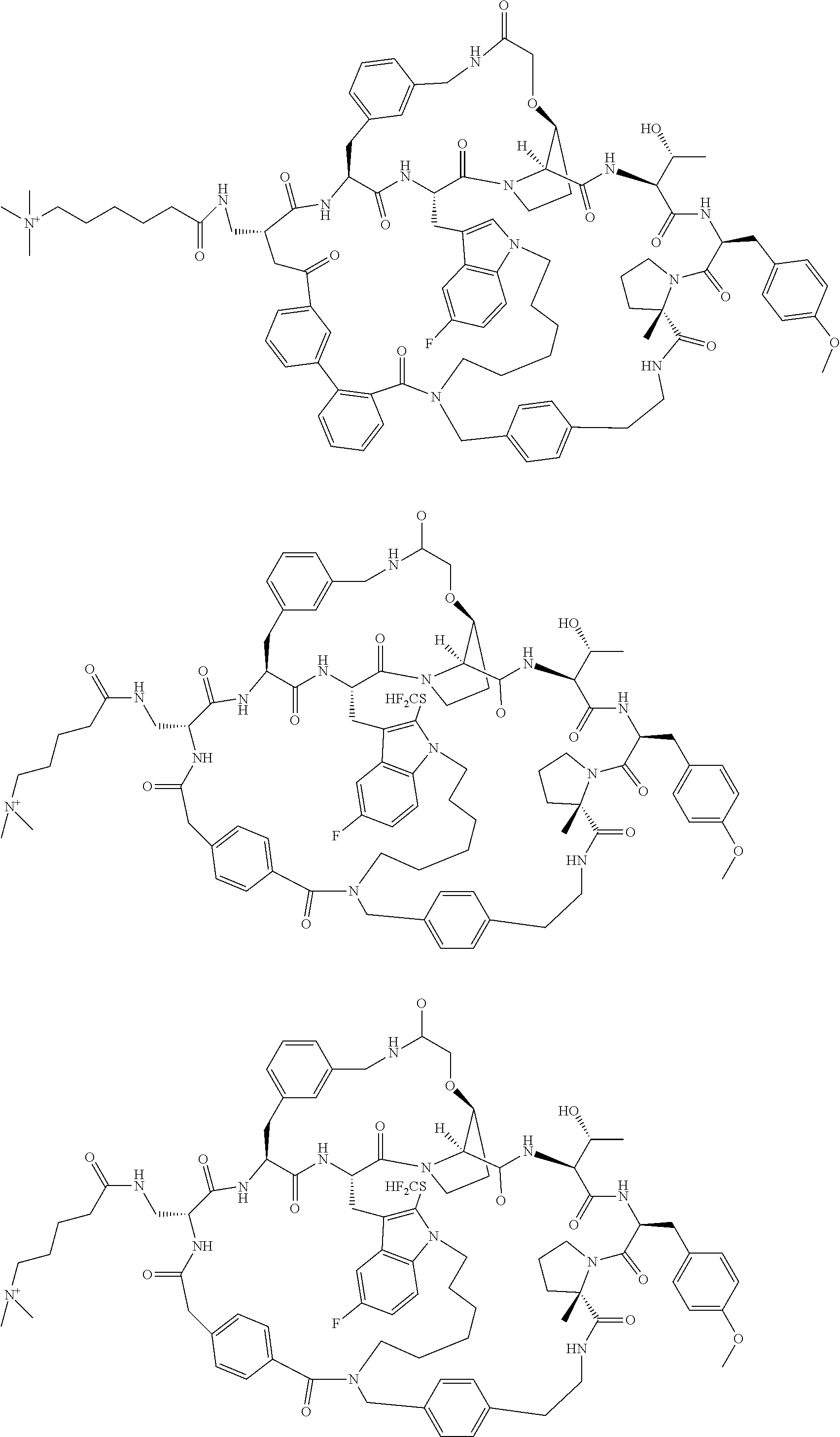

Further, formulas of the compounds are selected from the following:

| Nos. | Structure |
|---|---|
| 2401 | 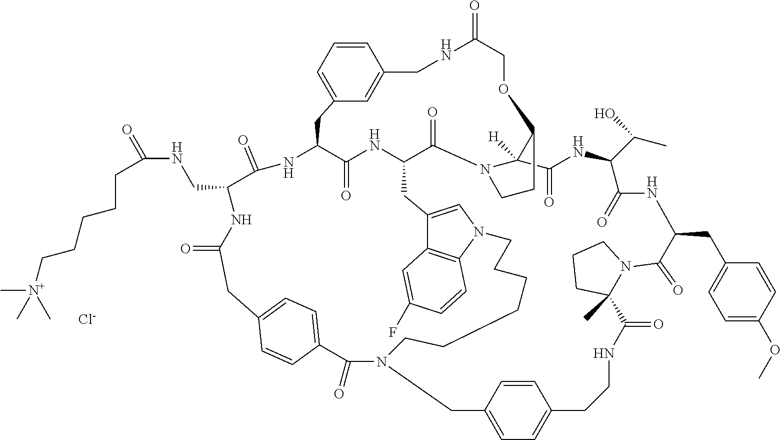 |
| 2450 | 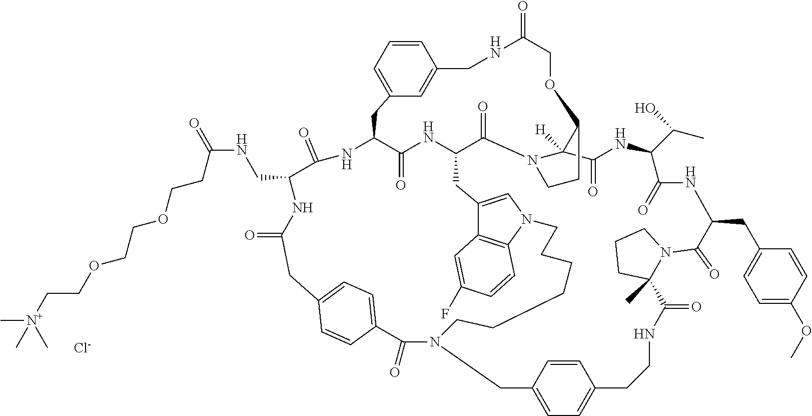 |
| 2451 | 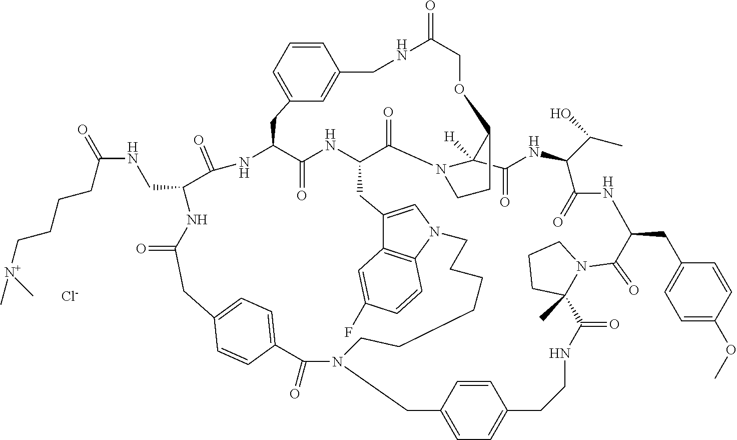 |
| 2455 | 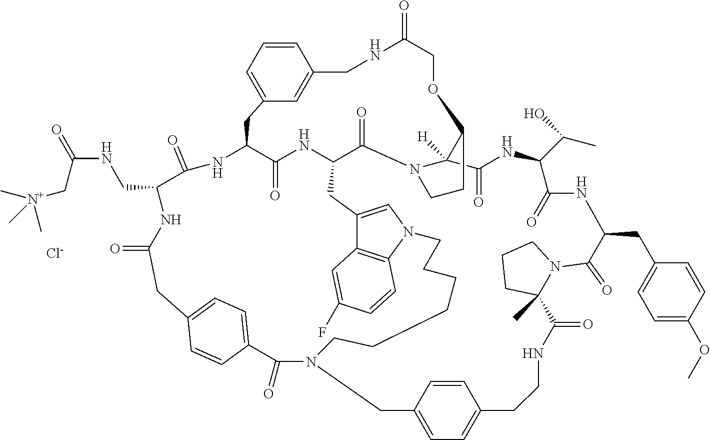 |
| 2457 | 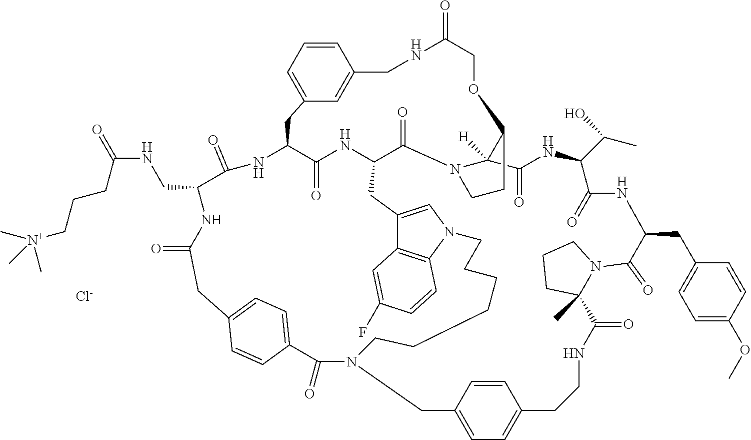 |
| 2460 | 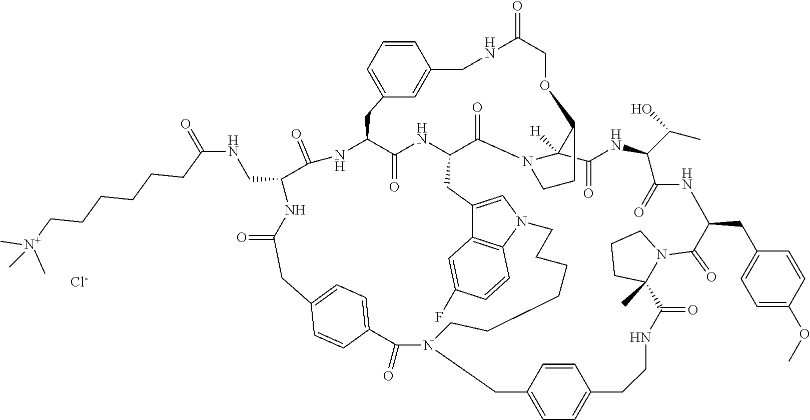 |
| 2461 | 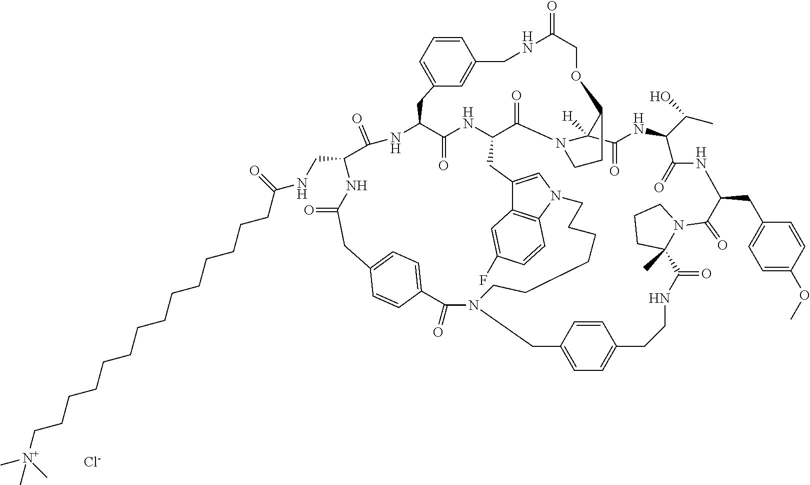 |
| 2402A | 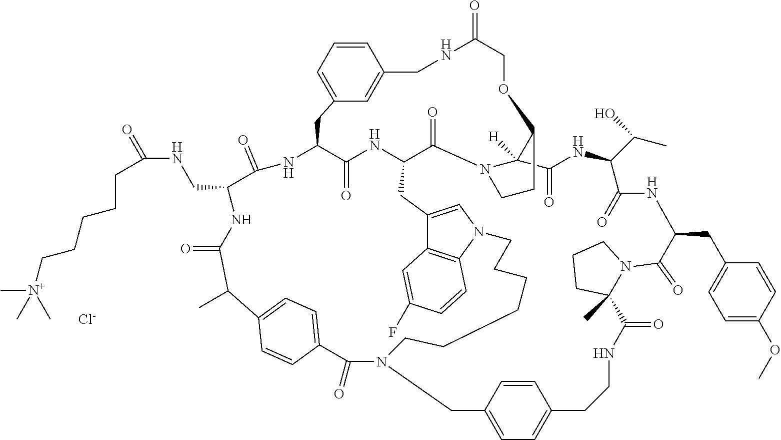 |
| 2402B | 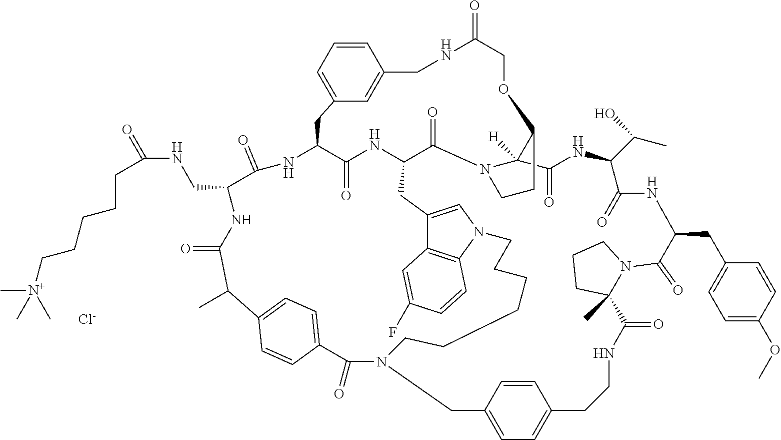 |
| 204B | 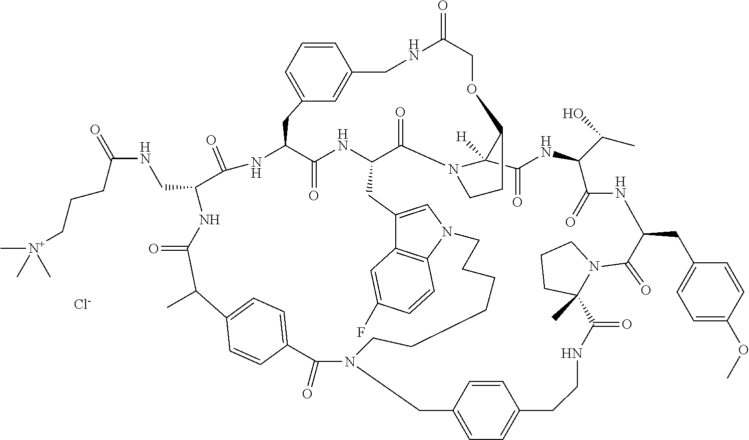 |
| 205A | 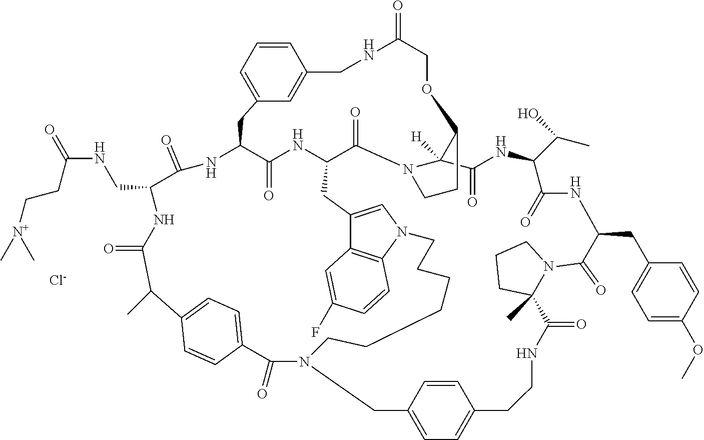 |
| 205B | 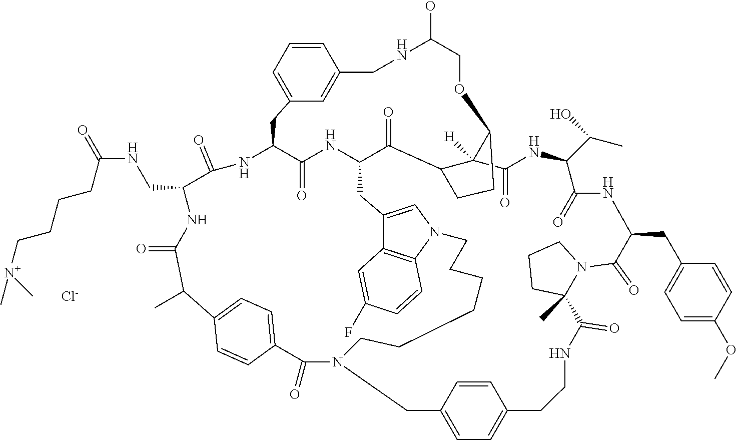 [SMILES: COc1ccc(C[C@@H]2NC(=O)[C@H]([C@@H](C)O)NC(=O)[C@H]3C4CC[C@@H]3OCC(O)NCc3cccc(c3)C[C@@H]3NC(=O)[C@@H](CNC(=O)CCCC[NH+](C)C)NC(=O)C(C)c5ccc(cc5)C(=O)N(CCCCCCn5cc(c6cc(F)ccc65)C[C@H](NC3=O)C4=O)Cc3ccc(cc3)CCNC(=O)[C@]3(C)CCCN3C2=O)cc1 ; [Cl-]] |
| 211B | 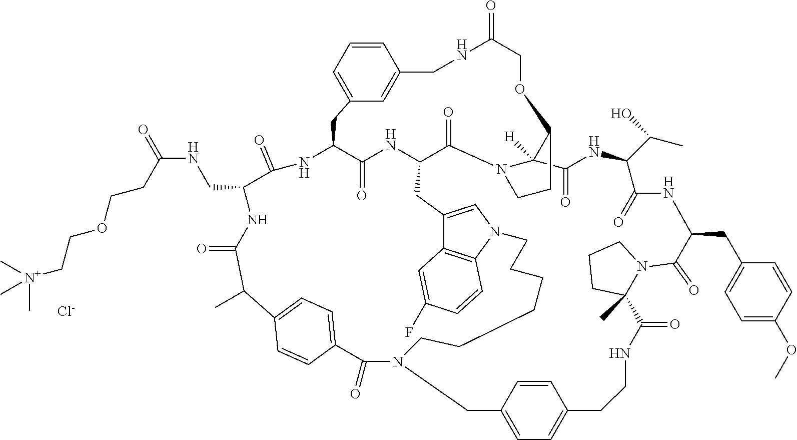 |
| 212A | 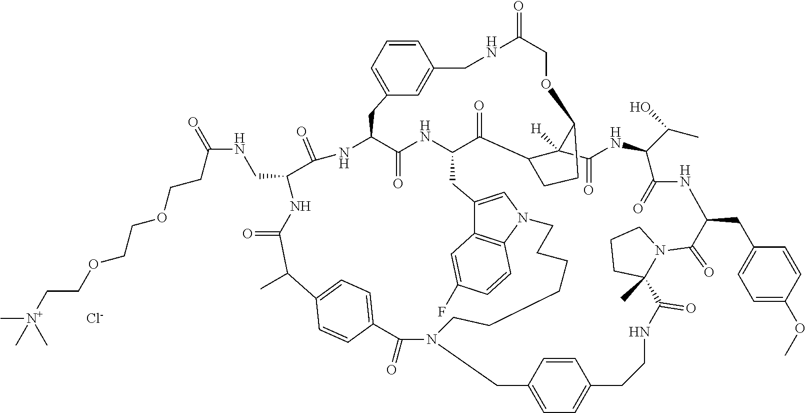 [SMILES: COc1ccc(C[C@@H]2NC(=O)[C@H]([C@@H](C)O)NC(=O)[C@H]3C4CC[C@@H]3OCC(=O)NCc3cccc(c3)C[C@@H]3NC(=O)[C@@H](CNC(=O)CCOCCOCC[N+](C)(C)C)NC(=O)C(C)c5ccc(cc5)C(=O)N(CCCCCCn5cc(c6cc(F)ccc65)C[C@H](NC3=O)C4=O)Cc3ccc(cc3)CCNC(=O)[C@]3(C)CCCN3C2=O)cc1 ; [Cl-]] |
| 212B | 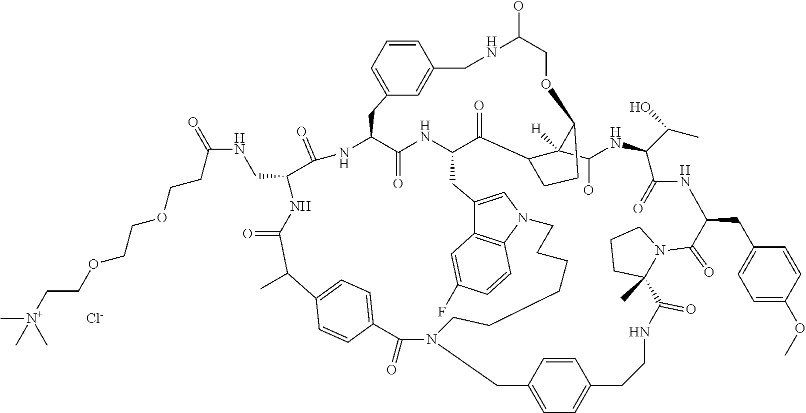 [SMILES: COc1ccc(C[C@@H]2NC(=O)[C@H]([C@@H](C)O)NC(O)[C@H]3C4CC[C@@H]3OCC(O)NCc3cccc(c3)C[C@@H]3NC(=O)[C@@H](CNC(=O)CCOCCOCC[N+](C)(C)C)NC(=O)C(C)c5ccc(cc5)C(=O)N(CCCCCCn5cc(c6cc(F)ccc65)C[C@H](NC3=O)C4=O)Cc3ccc(cc3)CCNC(=O)[C@]3(C)CCCN3C2=O)cc1 ; [Cl-]] |
| 213B | 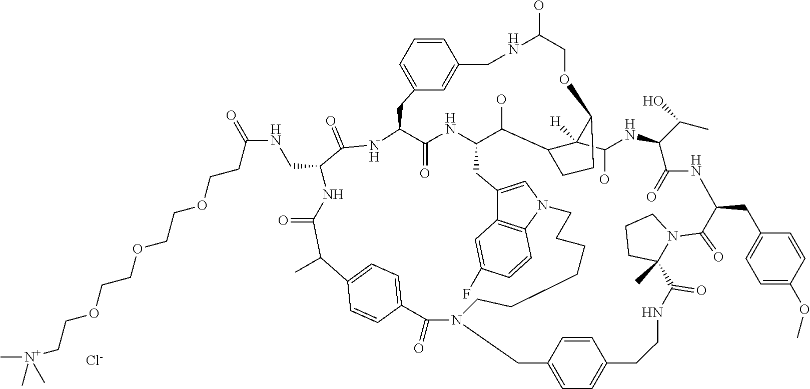 [SMILES: COc1ccc(C[C@@H]2NC(=O)[C@H]([C@@H](C)O)NC(O)[C@H]3C4CC[C@@H]3OCC(O)NCc3cccc(c3)C[C@@H]3NC(=O)[C@@H](CNC(=O)CCOCCOCCOCC[N+](C)(C)C)NC(=O)C(C)c5ccc(cc5)C(=O)N(CCCCCCn5cc(c6cc(F)ccc65)C[C@H](NC3=O)C4O)Cc3ccc(cc3)CCNC(=O)[C@]3(C)CCCN3C2=O)cc1 ; [Cl-]] |
| 2403 | 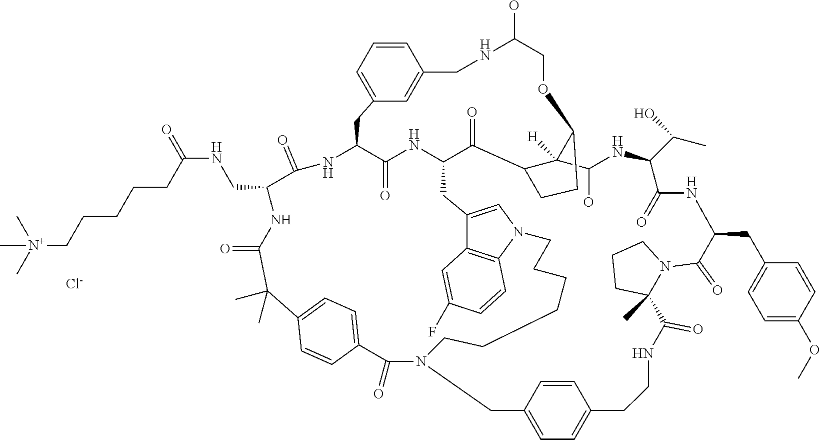 [SMILES: COc1ccc(C[C@@H]2NC(=O)[C@H]([C@@H](C)O)NC(O)[C@H]3C4CC[C@@H]3OCC(O)NCc3cccc(c3)C[C@@H]3NC(=O)[C@@H](CNC(=O)CCCCC[N+](C)(C)C)NC(=O)C(C)(C)c5ccc(cc5)C(=O)N(CCCCCCn5cc(c6cc(F)ccc65)C[C@H](NC3=O)C4=O)Cc3ccc(cc3)CCNC(=O)[C@]3(C)CCCN3C2=O)cc1 ; [Cl-]] |
| 2430 | 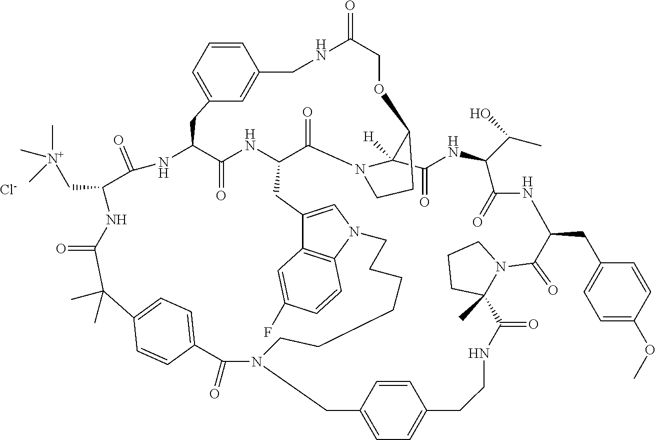 [SMILES: [Cl-] ; COc1ccc(C[C@@H]2NC(=O)[C@H]([C@@H](C)O)NC(=O)[C@@H]3[C@@H]4CCN3C(=O)[C@@H]3Cc5cn(c6ccc(F)cc56)CCCCCCN(Cc5ccc(cc5)CCNC(=O)[C@]5(C)CCCN5C2=O)C(=O)c2ccc(cc2)C(C)(C)C(=O)N[C@H](C[N+](C)(C)C)C(=O)N[C@@H](Cc2cccc(c2)CNC(=O)CO4)C(=O)N3)cc1] |
| 2404 | 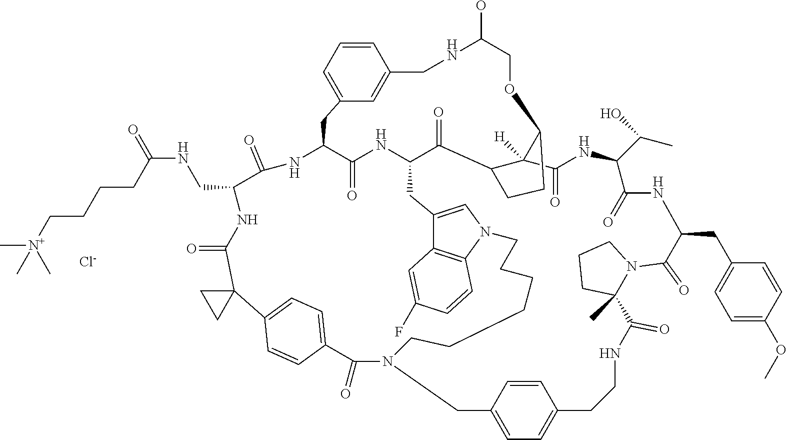 [SMILES: COc1ccc(C[C@@H]2NC(=O)[C@H]([C@@H](C)O)NC(=O)[C@H]3C4CC[C@@H]3OCC(O)NCc3cccc(c3)C[C@@H]3NC(=O)[C@@H](CNC(=O)CCCC[N+](C)(C)C)NC(=O)C5(CC5)c5ccc(cc5)C(=O)N(CCCCCCn5cc(c6cc(F)ccc65)C[C@H](NC3=O)C4=O)Cc3ccc(cc3)CCNC(=O)[C@]3(C)CCCN3C2=O)cc1 ; [Cl-]] |
| 2410 | 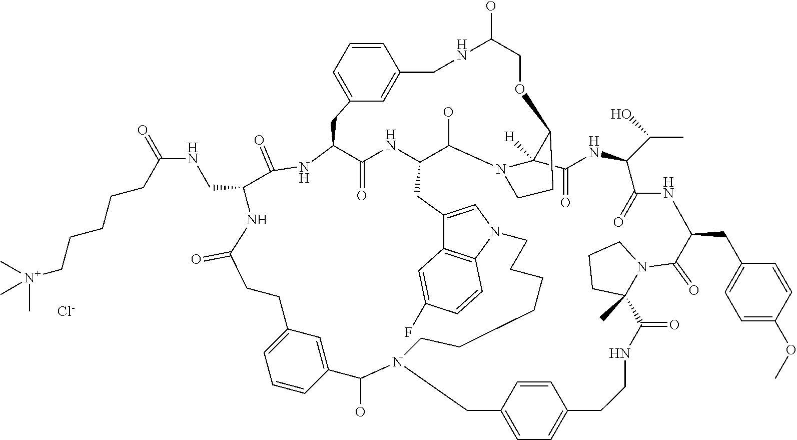 [SMILES: COc1ccc(C[C@@H]2NC(=O)[C@H]([C@@H](C)O)NC(=O)[C@@H]3[C@@H]4CCN3C(O)[C@@H]3Cc5cn(c6ccc(F)cc56)CCCCCCN(Cc5ccc(cc5)CCNC(=O)[C@]5(C)CCCN5C2=O)C(O)c2cccc(c2)CCC(=O)N[C@H](CNC(=O)CCCCC[N+](C)(C)C)C(=O)N[C@@H](Cc2cccc(c2)CNC(O)CO4)C(=O)N3)cc1 ; [Cl-]] |
| 2452 | 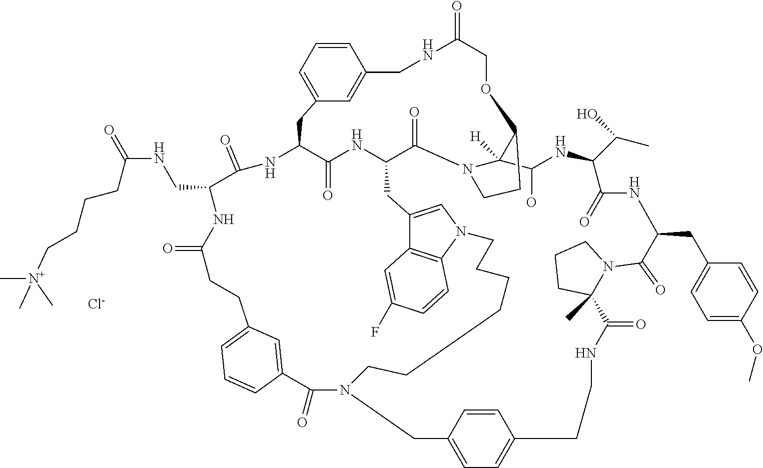 |
| 2412 | 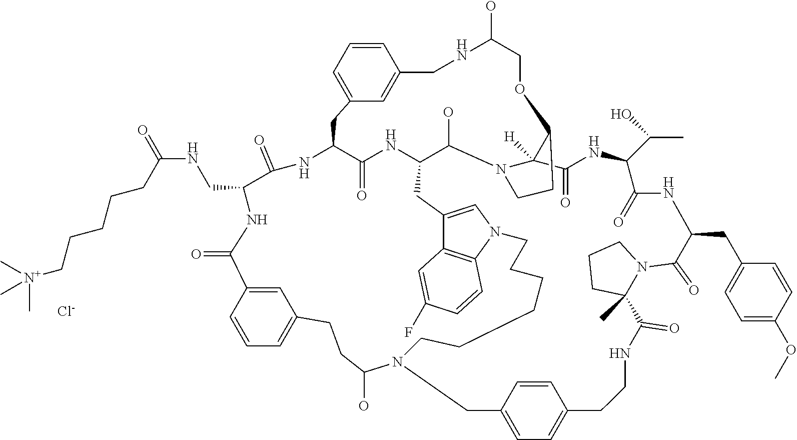 |
| 2453 | 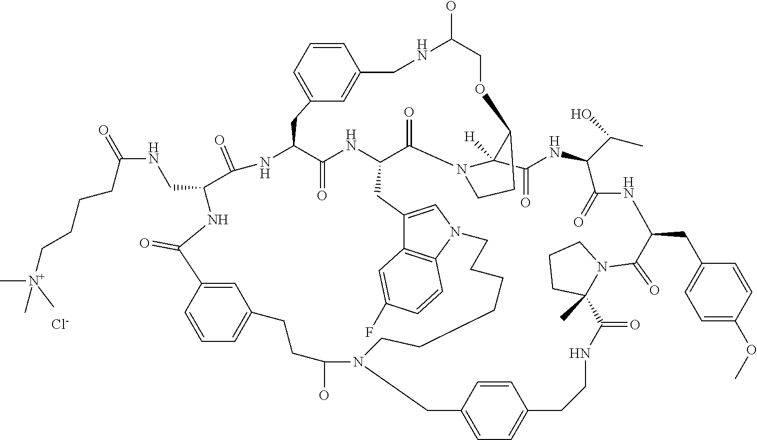 [SMILES: COc1ccc(C[C@@H]2NC(=O)[C@H]([C@@H](C)O)NC(=O)[C@@H]3[C@@H]4CCN3C(=O)[C@@H]3Cc5cn(c6ccc(F)cc56)CCCCCCN(Cc5ccc(cc5)CCNC(=O)[C@]5(C)CCCN5C2=O)C(O)CCc2cccc(c2)C(=O)N[C@H](CNC(=O)CCCC[N+](C)(C)C)C(=O)N[C@@H](Cc2cccc(c2)CNC(O)CO4)C(=O)N3)cc1 ; [Cl-]] |
| 2454 | 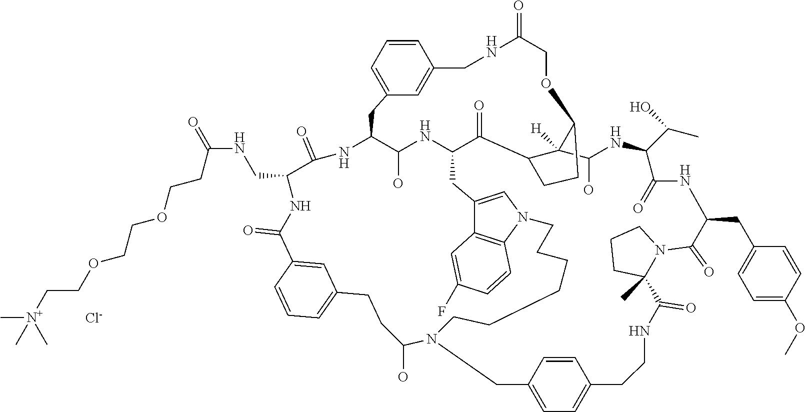 [SMILES: COc1ccc(C[C@@H]2NC(=O)[C@H]([C@@H](C)O)NC(O)[C@H]3C4CC[C@@H]3OCC(=O)NCc3cccc(c3)C[C@@H]3NC(=O)[C@@H](CNC(=O)CCOCCOCC[N+](C)(C)C)NC(=O)c5cccc(c5)CCC(O)N(CCCCCCn5cc(c6cc(F)ccc65)C[C@H](NC3O)C4=O)Cc3ccc(cc3)CCNC(=O)[C@]3(C)CCCN3C2=O)cc1 ; [Cl-]] |
| 2416 | 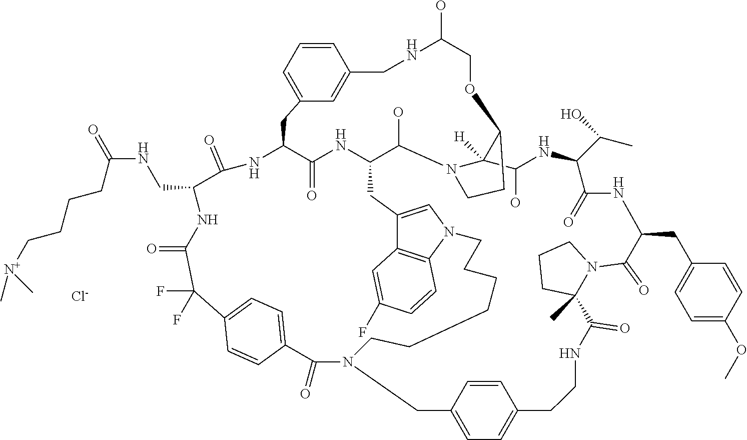 [SMILES: COc1ccc(C[C@@H]2NC(=O)[C@H]([C@@H](C)O)NC(O)[C@@H]3[C@@H]4CCN3C(O)[C@@H]3Cc5cn(c6ccc(F)cc56)CCCCCCN(Cc5ccc(cc5)CCNC(=O)[C@]5(C)CCCN5C2=O)C(=O)c2ccc(cc2)C(F)(F)C(=O)N[C@H](CNC(=O)CCCC[NH+](C)C)C(=O)N[C@@H](Cc2cccc(c2)CNC(O)CO4)C(=O)N3)cc1 ; [Cl-]] |
| 2421 | 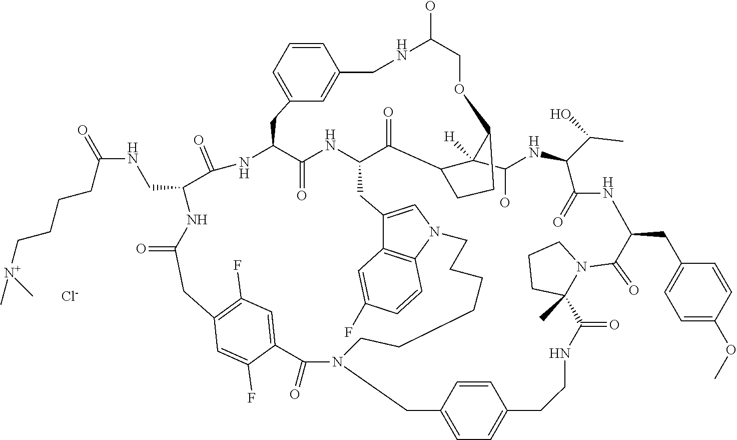 |
| 2427 | 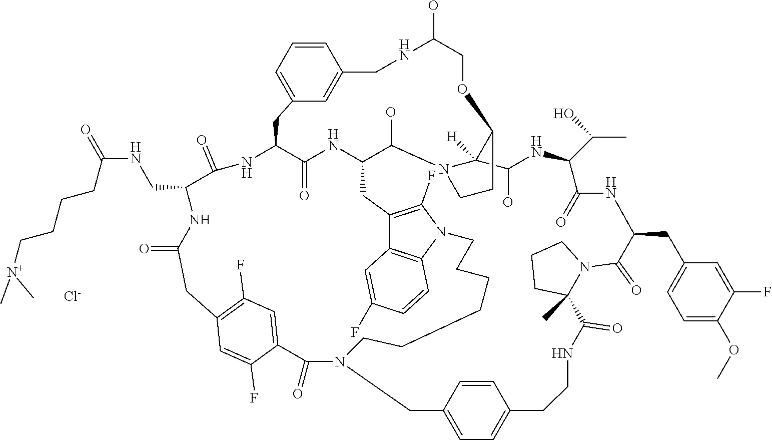 [SMILES: COc1ccc(C[C@@H]2NC(=O)[C@H]([C@@H](C)O)NC(O)[C@@H]3[C@@H]4CCN3C(O)[C@@H]3Cc5c(F)n(c6ccc(F)cc56)CCCCCCN(Cc5ccc(cc5)CCNC(=O)[C@]5(C)CCCN5C2=O)C(=O)c2cc(F)c(cc2F)CC(=O)N[C@H](CNC(=O)CCCC[NH+](C)C)C(=O)N[C@@H](Cc2cccc(c2)CNC(O)CO4)C(=O)N3)cc1F ; [Cl-]] |
| 2428 | 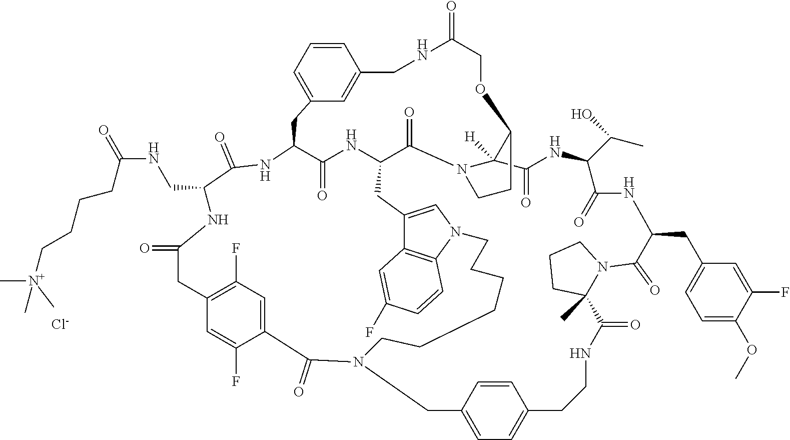 [SMILES: COc1ccc(C[C@@H]2NC(=O)[C@H]([C@@H](C)O)NC(=O)[C@@H]3[C@@H]4CCN3C(=O)[C@@H]3Cc5cn(c6ccc(F)cc56)CCCCCCN(Cc5ccc(cc5)CCNC(=O)[C@]5(C)CCCN5C2=O)C(=O)c2cc(F)c(cc2F)CC(=O)N[C@H](CNC(=O)CCCC[N+](C)(C)C)C(=O)N[C@@H](Cc2cccc(c2)CNC(=O)CO4)C(=O)N3)cc1F ; [Cl-]] |
| 2407 | 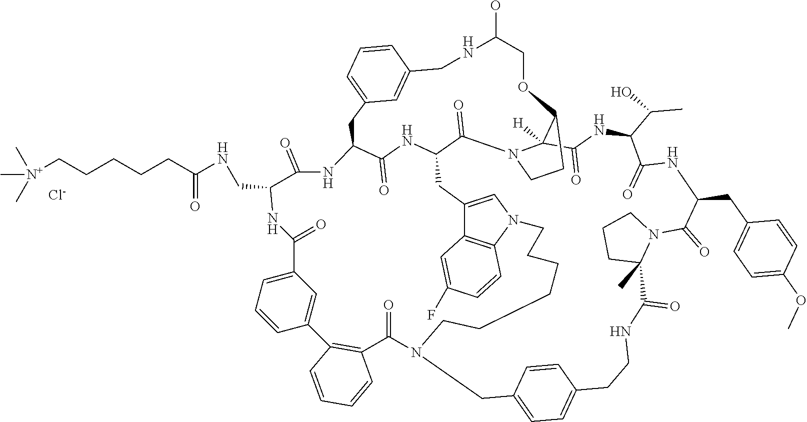 |
| 2462 | 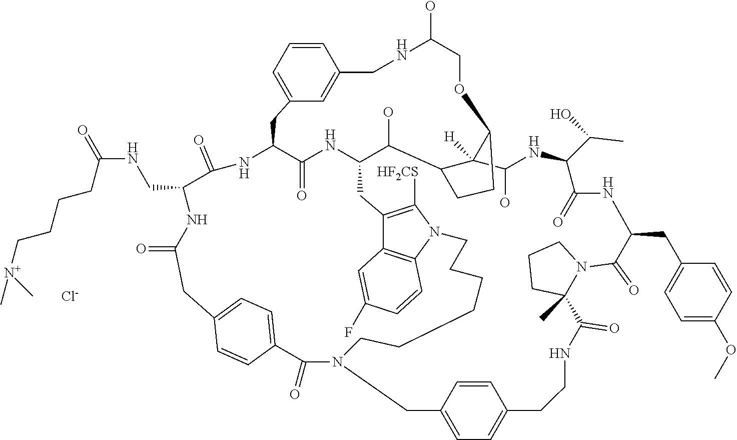 |
| 2463 | 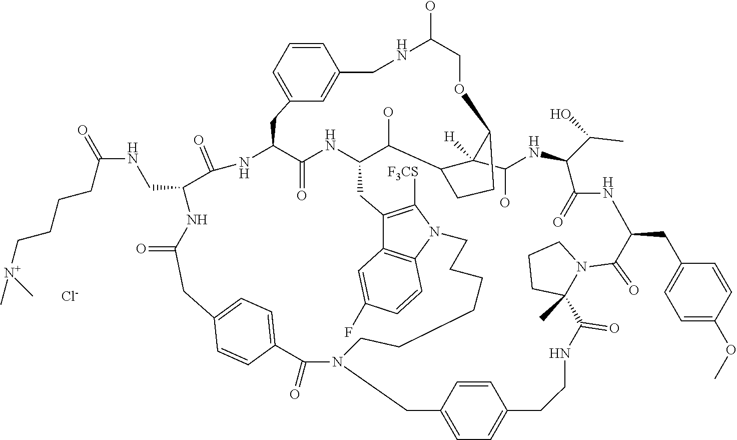 |

In one aspect, the present disclosure also provides a pharmaceutical composition comprising any of the aforementioned compounds or a pharmaceutically acceptable salt thereof,
wherein, the pharmaceutical composition is selected from an oral preparation or an injectable preparation.

The oral preparation form may be used as a preferred embodiment of the present disclosure.

In one aspect, the present disclosure also provides use of the aforementioned compounds or a pharmaceutically acceptable salt thereof in the preparation of PCSK9 inhibitors.

In one aspect, the present disclosure also provides a method for inhibiting PCSK9, comprising administering an effective dose of any of the aforementioned compounds or a pharmaceutically acceptable salt thereof to a patient.

In the present disclosure, the patient has hypercholesteroiemia.

In one aspect, the present disclosure also provides a method for treating the following diseases, comprising administering an effective dose of any of the aforementioned compounds or a pharmaceutically acceptable salt thereof to a patient; the disease is selected from atherosclerosis, and/or hypercholesterolemia, and/or coronary heart disease, and/or metabolic syndrome, and/or acute coronary syndrome, wherein such diseases are associated with PCSK9.

### DESCRIPTION OF DRAWINGS

FIG. 1 shows a chart of concentration value of LDL-C in animals on day 7 (compounds 2451, 2453, 205A, 2421 and compounds 2401, 2460, 2461, 2462, 2455, 2457, 2416).

FIG. 2 shows a chart of concentrations of LDL-C and TG in animals on day 17 after continuous oral administration for 14 days.

### DETAILED DESCRIPTION

In the following description, known structural representations are used, which include conventional stereochemical symbols for certain asymmetric carbon centers.

In most cases, the absolute configuration of the example compound has not yet been determined, but it has been assigned through simulation to specific example compounds with known stereochemical configurations (determined by X-ray crystallography) prepared using the same or similar reaction conditions and starting reagents and isolated under the same chromatographic conditions. Unless otherwise indicated in the presented materials, the specific assignment of the structural configurations presented herein means that the prepared specific compound is identified to have an excessive amount of a specific stereoisomer, and is not necessarily presented as a statement for an absolute determination of the stereochemical structure of the compound herein.

In the present disclosure, if the stereochemical configuration is not marked in the structural formula of a compound, it does not mean that the compound does not have a stereochemical configuration.

When a mixture of isomers is obtained, conventional methods may be used for preparation, such as chromatography or crystallization, or by using stereochemically homogeneous starting materials for the synthesis or by separating the mixture by stereoselective synthesis, to prepare individual stereoisomers in a significant percentage excess of enantiomers. Separation of stereoisomeric mixtures may be performed at intermediate steps during the synthesis of compounds, or it can be performed to the final racemic products.

Absolute stereochemistry is determined by X-ray crystallography of crystalline products or crystalline intermediates, which are derived from reagents containing stereosymmetric centers of known configurations as needed. Unless specific isomers, salts, solvates (including hydrates), or solvate salts of such racemates, enantiomers, or diastereomers are indicated, the present disclosure includes all such isomers as well as salts, solvates (including hydrates), and solvate salts of such racemates, enantiomers, diastereomers, and mixtures thereof.

The present disclosure also includes isotopic labeled compounds of the present disclosure, which are structurally equivalent to those described herein. The present disclosure intends to include all suitable isotopic variants of the compounds. Examples of preferred isotopes include isotopes of hydrogen, carbon, nitrogen, oxygen, phosphorus, iodine, fluorine, and chlorine, such as but not limited to: ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F and ³⁶Cl, ¹²³I and ¹²⁵I. In addition, other isotopes may also be incorporated through known means. Deuterium (D) is the main hydrogen isotope found in nature. Enriching deuterium can provide certain therapeutic advantages, such as increasing in vivo half-life or reducing dosage requirements, or providing compounds suitable as standards for characterizing biological samples.

When the wavy line terminates the regular bond (opposite to the two atoms within the connecting structure), it indicates the point through which it binds to the structure, for example:

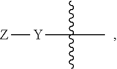

means that the Z moiety is bonded via Y moiety through the bond that is terminated with a wavy line. When letter symbols are used to describe substituent moieties, dashes are used to indicate the point through which the indicated substrate is bonded, for example: —Z—Y indicates that the Y moiety is bonded through the Z moiety.

When any variables or parts are expressed in a certain range, such as (—CH₂—)₁₋₄, it includes the two extreme values of the specified range (i.e, 1 and 4 in the example) and all integer values in between (i.e, 2 and 3 in the example).

According to conventional practice in the field, x denotes a halogen.

Unless otherwise specified at the point of use, the term ‘halogen(X)’ includes fluorine, chlorine, bromine, and iodine. As used herein, the term “subject” (or “patient”) refers to animals, preferably mammals, and particularly humans or non-human animals including livestock animals and domestic animals, including but not limited to cows, horses, sheep, pigs, goats, rabbits, cats, dogs, and other mammals in need of treatment.

In some embodiments, the subject is preferably human. As used herein, “administration” and its variants (e.g., “administration” of a compound) refer to providing a compound or a pharmaceutically acceptable salt thereof to a subject in need of treatment.

In the present disclosure, “—C(O)—”, represents carbonyl group, i.e.,

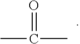

The term “absence” as described in the present disclosure, represents “none”. For example, when R₁′ is selected from absence, “—NR₁R₁′R₁″” is “—NR₁R₁″”. When R₁″ is selected from absence, “—NR₁R₁′R₁″” is “—NR₁R₁”.

In the present disclosure, R₁′ and R₁″ cannot be “absence” at the same time. That is, when R₁′ is selected from absence, R₁″ is selected from hydrogen or methyl. When R₁″ is selected from absence, R₁′ is selected from hydrogen or methyl.

In the present disclosure, n₁ may be selected from any integer of 1 to 14, e.g., 1, 2, 3, 4, 5, 6, 7, 8, 9, 10, 11, 12, 13, or 14. n₁ may also be selected from the following range: 1-13, 1-12, 1-11, 1-10, 1-9, 1-8, 1-7, 1-6, 1-5, 1-4, 1-3, 1-2, 2-14, 2-13, 2-12, 2-11, 2-10, 2-9, 2-8, 2-7, 2-6, 2-5, 2-4, 2-3, 3-14, 3-13, 3-12, 3-11, 3-10, 3-9, 3-8, 3-7, 3-6, 3-5, 3-4, 4-14, 4-13, 4-12, 4-11, 4-10, 4-9, 4-8, 4-7, 4-6, 4-5, 5-14, 5-13, 5-12, 5-11, 5-10, 5-9, 5-8, 5-7, 5-6, 6-14, 6-13, 6-12, 6-11, 6-10, 6-9, 6-8, 6-7, 7-14, 7-13, 7-12, 7-11, 7-10, 7-9, 7-8, 8-14, 8-13, 8-12, 8-11, 8-10, 8-9, 9-14, 9-13, 9-12, 9-11, 9-10, 10-14, 10-13, 10-12, 10-11, 11-14, 11-13, 11-12, 12-14, 12-13, 13-14.

In the present disclosure, n₂ may be selected from any integer of 1 to 5, e.g., 1, 2, 3, 4, or 5. n₂ may also be selected from the following range: 1-4, 1-3, 1-2, 2-5, 2-4, 2-3, 3-5, 3-4, or 4-5.

In the present disclosure, “a bond” refers to a connecting bond. For example, in —Z—Y—W, Y is selected from a bond, i.e., —Z—W.

In the present disclosure, “a linear C₁-C₅ alkyl” includes at least —CH₃, —CH₂CH₃, —CH₂CH₂CH₃, —CH₂CH₂CH₂CH₃, or —CH₂CH₂CH₂CH₂CH₃.

In the present disclosure, “a C₁-C₃ alkyl” includes at least —CH₃, —CH₂CH₃, —CH₂CH₂CH₃, or —CH₂(CH₃)₂.

In the present disclosure, “a C₃-C₅ cycloalkyl” includes at least

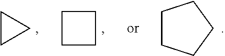

In the present disclosure,

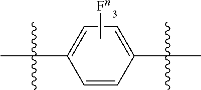

may include at least

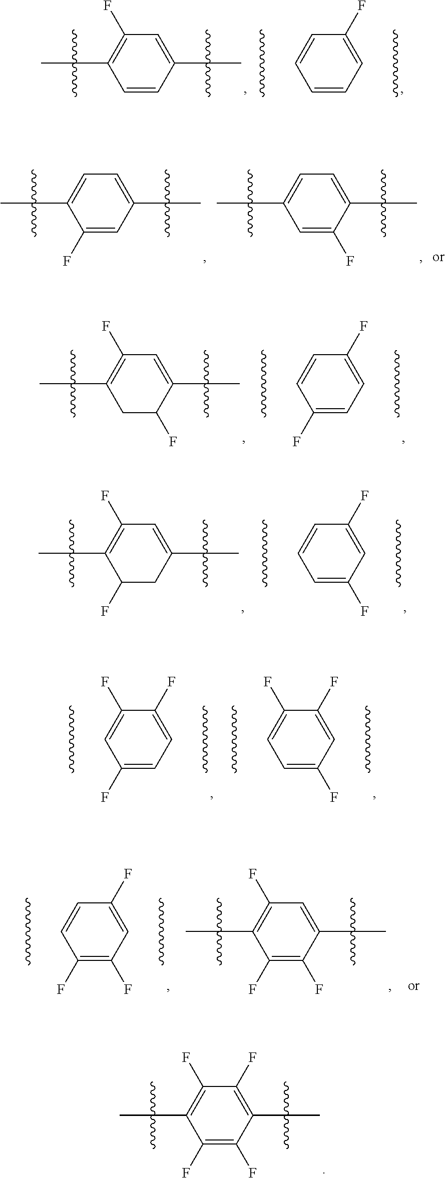

In the present disclosure, unless otherwise specified, the structure or group represented by A is connected in a left to right manner in the general formula of the compound, the connection manner is as follows as follows:

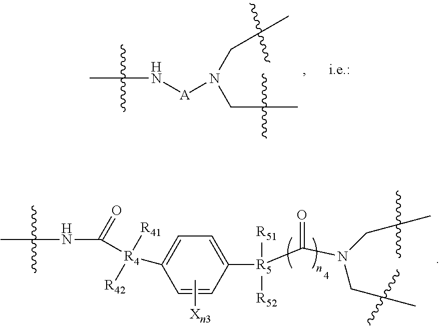

The compound of the present disclosure should also include the following structures:

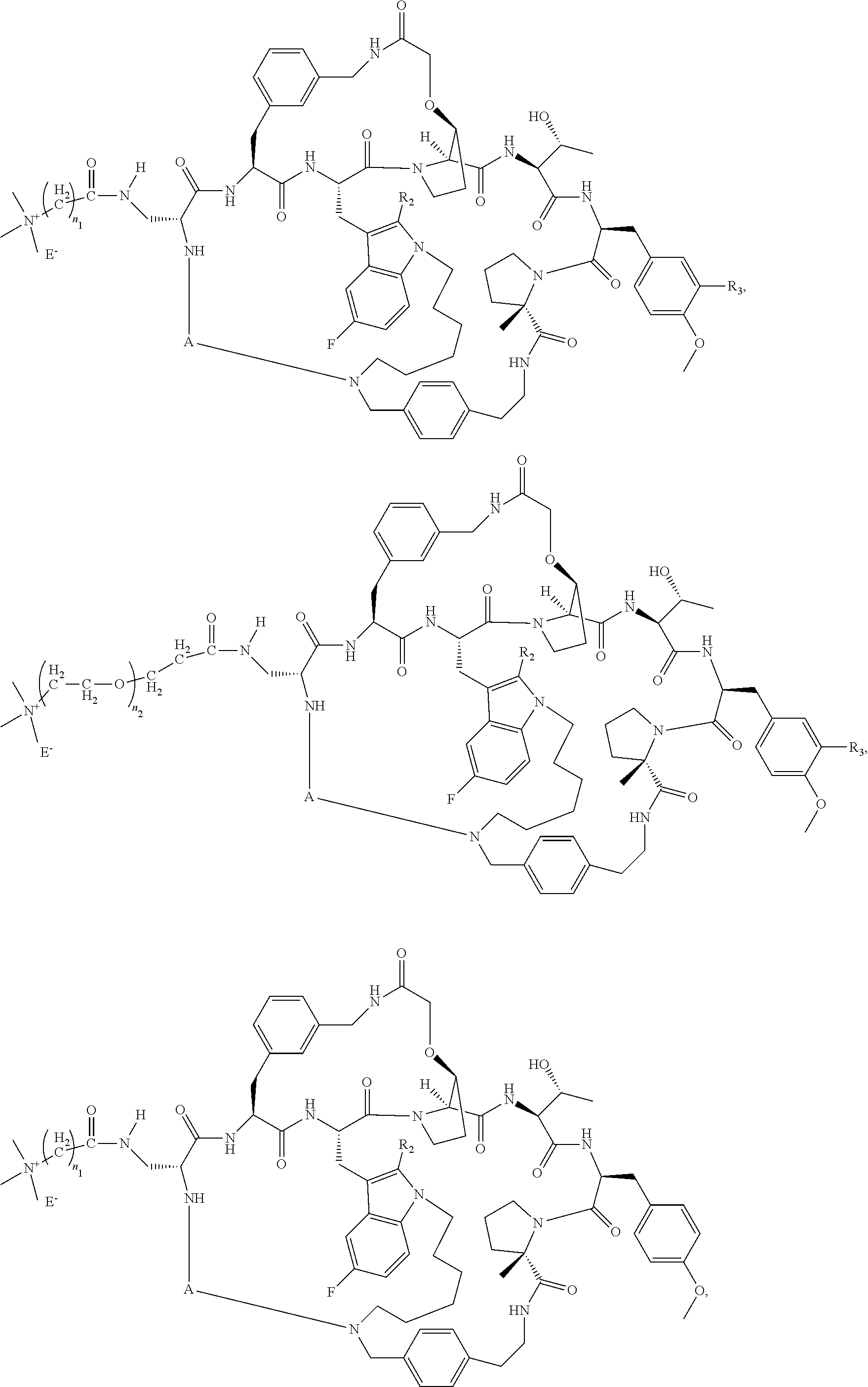

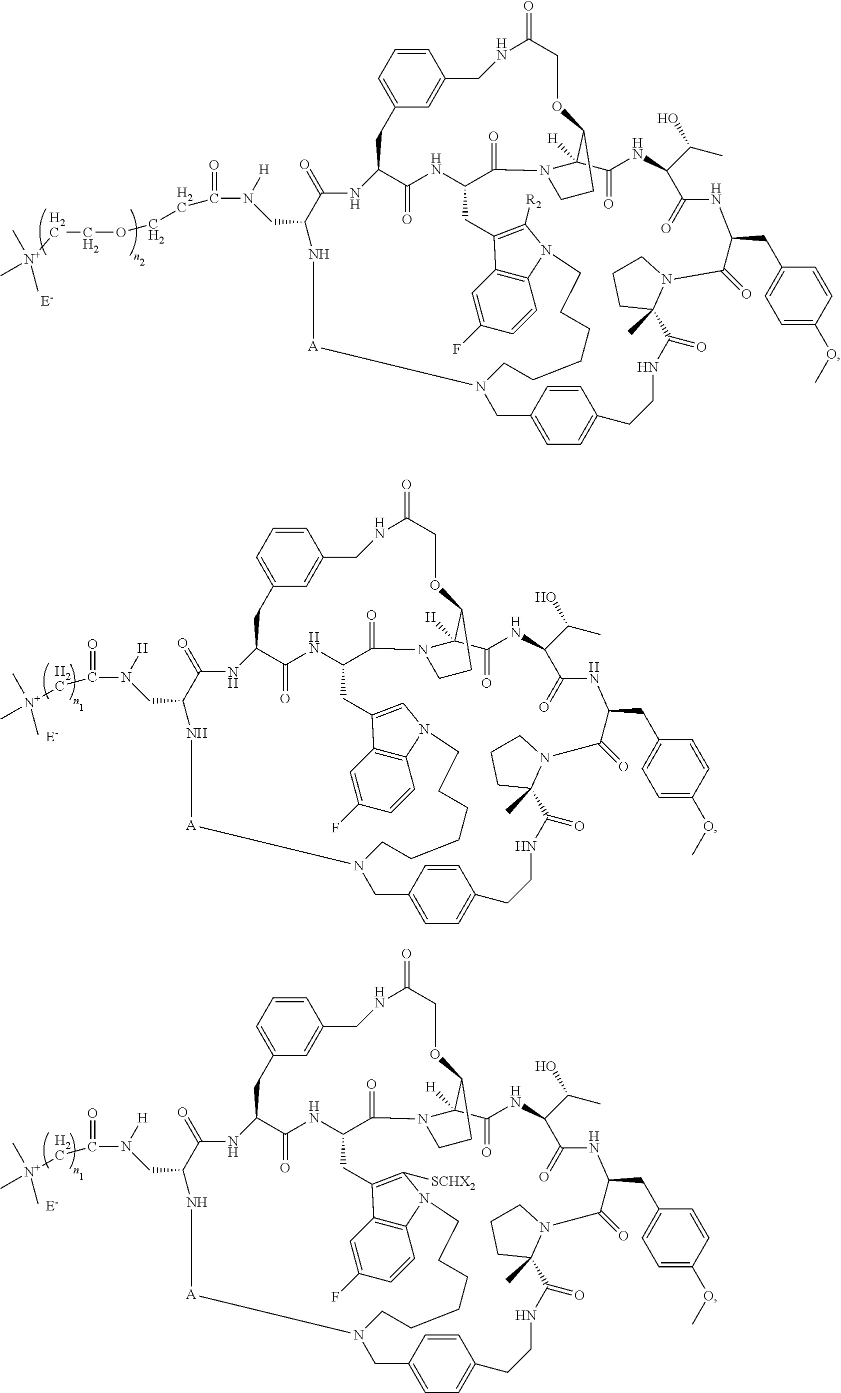

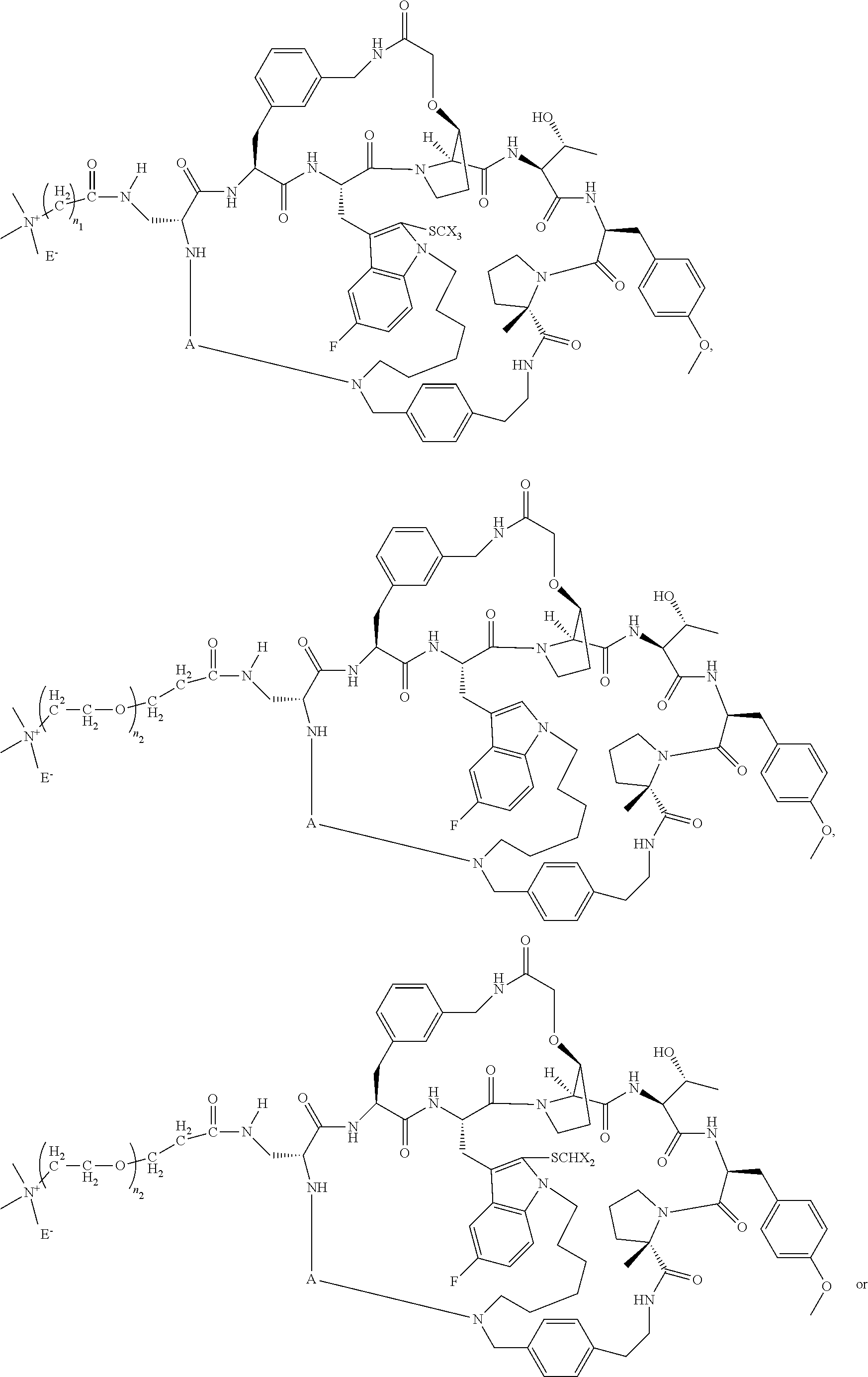

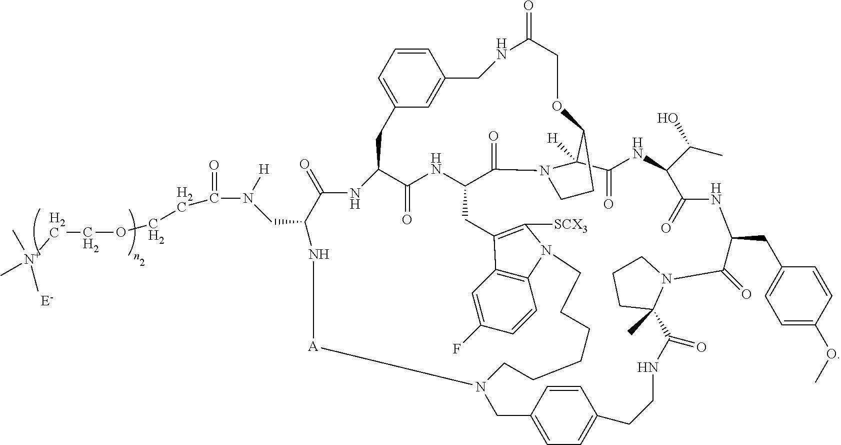

The compound of the present disclosure should further include the following structures:

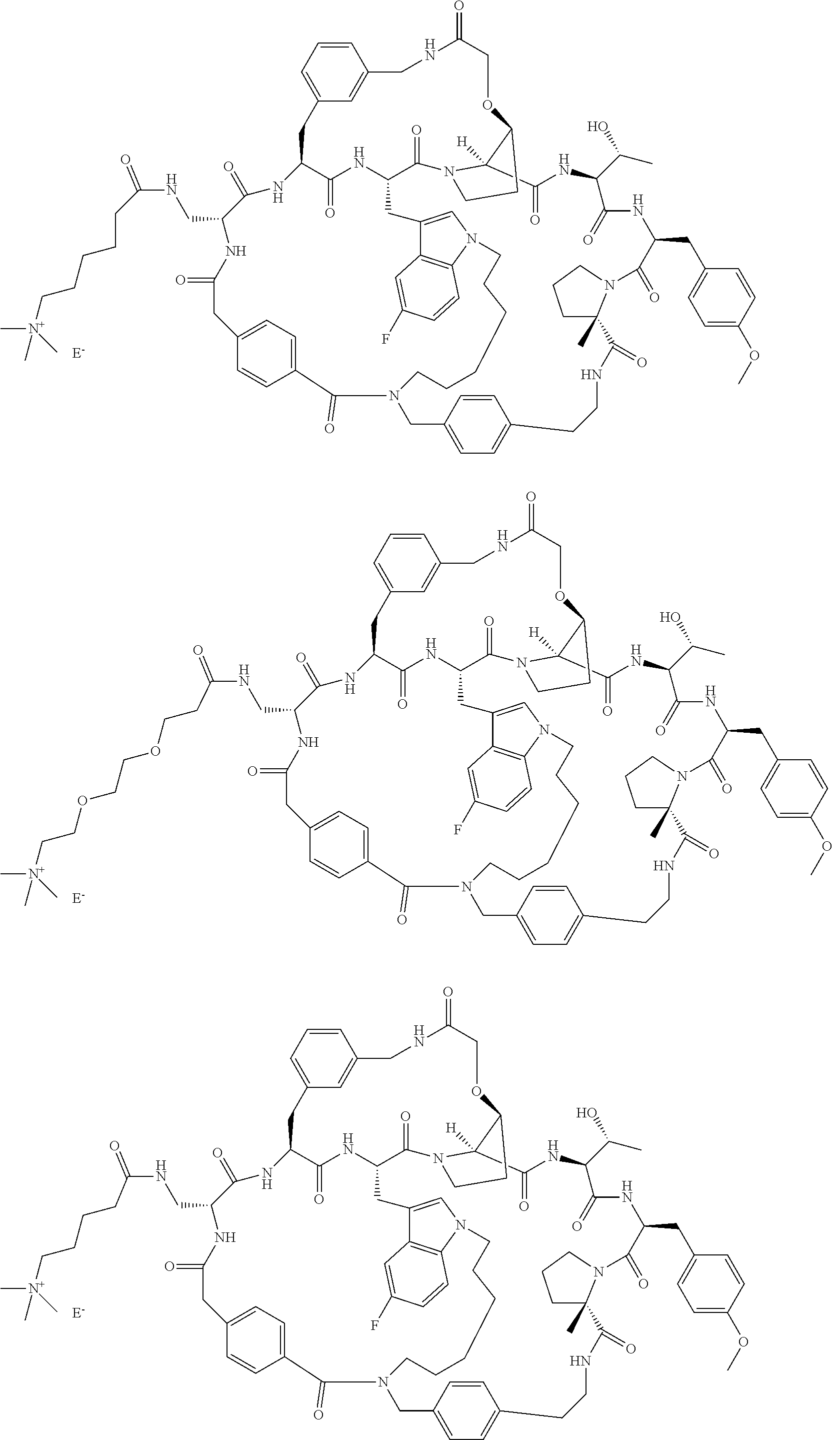

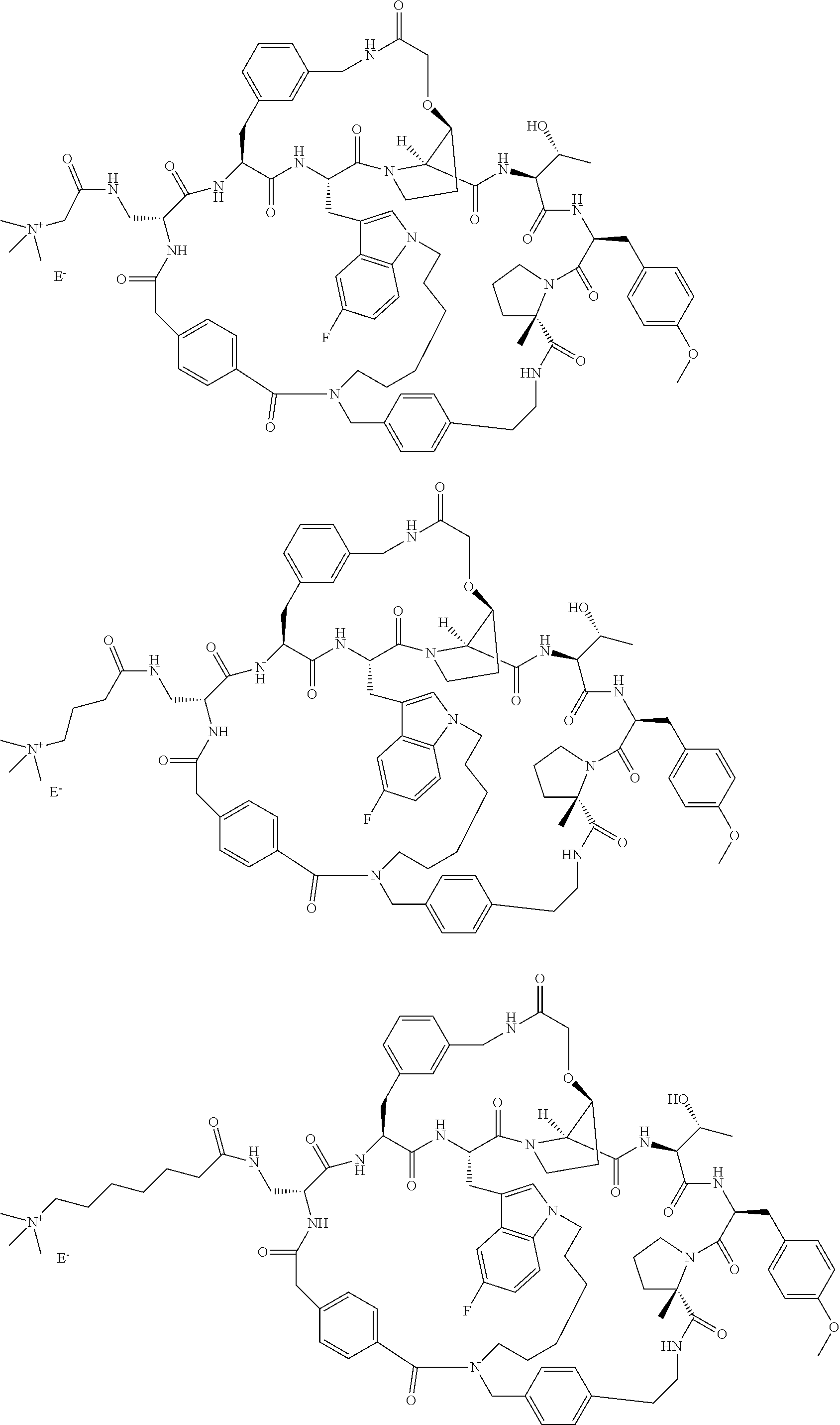

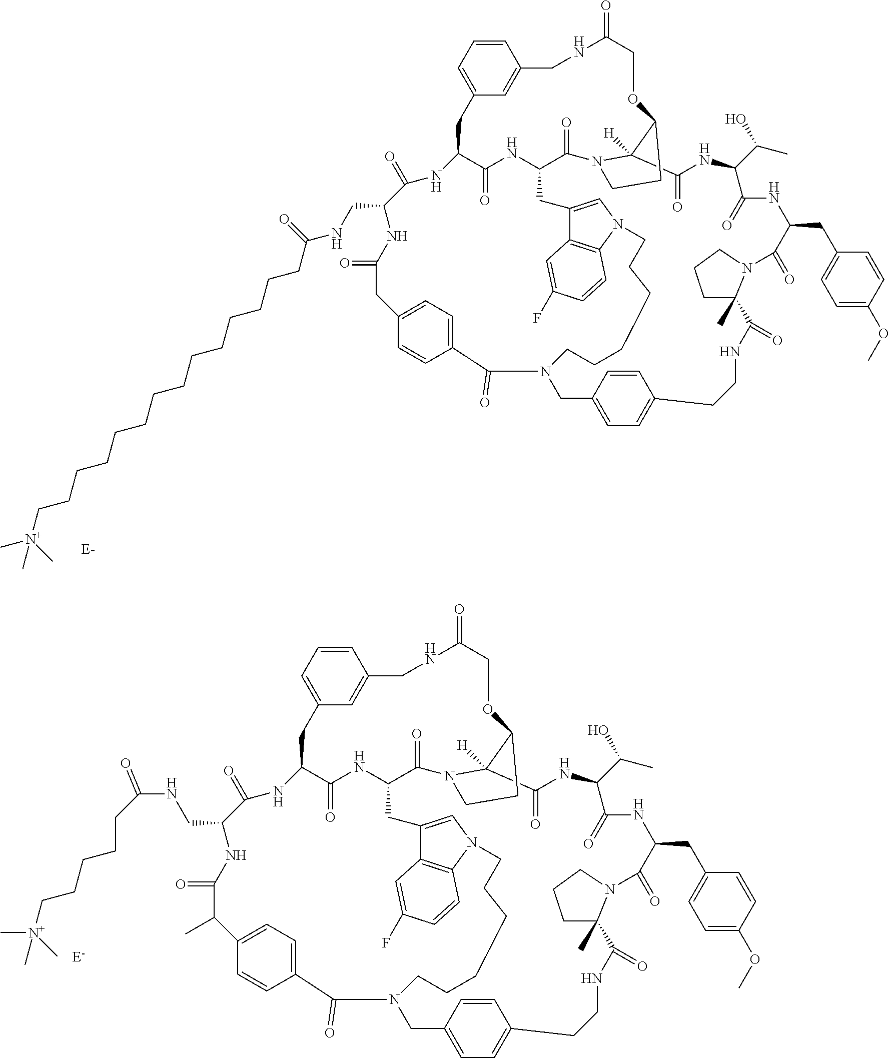 [SMILES: COc1ccc(C[C@@H]2NC(=O)[C@H]([C@@H](C)O)NC(=O)[C@@H]3[C@@H]4CCN3C(=O)[C@@H]3Cc5cn(c6ccc(F)cc56)CCCCCCN(Cc5ccc(cc5)CCNC(=O)[C@]5(C)CCCN5C2=O)C(=O)c2ccc(cc2)CC(=O)N[C@H](CNC(=O)CCCCCCCCCCCCCC[N+](C)(C)C)C(=O)N[C@@H](Cc2cccc(c2)CNC(=O)CO4)C(=O)N3)cc1 ; COc1ccc(C[C@@H]2NC(=O)[C@H]([C@@H](C)O)NC(=O)[C@@H]3[C@@H]4CCN3C(=O)[C@@H]3Cc5cn(c6ccc(F)cc56)CCCCCCN(Cc5ccc(cc5)CCNC(=O)[C@]5(C)CCCN5C2=O)C(=O)c2ccc(cc2)C(C)C(=O)N[C@H](CNC(=O)CCCCC[N+](C)(C)C)C(=O)N[C@@H](Cc2cccc(c2)CNC(=O)CO4)C(=O)N3)cc1]

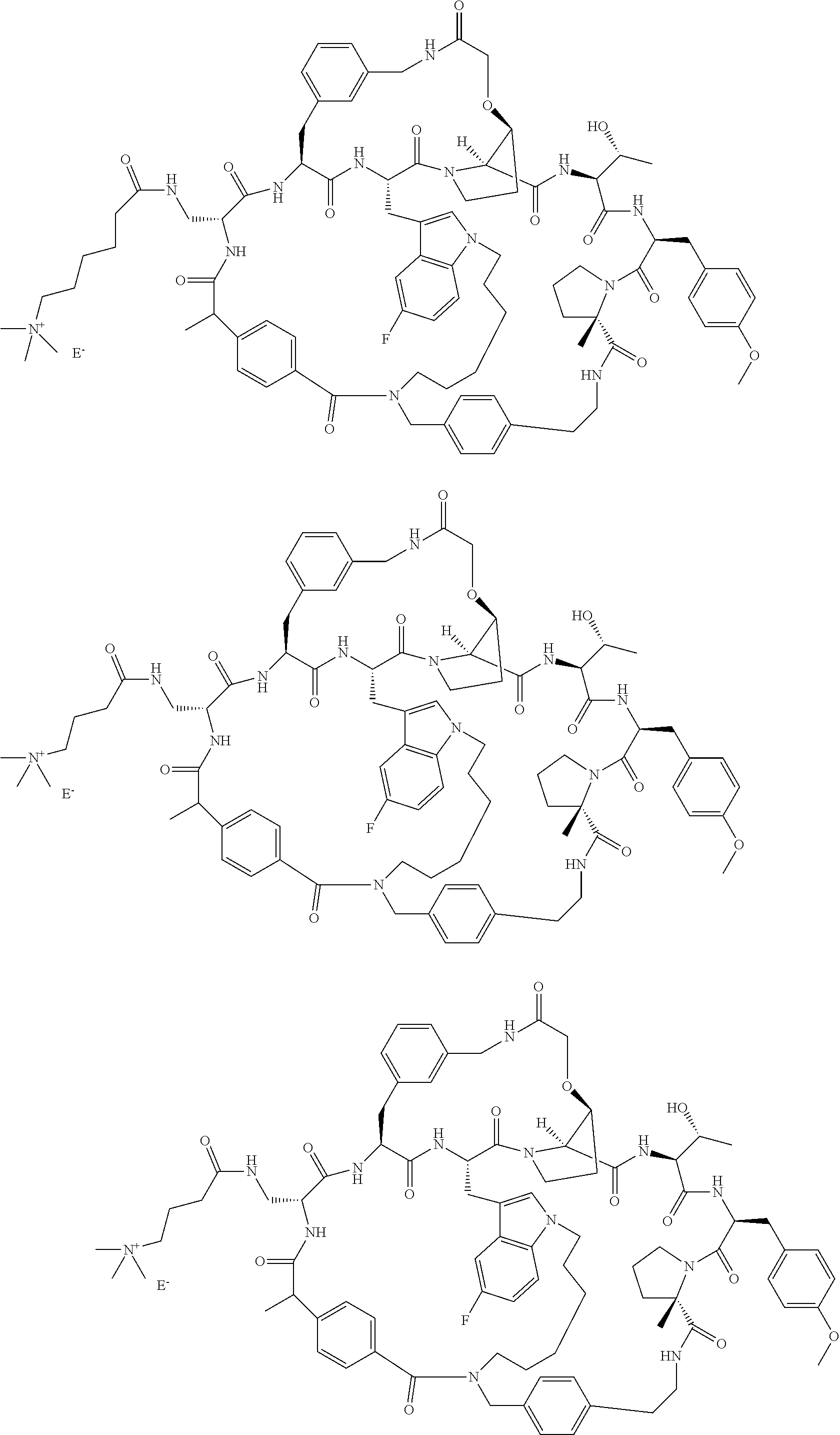

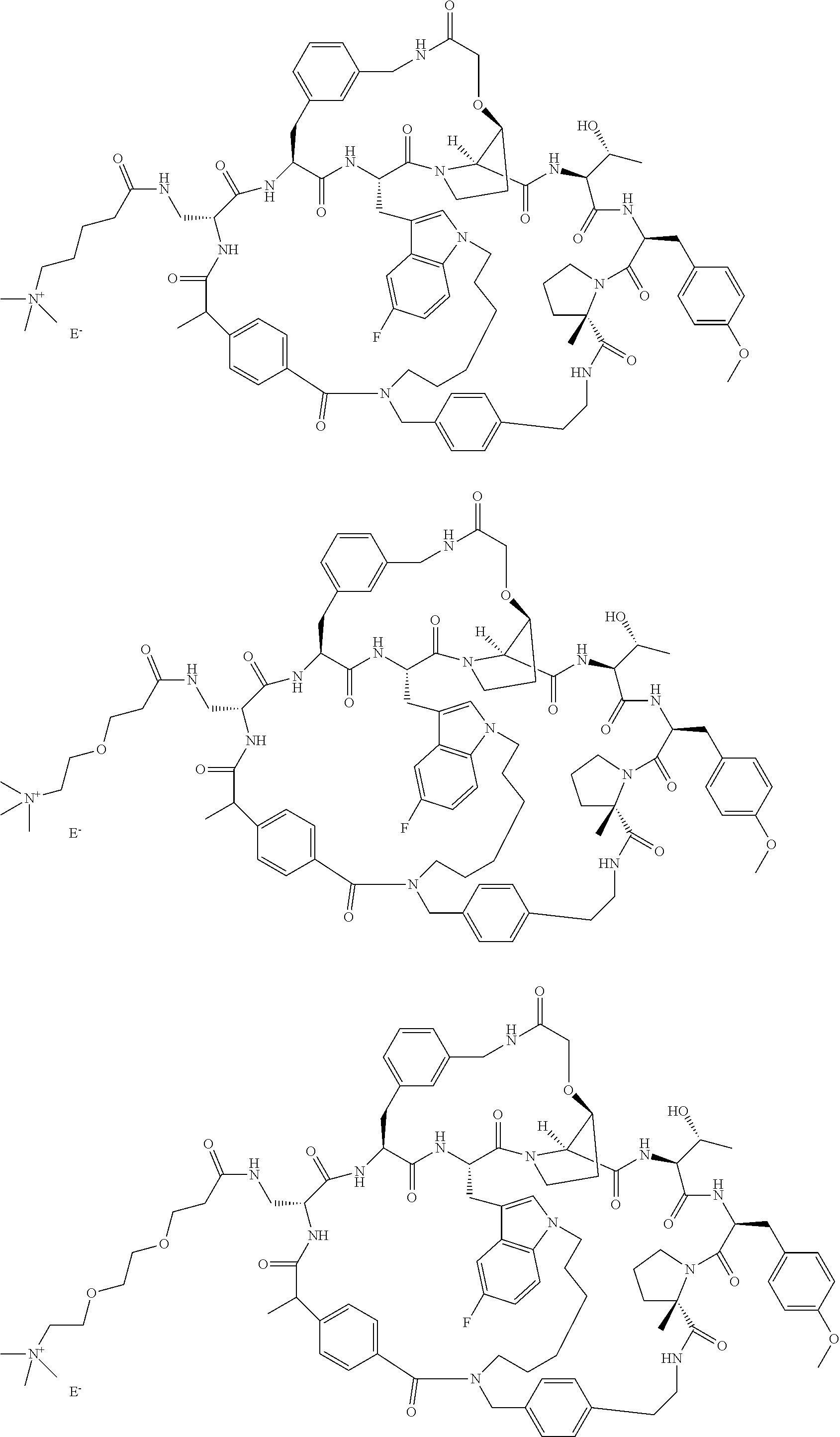

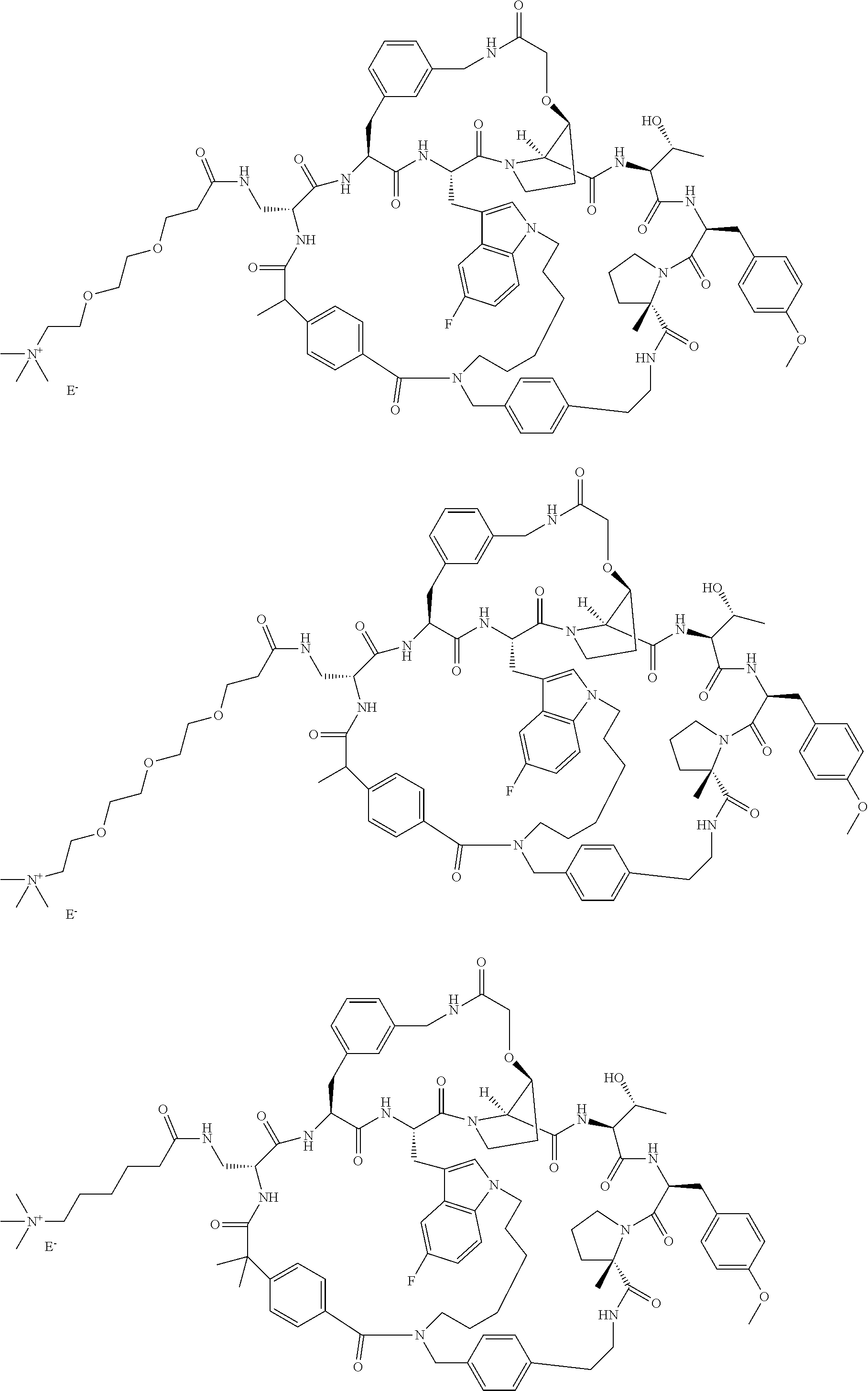

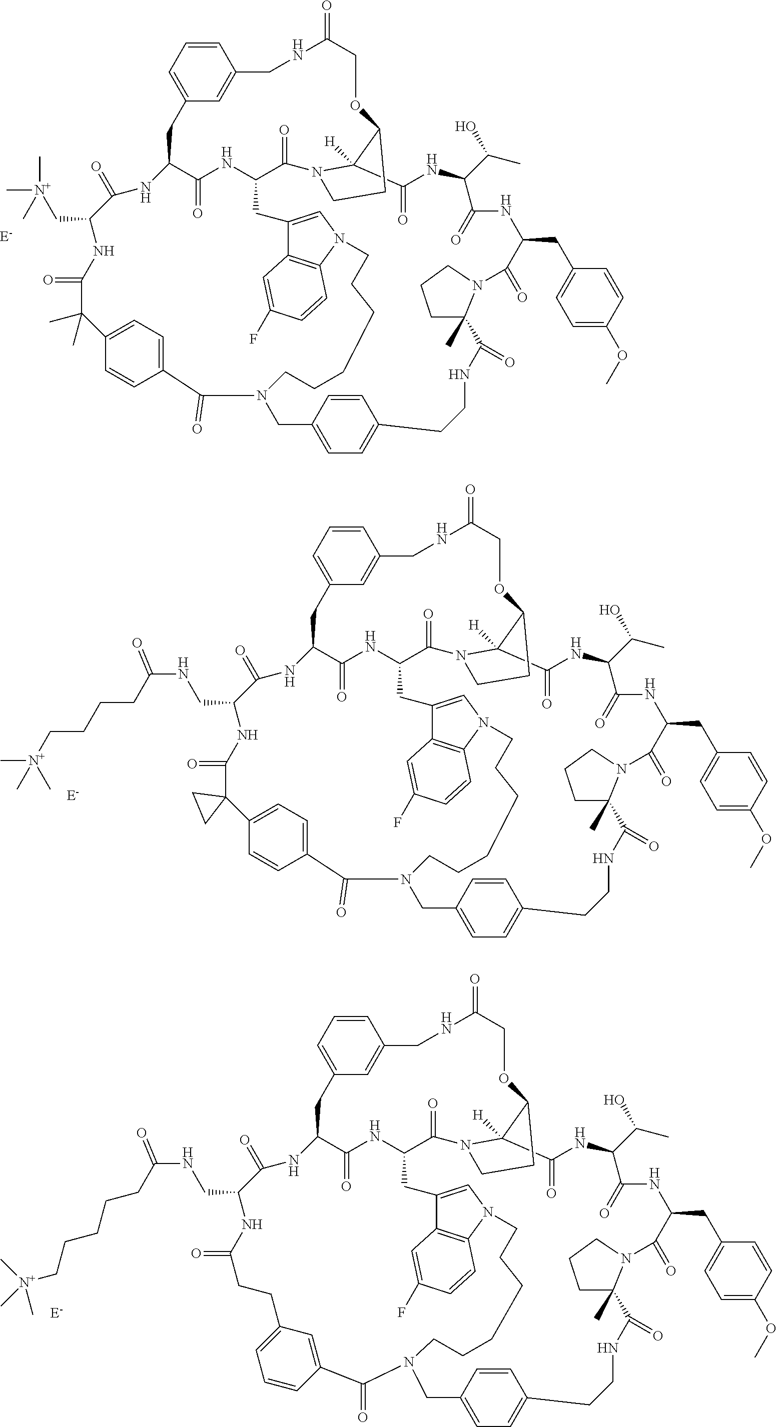

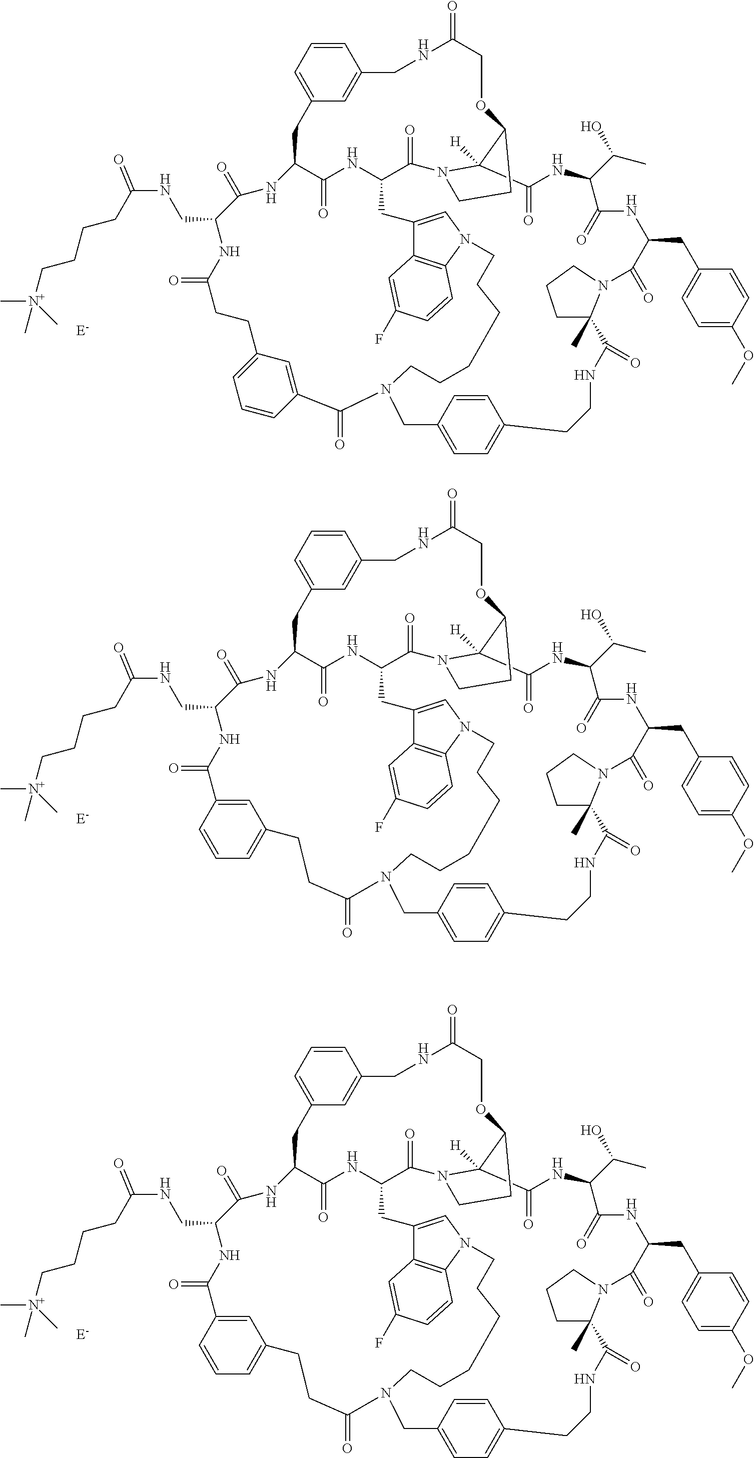

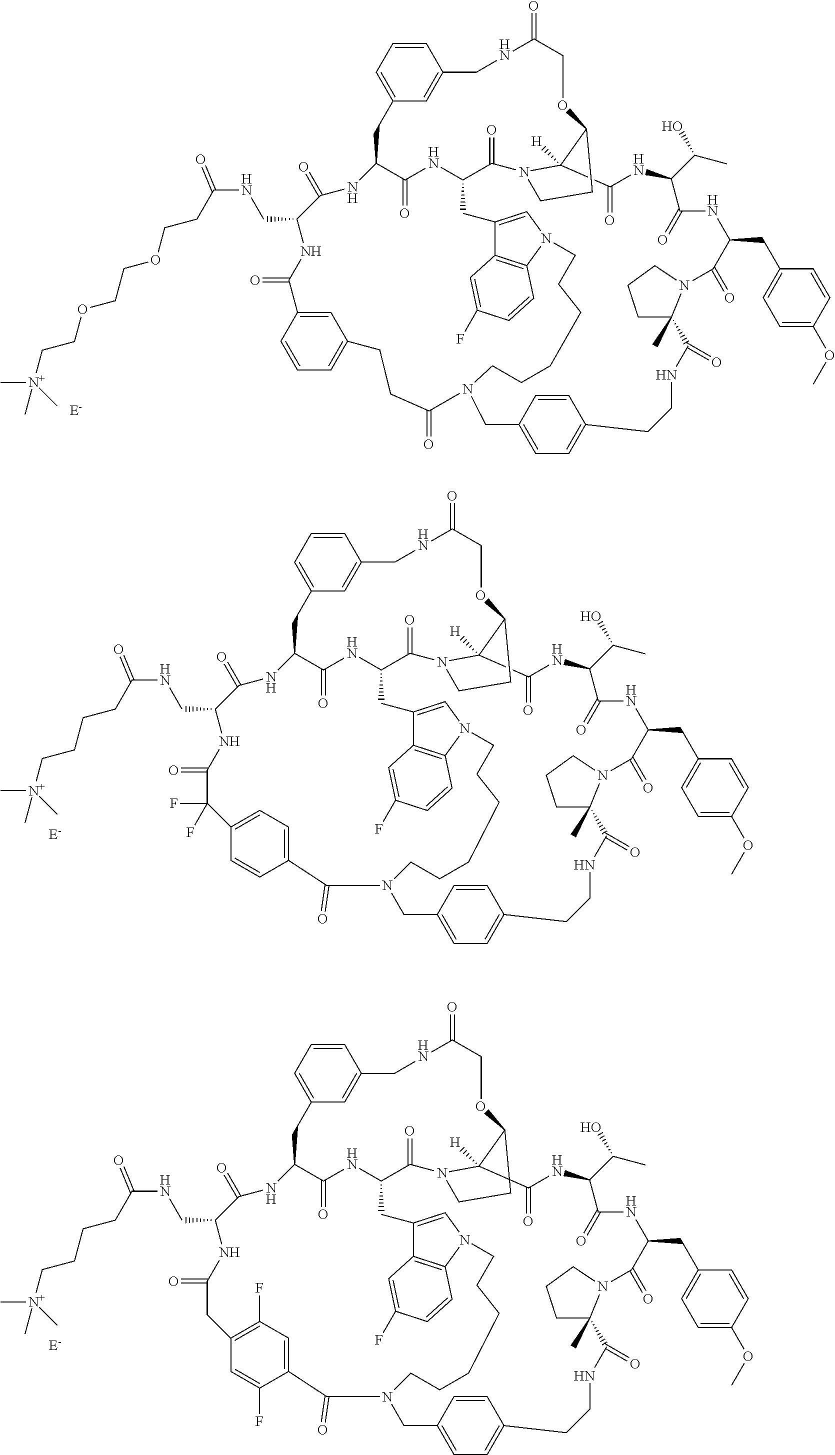

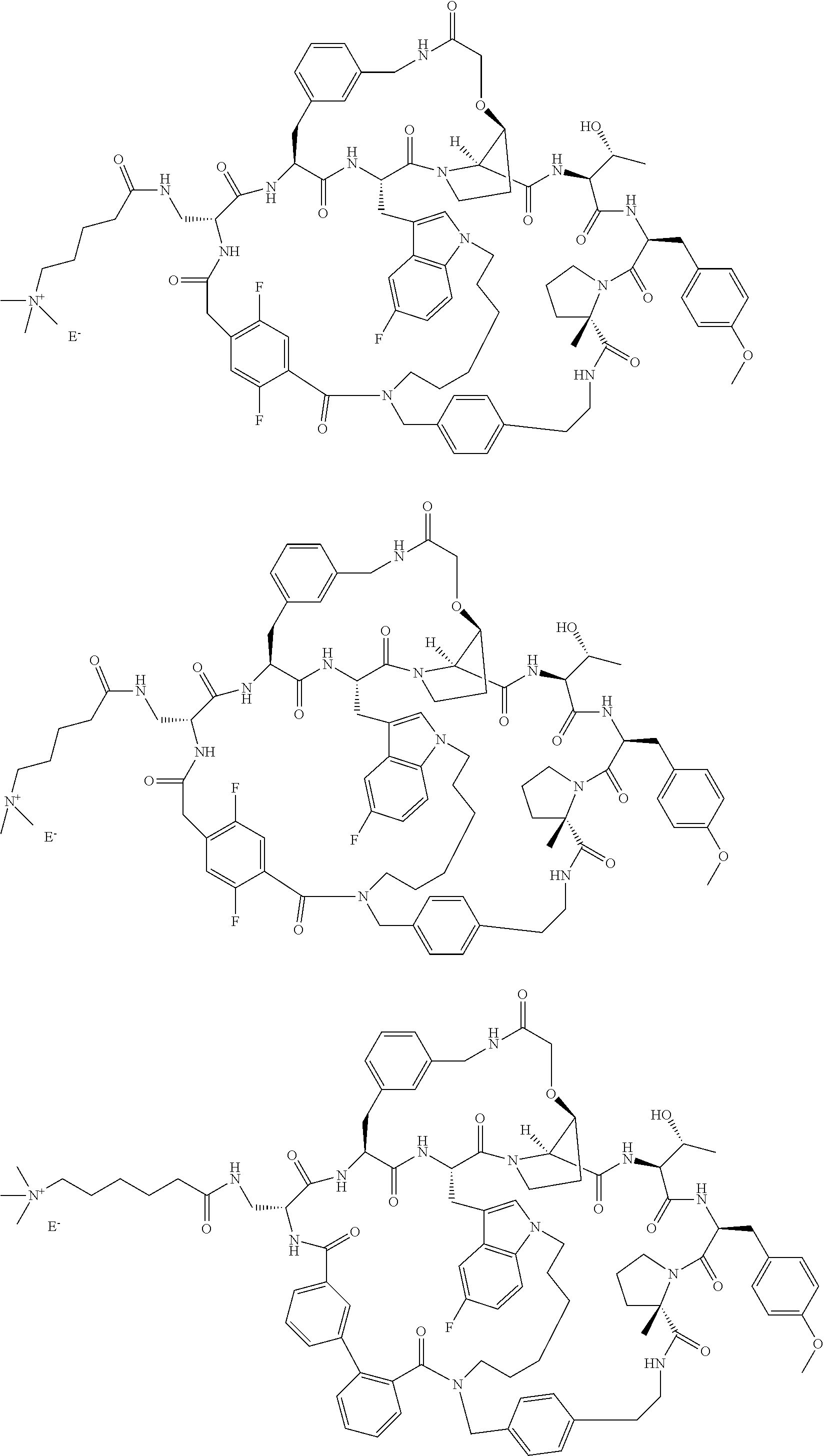

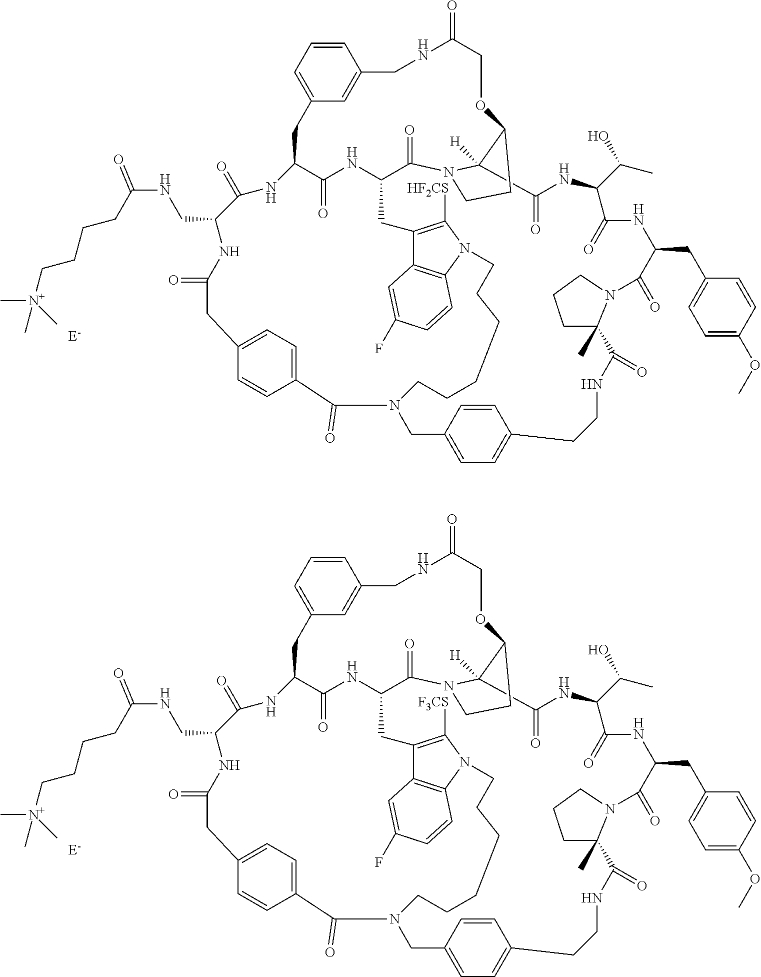

Among them, “E⁻” is a pharmaceutically acceptable anion.

The term “salt” as used herein and its use in phrase “a pharmaceutically acceptable salt” include any of the following: acidic salts formed with inorganic acids and/or organic acids, alkalic salts formed with inorganic bases and/or organic bases, zwitterionic and quaternary ammonium complexes.

The salts of the compounds of the present disclosure can be formed by methods known to those skilled in the art, such as by reacting to the compounds of the present disclosure with a certain amount (e.g., a certain equivalent) of acid or base in a medium such as salt precipitation or aqueous medium, followed by freeze-drying.

The compounds of the present disclosure contain tricoordinated nitrogen atoms, such as primary, secondary, or tertiary amino moieties, wherein, as known, lone electron pairs present on the nitrogen atom may be protonated with appropriate acids or alkylated with appropriate reagents (e.g., alkyl bromides) under appropriate reaction conditions to provide a tetracoordinated charged nitrogen stabilized by anions (e.g., halide ions or conjugated bases) generated during the process. Therefore, the compounds of the present disclosure may be prepared in the form of free bases or isolated in the form of quaternary or salt complexes. In some cases, the formation of basic nitrogen near zwitterionic complexes with appropriate acidic protons is possible. When terms are used herein, salts of the compounds of the present disclosure are included within the scope of the compounds of the present disclosure described herein, whether they are acidic salts formed with inorganic acids and/or organic acids, basic salts formed with inorganic bases and/or organic bases, salts formed with zwitterionic properties (i.e., where compounds contains basic moieties, such as but not limited to nitrogen atoms, e.g., amines, pyridines, or imidazoles; and acidic moieties, such as but not limited to carboxylic acids), and quaternary ammonium complexes.

Examples of pharmaceutically acceptable acidic salts include but are not limited to acetate (including trifluoroacetate), adipate, alginate, ascorbate, aspartate, benzoate, benzenesulfonate, bisulfate, borate, butyrate, citrate, camphorate, camsilate, cyclopentane propionate, digluconate, dodecyl sulfate, ethanesulfonate, fumarate, glucoheptonate, glycerophosphate, hemisulphate, heptanoate, hexanoate, hydrochloride, hydrobromide, hydroiodate, 2-hydroxyethanesulfonate, lactate, maleate, methanesulfonate, methylsulfate, 2-naphthalenesulfonate, nicotinate, nitrate, oxalate, pamoate, pectate, persulfate, 3-phenpropionate, phosphate, picrate, pivalate, propionate, salicylate, succinate, sulfate, sulfonate (e.g., those mentioned herein), tartrate, thiocyanate, toluenesulfonate (also known as tosylate, undecanoate, etc.).

Examples of pharmaceutically acceptable basic salts include but are not limited to ammonium salts, alkali metal salts (e.g., sodium, lithium, and potassium salts), alkaline earth metal salts (e.g., calcium and magnesium salts, aluminum salts, zinc salts), salts with organic bases (e.g., organic amines) (e.g., benzathines), diethylamine, dicyclohexylamine, hydrabamines (formed from N,N-bis(dehydrorosinyl)ethylenediamine), N-methyl-D-glucosamine, N-methyl-D-glucosamine, tert-butylamine, piperazine, phenylcyclohexylamine, choline, and tromethamine), and salts having amino acids (e.g., arginine and lysine). Alkaline nitrogen-containing groups can be converted into ammonium ions or quaternized with following reagents such as lower alkyl halides (e.g., methyl, ethyl, propyl, and butyl chlorides, bromides, and iodides), dialkyl sulfates (e.g., dimethyl, diethyl, dibutyl, and dipentyl sulfates), long-chain halides (e.g., decyl, dodecyl, tetradecyl, and octadecyl chlorides, bromides, and iodides), arylalkyl halides (e.g., benzyl and phenethyl bromides), and other reagents.

“Pharmaceutically acceptable anions” refer to anions suitable for forming pharmaceutically acceptable salts.

Other examples of pharmaceutically acceptable salts that may be used with the present disclosure include, but are not limited to, fluorides, chlorides, bromides, iodides, acid radicals, and the like.

The present disclosure provides a pharmaceutical composition comprising one or more compounds of the present disclosure. As used herein, the term “pharmaceutical composition” includes at least one pharmaceutically active compound and at least one excipient.

Excipient is any ingredient that enables a composition to adapt to a specific route of administration or aid in processing the composition into a dosage form without exerting its own active pharmaceutical effects. Generally, depending on the route of application and the characteristics of the active agent being administered, the composition comprises more than one excipient. Examples of excipients that endow the composition with characteristics that make it easier to handle or process include, but are not limited to, lubricants or pressing aids in the powdered drug intended to be made into tablets, and emulsion stabilizers in the composition, in which the active agent exists in the form of emulsion. Examples of excipients that adapt the composition to the intended route of administration include, but are not limited to, for oral administration, absorption enhancers that promote absorption from the gastrointestinal tract; and for percutaneous or transmucosal administration, penetration enhancers such as those used for adhesive skin “patches” or compositions used for buccal administration.

The solid dosage forms used for oral administration include capsules, tablets, pellets, powders, and granules. In these solid dosage forms, the active compound is mixed with at least one conventional inert excipient (or carrier), such as sodium citrate or dicalcium phosphate, or mixed with the following components: (a) fillers or extenders, such as starch, lactose, sucrose, glucose, mannitol, and silicic acid; (b) binders, such as hydroxymethylcellulose, alginate, gelatin, polyvinylpyrrolidone, sucrose, and arabic gum; (c) humectants, such as glycerol; (d) disrupting agents, such as agar, calcium carbonate, potato starch or cassava starch, alginic acid, certain composite silicates, and sodium carbonate; (e) dissolution retarders, such as paraffin; (f) absorption accelerators, such as quaternary ammonium compounds; (g) wetting agents, such as cetyl alcohol and glycerol monostearate; (h) adsorbents, such as kaolin; and (i) lubricants, such as talc, calcium stearate, magnesium stearate, solid polyethylene glycol, sodium lauryl sulfate, or mixtures thereof. Buffers may also be included in the dosage forms of capsules, tablets, and pellets.

Solid dosage forms such as tablets, sugar pellets, capsules, pellets, and granules may be prepared with coating and shell materials, such as enteric coating and other materials well-known in the art. They may contain opacifying agents, and the active compounds or compounds in such compositions may be released in a certain part of the digestive tract in a delayed manner. Examples of embedding components that may be used are polymeric substances and waxy substances. If necessary, the active compound may also form microcapsule forms with one or more of the above-mentioned excipients.

Liquid dosage forms for oral administration include pharmaceutically acceptable emulsions, solutions, suspensions, syrups or tinctures. In addition to active compounds, liquid dosage forms may include inert diluents commonly used in the art, such as water or other solvents, solubilizers, and emulsifiers, such as ethanol, isopropanol, ethyl carbonate, ethyl acetate, propylene glycol, 1,3-butanediol, dimethylformamide, and oils, particularly cottonseed oil, peanut oil, corn germ oil, olive oil, castor oil, and sesame oil or mixtures of these substances, etc.

In addition to these inert diluents, the composition may also include auxiliaries such as wetting agents, emulsifiers and suspensions, sweeteners, corrigents, and flavouring agents.

In addition to active compounds, suspensions may include suspension agents, such as ethoxylated isooctadecanol, polyoxyethylene sorbitan and sorbitan, microcrystalline cellulose, aluminum methoxide and agar, or mixtures of these substances, etc.

Compositions for parenteral injection may include physiologically acceptable sterile aqueous or anhydrous solutions, dispersions, suspensions or emulsions, and sterile powders for redissolution into sterile injectable solutions or dispersions. Suitable aqueous and non-aqueous carriers, diluents, solvents or excipients include water, ethanol, polyols, and suitable mixtures thereof.

The dosage forms of the compounds of the present disclosure for topical administration include ointments, powders, patches, sprays, and inhalants. The active ingredients are mixed with physiologically acceptable carriers and any preservatives, buffering agents, or propellants that may be needed if necessary, under sterile conditions.

The compound of the present disclosure can also be used for injection formulations. Among them, the injections are selected from liquid injections (water injections), sterile powder for injection (powder injections), or tablets for injection (which refer to moulded tablets or machine pressed tablets made by aseptic techniques for drugs, which are dissolved in water for injection when in use for subcutaneous or intramuscular injection).

The term “inhibition”, i.e., “antagonism” as used in this application refers to a substance that provides an action against PCSK9 in the affected tissues, and inhibits, counteracts, neutralizes, or reduces one or more functions of PCSK9 in the affected tissues. Inhibition or antagonism of one or more functional characteristics associated with PCSK9 may be easily determined using methods known in the art (see, for example, Barak and Webb, 1981, J. Cell Biol. 90:595-604; Stephan&Yurachek, 1993, J. Lipid Res. 34: 325-330; and McNamara et al., 2006, ClinicaChimica Acta, 369: 158-167.) and those methods described herein. Inhibition or antagonism will result in a decrease in activity of PCSK9 compared to those observed in the absence of an antagonist or, for example, compared to the activity observed when a control antagonist with unrelated specificity is present.

The present disclosure provides a method for inhibiting or antagonizing the activity of PCSK9 in a subject, comprising administering a therapeutically effective dose of a compound of the present disclosure to the subject.

The term “therapeutic method” refers to the acting process of causing changes in at least one symptom of a disease status that may be essentially preventive or therapeutic in nature. In some embodiments, the present disclosure relates to a therapeutic method for a condition associated with and/or attributed to PCSK9 activity or a condition in which the function of PCSK9 is contraindicated for a specific individual, comprising administering a therapeutically effective amount of a PCSK9 antagonist compound of formula I or a pharmaceutically acceptable salt thereof to the individual. In some embodiments, the condition may be atherosclerosis, hypercholesterolemia, coronary heart disease, metabolic syndrome, acute coronary syndrome or related cardiovascular disease and cardiometabolic disease, or it may be a disease status or condition in which PCSK9 activity is contraindicated.

The therapeutic method according to the present disclosure comprises administering a therapeutic (or prophylactic) effective amount of the PCSK9 specific antagonist of the present disclosure to an individual. The term “therapeutically effective” or “prophylactically effective” used with reference to a certain amount refers to the necessary amount at the expected dose for the expected duration of time to achieve the desired therapeutic and/or preventive effect. The expected effect may be, for example, alleviation, mitigation, reduction, or cessation of at least one symptom associated with the treated conditions. As will be understood by those skilled in the art, these amounts will vary depending on various factors, including but not limited to disease status, age, gender, individual weight, and the ability of PCSK9 specific antagonists to induce the desired effect in individuals. Responses may be recorded through in vitro assays, in vivo non-human animal studies, and/or further supported by clinical trials.

The term “effective amount” or “effective dose” refers to the amount of active compounds sufficient to antagonize PCSK9 and thereby inducing the desired response (i.e., the therapeutic response induced in the treatment or management of the symptoms associated with or affected by PCSK9 function, the conditions include but are not limited to atherosclerosis, hypercholesterolemia, coronary heart disease, metabolic syndrome, acute coronary syndrome or related cardiovascular diseases and cardiometabolic disease, and related cardiovascular diseases and cardiometabolic symptoms in animals or humans).

The compounds of the present disclosure may be prepared using readily available starting materials, reagents, and conventional synthetic procedures according to the following reaction schemes and examples or their modifications. In these reactions, it is also possible to utilize known variants. Given the following reaction schemes and examples, other methods for preparing the compounds of the present disclosure will be apparent to those of ordinary skill in art.

### Abbreviations of Compounds are Shown Below

EA: Ethyl acetate
Cbz-Cl: Benzyl chloroformate
THF: Tetrahydrofuran
DCC: N,N′-dicyclohexylcarbodiimide
DMAP: 4-dimethylaminopyridine
EtOH: Ethanol
DMF: N,N-dimethylformamide
DCM: Dichloromethane
TMSOTf: Trimethylsilyl trifluoromethanesulfonate
t-BuOH: Tertiary butanol
BOC: Tert-butoxycarbonyl
dppb: 1,4-bis(diphenylphosphino)butane
Raney Ni: Raney nickel
Fmoc-Cl: 9-fluorenylmethyl chloroformate
MTBE: Methyl tert-butyl ether
PE: Petroleum ether
DIPEA: N,N-diisopropylethylamine
HATU: 2-(7-azabenzotriazol)-N,N,N′,N′-tetramethyluronium hexafluorophosphate
TMSI Trimethylsilyl iodide
TFA: Trifluoroacetic acid
LiHMDS: Lithium hexamethyldisilazide
Xantphos: 4,5-Bis(diphenylphosphino)-9,9-dimethylxanthene
Pd(dppf)Cl₂: 1,1′-Bis(diphenylphosphino)ferrocene]dichloropalladium
Me₃N: Trimethylamine

### Example 1 Synthesis of Key Fragments

### 1. Synthesis of Pro Fragments

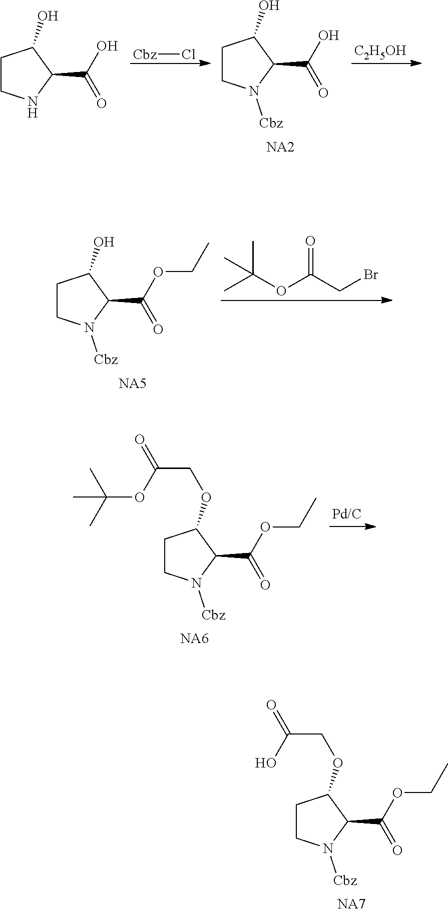

### 1). Preparation of NA2

Trans-3-hydroxy-L-proline (100 g, 762.6 mmol) was dissolved in 800 mL of 1,4-dioxane, and a solution of sodium bicarbonate (141 g, 1677.7 mmol) in 1200 mL of water was added at 0° C. Then benzyl chloroformate (195.14 g, 1143.9 mmol) was added dropwise, and reacted at room temperature for 12 hours. The reaction was monitored via LC/MS until it was completed. Dilute hydrochloric acid was added dropwise at 0° C. to adjust pH to 3. Then the mixture was extracted with 400 mL of EA for three times, dried over anhydrous Na₂SO₄ and filtered. The filtrate was rotary evaporated to dryness under reduced pressure to obtain crude product, which was purified by silica gel column chromatography to obtain 184.4 g of NA2 as an off-white solid with a yield of 91%.

### 2). Preparation of NA5

Intermediate NA2 (10 g, 37.7 mmol) was dissolved in 100 mL of THF, and DCC (8.54 g, 41.5 mmol) and 10% DMAP (460 mg, 3.77 mmol) were added, which was stirred for 0.5 hours, then EtOH (3.47 g, 75.4 mmol) was added. The reaction was terminated when the conversion of raw material NA2 was complete. Then, 100 mL of EA was added to the reaction solution for dilution, which was washed with water and saturated saline solution, dried over anhydrous Na₂SO₄ and filtered. The filtrate was rotary evaporated to dryness under reduced pressure to obtain crude product, which was purified by silica gel column chromatography to obtain 6.73 g of NA5 as a white solid with a yield of 68%.

### 3). Preparation of NA6

NA5 (50.0 g, 0.170 mol) was dissolved in 400 mL of DMF. Cs₂CO₃ (83.3 g, 0.255 mol) and sodium iodide (1.27 g, 8.5 mmol) were added, after the solution was stirred at room temperature for 5 minutes, tert-butyl bromoacetate (33.2 g, 0.170 mol) was added, and reacted at 60° C. for 12 hours. The reaction was monitored via LC/MS until NA5 was consumed completely. The reaction solution was suction filtered, and 200 mL of EA and 400 mL of water were added to the filtrate, which was extracted with a small amount of EA. The organic phases were combined, dried over anhydrous Na₂SO₄ and filtered. The filtrate was rotary evaporated to dryness under reduced pressure to obtain crude product, which was purified by silica gel column chromatography to obtain 54.1 g of NA6 as a pale-yellow oil with a yield of 78%.

### 4). Preparation of NA7

NA6 (15.76 g, 24.09 mmol) was dissolved in 100 mL of DCM, 2,6-dimethylpyridine (8.04 g, 75.00 mmol) was added at 0° C. Then TMSOTf (15.16 g, 68.18 mmol) was added dropwise. The solution was reacted at room temperature for 2 hours. The reaction was monitored via LC/MS until it was completed. The reaction solution was washed twice with 20 mL of water, the organic phase was dried over anhydrous Na₂SO₄ and filtered. The filtrate was rotary evaporated to dryness under reduced pressure to obtain crude product, which was purified by preparative chromatography to obtain 9.86 g of NA7 as a yellow oil with a yield of 62%.

### 2. Synthesis of Trp Fragments

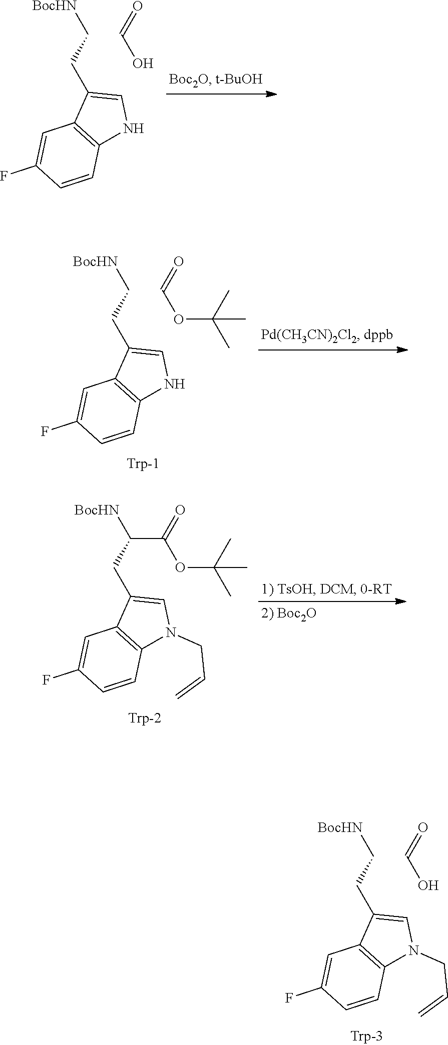

### 1). Preparation of Intermediate Trp-1

Raw material 5-F-N-Boc-tryptophan (120 g, 373 mmol) was dissolved in 1200 mL of t-BuOH, 5% DMAP (6.1 g, 18.6 mmol) was added, then Boc₂O (90 g, 447.6 mmol) was added dropwise. The solution was reacted at 35° C. until the raw materials were consumed completely. Then tert-BuOH was removed by rotary evaporation to obtain the crude product, which can be directly used for the next reaction step without purification. LC/MS: [M−H]⁻: 377.3.

### 2). Preparation of Intermediate Trp-2

Crude Trp-1 was dissolved in 1000 mL of THF, then allyl methyl carbonate (51.9 g, 447.6 mmol), K₂CO₃ (102.9 g, 746 mmol), 20% dppb (31.78 g, 74.6 mmol) and 10% Pd(CH₃CN)₂Cl₂ (9.66 g, 37.3 mmol) were added, the solution was reacted at 35° C. under nitrogen atmosphere until the raw materials were consumed completely. Then, the reaction solution was added dropwise into 1000 mL of water. The solution was extracted with 500 mL of EA for three times. The organic phases were combined, washed with saturated saline twice, then dried over anhydrous Na₂SO₄ and filtered. The filtrate was rotary evaporated to dryness under reduced pressure to obtain crude product, which was purified by silica gel column chromatography to obtain 98 g of Trp-2 as a pale-yellow solid with a two-step yield of 63%.

### 3). Preparation of Intermediate Trp-3

Intermediate Trp-2 (98 g, 234 mmol) was dissolved in 1000 mL of DCM. The solution was cooled to 0° C., then p-TsOH·H₂O (210 g, 1.17 mol) was added. The reaction was monitored via LC/MS until the raw materials were consumed completely. Then, the reaction solution was rotary evaporated to dryness, and redissolved in 1000 mL of THF/water (v/v=2/1). Boc₂O (70 g, 351 mmol) was added, and reacted until the raw materials were consumed completely. Then the reaction solution was rotary evaporated to dryness under reduced pressure to remove THF.

The crude product was purified by preparative chromatography to obtain 79 g of Trp-3 as a pale-yellow solid with a yield of 93%.

### 3. Synthesis of Phe Fragments

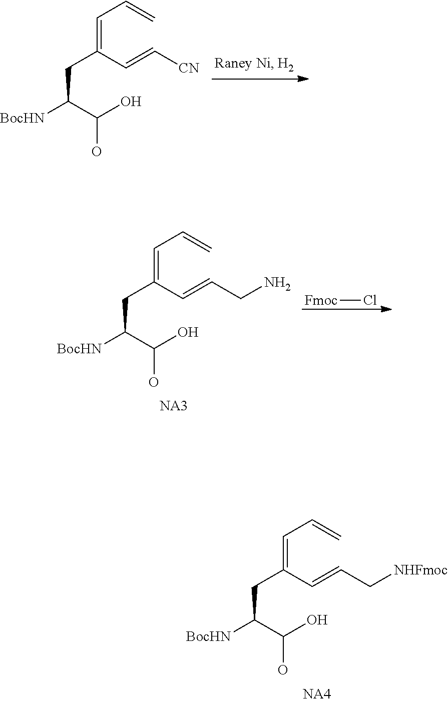

### 1). Preparation of NA3

Raw material Boc-L-3-cyanophenylalanine (50.0 g, 0.172 mol) was dissolved in a mixture of 300 mL methanol and 50 mL concentrated ammonia hdroxide solution, Raney-Ni (10.1 g, 0.172 mol) was added, and then the solution was purged with hydrogen for three times and reacted at room temperature for 12 hours. The reaction was monitored via LC/MS until the raw materials were consumed completely. Most of the methanol was rotary evaporated, and an aqueous solution of 5% phosphoric acid was added to adjust pH to 4. The mixture was stirred for 30 minutes and then left to stand at −20° C. for 12 hours. A large amount of solid crude product precipitated out, and the crude product was purified by preparative chromatography to obtain 32.9 g of NA3 as a white solid with a yield of 65%.

### 2). Preparation of NA4

NA3 (32.9 g, 0.112 mol) was dissolved in a mixture of 300 mL water and 150 mL ethanol, then NaHCO₃ (23.5 g, 0.280 mol) was added at room temperature. After the completion of addition, the solution was cooled to 2-8° C., and Fmoc-Cl (30.3 g, 0.117 mol) was added in portions, then the solution was slowly warmed and reacted under nitrogen atmosphere for 12 hours. The reaction was monitored via LC/MS until NA3 consumed completely. Most of the solvent was rotary evaporated off, and the aqueous phase was extracted three times with 300 mL of a mixed solvent (PE/MTBE, v/v=1:1). The lower aqueous phase was collected, and the system was cooled to 10-15° C., and adjusted the pH to 5 with 1 M acetic acid, then the aqueous phase was extracted with 100 mL of EA for three times. The organic phases were combined, dried over anhydrous Na₂SO₄ and filtered. The filtrate was rotary evaporated to dryness under reduced pressure to obtain crude product, which was purified by preparative chromatography to obtain 42.5 g of NA4 as a white solid with a yield of 73%.

### 4. Synthesis of NA Fragments

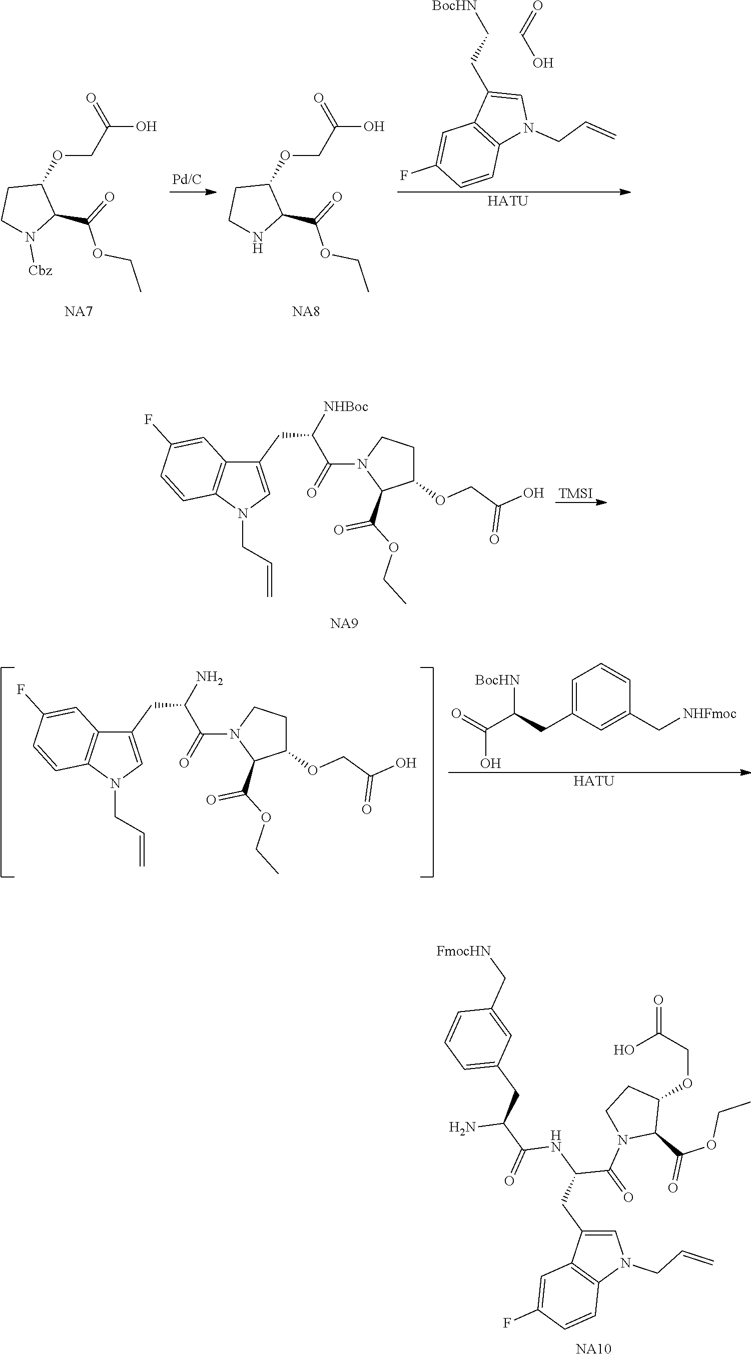

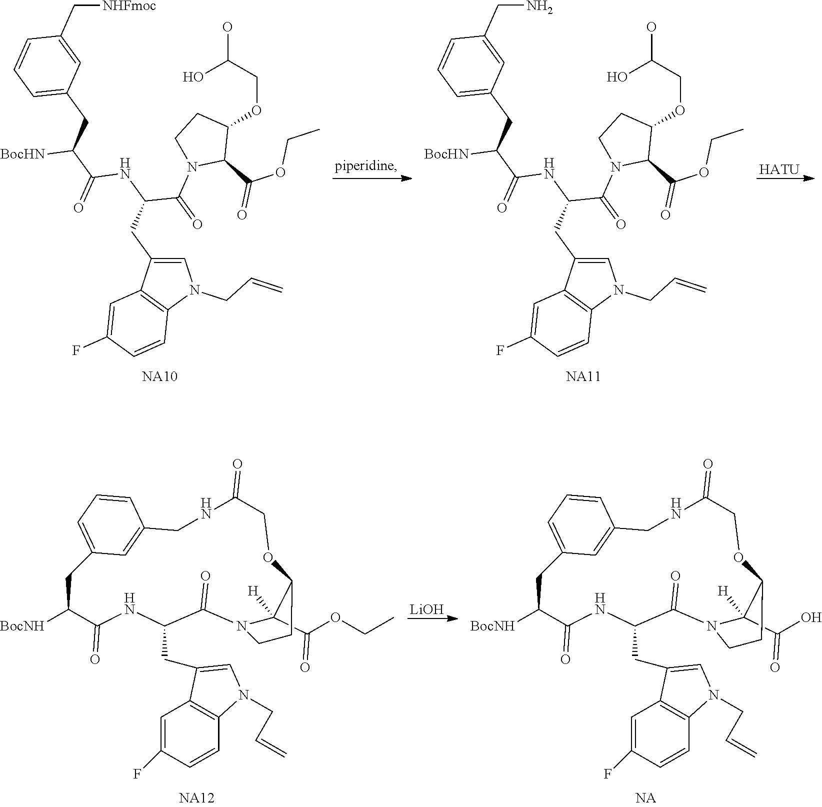

### 1). Preparation of Intermediate NA8

Intermediate NA7 (100 g, 0.28 mol) was dissolved in 800 mL of ethanol. Then 10% Pd/C was added, and the mixture was reacted under a hydrogen atmosphere until the raw materials were consumed completely. The reaction solution was filtered and rotary evaporated to dryness to obtain crude intermediate NA8, which was used directly for the next reaction step without purification. LC/MS: [M+H]⁺: 218.1.

### 2). Preparation of Intermediate NA9

Intermediate Trp-2 (78 g, 0.21 mol) was dissolved in 780 mL of DMF, and DIPEA (95 mL, 0.55 mol) was added. The solution was cooled to 0° C., and HATU (85.4 g, 0.22 mol) was added, then reacted until the raw materials were consumed completely, the above intermediate NA8 was added to the reaction solution. Then the temperature rose to room temperature spontaneously and reacted until the raw materials were consumed completely. Then, the reaction solution was added dropwise into 1 M acetic acid solution at 0° C., which was then extracted with 500 mL of EA for three times, washed with saturated saline, dried over anhydrous Na₂SO₄ and filtered. The filtrate was rotary evaporated to dryness under reduced pressure to obtain crude product, which was purified by preparative chromatography to obtain 43.2 g of NA9 as a pale-yellow oil with a yield of 36%.

### 3). Preparation of Intermediate NA10

Intermediate NA4 (39.6 g, 76.9 mmol) was dissolved in 400 mL of DMF, and DIPEA (80 mL, 461.4 mmol) was added. The solution was cooled to 0° C., and HATU (31.3 g, 80.7 mmol) was added. Then reacted until the raw materials were consumed completely.

Intermediate NA9 (43.2 g, 76.9 mmol) was dissolved in 300 mL of CH₃CN. The solution was cooled to 0° C., and TMSI (38.4 g, 192.2 mmol) was added dropwise. The solution was reacted until the raw materials were consumed completely. Then it was added dropwise into the solution of NA4 prepared above until the raw materials were consumed completely. Subsequently, the reaction solution was added dropwise into 1 M acetic acid solution at 0° C., which was then extracted with 200 mL of EA for three times, washed with saturated saline, dried over anhydrous Na₂SO₄ and filtered. The filtrate was rotary evaporated to dryness under reduced pressure to obtain crude product, which was purified by preparative chromatography to obtain 40.2 g of NA10 as a pale-yellow oil with a yield of 54.3%.

### 4). Preparation of Intermediate NA11

Intermediate NA10 (40.2 g, 41.8 mmol) was dissolved in 500 mL of CH₃CN. The solution was cooled to 0° C., then piperidine (17.8 g, 209 mmol) was added dropwise, which was reacted until the raw materials were consumed completely. Then, the reaction solution was rotary evaporated to remove the solvent and residual piperidine. The crude reaction solution was directly used for the next reaction step after PE pulping and filtration. LC/MS: [M+H]⁺: 738.4.

### 5). Preparation of Intermediate NA12

The above crude NA11 was dissolved in 2000 mL of DCM, and DIPEA (58 mL, 334.4 mmol) was added. The solution was cooled to 0° C., and HATU (81.1 g, 209 mmol) was added, then reacted until the raw materials were consumed completely. The reaction solution was added dropwise into ice-water mixture, which was extracted with EA, washed with saturated saline, dried over anhydrous Na₂SO₄ and filtered. The filtrate was rotary evaporated to dryness under reduced pressure to obtain crude product, which was purified by preparative chromatography to obtain 15.4 g of NA12 as a white solid with a two-step yield of 51%.

### 6). Preparation of Intermediate NA

Intermediate NA12 (15.4 g, 21.3 mmol) was dissolved in 800 mL of THF/MeOH/H₂O (v/v/v=6/3/2), and then 91.6 mL of 1M LiOH aqueous solution was added dropwise at 0° C. After 36 hours of reaction, the raw materials were consumed completely, the solution was adjusted to pH=6 with 1 M acetic acid. Then the mixture was extracted with 200 mL of EA for three times, the organic phase was dried over anhydrous Na₂SO₄ and filtered. The filtrate was rotary evaporated to dryness under reduced pressure to obtain crude product, which was purified by preparative chromatography to obtain 12.6 g of NA as a white solid with a yield of 85%.

### 5. Synthesis of NC Fragments

### 1). Preparation of Intermediate NC1

S1 (10.0 g, 33.9 mmol) was dissolved in 100 mL of DMF, then DIPEA (6.48 mL, 37.29 mmol) and HATU (14.17 g, 7.3 mmol) were added at 0° C. The solution was stirred for 1 hour with an ice cooling bath before methyl 2-methyl-L-prolinate hydrochloride (6.068 g, 33.9 mmol) was added and then stirred for another 10 minutes. Then the solution was warmed to room temperature and stirred for about 12 hours. The reaction was monitored via LC/MS until S1 was consumed completely. The reaction was quenched and extracted with 30 mL of EA for three times. The organic phases were dried over anhydrous Na₂SO₄ and filtered. The filtrate was rotary evaporated to dryness under reduced pressure to obtain crude product, which was purified by silica gel column chromatography to obtain 10.88 g of NC1 as a yellow oil with a yield of 77%.

### 2). Preparation of Intermediate NC2

NC1 (10.88 g, 25.9 mmol) was dissolved in 100 mL of DCM. 25 mL of TFA was added at 0° C., the solution was stirred with an ice bath until LC/MS showed that NC1 was consumed completely. The pH of solution was adjusted to 7-8 with DIPEA under ice bath conditions, which was then used in the next step directly.

LC/MS: [M+H]⁺: 321.4.

### 3). Preparation of Intermediate NC3

N-Boc-tert-butoxycarbonyl-O-tert-butyl-L-threonine (7.124 g, 25.9 mmol) was dissolved in 70 mL of DCM. DIPEA (5 mL, 28.5 mmol) and HATU (11.06 g, 28.5 mmol) were added at 0° C. The solution was stirred under ice bath conditions for 1 hour, and then added to the above NC2 solution of which the pH was adjusted, and stirred at 0° C. for 10 minutes before warming up to room temperature, and then stirred at room temperature for 4 hours.100 mL of water was added to the reaction solution, which was extracted with 100 mL of DCM for three times The organic phases were combined, which was then dried over anhydrous Na₂SO₄ and filtered. The filtrate was rotary evaporated to dryness under reduced pressure to obtain crude product, which was purified by silica gel column chromatography to obtain 10.42 g of NC3 as a yellow oil with a yield of 70%.

### 4). Preparation of Intermediate NC

NC3 (10.42 g, 18.06 mmol) was dissolved in 660 mL of THF/MeOH/H₂O (v/v/v=6:3:2), and LiOH aqueous solution (3.79 g, 90.3 mmol) was added at 0° C. The reaction was monitored via LC/MS until NC3 was consumed completely. The pH was adjusted with 1 M acetic acid solution, then the organic phase was dried over anhydrous Na₂SO₄ and filtered. The filtrate was rotary evaporated to dryness under reduced pressure to obtain crude product, which was purified by preparative chromatography to obtain 6.09 g of NC as a white solid with a yield of 60%.

TABLE 1
Structural Identification Data of Some Compounds
In the Synthesis Route of Key Fragments

| Nos. | Structural identification data |
|---|---|
| NA2 | LC/MS: [M + H]⁺: 266.2. ¹H NMR (400 MHz, CD₃OD) δ: 7.23-7.27 |
| | (m, 2H), 7.11 (s, 1H), 6.89 (td, J₁ = 9.2 Hz, J₂ = 2.0 Hz, 1H), 5.94-6.03 (m, 1H), |
| | 5.13 (d, J = 10.4 Hz, 1H), 4.96 (d, J = 18.0 Hz, 1H), 4.73 (dt, J₁ = 5.2 Hz, J₂ = 1.6 |
| | Hz, 2H), 4.38 (dd, J₁ = 7.2 Hz, J₂ = 5.2 Hz, 1H), 3.20-3.27 (m, 1H), 3.07 (dd, |
| | J₁ = 14.8 Hz, J₂ = 7.6 Hz, 1H), 1.38 (s, 1H). |
| NA5 | LC/MS: [M + H]⁺: 294.2. ¹H NMR (400 MHz, DMSO-d₆) δ: 7.27-7.38 |
| | (m, 5H), 4.96-5.12 (m, 2H), 4.34 (d, J = 16.0 Hz, 1H), 4.17 (dd, J₁ = 16.4 Hz, |
| | J₂ = 4.0 Hz, 1H), 4.11 (d, J = 6.8 Hz, 1H), 4.06-4.09 (m, 2H), 4.02-4.07 (m, |
| | 1H), 3.49-3.58 (m, 1H), 3.39-3.49 (m, 1H), 1.91-2.06 (m, 2H), 1.43 (s, |
| | 4.5H), 1.42 (s, 4.5H), 1.18 (t, J = 7.2 Hz, 1.5H), 1.09 (t, J = 7.2 Hz, 1.5H). |
| NA6 | LC/MS: [M + H]⁺: 408.7. ¹H NMR (400 MHz, DMSO-d₆) δ: 7.27-7.38 |
| | (m, 5H), 4.96-5.11 (m, 2H), 4.34 (d, J = 16.0 Hz, 1H), 4.18 (dd, J₁ = 4.0 Hz, |
| | J₂ = 12.0 Hz, 1H), 4.11-4.13 (m, 1H), 4.07-4.08 (m, 2H), 4.02-4.06 (m, 1H), |
| | 3.49-3.58 (m, 1H), 3.39-3.49 (m, 1H), 1.88-2.06 (m, 2H), 1.43 (s, 4H), 1.42 |
| | (s, 5H), 1.18 (t, J = 7.2 Hz, 1.5H), 1.09 (t, J = 7.2 Hz, 1.5H). |
| NA7 | LC/MS: [M + H]⁺: 352.3. ¹H NMR (400 MHz, DMSO-d₆) δ: 12.75 (brs, |
| | 1H), 7.27-7.38 (m, 5H), 4.96-5.13 (m, 2H), 4.34 (d, J = 14.8 Hz, 1H), 4.18- |
| | 4.22 (m, 1H), 4.08-4.16 (m, 3H), 4.00-4.07 (m, 1H), 3.40-3.55 (m, 2H), 1.91- |
| | 2.07 (m, 2H), 1.18 (t, J = 7.2 Hz, 1.5H), 1.09 (t, J = 7.2 Hz, 1.5H). |
| Trp-2 | LC/MS: [M + H]⁺: 419.4. ¹H NMR (500 MHz, CDCl₃) δ: 7.23 (dd, J₁ = 2.5 |
| | Hz, J₂ = 9.5 Hz, 1H), 7.17 (dd, J₁ = 4.5 Hz, J₂ = 9.0 Hz, 1H), 6.91-6.97 (m, 2H), |
| | 5.90-5.98 (m, 1H), 5.19 (d, J = 11.0 Hz, 1H), 5.03-5.08 (m, 2H), 4.65 (d, J = 5.5 |
| | Hz, 2H), 4.49-4.52 (m, 1H), 3.19 (qd, J₁ = 5.5 Hz, J₂ = 15.0 Hz, 1H), 1.44 (s, |
| | 9H), 1.38 (s, 9H). |
| Trp-3 | LC/MS: [M + H]⁺: 363.3. ¹H NMR (400 MHz, CD₃OD) δ: 7.25 (dd, |
| | J₁ = 2.4 Hz, J₂ = 9.6 Hz, 1H), 7.10 (s, 1H), 6.89 (td, J₁ = 2.4 Hz, J₂ = 9.2 Hz, 1H), |
| | 5.93-6.03 (m, 1H), 5.13 (d, J = 10.4 Hz, 1H), 4.97 (d, J = 17.2 Hz, 1H), 4.71 |
| | (d, J = 5.2 Hz, 2H), 4.39 (dd, J₁ = 5.2 Hz, J₂ = 7.6 Hz, 1H), 3.26 (dd, J₁ = 5.2 Hz, |
| | J₂ = 14.8 Hz, 1H), 3.07 (dd, J₁ = 8.0 Hz, J₂ = 14.8 Hz, 1H), 1.38 (s, 9H). |
| NA3 | LC/MS: [M + H]⁺: 295.4. ¹H NMR (500 MHz, D₂O) δ: 7.35-7.44 (m, |
| | 1H), 7.28-7.32 (m, 3H), 7.34 (dd, J₁ = 5.0 Hz, J₂ = 9.5 Hz, 1H), 4.12-4.13 (m, |
| | 2H), 3.17-3.21 (m, 1H), 2.92 (dd, J₁ = 9.5 Hz, J₂ = 13.5 Hz, 1H), 1.28 (m, 9H). |
| NA4 | LC/MS: [M + H]⁺: 517.6. ¹H NMR (500 MHz, CD₃OD) δ: 7.78 (d, J = 8.0 Hz, |
| | 2H), 7.64 (d, J = 7.5 Hz, 2H), 7.38 (t, J = 7.5 Hz, 2H), 7.29 (t, J = 7.5 Hz, 2H), |
| | 7.23 (t, J = 7.5 Hz, 1H), 7.15 (s, 1H), 7.12 (, J = 7.0 Hz, 2H), 4.37 (d, J = 6.5 Hz, |
| | 2H), 4.27 (s, 2H), 4.19 (t, J = 7.0 Hz, 1H), 7.29 (q, J = 7.0 Hz, 1H), 3.20 (s, |
| | 2H), 3.15 (dd, J₁ = 5.0 Hz, J₂ = 14.0 Hz, 1H), 2.89 (dd, J₁ = 9.5 Hz, J₂ = 14.0 Hz, |
| | 1H), 1.18 (m, 9H). |
| NA9 | LC/MS: [M + H]⁺: 561.9. ¹H NMR (400 MHz, CD₃OD) δ: 7.25-7.38 (m, |
| | 2H), 7.07-7.18 (m, 1H), 6.88-6.92 (m, 1H), 5.93-6.05 (m, 1H), 5.14 (d, J = 10 |
| | Hz, 1H), 5.00-5.08 (m, 1H), 4.50-4.72 (m, 4H), 4.15-4.21 (m, 4H), 3.98-4.15 |
| | (m, 1H), 3.60-3.80 (m, 1H), 3.36-3.52 (m, 1H), 3.07-3.18 (m, 1H), 2.93-3.06 |
| | (m, 1H), 2.01-2.15(m, 1H), 1.86-2.01(m, 1H), 1.38-1.41 (m, 3H), 1.35-1.38 |
| | (m, 4H), 1.25-1.29 (m, 5H). |
| NA10 | LC/MS: [M + H]⁺: 960.9. ¹H NMR (400 MHz, CD₃OD) δ: 7.79 (d, J = 7.6 |
| | Hz, 2H), 7.65 (d, J = 7.6 Hz, 2H), 7.34-7.40 (m, 3H), 7.16-7.33 (m, 5H), 7.06- |
| | 7.13 (m, 3H), 6.91 (qd, J₁ = 2.4 Hz, J₂ = 9.2 Hz, 1H), 5.92-6.02 (m, 1H), 5.10- |
| | 5.14 (m, 1H), 5.00-5.05 (m, 1H), 4.68-4.72 (m, 2H), 4.51 (s, 1H), 4.35-4.41 |
| | (m, 1H), 4.25-4.29 (m, 2H), 4.14-4.22 (m, 4H), 4.09-4.13 (m, 2H), 3.57-3.62 |
| | (m, 1H), 3.15-3.21 (m, 1H), 2.98-3.10 (m, 2H), 2.73-2.78 (m, 1H), 2.19 (dd, |
| | J = 7.6 Hz, 1H), 2.00-2.06 (m, 2H), 1.82-1.94 (m, 1H), 1.58-1.62 (m, 1H), |
| | 1.36 (s, 9H), 1.25 (t, J = 6.8 Hz, 3H). |
| NA12 | LC/MS: [M + H]⁺: 720.04. ¹H NMR (500 MHz, CD₃OD) δ: 7.31 (d, |
| | J = 2.5 Hz, 1H), 7.23-7.29 (m, 2H), 7.12-7.16 (m, 3H), 6.87-6.91 (m, 2H), |
| | 5.96-6.03 (m, 1H), 5.13 (dq, J₁ = 1.5 Hz, J₂ = 10.0 Hz, 1H), 5.01 (dq, J₁ = 1.5 |
| | Hz, J₂ = 17.0 Hz, 1H), 4.94 (t, J = 7.0 Hz, 1H), 4.72-4.74 (m, 2H), 4.66 (d, |
| | J = 14.5 Hz, 1H), 4.58 (s, 1H), 4.38 (dd, J₁ = 3.5 Hz, J₂ = 9.5 Hz, 1H), 4.13-4.22 |
| | (m, 4H), 4.02 (d, J = 14.5 Hz, 1H), 3.96 (d, J = 15.5 Hz, 1H), 3.83-3.89 (m, |
| | 1H), 3.15-3.20 (m, 2H), 3.00-3.04 (m, 2H), 2.95 (dd, J₁ = 3.5 Hz, J₂ = 13.5 Hz, |
| | 1H), 2.06 (dd, J₁ = 7.0 Hz, J₂ = 14.0 Hz, 1H), 1.87-1.94 (m, 1H), 1.47 (s, 9H), |
| | 1.27 (t, J = 7.0 Hz, 3H). |
| NA | LC/MS: [M + H]⁺: 691.97. ¹H NMR (400 MHz, CDCl₃) δ: 8.80 (brs, 1H), |
| | 7.95 (s, 1H), 7.26 (d, J = 9.2 Hz, 1H), 7.10-7.18 (m, 2H), 7.01-7.07 (m, 2H), |
| | 6.96 (s, 1H), 6.78-6.83 (m, 2H), 6.48-6.50 (m, 1H), 5.75-5.84 (m, 1H), 5.69 |
| | (d, J = 7.6 Hz, 1H), 5.05 (d, J = 10.4 Hz, 1H), 4.90-4.95 (m, 2H), 5.69 (dd, |
| | J₁ = 8.0 Hz, J₂ = 15.2 Hz, 1H), 4.61 (s, 1H), 4.44-4.53 (m, 3H), 4.11 (d, J = 16.0 |
| | Hz, 2H), 3.99 (d, J = 15.6 Hz, 1H), 3.89 (d, J = 14.8 Hz, 1H), 3.41-3.47 (m, |
| | 1H), 2.98-3.11 (m, 4H), 2.73 (t, J = 9.2 Hz, 1H), 1.72-1.89 (m, 2H), 1.44 (s, |
| | 9H). |
| NC1 | LC/MS: [M + H]⁺: 421.5. ¹H NMR (400 MHz, CD₃OD) δ: 7.18 (d, J = 8.4 |
| | Hz, 2H), 6.85 (d, J = 8.4 Hz, 2H), 4.52 (t, J = 7.2 Hz, 1H), 3.80-3.86 (m, 1H), |
| | 3.77 (s, 3H), 3.68 (s, 3H), 3.35-3.41 (m, 1H), 2.94 (dd, J₁ = 13.6 Hz, J₂ = 6.4 |
| | Hz, 1H), 2.69 (dd, J₁ = 13.6 Hz, J₂ = 8.0 Hz, 1H), 1.87-2.10 (m, 4H), 1.50-1.56 |
| | (m, 3H), 1.34-1.42 (m, 9H). |
| NC3 | LC/MS: [M + H]⁺: 578.3. ¹H NMR (400 MHz, CD₃OD) δ: 7.19 (d, J = 8.4 |
| | Hz, 2H), 6.86 (d, J = 8.4 Hz, 2H), 4.80-4.83 (m, 1H), 3.90-4.02 (m, 2H), 3.80- |
| | 3.86 (m, 1H), 3.76 (s, 3H), 3.68 (s, 3H), 3.26-3.31 (m, 2H), 3.02 (dd, J₁ = 13.6 |
| | Hz, J₂ = 7.6 Hz, 1H), 2.79 (dd, J₁ = 13.6 Hz, J₂ = 6.8 Hz, 1H), 2.03-2.10 (m, 1H), |
| | 1.85-2.00 (m, 3H), 1.47 (s, 3H), 1.45 (s, 9H), 1.15 (s, 9H), 1.08 (d, J = 5.6 Hz, |
| | 3H). |
| NC | LC/MS: [M + H]⁺: 564.4. ¹H NMR (400 MHz, CD₃OD) δ: 7.19 (d, J = 8.8 |
| | Hz, 2H), 6.83 (d, J = 8.8 Hz, 2H), 4.78-4.82 (m, 2H), 3.97-4.02 (m, 2H), 3.77- |
| | 3.81 (m, 1H), 3.75 (s, 3H), 3.04 (dd, J₁ = 14.0 Hz, J₂ = 7.2 Hz, 1H), 2.79 (dd, |
| | J₁ = 7.2 Hz, J₂ = 14.0 Hz, 1H), 2.11-2.18 (m, 1H), 1.86-1.97 (m, 3H), 1.49 (s, |
| | 3H), 1.45 (s, 9H), 1.42 (s, 9H), 1.08 (d, J = 6.0 Hz, 3H). |

### Example 2 Synthesis of Intermediate BD

### 1. Synthesis of Intermediate BD02

### 1). Preparation of Intermediate ND-OTos

TsCl (2.9 g, 383.6 mmol) and DMAP (4.29 g, 34.8 mmol) were dissolved in 300 mL of DCM, and triethylamine (52.8 g, 523.2 mmol) and 4-penten-1-ol (30 g, 348.8 mmol) were added at 0° C., respectively. Then the solution was warmed to room temperature and stirred for 3 hours. The reaction was monitored via LC/MS until 4-penten-1-ol was consumed completely. The reaction was extracted with 150 mL of DCM for three times and the organic phases were combined, washed twice with 500 mL of saturated saline solution, dried over anhydrous Na₂SO₄ and filtered. The filtrate was rotary evaporated to dryness under reduced pressure to obtain crude product, which was purified by silica gel column chromatography to obtain 83 g of ND-OTos as a yellow oil with a yield of 99%.

### 2). Preparation of Intermediate ND3

ND-OTos (52.37 g, 217.2 mmol) and ND2 (60 g, 239 mmol) were dissolved in 800 mL of CH₃CN. K₂CO₃ (89.96 g, 651.6 mmol) was added at 0° C., and then the solution was heated to 85° C. and stirred for 12 hours. The reaction was monitored via LC/MS until ND-OTos was consumed completely. K₂CO₃ was filtered and rinsed with 500 mL of EA, and the filtrate was rotary evaporated to dryness under reduced pressure to obtain crude product, which was purified by silica gel column chromatography to obtain 35 g of ND3 as a yellow oil with a yield of 50%.

### 3). Preparation of Intermediate BD01-1

Intermediate ND3 (13 g, 40.8 mmol) and methyl 4-carboxyphenylacetate (8.72 g, 53 mmol) were dissolved in 100 mL of DMF, and DIPEA (21.3 mL, 122.4 mmol) was added at 0° C. Then the solution was stirred for 15 minutes, and then HATU (18.6 g, 49 mmol) was added. The solution was slowly warmed to room temperature reacted for 5 hours. The reaction was monitored via LC/MS until intermediate ND3 was consumed completely. 200 mL of water and 200 mL of EA were added to the reaction solution, which was then extracted with 100 mL of EA for three times. The organic phases were combined, washed twice with saturated saline solution, then dried over anhydrous Na₂SO₄ and filtered. The filtrate was rotary evaporated to dryness under reduced pressure to obtain crude product, which was purified by silica gel column chromatography to obtain 16.8 g of BD01-1 as a yellow oil with a yield of 83%.

### 4). Preparation of Intermediate BD01-2

BD01-1 (12.5 g, 25.2 mmol) was dissolved in 100 mL of DCM, 20 mL of TFA was added dropwise at 0° C. The reaction mixture was stirred for 2 hours, The reaction was monitored via LC/MS until BD01-1 was consumed completely The reaction solution was rotary evaporated to dryness under reduced pressure to obtain crude BD01-2, which can be directly used for the next reaction step without purification. LC/MS: [M+H]⁺: 395.2.

### 5). Preparation of Intermediate BD01

The above crude BD01-2 was dissolved in 160 mL of THF/H₂O (v/v=1:1), and an aqueous solution of LiOH (10.6 g, 252 mmol) was added at 0° C. The reaction mixture was slowly warmed to room temperature and continuously reacted for 5 hours, which was monitored via LC/MS until starting materials were consumed completely. The pH was adjusted to 5-6 with 1 M acetic acid, and then THE was rotary evaporated off under reduced pressure, and the remaining aqueous phase was purified by preparative chromatography to obtain 8.6 g of BD01 as a white solid with a yield of 86%.

### 2. Synthesis of Intermediate BD02

### 1). Preparation of Intermediate BD02-1

Raw material methyl 4-carboxyphenylacetate (40.0 g, 206.2 mmol) was dissolved in 200 mL of dry THF, which was purged with nitrogen for three times, and 1M LiHMDS (412.4 mL, 412.4 mmol) was slowly added dropwise at 0° C. The reaction mixture was reacted at 0° C. for 2 hours, followed by dropwise addition of CH₃I (12.44 mL, 195.8 mmol), and then gradually warmed to room temperature, and reacted at room temperature for 12 hours. Then, 0.1 M acetic acid aqueous solution was added to the reaction mixture, which was then extracted with 100 mL of EA for three times. The organic phases were combined, and then dried over anhydrous Na₂SO₄ and filtered. The filtrate was rotary evaporated to dryness under reduced pressure to obtain crude product, which was purified by preparative chromatography to obtain 9.86 g of BD02-1 as a pale-yellow oil with a yield of 23%.

### 2). Preparation of Intermediate BD02-2

BD02-2 was prepared with reference to the synthetic method of BD01-1. The crude product was purified by silica gel column chromatography to obtain 14.2 g of BD02-2 as a yellow oil with a yield of 87%.

### 3). Preparation of Intermediate BD02-3

BD02-3 was prepared with reference to the synthetic method of BD01-2. The organic solvent was rotary evaporated to dryness under reduced pressure to obtain crude product, which was directly used for the next reaction without purification. LC/MS: [M+H]⁺: 409.4.

### 4). Preparation of Intermediate BD02

BD02 was prepared with reference to the synthetic method of BD01. THE was rotary evaporated off under reduced pressure, and the remaining aqueous phase was purified by preparative chromatography to obtain 10.4 g of BD02 as a white solid with a yield of 66%.

### 3. Synthesis of Intermediate BD03

### 1). Preparation of Intermediate BD03-1

Raw material methyl 4-carboxyphenylacetate (20.0 g, 103.1 mmol) was dissolved in 100 mL of dry THF, which was degassed with nitrogen for three times, and 1M LiHMDS (464.0 mL, 464.0 mmol) was slowly added dropwise at 0° C. The reaction mixture was reacted for 1 hours, followed by dropwise addition of CH₃I (17.97 mL, 288.7 mmol), and then gradually warmed to room temperature, and reacted at room temperature for 12 hours. The reaction was monitored via LC/MS until the raw materials were consumed completely. 0.1 M acetic acid aqueous solution was added dropwise to the reaction system, which was then extracted with 50 mL of EA for three times. The organic phases were combined, then dried over anhydrous Na₂SO₄ and filtered. The filtrate was rotary evaporated to dryness under reduced pressure to obtain crude product, which was purified by preparative chromatography to obtain 8.0 g of BD03-1 as a pale-yellow oil with a yield of 35%.

### 2). Preparation of Intermediate BD03-2

BD03-2 was prepared with reference to the synthetic method of BD01-1. The crude product was purified by silica gel column chromatography to obtain 9.41 g of BD03-2 as a yellow oil with a yield of 80%.

### 3). Preparation of Intermediate BD03-3

BD03-3 was prepared with reference to the synthetic method of BD01-2. The reaction solution was rotary evaporated to dryness to obtain crude product, which was used directly for the next reaction step without purification. LC/MS: [M+H]⁺: 423.4.

### 4). Preparation of Intermediate BD03

BD03 was prepared with reference to the synthetic method of BD01. THE was rotary evaporated off under reduced pressure, and the remaining aqueous phase was purified by preparative chromatography to obtain 7.72 g of white solid with a yield of 84%.

### 4. Synthesis of Intermediate BD04

### 1). Preparation of Intermediate BD04-1

Pd(OAc)₂ (850 mg, 3.75 mmol) and Xantphos (6.5 g, 11.26 mmol) were dissolved in 95 mL of DMF. The solution was degassed with nitrogen for three times, and then formic acid (19.6 mL, 525.85 mmol), S1 (19 g, 75.12 mmol), and Et₃N (20.8 mL, 150.24 mmol) were added in sequence. A solution of DCC (31 g, 150.24 mmol) in 100 mL of DMF was added dropwise at 0° C. Then the reaction temperature was slowly warmed to 80° C. and reacted for 18 hours. Then the reaction was monitored via LC/MS until the amount of BD04-1 was very low. The reaction solution was cooled to room temperature, and degassed with nitrogen. Pd(OAc)₂ (850 mg, 3.75 mmol), formic acid (9.8 mL, 262.92 mmol), and Et₃N (10.4 mL, 75.12 mmol) were supplemented, and DCC was added dropwise under ice bath condition. The mixture was reacted at 80° C. for 24 hours, and the reaction was monitored via LC/MS until the raw material S1 was consumed completely. The reaction solution was diluted with 50 mL of EA, and filtered. The filter cake was rinsed with 30 mL of EA, and 100 mL of water was added to the filtrate, which was extracted twice with 50 mL of EA. The organic phases were combined, washed twice with saline solution, dried over anhydrous Na₂SO₄, and filtered. The filtrate was rotary evaporated to dryness under reduced pressure to obtain crude product, which was purified by silica gel column chromatography to obtain 7.3 g of BD04-1 as a white solid with a yield of 44%.

### 2). Preparation of Intermediate BD04-2

BD04-2 was prepared with reference to the synthetic method of BD01-1. The crude product was purified by silica gel column chromatography to obtain 11.4 g of BD04-2 as a white solid with a yield of 66%.

### 3). Preparation of Intermediate BD04-3

BD04-3 was prepared with reference to the synthetic method of BD01-2. The organic solvent was rotary evaporated off under reduced pressure to obtain crude product, which was directly used for the next reaction without purification. LC/MS: [M+H]⁺: 421.3.

### 4). Preparation of Intermediate BD04

BD04 was prepared with reference to the synthetic method of BD01. THE was rotary evaporated off under reduced pressure, and the remaining aqueous phase was purified by preparative chromatography to obtain 6.2 g of white solid with a yield of 72%.

### 5. Synthesis of Intermediate BD16

### 1). Preparation of Intermediate BD16-1

SM1 (27 g, 121.59 mmol), Xantphos (7 g, 12.15 mmol), and K₂CO₃ (67 g, 406.37 mmol) were dissolved in 270 mL of 1,4-dioxane. The solution was degassed with nitrogen for three times, followed by addition of Pd(PPh₃)₄ (7 g, 6.07 mmol) and CuI (1.15 g, 6.07 mmol), and degassed with nitrogen for another three times, then reacted for 12 hours in the oil bath at 80° C. After the reaction was completed, the reaction system was diluted with water to 800 mL, and the mixture was extracted with 150 mL of EA for three times. The organic phases were combined, and then dried over anhydrous Na₂SO₄ and filtered. The filtrate was rotary evaporated to dryness under reduced pressure to obtain crude product, which was purified by preparative chromatography to obtain 10.3 g of BD16-1 as a yellow oil with a yield of 28%.

### 2). Preparation of Intermediate BD16-2

BD16-1 (7.59 g, 25.3 mmol) was dissolved in DCM (40 mL), TFA (8 mL, 104.5 mmol) was added at 0° C. The reaction mixture was reacted at room temperature for 2 hours and monitored via LC/MS until BD16-1 was consumed completely. DCM and TFA were rotary evaporated off under reduced pressure to obtain 6.1 g of crude BD16-2 as a yellow oil, which was used directly for the next reaction without purification. 3). Preparation of Intermediate BD16-3 BD16-3 was prepared with reference to the synthetic method of BD01-1. The crude product was purified by silica gel column chromatography to obtain 8.71 g of BD16-3 as a yellow oil with a yield of 64%.

### 4). Preparation of Intermediate BD16-4

BD16-4 was prepared with reference to the synthetic method of BD01-2. The reaction solvent was rotary evaporated to dryness under reduced pressure to obtain 7.52 g of BD16-4 as a yellow oil, and the product was used directly for the next reaction without purification. LC/MS: [M+H]⁺: 445.2.

### 5). Preparation of Intermediate BD16

BD16 was prepared with reference to the synthetic method of BD02. THE was rotary evaporated off under reduced pressure, and the remaining aqueous phase was purified by preparative chromatography to obtain 6.9 g of BD16 as a white solid with a yield of 97%.

### 6. Synthesis of Intermediate BD10

### 1). Preparation of Intermediate BD10-1

3-iodobenzoate (15.0 g, 57.24 mmol) was dissolved in 150 mL of DMF, and tert-butyl acrylate (36.68 g, 286.2 mmol), palladium acetate (1.3 g, 5.724 mmol), triethylamine (17.38 g, 171.72 mmol), and tetrabutylammonium iodide (2.11 g, 5.724 mmol) were added, the solution was reacted at 120° C. for 3 hours. The reaction was monitored to be ended via LC/MS. The reaction solution was filtered, the filtrate was rotary evaporated to dryness under reduced pressure to obtain crude product, which was purified by silica gel column chromatography to obtain 12.91 g of yellow oil with a yield of 86%.

### 2). Preparation of Intermediate BD10-2

BD10-1 (10.1 g, 28.88 mmol) was dissolved in 100 mL of methanol, and 2.0 g of 10% Pd/C was added, the system was replaced with hydrogen for three times, and reacted at room temperature for 12 hours, the reaction was monitored to be complete via LC/MS, then the reaction solution was filtered, the filtrate was rotary evaporated to dryness under reduced pressure to obtain crude product, which was purified by silica gel column chromatography to obtain 9.26 g of yellow oil with a yield of 91%.

### 3). Preparation of Intermediate BD10-3

BD10-2 (8.0 g, 30.29 mmol) was dissolved in a mixed solvent of 75 mL acetonitrile and 3 mL water, and LiBr (15.78 g, 181.74 mmol) and DIPEA (23.49 g, 181.74 mmol) were added, the solution was reacted at 85° C. for 3 hours. The raw materials were monitored to be consumed completely via LC/MS. Then, the reaction solution was adjusted to be pH=5-6 with 1 M acetic acid aqueous solution. Then the mixture was extracted with 30 mL of EA for three times, dried over anhydrous Na₂SO₄, the organic solvent was rotary evaporated to dryness to obtain crude product, which was purified by silica gel column chromatography to obtain 6.7 g of yellow oil with a yield of 88%.

### 4). Preparation of Intermediate BD10-4

BD10-4 was prepared with reference to the synthetic method of BD01-1. The organic solvent was rotary evaporated to dryness to obtain crude product, which was purified by silica gel column chromatography to obtain 10 g of BD10-4 as a yellow oil with a yield of 83%.

### 5). Preparation of Intermediate BD10

BD10 was prepared with reference to the synthetic method of BD01. THE was rotary evaporated off under reduced pressure, and the remaining aqueous phase was purified by preparative chromatography to obtain 6.79 g of BD10 as a yellow oil with a yield of 96%.

### 7. Synthesis of Intermediate BD12

### 1). Preparation of Intermediate BD12-3

BD10-2 (49 g, 185.4 mmol) was dissolved in 500 mL of DCM, and 100 mL of TFAwas added in an ice bath, the solution was reacted at room temperature for 2 hours. The reaction was monitored to be ended via LC/MS. The reaction solution was rotary evaporated to dryness and then used directly for the next reaction step. LC/MS: [M+H]⁺: 209.1.

### 2). Preparation of Intermediate BD12-4

The above crude product BD12-3 was dissolved in 500 mL of DMF, DIPEA (65.35 g, 505.65 mmol) and HATU (76.9 g, 202.26 mmol) were added in an ice bath, the solution was reacted for 30 minutes, ND₃ (53.68 g, 168.55 mmol) was added, and then reacted at room temperature for 3 hours. The reaction was monitored to be ended via LC/MS. The reaction solution was added dropwise into water, the solution was extracted with 300 mL of EA for three times, the organic phase was washed twice with semi-saturated ammonium chloride aqueous solution and twice with saturated saline solution, the organic phases were combined, and then dried over anhydrous Na₂SO₄ and filtered. The filtrate was rotary evaporated to dryness under reduced pressure to obtain crude product, which was purified by silica gel column chromatography to obtain 87 g of yellow oil with a yield of 92%.

### 3). Preparation of Intermediate BD12-5

BD12-4 (86.5 g, 168.55 mmol) was dissolved in 800 mL of DCM, and 160 mL of TFA was added in an ice bath, the solution was reacted at room temperature for 2 hours. The reaction was monitored to be ended via LC/MS. The reaction solution was rotary evaporated to dryness and then used directly for the next reaction step. LC/MS: [M+H]⁺: 409.4.

### 4). Preparation of Intermediate BD12

BD12 was prepared with reference to the synthetic method of BD01. THF was rotary evaporated off under reduced pressure, and the remaining aqueous phase was purified by preparative chromatography to obtain 57.1 g of BD12 as a yellow oil with a yield of 85%.

### 8. Synthesis of Intermediate BD21

### 1). Preparation of Intermediate BD21-1

S1 (37.54 g, 173.8 mmol) was dissolved in 220 mL of THF, and 60% NaH (5.55 g, 138.8 mmol) was added in batches at 0° C., the solution was reacted for 2 hours at 0° C., and then S2 (22.0 g, 115.7 mmol) was added dropwise, the system was slowly returned to room temperature and reacted for 12 hours. The reaction was monitored via LC/MS until S1 was consumed completely, 0.1 M acetic acid solution was added to the system to adjust the pH to 6-7. Then the mixture was extracted with 150 mL of EA for three times. The organic phases were combined, and then dried over anhydrous Na₂SO₄ and filtered. The filtrate was rotary evaporated to dryness under reduced pressure to obtain 19.78 g of crude BD21-1 as a yellow oil. The crude product was used directly for the next reaction step without purification. LC/MS: [M+H]⁺: 387.2.

### 2). Preparation of Intermediate BD21-2

The above crude product BD21-1 (16.50 g, 42.7 mmol) was dissolved in 300 mL of THF, and 300 mL aqueous solution of NaOH (2.66 g, 63.8 mmol) was added at 0° C., the solution was slowly returned to room temperature and reacted. The reaction was monitored via LC/MS until BD21-1 was consumed completely after 12 hours, 0.1 M acetic acid solution, water and EA were added to the reaction solution to adjust the pH to 6. Then the mixture was extracted with 150 mL of EA for three times. The organic phases were combined, and then dried over anhydrous Na₂SO₄ and filtered. The filtrate was rotary evaporated to dryness under reduced pressure to obtain crude product, which was purified by silica gel column chromatography to obtain 10.37 g of BD21-2 as a transparent oil with a yield of 66%.

### 3). Preparation of Intermediate BD21-3

BD21-3 was prepared with reference to the synthetic method of BD01-1. The crude product was purified by silica gel column chromatography to obtain 11.43 g of BD21-3 as a yellow oil with a yield of 82%.

### 4). Preparation of Intermediate BD21

BD21-3 (11.28 g, 16.8 mmol) was dissolved in DCM (100 mL), TFA (30 mL, 391.8 mmol) was added at 0° C., and then the solution was stirred at room temperature for 24 hours, the reaction was monitored via LC/MS until BD21-3 was consumed completely. The reaction was stopped, DCM and TFA were rotary evaporated off under reduced pressure, and excess sodium bicarbonate aqueous solution was further added to pH 8-9, the aqueous phase was purified by preparative chromatography to obtain 4.25 g of BD21 as a white solid with a yield of 61%.

### 9. Synthesis of Intermediate BD07

### 1). Preparation of Intermediate BD07-1

Tert-butyl 2-bromobenzoate (15.0 g, 58.3 mmol) was dissolved in 100 mL of 1,4-dioxane and 100 mL of water, and K₂CO₃ (16.1 g, 116.6 mmol), Pd(dppf)Cl₂ (4.26 g, 5.83 mmol), and 3-methoxycarbonylphenylboronic acid pinacol ester (16 g, 61.2 mmol) were added, respectively, the solution was degassed with nitrogen for four times and reacted at 80° C. for 3 hours. The reaction was monitored via LC/MS until tert-butyl 2-bromobenzoate was consumed completely. Water and EA were added to the reaction system, which was then extracted with 100 mL of EA for three times. The organic phases were combined, and then washed twice with saturated saline solution, then dried over anhydrous Na₂SO₄ and filtered. The filtrate was rotary evaporated to dryness under reduced pressure to obtain crude product, which was purified by silica gel column chromatography to obtain 18 g of BD07-1 as a yellow oil with a yield of 98%.

### 2). Preparation of Intermediate BD07-2

Intermediate BD07-1 (18 g, 57.7 mmol) was dissolved in DCM (180 mL), TMSI (17.3 g, 86.5 mmol) was added at 0° C., and then the solution was stirred at room temperature for 3 hours, the reaction was monitored via LC/MS until intermediate BD07-1 was consumed completely. The reaction was quenched with water at 0° C., DCM was rotary evaporated to dryness under reduced pressure, and then extracted with 50 mL of EA for three times. The organic phases were combined, and then washed twice with saturated saline solution, then dried over anhydrous Na₂SO₄, and concentrated to obtain 16.8 g of crude BD07-2 as a yellow oil, which was used directly for the next reaction step without purification. LC/MS:[M−H]⁻:255.0.

### 3). Preparation of Intermediate BD07-3

Intermediate ND3 (14 g, 43.9 mmol) and intermediate BD07-2 (16.8 g, 65.85 mmol) were dissolved in 200 mL of DMF, and DIPEA (30.5 mL, 175.6 mmol) was added at 0° C. Then the solution was stirred for 15 minutes under an ice bath, and then HATU (25 g, 65.85 mmol) was further added, the solution was slowly returned to room temperature and reacted for 5 hours, the reaction was monitored via LC/MS to find that ND3 was consumed completely. 500 mL of water and 500 mL of EA were added to the system, which was then extracted with 300 mL of EA for three times. The organic phases were combined, and then washed twice with saturated saline solution, then dried over anhydrous Na₂SO₄, the organic solvent was rotary evaporated to dryness to obtain crude product, which was purified by silica gel column chromatography to obtain 24 g of intermediate BD07-3 as a yellow oil with a yield of 98%. LC/MS: [M+H]⁺: 557.4.

### 4). Preparation of Intermediate BD07-4

Intermediate BD07-3 (12 g, 21.58 mmol) was dissolved in 100 mL of DCM, 20 mL of TFA was added dropwise at 0° C., the solution was then continuously stirred for 2 hours, the reaction was monitored via LC/MS to find that BD07-3 was consumed completely, the organic solvent was rotary evaporated to dryness to obtain crude BD07-4. LC/MS: [M+H]⁺: 457.4.

### 5). Preparation of Intermediate BD07

The above crude BD07-4 was dissolved in 160 mL of THF/H₂O (v/v=1:1), and an aqueous solution of LiOH (9.06 g, 215.8 mmol) was added at 0° C., the solution was slowly returned to room temperature and reacted for 5 hours, the reaction was monitored via LC/MS until the raw materials were consumed completely, THF was then rotary evaporated to dryness, and the remaining aqueous phase was purified by preparative chromatography to obtain 7.5 g of BD07 as a white solid with a yield of 78%.

### Synthesis of Intermediate E

### 1. Synthesis of Intermediate NE2C

### 1). Preparation of Intermediate NE2C-1

Tert-butyl 2-bromoacetate (10 g, 51.28 mmol) was dissolved in 100 mL of acetonitrile, a solution of 2 M trimethylamine in methanol (76.92 mL, 153.85 mmol) was added at room temperature, the solution was reacted at 50° C. for 12 hours, the reaction was monitored via LC/MS to find that NE2C-1 was consumed completely, the solvent methanol and acetonitrile in the reaction system were rotary evaporated to dryness to obtain the crude product, which was used directly for the next reaction step without purification. LC/MS: [M]⁺: 174.17.

### 2). Preparation of Intermediate NE2C

The above crude NE2C-2 was dissolved in DCM, a solution of 4 M 1,4-dioxane in hydrochloric acid (64 mL, 256.41 mmol) was added at room temperature, the solution was stirred at room temperature, the reaction was monitored via LC/MS to find that NE2C-1 was consumed completely, DCM was rotary evaporated to dryness to obtain the crude product, which was purified by preparative chromatography to obtain 8.53 g of NE2C as a white solid with a two-step yield of 84%.

### 2. Synthesis of Intermediate NE4C

### 1). Preparation of Intermediate NE4C-1

NE4C-1 was prepared with reference to the synthetic method of NE2C-1, where tert-butyl 4-bromopropionate was used instead of tert-butyl 2-bromoacetate. LC/MS: [M]⁺: 202.16.

### 2). Preparation of Intermediate NE4C

NE4C was prepared with reference to the synthetic method of NE2C, where NE4C-1 was used instead of NE2C-1. The crude product was purified by preparative chromatography to obtain 7.12 g of NE4C as a white solid with a two-step yield of 70%.

### 3. Synthesis of Intermediate NE5C

### 1). Preparation of Intermediate NE5C-1

NE5C-1 was prepared with reference to the synthetic method of NE2C-1, where tert-butyl 3-bromovalerate was used instead of tert-butyl 2-bromoacetate. LC/MS: [M]⁺: 216.18.

### 2). Preparation of Intermediate NE5C

NE5C was prepared with reference to the synthetic method of NE2C, where NE5C-1 was used instead of NE2C-1. The crude product was purified by preparative chromatography to obtain 7.53 g of NE5C as a white solid with a two-step yield of 74%.

### 4. Synthesis of Intermediate NE6C

### 1). Preparation of Intermediate NE6C-1

NE6C-1 was prepared with reference to the synthetic method of NE2C-1, where tert-butyl 3-bromohexanoate was used instead of tert-butyl 2-bromoacetate. LC/MS: [M]⁺: 230.2.

### 2). Preparation of Intermediate NE6C

NE6C was prepared with reference to the synthetic method of NE2C, where NE6C-1 was used instead of NE2C-1. The crude product was purified by preparative chromatography to obtain 6.79 g of NE6C as a white solid with a two-step yield of 67%.

### 5. Synthesis of Intermediate NE7C

### 1). Preparation of Intermediate NE7C-1

NE7C-1 was prepared with reference to the synthetic method of NE2C-1, where tert-butyl 3-bromoheptanoate was used instead of tert-butyl 2-bromoacetate. LC/MS: [M]⁺: 244.2.

### 2). Preparation of Intermediate NE7C

NE7C was prepared with reference to the synthetic method of NE2C, where NE7C-1 was used instead of NE2C-1. The crude product was purified by preparative chromatography to obtain 7.09 g of NE7C as a white solid with a two-step yield of 72%.

### 6. Synthesis of Intermediate NE15C

### 1). Preparation of Intermediate NE15C-1

NE15C-1 was prepared with reference to the synthetic method of NE2C-1, where tert-butyl 3-bromopentadecanoate was used instead of tert-butyl 2-bromoacetate. LC/MS: [M]⁺: 356.3.

### 2). Preparation of Intermediate NE15C

NE15C was prepared with reference to the synthetic method of NE2C, where NE15C-1 was used instead of NE2C-1. The crude product was purified by preparative chromatography to obtain 8.31 g of NC15C as a white solid with a two-step yield of 82%.

### 7. Synthesis of Intermediate E10

### 1). Preparation of Intermediate E10-1

SM1 (9.0 g, 67.59 mmol) was dissolved in 180 mL of THF, K₂CO₃ (46.6 g, 337.96 mmol) was added, and then CH₃I (21 mL, 337.96 mmol) was further added, the tube was sealed, and the solution was reacted overnight at room temperature. The reaction was monitored via LC/MS until SM1 was consumed completely. The reaction solution was filtered, the filter cake was rinsed with THF, the filtrate was rotary evaporated to dryness to obtain 22 g of crude E10-1. LC/MS: [M]⁺: 190.07.

### 2). Preparation of Intermediate E10

The crude E10-1 (22 g, 69.36 mmol) was dissolved in 200 mL of H₂O, and NaOH (4.16 g, 69.36 mmol) was added at 0° C., the solution was reacted at room temperature for 10 minutes. The reaction was monitored via LC/MS until E10-1 consumed completely. The reaction solution was purified by preparative chromatography to obtain 8.20 g of E10 as a white solid with a yield of 57%.

### 8. Synthesis of Intermediate E20

### 1). Preparation of Intermediate E20-1

E20-1 was prepared with reference to the synthetic method of E10-1, where SM2 was used instead of SM1. LC/MS: [M]⁺: 276.2.

### 2). Preparation of Intermediate E20

E20 was prepared with reference to the synthetic method of E10, where E20-1 was used instead of E10-1. The crude product was purified by preparative chromatography to obtain 7.68 g of E20 as a white solid with a two-step yield of 57%.

### 9. Synthesis of Intermediate E30

### 1). Preparation of Intermediate E30-1

E30-1 was prepared with reference to the synthetic method of E10-1, where SM3 was used instead of SM1. LC/MS: [M]⁺: 320.1.

### 2). Preparation of Intermediate E30

E30 was prepared with reference to the synthetic method of E10, where E30-1 was used instead of E30-1. The crude product was purified by preparative chromatography to obtain 8.66 g of E20 as a white solid with a two-step yield of 68%.

TABLE 2
Structural Identification Data In the Synthesis Route of Intermediates

| Nos. | LC/MS and ¹H NMR data |
|---|---|
| ND- | ¹H NMR (400 MHz, CD₃OD) δ: 7.77-7.79 (m, 1H), 7.70-7.72 (m, 1H), |
| OTos | 7.45 (d, J = 8.5 Hz, 1H), 7.24 (d, J = 8.0 Hz, 1H), 5.67-5.75 (m, 1H), 4.91-4.96 |
| | (m, 2H), 4.03 (t, J = 6.5 Hz, 2H), 2.46 (s, 3H), 2.03-2.08 (m, 2H), 1.68-1.73 |
| | (m, 2H). |
| ND3 | LC/MS: [M + H]⁺: 319.3. ¹H NMR (400 MHz, CD₃OD) δ: 7.42 (d, J = 6.0 |
| | Hz, 2H), 7.33 (d, J = 6.0 Hz, 2H), 5.77-5.87 (m, 1H), 5.02-5.12 (m, 2H), 4.17 |
| | (s, 2H), 3.26 (t, J = 9.0 Hz, 2H), 3.00-3.04 (m, 2H), 2.80 (t, J = 9.0 Hz, 2H), |
| | 2.16 (q, J = 9.0 Hz, 2H), 1.77-1.84 (m, 2H), 1.42 (s, 9H). |
| BD01-1 | LC/MS: [M + H]⁺: 495.2. ¹H NMR (400 MHz, CD₃OD) δ: 7.30-7.39 |
| | (m, 5H), 7.22 (t, J = 8.0 Hz, 2H), 7.09 (d, J = 7.2 Hz, 1H), 5.50-5.88 (m, 1H), |
| | 4.95-5.06 (m, 1H), 4.80-4.85 (m, 1H), 4.74 (s, 1H), 4.52 (s, 1H), 3.67-3.72 |
| | (m, 5H), 3.43 (t, J = 7.2 Hz, 1H), 3.24-3.26 (m, 2H), 3.17-3.21 (m, 1H), 2.72- |
| | 2.78 (m, 2H), 2.07-2.10 (m, 1H), 1.79-1.83 (m, 1H), 1.72-1.76 (m, 1H), |
| | 1.57-1.62 (m, 1H), 1.41 (s, 9H). |
| BD01 | LC/MS: [M + H]⁺: 381.2. ¹H NMR (400 MHz, CD₃OD) δ: 7.31-7.44 |
| | (m, 5H), 7.11-7.24 (m, 3H), 5.54-5.87 (m, 1H), 4.82-5.05 (m, 2H), 4.74 (s, |
| | 1H), 4.54 (s, 1H), 3.51 (d, J = 8.8 Hz, 2H), 3.41 (t, J = 6.0 Hz, 1H), 3.22 (t, |
| | J = 6.0 Hz, 1H), 2.74-2.85 (m, 4H), 2.08-2.09 (m, 1H), 1.60-1.82 (m, 3H). |
| BD02-1 | LC/MS: [M − H]⁻: 207.1. ¹H NMR (500 MHz, CDCl₃) δ: 7.97 (dt, J₁ = 2.0 |
| | Hz, J₂ = 8.5 Hz, 2H), 7.39 (dt, J₁ = 2.0 Hz, J₂ = 8.5 Hz, 2H), 3.86 (q, J = 7.0 Hz, |
| | 1H), 3.65 (s, 3H), 1.48 (d, J = 7.0 Hz, 3H). |
| BD02-2 | LC/MS: [M-^{t}Bu + 2H]⁺: 453.4. ¹H NMR (500 MHz, CD₃OD) δ: 7.34- |
| | 7.42 (m, 4H), 7.30 (d, J = 9.5 Hz, 1H), 7.21 (t, J = 10.0 Hz, 2H), 7.09 (d, J = 10.0 |
| | Hz, 1H), 5.50-5.83 (m, 1H), 4.94-5.06 (m, 1H), 4.78 (s, 1H), 4.74 (s, 1H), |
| | 4.51 (s, 1H), 3.80-3.85 (m, 1H), 3.64 (d, J = 5.5 Hz, 1H), 3.42 (t, J = 10.0 Hz, |
| | 1H), 3.25 (t, J = 10.0 Hz, 1H), 3.18 (t, J = 10.0 Hz, 1H), 2.73-2.78 (m, 2H), |
| | 2.06-2.11 (m, 1H), 1.72-1.84 (m, 2H), 1.57-1.61 (m, 1H), 1.45-1.49 (m, 3H), |
| | 1.41 (s, 9H). |
| BD02 | LC/MS: [M + H]⁺: 394.97. ¹H NMR (500 MHz, CD₃OD) δ: 7.48 (d, |
| | J = 8.0 Hz, 1H), 7.43 (d, J = 8.0 Hz, 1H), 7.32-7.35 (m, 3H), 7.23 (dd, J₁ = 8.0 |
| | Hz, J₂ = 15.5 Hz, 2H), 7.11 (d, J = 7.5 Hz, 1H), 5.53-5.86 (m, 1H), 4.94-5.06 |
| | (m, 1H), 4.82-4.85 (m, 1H), 4.74 (s, 1H), 4.53 (s, 1H), 3.58-3.65 (m, 1H), |
| | 3.41 (t, J = 8.0 Hz, 1H), 3.21 (t, J = 8.0 Hz, 1H), 2.89-2.94 (m, 2H), 2.76-2.81 |
| | (m, 2H), 2.06-2.11 (m, 1H), 1.79-1.83 (m, 1H), 1.70-1.76 (m, 1H), 1.57- |
| | 1.63 (m, 1H), 1.40-1.44 (m, 3H). |
| BD03-1 | LC/MS: [M + H]⁺: 223.1. ¹H NMR (500 MHz, CD₃OD) δ: 7.97 (d, |
| | J = 10.5 Hz, 2H), 7.43 (d, J = 10.5 Hz, 2H), 3.65 (s, 3H), 1.57 (s, 6H). |
| BD03-2 | LC/MS: [M-^{t}Bu + 2H]⁺: 467.4. 1H NMR (500 MHz, CD₃OD) δ: 7.40- |
| | 7.46 (m, 4H), 7.08-7.31 (m, 4H), 5.51-5.85 (m, 1H), 4.95-5.06 (m, 1H), |
| | 4.74-4.80 (m, 2H), 4.51(s, 1H), 3.64 (d, J = 5.0 Hz, 3H), 3.43 (t, J = 7.0 Hz, |
| | 1H), 3.15-3.28 (m, 3H), 2.73-2.78 (m, 2H), 2.06-2.12 (m, 1H), 1.72-1.84 |
| | (m, 2H), 1.57 (d, J = 14.5 Hz, 6H), 1.54-1.62 (m, 1H), 1.41 (s, 9H)). |
| BD03 | LC/MS: [M + H]⁺: 409.4. ¹H NMR (500 MHz, CD₃OD) δ: 7.48-7.52 |
| | (m, 2H), 7.12-7.35 (m, 6H), 5.58-5.82 (m, 1H), 4.94-5.05 (m, 1H), 4.83 (s, |
| | 1H), 4.74 (s, 1H), 4.54 (s, 1H), 3.39-3.43 (m, 1H), 3.21-3.25 (m, 1H), 2.84- |
| | 2.88 (m, 2H), 2.73-2.78 (m, 2H), 2.06-2.12 (m, 1H), 1.72-1.84 (m, 2H), |
| | 1.58-1.63 (m, 1H), 1.50 (s, 6H). |
| BD04-1 | LC/MS: [M − H⁻] : 219.4. ¹H NMR (500 MHz, CDCl₃) δ: 7.98 (t, J = 2.0 |
| | Hz, 2H), 7.39 (dt, J₁ = 2.0 Hz, J₂ = 8.5 Hz, 2H), 3.60 (s, 3H), 1.62 (q, J = 4.0 |
| | Hz, 2H), 1.19 (q, J = 4.0 Hz, 2H). |
| BD04-2 | LC/MS: [M-^{t}Bu + 2H]⁺: 465.5. ¹H NMR (500 MHz, CD₃OD) δ: 7.46 |
| | (d, J = 7.5 Hz, 1H), 7.40 (t, J = 8.5 Hz, 1H), 7.36-7.38 (m, 2H), 7.31 (d, J = 7.5 |
| | Hz, 1H), 7.22 (dd, J₁ = 7.0 Hz, J₂ = 12.0 Hz, 2H), 5.51-5.85 (m, 1H), 4.95-5.06 |
| | (m, 1H), 4.80-4.84 (m, 1H), 4.74 (s, 1H), 4.52 (s, 1H), 3.60 (d, J = 9.5 Hz, |
| | 3H), 3.42 (t, J = 7.5 Hz, 1H), 3.25 (q, J = 8.0 Hz, 2H), 3.20 (t, J = 7.5 Hz, 1H), |
| | 2.75 (q, J = 8.5 Hz, 2H), 2.10 (q, J = 7.0 Hz, 1H), 1.83 (q, J = 7.0 Hz, 1H), 1.74 |
| | (quintet, J = 7.5 Hz, 1H), 1.57-1.62 (m, 3H), 1.41 (s, 9H), 1.0-1.25 (m, 2H). |
| BD04 | LC/MS: [M + H]⁺: 407.4. ¹H NMR (500 MHz, CD₃OD) δ: 7.35(d, J = 8.0 |
| | Hz, 1H), 7.26-7.29 (m, 3H), 7.23 (dd, J₁ = 2.5 Hz, J₂ = 8.5 Hz, 2H), 7.15 (d, |
| | J = 8.0 Hz, 1H), 7.06 (d, J = 7.5 Hz, 1H), 5.48-5.81 (m, 1H), 4.88-4.98 (m, |
| | 1H), 4.66-4.68 (m, 1H), 4.64 (s, 1H), 4.49 (s, 1H), 3.34 (t, J = 7.5 Hz, 1H), |
| | 3.19 (t, J = 7.5 Hz, 1H), 3.07 (dt, J₁ = 7.0 Hz, J₂ = 18.0 Hz, 2H), 2.85 (t, J = 7.0 |
| | Hz, 1H), 2.81 (t, J = 7.0 Hz, 1H), 2.00 (q, J = 7.0 Hz, 1H), 1.73 (q, J = 7.0 Hz, |
| | 1H), 1.64 (quintet, J = 7.5 Hz, 1H), 1.52 (quintet, J = 7.5 Hz, 1H), 1.27 (q, |
| | J = 4.0 Hz, 1H), 1.23 (q, J = 4.0 Hz, 1H), 0.96 (q, J = 4.0 Hz, 1H), 0.91 (q, J = 4.0 |
| | Hz, 1H). |
| BD16-1 | ¹H NMR (500 MHz, CD₃OD) δ: 8.04-8.08 (m, 2H), 7.68 (dd, J₁ = 6.0 |
| | Hz, J₂ = 8.5 Hz, 2H), 4.30 (q, J = 7.0 Hz, 2H), 1.60 (s, 9H), 4.30 (q, J = 7.0 Hz, |
| | 2H), 1.60 (s, 9H), 1.26 (t, J = 7.0 Hz, 3H). |
| BD16-3 | LC/MS: [M + H]⁺: 545.5. ¹H NMR (500 MHz, CD₃OD) δ: 7.00-7.75 |
| | (m, 2H), 7.49(s, 2H), 7.31 (d, J = 8.0 Hz, 1H), 7.22 (dd, J₁ = 8.0 Hz, J₂ = 17.0 |
| | Hz, 2H), 7.09 (d, J = 7.5 Hz, 1H), 5.49-5.87 (m, 1H), 4.95-5.06 (m, 1H), 4.81- |
| | 4.84 (m, 1H), 4.75 (s, 1H), 4.48 (s, 1H), 3.43 (t, J = 7.5 Hz, 1H), 3.25 (q, |
| | J = 7.0 Hz, 2H), 3.16 (t, J = 8.0 Hz, 1H), 2.73-2.78 (m, 2H), 2.10 (q, J = 7.5 Hz, |
| | 1H), 1.72-1.83 (m, 2H), 1.57-1.63 (m, 1H), 1.42 (s, 9H). |
| BD16 | LC/MS: [M + H]⁺: 417.2. ¹H NMR (400 MHz, CD₃OD) δ: 7.75 (d, J = 8.0 |
| | Hz, 1H), 7.69 (d, J = 8.0 Hz, 1H), 7.48 (d, J = 8.0 Hz, 2H), 7.30-7.34 (m, 1H), |
| | 7.23 (dd, J₁ = 8.0 Hz, J₂ = 13.6 Hz, 2H), 7.10 (d, J = 7.6 Hz, 1H), 5.49-5.87 (m, |
| | 1H), 4.94-5.06 (m, 1H), 4.82 (d, J = 14.0 Hz, 1H), 4.75(s, 1H), 4.49 (s, 1H), |
| | 3.44 (t, J = 8.0 Hz, 1H), 3.18 (t, J = 8.0 Hz, 1H), 2.83-2.89 (m, 2H), 2.72-2.78 |
| | (m, 2H), 2.10 (q, J = 7.2 Hz, 1H), 1.69-1.83(m, 2H), 1.59 (quintet, J = 7.6 Hz, |
| | 1H). |
| BD10-1 | ¹H NMR (500 MHz, CDCl₃) δ: 8.17-8.19 (m, 1H), 8.01-8.03 (m, 1H), |
| | 8.02 (d, J = 8.0 Hz, 1H), 7.67 (d, J = 8.0 Hz, 1H), 7.45 (td, J₁ = 2.5 Hz, J₂ = 8.0 |
| | Hz, 1H), 6.44 (dd, J₁ = 2.0 Hz, J₂ = 16.0 Hz, 1H), 3.93 (s, 3H), 1.53 (s, 9H). |
| BD10-2 | ¹H NMR (500 MHz, CD₃OD) δ: 7.85 (t, J = 2.0 Hz, 1H), 7.82 (dt, J₁ = 2.0 |
| | Hz, J₂ = 10 Hz, 1H), 7.43 (dt, J₁ = 2.0 Hz, J₂ = 10.0 Hz, 1H), 7.35 (t, J = 9.5 Hz, |
| | 1H), 3.86 (s, 3H), 2.91 (t, J = 9.5 Hz, 2H), 2.53 (t, J = 9.5 Hz, 2H), 1.37 (s, |
| | 9H). |
| BD10-3 | LC/MS: [M − H⁻] : 249.0. ¹H NMR (500 MHz, CD₃OD) δ: 7.82-7.87 (m, |
| | 2H), 7.33-7.40 (m, 2H), 2.93 (t, J = 9.5 Hz, 1H), 2.56 (t, J = 9.5 Hz, 2H), 1.39 |
| | (s, 9H). |
| BD10-4 | LC/MS: [M + H]⁺: 551.3. ¹H NMR (500 MHz, CD₃OD) δ: 7.30-7.41 |
| | (m, 3H), 7.20-7.27 (m, 4H), 7.08 (d, J = 9.5 Hz, 1H), 5.51-5.88 (m, 1H), 4.95- |
| | 5.07 (m, 1H), 4.81 (s, 1H), 4.74 (s, 1H), 4.49 (s, 1H), 3.44 (t, J = 9.0 Hz, 1H), |
| | 3.23-3.28 (m, 2H), 3.15-3.19 (m, 1H), 2.94 (t, J = 9.0 Hz, 1H), 2.86-2.87 (m, |
| | 1H), 2.76(q, J = 9.0 Hz, 2H), 2.57(t, J = 9.0 Hz, 1H), 2.49 (t, J = 9.0 Hz, 1H), |
| | 2.10 (q, J = 9.0 Hz, 1H), 1.82 (q, J = 9.0 Hz, 1H), 1.70-1.76 (m, 1H), 1.55-1.64 |
| | (m, 1H), 1.36-1.41 (m, 18H). |
| BD10 | LC/MS: [M + H]⁺: 395.3. ¹H NMR (500 MHz, CD₃OD) δ: 7.36-7.42 (m, |
| | 2H), 7.23-7.34 (m, 5H), 7.16 (d, J = 9.5 Hz, 1H), 5.51-5.88 (m, 1H), 4.95- |
| | 5.07 (m, 1H), 4.75 (s, 1H), 4.52 (s, 1H), 3.45 (t, J = 10.0 Hz, 1H), 3.14-3.22 |
| | (m, 3H), 2.88-2.99 (m, 4H), 2.51-2.67 (m, 2H), 2.08-2.14 (m, 1H), 1.72- |
| | 1.85 (m, 2H), 2.76 (quintet, J = 9.5 Hz, 1H). |
| BD12-4 | LC/MS: [M + Na]⁺: 531.4. ¹H NMR (500 MHz, CD₃OD) δ: 7.81-7.93 |
| | (m, 2H), 7.35-7.51 (m, 2H), 7.15 (d, J = 7.5 Hz, 1H), 7.11 (d, J = 8.0 Hz, 1H), |
| | 7.04 (d, J = 8.0 Hz, 1H), 6.90 (d, J = 8.5 Hz, 1H), 5.68-5.81 (m, 1H), 4.91-5.01 |
| | (m, 2H), 4.53 (s, 1H), 4.49 (s, 1H), 3.89 (d, J = 6.5 Hz, 3H), 3.31-3.33 (m, |
| | 1H), 3.21-3.24 (m, 2H), 3.16-3.19 (m, 1H), 3.05 (t, J = 7.5 Hz, 1H), 2.96 (t, |
| | J = 7.5 Hz, 1H), 2.78 (t, J = 7.0 Hz, 1H), 2.71-2.74 (m, 2H), 2.69 (t, J = 7.5 Hz, |
| | 1H), 1.93-2.00 (m, 2H), 1.55-1.61 (m, 1H), 1.49-1.55 (m, 1H), 1.39-1.42 |
| | (m, 9H). |
| BD12 | LC/MS: [M + H]⁺: 395.3. ¹H NMR (500 MHz, CD₃OD) δ: 7.66-7.942 |
| | (m, 2H), 6.92-7.07 (m, 2H), 6.87 (d, J = 12.5 Hz, 2H), 6.58-6.73 (m, 2H), |
| | 5.59-5.75 (m, 1H), 4.87-4.96 (m, 2H), 4.37 (s, 1H), 4.26 (s, 1H), 3.48 (brs, |
| | 2H), 3.34-3.38 (m, 1H), 3.23-3.26 (m, 1H), 2.93-2.97 (m, 1H), 2.72-2.82 |
| | (m, 4H), 2.52 (t, J = 7.0 Hz, 2H), 2.37 (t, J = 7.5 Hz, 1H), 1.94 (q, J = 7.0 Hz, |
| | 1H), 1.86 (q, J = 7.0 Hz, 1H), 1.86 (quintet, J = 8.0 Hz, 1H), 1.42 (quintet, |
| | J = 8.0 Hz, 1H). |
| BD21-2 | LC/MS: [M + H]⁺: 373.1. ¹H NMR (500 MHz, CD₃OD) δ: 7.66 (dd, |
| | J₁ = 6.0 Hz, J₂ = 10.0 Hz, 1H), 7.31 (dd, J₁ = 6.0 Hz, J₂ = 11.0 Hz, 1H), 4.83 (s, |
| | 1H), 1.48 (s, 18H). |
| BD21-3 | LC/MS: [M + H]⁺: 673.4. ¹H NMR (500 MHz, CD₃OD) δ: 7.28-7.36 |
| | (m, 2H), 7.07-7.27 (m, 4H), 5.47-5.86 (m, 1H), 4.82-5.06 (m, 2H), 4.77- |
| | 4.81 (m, 1H), 4.58-4.60 (m, 1H), 4.45(s, 1H), 3.47-3.50 (m, 1H), 3.22-3.28 |
| | (m, 2H), 3.11-3.16 (m, 1H), 2.72-2.78 (m, 2H), 2.02-2.12 (m, 1H), 1.84 (q, |
| | J = 7.0 Hz, 1H), 1.73 (q, J = 7.0 Hz, 1H), 1.55-1.63 (m, 1H), 1.49 (s, 9H), |
| | 1.46-1.47 (m, 9H), 1.40-1.43 (m, 9H). |
| BD21 | LC/MS: [M + H]⁺: 417.2. ¹H NMR (500 MHz, CDCl₃) δ: 7.37(d, J = 8.0 |
| | Hz, 1H), 7.31 (d, J = 8.0 Hz, 1H), 7.22-7.28 (m, 2H), 7.13-7.21 (m, 2H), 5.49- |
| | 5.86 (m, 1H), 5.02-5.06 (m, 1H), 4.95-4.98 (m, 1H), 4.82-4.86 (m, 2H), 4.50 |
| | (s, 1H), 3.70-3.75 (m, 2H), 3.143-3.20 (m, 3H), 2.94-2.99 (m, 2H), 7.13- |
| | 7.21 (m, 2H), 2.10 (q, J = 7.5 Hz, 1H), 1.86 (q, J = 7.0 Hz, 1H), 1.73 (quintet, |
| | J = 7.5 Hz, 1H), 1.60 (quintet, J = 7.0 Hz, 1H). |
| BD07-1 | ¹H NMR (400 MHz, CDCl₃) δ: 8.04 (td, J₁ = 1.6 Hz, J₂ = 7.6 Hz, 1H), |
| | 8.02 (t, J = 1.6 Hz, 1H), 7.84 (dd, J₁ = 1.6 Hz, J₂ = 7.6 Hz, 1H), 7.48-7.53 (m, |
| | 3H), 7.41-7.46 (m, 1H), 7.33 (dd, J₁ = 1.6 Hz, J₂ = 7.6 Hz, 1H), 3.92 (s, 3H), |
| | 1.25 (s, 9H). |
| BD07 | LC/MS: [M + H]⁺: 443.4. ¹H NMR (400 MHz, CD₃OD) δ: 8.01-8.11 (m, |
| | 2H), 7.33-7.57 (m, 6H), 7.08 (t, J = 7.6 Hz, 2H), 6.82 (dd, J₁ = 7.6 Hz, J₂ = 3.2 |
| | Hz, 2H), 5.40-5.72 (m, 1H), 5.00-5.03 (m, 1H), 4.75-4.79 (m, 1H), 4.10- |
| | 4.25 (m, 1H), 3.54-3.78 (m, 1H), 2.92-3.25 (m, 2H), 2.53-2.84 (m, 3H), |
| | 1.65-1.81 (m, 2H), 1.29-1.40 (m, 2H). |
| NE2C | LC/MS: [M]⁺: 118.07. ¹H NMR (500 MHz, D₂O) δ: 3.88 (s, 2H), |
| | 3.24 (s, 9H). |
| NE4C | LC/MS: [M]⁺: 146.10. ¹H NMR (500 MHz, D₂O) δ: 3.33-3.37 (m, 2H), |
| | 3.12 (s, 9H), 2.49 (t, J = 7.0 Hz, 2H), 2.04-2.10 (m, 2H). |
| NE5C | LC/MS: [M]⁺: 160.11. ¹H NMR (500 MHz, CD₃OD) δ: 3.36-3.39 (m, |
| | 2H), 3.31 (quintet, J = 7.0 Hz, 2H), 3.14 (s, 9H), 2.42 (t, J = 7.0 Hz, 2H), |
| | 1.67 (quintet, J = 7.5 Hz, 2H). |
| NE6C | LC/MS: [M]⁺: 174.1. ¹H NMR (400 MHz, D₂O) δ: 3.29-3.34 (m, 2H), |
| | 3.10 (s, 9H), 2.41 (t, J = 7.2 Hz, 2H), 1.77-1.85 (m, 2H), 1.66 (quintet, J = 7.6 |
| | Hz, 2H), 1.40 (quintet, J = 7.6 Hz, 2H). |
| NE7C | LC/MS: [M]⁺: 188.1. ¹H NMR (400 MHz, DMSO-d₆) δ: 3.28-3.32 |
| | (m, 2H), 3.06 (s, 9H), 2.18 (t, J = 7.2 Hz, 2H), 1.66 (quintet, J = 7.2 Hz, 2H), |
| | 1.50 (quintet, J = 7.2 Hz, 2H), 1.22-1.35 (m, 4H). |
| NE15C | LC/MS: [M]⁺: 300.25. ¹H NMR (500 MHz, DMSO-d₆) δ: 3.11-3.14 |
| | (m, 2H), 2.92 (s, 9H), 2.18 (t, J = 7.5 Hz, 2H), 1.56-1.62 (m, 2H), 1.41-1.44 |
| | (m, 2H), 1.15-1.21 (m, 20H). |
| E10 | LC/MS: [M]⁺: 176.3. ¹H NMR (500 MHz, D₂O) δ: 3.91-3.94 (m, |
| | 2H), 3.75 (t, J = 6.0 Hz, 2H), 3.55-3.57 (m, 2H), 3.16 (s, 9H), 2.44 (t, J = 6.0 |
| | Hz, 2H). |
| E20 | LC/MS: [M]⁺: 220.1. ¹H NMR (500 MHz, CD₃OD) δ: 3.92-3.95 (m, |
| | 2H), 3.73 (t, J = 6.0 Hz, 2H), 3.66-3.68 (m, 2H), 3.62-3.64 (m, 2H), 3.58- |
| | 3.60 (m, 2H), 3.20 (s, 9H), 2.53 (t, J = 6.0 Hz, 2H). |
| E30 | LC/MS: [M]⁺: 264.08. ¹H NMR (500 MHz, D₂O) δ: 3.93-3.96 (m, |
| | 2H), 3.67-3.69 (m, 4H), 3.63-3.66 (m, 4H), 3.55-3.57 (m, 2H), 3.16 (s, |
| | 9H), 2.60 (t, J = 6.0 Hz, 2H). |

### Example 3 Synthesis of Target Molecules

### 1. Synthesis of Target Molecule 2401

### 1). Preparation of Intermediate T2401-1

Intermediate NC (4.13 g, 7.3 mmol) was dissolved in 40 mL of EA, DIPEA (3.82 mL, 21.9 mmol) and HATU (3.04 g, 8.0 mmol) were added at 0° C., the solution was reacted for 1 hour, then intermediate BD01 (3.5 g, 9.1 mmol) was dissolved in 30 mL of DMF and added dropwise into the above reaction system, and reacted for 4 hours at 0° C. Then, 50 mL of water and 50 mL of EA were added to the reaction solution, which was adjusted the pH to 6 with 0.1 M acetic acid solution, and extracted with 30 mL of EA for three times. The organic phases were combined, washed twice with 0.1 M acetic acid solution, and then dried over anhydrous Na₂SO₄ and filtered. The filtrate was rotary evaporated to dryness under reduced pressure to obtain crude product, which was purified by preparative chromatography to obtain 5.4 g of T2401-1 as a white solid with a yield of 79%.

### 2). Preparation of Intermediate T2401-2

NB (1.97 g, 5.8 mmol) was dissolved in 20 mL of DCM and p-TsOH·H₂O (3.32 g, 17.5 mmol) was added at 0° C., the solution was reacted at 0° C. for 4 hours, the reaction was monitored via LC/MS to find that NB was consumed completely, the organic solvent was rotary evaporated to obtain crude product, which was dissolved in 20 mL of DCM.

T2401-1 (3.6 g, 3.9 mmol) was dissolved in 36 mL of DMF, and DIPEA (2.7 mL, 15.6 mmol) was added at 0° C. Then HATU (1.67 g, 4.3 mmol) was further added, the solution was reacted at 0° C. for 1 hour, the reaction was monitored via LC/MS to find that T2401-1 was consumed completely. Then it was added dropwise into the above reaction system and reacted at 0° C. for 4 hours. Then, 50 mL of water and 50 mL of EA were added to the reaction system, which was adjusted the pH to 6 with 0.1 M acetic acid solution, and extracted with 50 mL of EA for three times. The organic phases were combined, washed twice with 50 mL of 0.1 M acetic acid solution, and then dried over anhydrous Na₂SO₄ and filtered. The filtrate was rotary evaporated to dryness under reduced pressure to obtain crude product, which was purified by preparative chromatography to obtain 2.45 g of T2401-2 as a white solid with a yield of 53%.

### 3). Preparation of Intermediate T2401-3

T2401-2 (2.4 g, 2.1 mmol) was dissolved in DCM (240 mL), and p-TsOH·H₂O (2.38 g, 12.6 mmol) was added at 0° C., and then the solution was stirred at room temperature for 24 hours, the reaction was monitored via LC/MS until T2401-2 was consumed completely. Excess sodium bicarbonate aqueous solution was added dropwise at 0° C. until pH=8, DCM was rotary evaporated off under reduced pressure, the remaining aqueous phase was purified by preparative chromatography to obtain 1.88 g of T2401-3 as a white solid with a yield of 90%.

### 4). Preparation of Intermediate T2401-4

Intermediate NA (0.9 g, 1.3 mmol) was dissolved in 9 mL of DMF, and DIPEA (0.68 mL, 3.9 mmol) was added at 0° C. Then HATU (0.543 g, 1.4 mmol) was further added, the solution was reacted at 0° C. for 1 hour, the reaction was monitored via LC/MS to find that NA was consumed completely. Then, T2401-3 (1.48 g, 1.5 mmol) was dissolved in 15 mL of DMF, which was added dropwise into the reaction system, and reacted at 0° C. for 12 hours. After the reaction was completed, 20 mL of water, 20 mL of EA and 0.1 M acetic acid solution were added to the reaction system, which was adjusted the pH to 6 and extracted with 20 mL of EA for three times. The organic phases were combined, washed twice with 0.1 M acetic acid solution, and then dried over anhydrous Na₂SO₄ and filtered. The filtrate was rotary evaporated to dryness under reduced pressure to obtain crude product, which was purified by silica gel column chromatography to obtain 1.5 g of T2401-4 as a pale-yellow solid with a yield of 69%.

### 5). Preparation of Intermediate T2401-5

T2401-4 (1.4 g, 0.8 mmol) was dissolved in DCM (140 mL), and p-TsOH·H₂O (0.798 g, 4 mmol) was added and then the solution was stirred at room temperature for 12 hours, the reaction was monitored via LC/MS until T2401-4 was consumed completely. DCM was rotary evaporated offunder reduced pressure, leaving a large amount of solid. Excess water was added in an ice bath and sonicated, resulting in the precipitation of a large amount of solid. The solid was filtered, dissolved in 20 mL of THF, and dried over anhydrous Na₂SO₄. THE was rotary evaporated to dryness under reduced pressure to obtain 2 g of crude T24011-5 as a pale-yellow solid, which can be used directly for the next reaction without purification. LC/MS: [M+H]⁺: 1564.6.

### 6). Preparation of Intermediate T2401-6

The above crude T2401-5 was dissolved in 250 mL of DCM, and DIPEA (1.33 mL, 4.8 mmol) was added at 0° C. Then a solution of HATU (1.24 g, 3.2 mmol) in 60 mL of DCM was further added, the solution was reacted overnight at 0° C., the reaction was monitored via LC/MS to find that T2401-5 was consumed completely. 30 mL of water was added to the reaction mixture, which was then extracted once with 30 mL of DCM, then the aqueous phase was further extracted with 20 mL of EA twice, the organic phases were combined, and dried over anhydrous Na₂SO₄ and filtered. The filtrate was rotary evaporated to dryness under reduced pressure to obtain crude product, which was purified by silica gel column chromatography to obtain 0.6 g of T2401-5 as a yellow solid with a yield of 46%.

### 7). Preparation of Intermediate T2401-7

T2401-6 (0.5 g, 0.3 mmol) was dissolved in 200 mL of DCM and 6.6 mL of glacial acetic acid, and Zhan's catalyst-1B (23.7 mg, 0.03 mmol) was added, the solution was degassed with nitrogen for four times and reacted at 55° C. for 12 hours. The reaction was monitored via LC/MS until T2401-6 was consumed completely, saturated sodium bicarbonate solution was added to the reaction solution to adjust the pH to 8, the aqueous phase was then extracted with 30 mL of DCM for three times. The organic phases were combined, and then dried over anhydrous Na₂SO₄ and filtered. The filtrate was rotary evaporated to dryness under reduced pressure to obtain crude product, which was purified by silica gel column chromatography to obtain 0.33 g of T2401-7 as a pale red solid with a yield of 67%. LC/MS: [M+H]⁺: 1518.1.

### 8). Preparation of Intermediate T2401-8

T2401-7 (100 mg, 0.065 mmol) was dissolved in 10 mL of THF, 10 mg of 10% Pd/C was added, the solution was purged with hydrogen for four times, and reacted at room temperature for 12 hours. The reaction was monitored via LC/MS until T2401-7 was substantially consumed completely. Pd/C was filtered out and washed with 5 mL of THE for three times, the organic solvent was rotary evaporated to dryness to obtain T2401-8, which was directly used for the next reaction without purification. LC/MS: [M+H]⁺: 1386.2.

### 9). Synthesis of Target Molecule 2401

Intermediate NE6C (50 mg, 0.197 mmol) was dissolved in 5 mL of DMF, and DIPEA (0.045 mL, 0.262 mmol) was added at 0° C. Then AOP (102 mg, 0.229 mmol) was further added, the solution was reacted at 0° C. for 1 hour. After 1 hour of reaction, a solution of the above T2401-8 in 5 mL of DMF was added, and the solution was reacted at room temperature for 2 hours, the reaction was monitored via LC/MS to find that T2401-8 was consumed completely. The reaction solution was purified by preparative chromatography to obtain 23.1 mg of T2401 as a white solid with a yield of 22.8%.

### 2. Synthesis of Target Molecule 2450

2450 was prepared with reference to the synthetic method of 2401, where E20 was used instead of NE6C. The reaction solution was purified by preparative chromatography to obtain 40 mg of T2450 as a white solid with a yield of 38%.

### 3. Synthesis of Target Molecule 2451

2451 was prepared with reference to the synthetic method of 2401, where NE5C was used instead of NE6C. The reaction solution was purified by preparative chromatography to obtain 90.2 mg of 2451 as a white solid with a yield of 74%.

### 4. Synthesis of Target Molecule 2455

2455 was prepared with reference to the synthetic method of 2401, where NE2C was used instead of NE6C. The reaction solution was purified by preparative chromatography to obtain 86.7 mg of 2455 as a white solid with a yield of 54%.

### 5. Synthesis of Target Molecule 2457

2457 was prepared with reference to the synthetic method of 2401, where NE4C was used instead of NE6C. The reaction solution was purified by preparative chromatography to obtain 127.5 mg of 2457 as a white solid with a yield of 77%.

### 6. Synthesis of Target Molecule 2460

2460 was prepared with reference to the synthetic method of 2401, where NE7C was used instead of NE6C. The reaction solution was purified by preparative chromatography to obtain 76.6 mg of 2460 as a white solid with a yield of 68%.

### 7. Synthesis of Target Molecule 2461

2461 was prepared with reference to the synthetic method of 2401, where NE15C was used instead of NE6C. The reaction solution was purified by preparative chromatography to obtain 72.4 mg of 2461 as a white solid with a yield of 50%.

### 8. Synthesis of Target Molecule 2462

2451 (110 mg, 0.072 mmol) was dissolved in 3 mL of TFA, and SCF2 reagent (99 mg, 0.360 mmol) was added at 0° C., the solution was reacted at 30° C. for 16 hours. The reaction was monitored via LC/MS until T2451 was consumed completely. The reaction solution was purified by preparative chromatography to obtain 47.2 mg of T2462 as a white solid with a yield of 40%.

### 9. Synthesis of Target Molecule 2463

2451 (100 mg, 0.065 mmol) was dissolved in 3 mL of TFA, and SCF3 reagent (96.2 mg, 0.325 mmol) was added at 0° C. Then the solution was reacted at 30° C. for 16 hours. The reaction was monitored via LC/MS until T2451 was consumed completely. The reaction solution was purified by preparative chromatography to obtain 72.7 mg of 2463 as a white solid with a yield of 68%.

### 10. Synthesis of Target Molecule 2402A and 2402B

### 1). Preparation of Intermediate T2402-1

T2402-1 was prepared with reference to the synthetic method of T2401-1, where BD02 was used instead of BD01. The crude product was purified by preparative chromatography to obtain 16.5 g of T2402-1 with a yield of 84%.

### 2). Preparation of Intermediate T2402-2

T2402-2 was prepared with reference to the synthetic method of T2401-2, where T2402-1 was used instead of T2401-1. The crude product was purified by preparative chromatography to obtain 11.16 g of T2402-2 with a yield of 67%.

### 3). Preparation of Intermediate T2402-3

T2402-3 was prepared with reference to the synthetic method of T2401-3, where T2402-2 was used instead of T2401-2. The crude product was purified by preparative chromatography to obtain 8.6 g of T2402-3 with a yield of 90%.

### 4). Preparation of Intermediate T2402-4

T2402-4 was prepared with reference to the synthetic method of T2401-4, where T2402-3 was used instead of T2401-3. The crude product was purified by silica gel column chromatography to obtain 10.31 g of T2402-4 with a yield of 78%.

### 5). Preparation of Intermediate T2402-5

T2402-5 was prepared with reference to the synthetic method of T2401-5, where T2402-4 was used instead of T2401-4. LC/MS: [M+H]⁺: 1577.7.

### 6). Preparation of Intermediate T2402-6

T2402-6 was prepared with reference to the synthetic method of T2401-6, where T2402-5 was used instead of T2401-5. The crude product was purified by preparative chromatography to obtain two isomers, of which 1.6 g of T2402-6A was obtained with a yield of 18%; and 1.3 g of T2402-6B was obtained with a yield of 14%. LC/MS: [M+H]⁺: 1559.7.

### 7). Preparation of Intermediate T2402-7

T2402-7A: T2402-7A was prepared with reference to the synthetic method of T2401-7, where T2402-6A was used instead of T2401-6. The crude product was purified by preparative chromatography to obtain 1.07 g of T2402-7A as a white solid with a yield of 70%. LC/MS: [M+H]⁺: 1531.7.

T2402-7B: T2402-7B was prepared with reference to the synthetic method of T2401-7, where T2402-6B was used instead of T2401-6. The crude product was purified by preparative chromatography to obtain 760 mg of T2402-7B as a white solid with a yield of 60%. LC/MS: [M+H]⁺: 1531.7.

### 8). Preparation of Intermediate T2402-8

T2402-8A: T2402-8A was prepared with reference to the synthetic method of T2401-8, where T2402-7A was used instead of T2401-7. LC/MS: [M+H]⁺: 1399.7.

T2402-8B: T2402-8B was prepared with reference to the synthetic method of T2401-8, where T2402-7B was used instead of T2401-7. LC/MS: [M+H]⁺: 1399.7.

### 9). Synthesis of Target Molecule 2402A

2402A was prepared with reference to the synthetic method of 2401. The reaction solution was purified by preparative chromatography to obtain 32 mg of 2402A as a white solid with a yield of 31%.

### 10). Synthesis of Target Molecule 2402B

2402B was prepared with reference to the synthetic method of 2401. The reaction solution was purified by preparative chromatography to obtain 28 mg of 2402B as a white solid with a yield of 27%.

### 11. Synthesis of Target Molecule 204B

204B was prepared with reference to the synthetic method of 2401, where NE4C was used instead of NE6C. The reaction solution was purified by preparative chromatography to obtain 22 mg of 204B as a white solid with a yield of 22%.

### 12. Synthesis of Target Molecule 205B

205B was prepared with reference to the synthetic method of 2401, where NE5C was used instead of NE6C. The reaction solution was purified by preparative chromatography to obtain 83 mg of 205B as a white solid with a yield of 81%.

### 13. Synthesis of Target Molecule 205A

205A was prepared with reference to the synthetic method of 2401, where NE5C was used instead of NE6C. The reaction solution was purified by preparative chromatography to obtain 82 mg of 205A as a white solid with a yield of 80%.

### 14. Synthesis of Target Molecule 211B

211B was prepared with reference to the synthetic method of 2401, where E10 was used instead of NE6C. The reaction solution was purified by preparative chromatography to obtain 50 mg of 211B as a white solid with a yield of 48%.

### 15. Synthesis of Target Molecule 212B

212B was prepared with reference to the synthetic method of 2401, where E20 was used instead of NE6C. The reaction solution was purified by preparative chromatography to obtain 53 mg of 212B as a white solid with a yield of 50%.

### 16. Synthesis of Target Molecule 212A

212A was prepared with reference to the synthetic method of 2401, where E20 was used instead of NE6C. The reaction solution was purified by preparative chromatography to obtain 16 mg of 212A as a white solid with a yield of 15%.

### 17. Synthesis of Target Molecule 213B

213B was prepared with reference to the synthetic method of 2401, where E30 was used instead of NE6C. The reaction solution was purified by preparative chromatography to obtain 78 mg of 213B as a white solid with a yield of 72%.

### 18. Synthesis of Target Molecule 2403

### 1). Preparation of Intermediate T2403-1

T2403-1 was prepared with reference to the synthetic method of T2401-1, where BD03 was used instead of BD01. The crude product was purified by preparative chromatography to obtain 11.32 g of T2403-1 as a white solid with a yield of 85%.

### 2). Preparation of Intermediate T2403-2

T2403-2 was prepared with reference to the synthetic method of T2401-2, where T2403-1 was used instead of T2401-1. The crude product was purified by preparative chromatography to obtain 4.42 g of T2403-2 as a white solid with a yield of 52%.

### 3). Preparation of Intermediate T2403-3

T2403-3 was prepared with reference to the synthetic method of T2401-3, where T2403-2 was used instead of T2401-2. The crude product was purified by preparative chromatography to obtain 2.56 g of T2403-3 as a white solid with a yield of 70%.

### 4). Preparation of Intermediate T2403-4

T2403-4 was prepared with reference to the synthetic method of T2401-4, where T2403-2 was used instead of T2401-2. The crude product was purified by silica gel column chromatography to obtain 2.48 g of T2403-4 as a white solid with a yield of 92%.

### 5). Preparation of Intermediate T2403-5

T2403-5 was prepared with reference to the synthetic method of T2401-5, where T2403-4 was used instead of T2401-4. LC/MS: [M+H]⁺: 1591.7.

### 6). Preparation of Intermediate T2403-6

T2403-6 was prepared with reference to the synthetic method of T2401-6, where T2403-5 was used instead of T2401-5. The crude product was purified by preparative chromatography to obtain 1.32 g of T2403-6 as a white solid with a yield of 60%.

### 7). Preparation of Intermediate T2403-7

T2403-7 was prepared with reference to the synthetic method of T2401-7, where T2403-6 was used instead of T2401-6. The crude product was purified by preparative chromatography to obtain 0.61 g of T2403-7 as a pale-yellow solid with a yield of 50%. LC/MS: [M+H]⁺: 1545.5.

### 8). Preparation of Intermediate T2403-8

T2403-8 was prepared with reference to the synthetic method of T2401-8, where T2403-7 was used instead of T2401-7. LC/MS: [M+H]⁺: 1413.6.

### 9). Synthesis of Target Molecule 2403

2403 was prepared with reference to the synthetic method of 2401, and T2403-6 was prepared with reference to the synthetic method of T2401-6, where T2403-8 was used instead of T2401-8. The reaction solution was purified by preparative chromatography to obtain 208 mg of 2403 as a white solid with a yield of 72%.

### 19. Synthesis of Target Molecule 2430

The above reserved T2403-8 was dissolved in DMF (2.5 mL), add Na₂CO₃ (21 mg, 0.18 mmol) was added at 0° C. Then CH₃I (0.03 mL, 0.52 mmol) was further added, and the solution was reacted at 40° C. for 2 hours. The reaction was monitored via LC/MS until T2403-8 was consumed completely. The solution was filtered to remove sodium carbonate solid, which was rinsed twice with 5 mL of acetonitrile. The organic phases were combined, the organic solvent was rotary evaporated to dryness to obtain crude product, which was purified by preparative chromatography to obtain 68 mg of 2430 as a white solid with a yield of 87%.

### 20. Synthesis of Target Molecule 2404

### 1). Preparation of Intermediate T2404-1

T2404-1 was prepared with reference to the synthetic method of T2401-1, where BD04 was used instead of BD01. The crude product was purified by preparative chromatography to obtain 12.9 g of T2404-1 as a white solid with a yield of 95%.

### 2). Preparation of Intermediate T2404-2

T2404-2 was prepared with reference to the synthetic method of T2401-2, where T2404-1 was used instead of T2401-1. The crude product was purified by silica gel column chromatography to obtain 14.1 g of T2404-2 with a yield of 89%.

### 3). Preparation of Intermediate T2404-3

T2404-3 was prepared with reference to the synthetic method of T2401-3, where T2404-2 was used instead of T2401-2. The crude product was purified by preparative chromatography to obtain 8.67 g of T2404-3 as a white solid with a yield of 72%.

### 4). Preparation of Intermediate T2404-4

T2404-4 was prepared with reference to the synthetic method of T2401-4, where T2404-3 was used instead of T2401-3. The crude product was purified by silica gel column chromatography to obtain 7.57 g of T2404-4 as a white solid with a yield of 77%.

### 5). Preparation of Intermediate T2404-5

T2404-5 was prepared with reference to the synthetic method of T2401-5, where T2404-4 was used instead of T2401-4. LC/MS: [M+H]⁺: 1589.8.

### 6). Preparation of Intermediate T2404-6

T2404-6 was prepared with reference to the synthetic method of T2401-6, where T2404-5 was used instead of T2401-5. The crude product was purified by silica gel column chromatography to obtain 3.59 g of T2404-6 as a white solid with a yield of 52%.

### 7). Preparation of Intermediate T2404-7

T2404-7 was prepared with reference to the synthetic method of T2401-7, where T2404-6 was used instead of T2401-6. The crude product was purified by preparative chromatography to obtain 1.7 g of T2404-7 as a white solid with a yield of 55%. LC/MS: [M+H]⁺: 1543.9.

### 8). Preparation of Intermediate T2404-8

T2404-8 was prepared with reference to the synthetic method of T2401-8, where T2404-7 was used instead of T2401-7. LC/MS: [M+H]⁺: 1411.9.

### 9). Synthesis of Target Molecule 2404

2404 was prepared with reference to the synthetic method of 2401, where NE5C was used instead of NE6C. The reaction solution was purified by preparative chromatography to obtain 410 mg of 2404 as a white solid with a yield of 75%.

### 21. Synthesis of Target Molecule 2416

### 1). Preparation of Intermediate T2416-1

T2416-1 was prepared with reference to the synthetic method of T2401-1, where BD16 was used instead of BD01. The crude product was purified by silica gel column chromatography to obtain 8.3 g of T2416-1 as a white solid with a yield of 60%.

### 2). Preparation of Intermediate T2416-2

### Preparation of Intermediate NB-Me

S1 (20 g, 59.2 mmol) was dissolved in 200 mL of DCM, and DIPEA (30.86 mL, 177.5 mmol) was added at 0° C. Then CH₃I (7.37 mL, 118.3 mmol) was further added, the solution was gradually returned to room temperature and reacted for 12 hours. The reaction was monitored via LC/MS until S1 was consumed completely after 12 hours, 100 mL of water was added to the reaction system. Then the mixture was extracted with 100 mL of DCM for three times. The organic phases were combined, and then dried over anhydrous Na₂SO₄ and filtered. The filtrate was rotary evaporated to dryness under reduced pressure to obtain crude product, which was purified by silica gel column chromatography to obtain 10.35 g of NB-Me with a yield of 50%. LC/MS: [M+H]⁺: 353.2.

NB-Me (10.30 g, 29.3 mmol) was dissolved in 180 mL of DCM, and 36 mL of TFA was added at 0° C. Then gradually returned to room temperature and continuously reacted for 3 hours., the reaction was monitored via LC/MS to find that NB-Me was consumed completely. Then DCM and TFA were directly rotary evaporated off to obtain a yellow oil. The yellow oil was directly used for the next reaction without purification, the yellow oil was dissolved in 100 mL of DMF, and DIPEA (6.10 mL, 35.1 mmol), a solution of T2416-1 (11.25 g, 11.7 mmol) in 45 mL of DMF were added in sequence under an ice bath, and finally HATU (8.89 g, 23.4 mmol) was added, then the solution was reacted at room temperature for 48 hours. The reaction was monitored via LC/MS until T2416-1 was consumed completely, 50 mL of water and 50 mL of EA were added to the reaction system, and then extracted with 50 mL of EA for three times. The organic phases were combined, and then washed twice with saturated saline solution, then dried over anhydrous Na₂SO₄ and filtered. The filtrate was rotary evaporated to dryness under reduced pressure to obtain crude product, which was purified by silica gel column chromatography to obtain 11.49 g of T2416-2 with a yield of 82%.

### 3). Preparation of Intermediate T2416-3

T2416-2 (11.12 g, 9.3 mmol) was dissolved in DCM (350 mL), and TsOH·H₂O (10.61 g, 55.8 mmol) was added at 0° C., and then the solution was stirred at room temperature for 24 hours, the reaction was monitored via LC/MS until T2416-2 was consumed completely. DCM was rotary evaporated to dryness under reduced pressure, leaving a large amount of solid.

Excess water was added in an ice bath and sonicated, resulting in the precipitation of a large amount of solid. The solid was filtered, the liquid phase was discarded, the solid was dissolved in THF, and dried over anhydrous Na₂SO₄. THE was rotary evaporated to dryness under reduced pressure to obtain crude T2416-2-Me, which was used directly for the next reaction step without purification. The above crude product was dissolved in THE (150 mL), and LiOH·H₂O (3.91 g, 93 mmol) was dissolved in 150 mL of water, then added dropwise to the reaction system at 0° C., and then the solution was stirred at room temperature for 5 hours, the reaction was monitored via LC/MS until T2416-2-Me was consumed completely. The reaction was stopped, and the solution was adjusted to pH to 6 with 0.1 M phosphoric acid solution, THE was rotary evaporated off under reduced pressure, the remaining aqueous phase was purified by preparative chromatography to obtain 6.47 g of T2416-3 as a white solid with a yield of 68%.

### 4). Preparation of Intermediate T2416-4

T2416-4 was prepared with reference to the synthetic method of T2401-4, where T2416-3 was used instead of T2401-3. The crude product was purified by silica gel column chromatography to obtain 5.58 g of T2416-4 as a white solid with a yield of 76%.

### 5). Preparation of Intermediate T2416-5

T2416-5 was prepared with reference to the synthetic method of T2401-5, where T2416-4 was used instead of T2401-4. LC/MS: [M+H]⁺: 1599.7.

### 6). Preparation of Intermediate T2416-6

T2416-6 was prepared with reference to the synthetic method of T2401-6, where T2416-5 was used instead of T2401-5. The crude product was purified by preparative chromatography to obtain 2.62 g of T2416-6 as a white solid with a yield of 53%.

### 7). Preparation of Intermediate T2416-7

T2416-7 was prepared with reference to the synthetic method of T2401-7, where T2416-6 was used instead of T2401-6. The crude product was purified by preparative chromatography to obtain 1.5 g of T2416-7 as a pale-yellow solid with a yield of 60%. LC/MS: [M+H]⁺: 1553.6.

### 8). Preparation of Intermediate T2416-8

T2416-8 was prepared with reference to the synthetic method of T2401-8, where T2416-7 was used instead of T2401-7. LC/MS: [M+H]⁺: 1421.6.

### 9). Synthesis of Target Molecule 2416

2416 was prepared with reference to the synthetic method of 2401, where NE5C was used instead of NE6C. The reaction solution was purified by preparative chromatography to obtain 180 mg of 2416 as a white solid with a yield of 80%.

### 22. Synthesis of Target Molecule 2410

### Preparation of Intermediate T2410-1

T2410-1 was prepared with reference to the synthetic method of T2401-1, where BD10 was used instead of BD01. The crude product was purified by preparative chromatography to obtain 13.3 g of T2410-1 as a white solid with a yield of 99%.

### 1). Preparation of Intermediate T2410-2

T2410-2 was prepared with reference to the synthetic method of T2401-2, where T2410-1 was used instead of T2401-1. The crude product was purified by preparative chromatography to obtain 2.8 g of T2410-2 as a white solid with a yield of 39%.

### 2). Preparation of Intermediate T2410-3

T2410-3 was prepared with reference to the synthetic method of T2401-3, where T2410-2 was used instead of T2401-2. The crude product was purified by preparative chromatography to obtain 1.8 g of T2410-3 as a white solid with a yield of 74%.

### 3). Preparation of Intermediate T2410-4

T2410-4 was prepared with reference to the synthetic method of T2401-4, where T2410-3 was used instead of T2401-3. The crude product was purified by silica gel column chromatography to obtain 1.78 g of T2410-4 as a pale-yellow solid with a yield of 73%.

### 4). Preparation of Intermediate T2410-5

T2410-5 was prepared with reference to the synthetic method of T2401-5, where T2410-4 was used instead of T2401-4. LC/MS: [M+H]⁺: 1578.7.

### 5). Preparation of Intermediate T2410-6

T2410-6 was prepared with reference to the synthetic method of T2401-6, where T2410-5 was used instead of T2401-6. The crude product was purified by silica gel column chromatography to obtain 0.72 g of T2410-5 as a yellow solid with a yield of 46%.

### 6). Preparation of Intermediate T2410-7

T2410-7 was prepared with reference to the synthetic method of T2401-7, where T2410-6 was used instead of T2401-6. The crude product was purified by silica gel column chromatography to obtain 0.2 g of T2401-7 as a pale red solid with a yield of 31%. LC/MS: [M+H]⁺: 1531.6.

### 7). Preparation of Intermediate T2410-8

T2410-8 was prepared with reference to the synthetic method of T2401-8, where T2410-7 was used instead of T2401-7. LC/MS: [M+H]⁺: 1399.7.

### 8). Synthesis of Target Molecule 2410

2410 was prepared with reference to the synthetic method of 2401. The reaction solution was purified by preparative chromatography to obtain 68.8 mg of 2410 as a white solid with a yield of 68.8%.

### 23. Synthesis of Target Molecule 2452

2452 was prepared with reference to the synthetic method of 2401, where NE5C was used instead of NE6C. The reaction solution was purified by preparative chromatography to obtain 39.6 mg of 2452 as a white solid with a yield of 49%.

### 24. Synthesis of Target Molecule 2412

### 1). Preparation of Intermediate T2412-1

T2412-1 was prepared with reference to the synthetic method of T2401-1, where BD12 was used instead of BD01. The crude product was purified by silica gel column chromatography to obtain 18.5 g of T2412-1 as a yellow solid with a yield of 91%.

### 2). Preparation of Intermediate T2412-2

T2412-2 was prepared with reference to the synthetic method of T2401-2, where T2412-1 was used instead of T2401-1. The crude product was purified by preparative chromatography to obtain 2.9 g of T2412-2 as a white solid with a yield of 33%.

### 3). Preparation of Intermediate T2412-3

T2412-3 was prepared with reference to the synthetic method of T2401-3, where T2412-2 was used instead of T2401-2. The crude product was purified by preparative chromatography to obtain 1.7 g of T2412-3 as a white solid with a yield of 68%.

### 4). Preparation of Intermediate T2412-4

T2412-4 was prepared with reference to the synthetic method of T2401-4, where T2412-3 was used instead of T2401-3. The crude product was purified by silica gel column chromatography to obtain 1.62 g of T2412-4 as a pale-yellow solid with a yield of 74%.

### 5). Preparation of Intermediate T2412-5

T2412-5 was prepared with reference to the synthetic method of T2401-5, where T2412-4 was used instead of T2401-4. LC/MS: [M+H]⁺: 1577.8.

### 6). Preparation of Intermediate T2412-6

T2412-6 was prepared with reference to the synthetic method of T2401-6, where T2412-5 was used instead of T2401-5. The crude product was purified by silica gel column chromatography to obtain 0.67 g of T2412-6 as a pale yellow solid with a yield of 47%.

### 7). Preparation of Intermediate T2412-7

T2412-7 was prepared with reference to the synthetic method of T2401-7, where T2412-6 was used instead of T2401-6. The crude product was purified by silica gel column chromatography to obtain 0.26 g of T2412-7 as a pale red solid with a yield of 44%. LC/MS: [M+H]⁺: 1532.7.

### 8). Preparation of Intermediate T2412-8

T2412-8 was prepared with reference to the synthetic method of T2401-8, where T2412-7 was used instead of T2401-7. LC/MS: [M+H]⁺: 1400.7.

### 9). Synthesis of Target Molecule 2412

2412 was prepared with reference to the synthetic method of 2401, where T2412-8 was used instead of T2401-8. The reaction solution was purified by preparative chromatography to obtain 85.3 mg of 2412 as a white solid with a yield of 84%,

### 25. Synthesis of Target Molecule 2453

2453 was prepared with reference to the synthetic method of 2401, and the reaction solution was purified by preparative chromatography to obtain 35.1 mg of 2453 as a white solid with a yield of 35%,

### 26. Synthesis of Target Molecule 2454

2454 was prepared with reference to the synthetic method of 2401, and the reaction solution was purified by preparative chromatography to obtain 12.7 mg of 2454 as a white solid with a yield of 24%.

### 27. Synthesis of Target Molecule 2421

### 1). Preparation of Intermediate T2421-1

T2421-1 was prepared with reference to the synthetic method of T2401-1, where BD21 was used instead of BD01. The crude product was purified by preparative chromatography to obtain 4.66 g of T2421-1 as a white solid with a yield of 60%.

### 2). Preparation of Intermediate T2421-2

T2421-2 was prepared with reference to the synthetic method of T2401-2, where T2421-1 was used instead of T2401-1. The crude product was purified by preparative chromatography to obtain 5.5 g of T2421-2 as a white solid with a yield of 95%.

### 3). Preparation of Intermediate T2421-3

T2421-3 was prepared with reference to the synthetic method of T2401-3, where T2421-2 was used instead of T2401-2. The remaining aqueous phase was purified by preparative chromatography to obtain 2.97 g of T2421-3 as a white solid with a yield of 61%.

### 4). Preparation of Intermediate T2421-4

T2421-4 was prepared with reference to the synthetic method of T2401-4, where T2421-3 was used instead of T2401-3. The crude product was purified by silica gel column chromatography to obtain 2.99 g of T2421-4 as a white solid with a yield of 80%.

### 5). Preparation of Intermediate T2421-5

T2421-5 was prepared with reference to the synthetic method of T2401-5, where T2421-4 was used instead of T2401-4. LC/MS: [M+H]⁺: 1599.7.

### 6). Preparation of Intermediate T2421-6

T2421-6 was prepared with reference to the synthetic method of T2401-6, where T2421-5 was used instead of T2401-5. The crude product was purified by silica gel column chromatography to obtain 1.67 g of T2421-6 as a pale-yellow solid with a yield of 64%.

### 7). Preparation of Intermediate T2421-7

T2421-7 was prepared with reference to the synthetic method of T2401-7, where T2421-6 was used instead of T2401-6. The crude product was purified by preparative chromatography to obtain 1.05 g of T2421-7 as a white solid with a yield of 73%. LC/MS: [M+H]⁺: 1553.7.

### 8). Preparation of Intermediate T2421-8

T2421-8 was prepared with reference to the synthetic method of T2401-8, where T2421-7 was used instead of T2401-7. LC/MS: [M+H]⁺: 1421.7.

### 9). Synthesis of Target Molecule 2421

2421 was prepared with reference to the synthetic method of 2401, where NE5C1 was used instead of NE6C. The reaction solution was purified by preparative chromatography to obtain 65 mg of 2421 as a white solid with a yield of 65%.

### 28. Synthesis of Target Molecule 2427

T2421-8 (100 mg, 0.071 mmol) was dissolved in 10 mL of MeOH, and Selectfluor® fluorinating reagent (55 mg, 0.155 mmol) was added, the solution was degassed with nitrogen for three times and reacted at 65° C. for 10 hours. The reaction was monitored via LC/MS until T2421-8 was consumed completely, the reaction was stopped, and the organic solvent was rotary evaporated to dryness under reduced pressure to obtain crude product, which was purified by preparative chromatography to obtain 35 mg of T2427-8 as a white solid with a yield of 34%. LC/MS: [M+H]⁺: 1457.7.

NE5C (19 mg, 0.079 mmol) was dissolved in 1 mL of DMF, and DIPEA (0.018 mL, 0.102 mmol) was added at 0° C. Then AOP (40 mg, 0.091 mmol) was further added, and the solution was reacted at room temperature for 1 hour. Then, 1 mL of the above T2427-8 (33 mg, 0.023 mmol) in DMF was added, the solution was reacted at room temperature for 2 hours, the reaction was monitored via LC/MS to find that T2427-8 was consumed completely. The reaction solution was purified by preparative chromatography to obtain 22 mg of 2427 as a white solid with a yield of 60%.

### 29. Synthesis of Target Molecule 2428

T2421-8 (100 mg, 0.071 mmol) was dissolved in 8 mL of TFA, and S1 (98 mg, 0.355 mmol) was added, the solution was degassed with nitrogen for three times and reacted at 40° C. for 16 hours. The reaction was monitored via LC/MS until T2421-8 was consumed completely. Then TFA was rotary evaporated off under reduced pressure. Sodium bicarbonate aqueous solution was slowly added dropwise at 0° C. to adjust pH to 8-9, then methanol was added, solid precipitated at this time, which was filtered, and then most of the methanol was rotary evaporated off under reduced pressure to obtain crude product, which was purified by preparative chromatography to obtain 20 mg of T2428-8 as a white solid with a yield of 19%. LC/MS: [M+H]⁺: 1503.5.

NE5C (11 mg, 0.047 mmol) was dissolved in 0.5 mL of DMF, and DIPEA (0.011 mL, 0.060 mmol) was added at 0° C. Then AOP (24 mg, 0.053 mmol) was further added, the solution was reacted at room temperature for 1 hour. After 1 hour, 0.5 mL of the above T2428-8 (20 mg, 0.014 mmol) in DMF was added, the solution was reacted at room temperature for 2 hours, the reaction was monitored via LC/MS to find that T2428-8 was consumed completely. The reaction solution was purified by preparative chromatography to obtain 10 mg of 2428 as a white solid with a yield of 46%.

### 30. Synthesis of Target Molecule 2407

### 1). Preparation of Intermediate T2407-1

T2407-1 was prepared with reference to the synthetic method of T2401-1, where BD07 was used instead of BD01. The crude product was purified by preparative chromatography to obtain 4.54 g of T2407-1 as a white solid with a yield of 72%.

### 2). Preparation of Intermediate T2407-2

T2407-2 was prepared with reference to the synthetic method of T2401-2, where T2407-1 was used instead of T2401-1. The crude product was purified by preparative chromatography to obtain 3.5 g of T2407-2 as a white solid with a yield of 68%.

### 3). Preparation of Intermediate T2407-3

T2407-3 was prepared with reference to the synthetic method of T2401-3, where T2407-2 was used instead of T2401-1. The crude product was purified by preparative chromatography to obtain 2.2 g of T2407-3 as a white solid with a yield of 74%.

### 4). Preparation of Intermediate T2407-4

Intermediate NA (340 mg, 0.49 mmol) was dissolved in 5 mL of DMF, DIPEA (0.26 mL, 1.47 mmol) and HATU (210 mg, 0.54 mmol) were added at 0° C., then the solution was reacted at 0° C. for 1 hour, 5 mL of DMF solution in which T2407-3 (600 mg, 0.56 mmol) was dissolved was added dropwise into the reaction solution, which was reacted at 0° C. for 4 hours. Then, 10 mL of water, 10 mL of EA and 0.1 M acetic acid solution were added to the reaction system to adjust the pH to 6, which was then extracted with EA for three times. The organic phases were combined, washed twice with 10 mL of 0.1 M acetic acid solution, and then dried over anhydrous Na₂SO₄ and filtered. The filtrate was rotary evaporated to dryness under reduced pressure to obtain crude product, which was purified by silica gel column chromatography to obtain 550 mg of T2407-4 as a pale yellow solid with a yield of 64%. LC/MS: [M+H]⁺: 1726.7.

### 5). Preparation of Intermediate T2407-5

T2407-4 (550 mg, 0.31 mmol) was dissolved in DCM (30 mL), and p-TsOH·H₂O (302 mg, 1.55 mmol) was added and then the solution was stirred overnight at room temperature, the reaction was monitored via LC/MS until T2407-4 was consumed completely. DCM was rotary evaporated to dryness under reduced pressure, leaving a large amount of solid. Excess water was added in an ice bath and sonicated, resulting in the precipitation of a large amount of solid. The solid was filtered, the liquid phase was discarded, the solid was dissolved in THF, dried over anhydrous Na₂SO₄, THE was rotary evaporated to dryness under reduced pressure to obtain 830 mg of T2407-5 as a pale-yellow crude product, which can be used directly for the next reaction without purification. LC/MS: [M+H]⁺: 1626.6.

### 6). Preparation of Intermediate T2407-6

The above crude T2407-5 (830 mg, 0.51 mmol) was dissolved in 70 mL of DCM, and DIPEA (0.7 mL, 4.08 mmol) was added at 0° C. Then HATU (792 mg, 2.04 mmol) was added, the solution was reacted overnight at 0° C. while stirring, the reaction was monitored via LC/MS to find that T2407-5 was consumed completely. 10 mL of water and 10 mL of DCM were added to the system and extracted once, then the aqueous phase was further extracted twice with 10 mL of EA, the organic phases were combined, and dried over anhydrous Na₂SO₄ and filtered. The filtrate was rotary evaporated to dryness under reduced pressure to obtain crude product, which was purified by silica gel column chromatography to obtain 430 mg of T2407-6 as a yellow solid with a yield of 84%. LC/MS: [M+H]⁺: 1608.6.

### 7). Preparation of Intermediate T2407-7

T2407-6 (430 mg, 0.26 mmol) was dissolved in 150 mL of DCM and 5 mL of acetic acid, and Zhan's catalyst-1B (19.6 mg, 0.026 mmol) was added, the solution was degassed with nitrogen for four times and reacted at 55° C. for 12 hours. The reaction was monitored via LC/MS until T2407-6 was consumed completely, saturated sodium bicarbonate solution was added to the reaction system to adjust the pH to 8, the aqueous phase was then extracted with 10 mL of DCM for three times. The organic phases were combined, and then dried over anhydrous Na₂SO₄ and filtered. The filtrate was rotary evaporated to dryness under reduced pressure to obtain crude product, which was purified by preparative chromatography to obtain 100 mg of T2407-7 as a pale red solid with a yield of 23%. LC/MS: [M+H]⁺: 1580.4.

### 8). Preparation of Intermediate T2407-8

T2401-7 (100 mg, 0.063 mmol) was dissolved in 10 mL of THF, 10 mg of 10% Pd/C was added, the solution was replaced with hydrogen for four times, and reacted overnight at room temperature. The reaction was monitored via LC/MS until T2407-7 was substantially consumed completely. Pd/C was filtered out, and washed with THE for three times, then the organic phase was dried over anhydrous Na₂SO₄ and filtered. The filtrate was rotary evaporated to dryness under reduced pressure to obtain crude T2407-8, which can be used directly for the next reaction without purification. LC/MS: [M+H]⁺: 1446.5.

### 9). Synthesis of Target Molecule 2407

Intermediate NE6C (48 mg, 0.189 mmol) and the above crude T2407-8 was dissolved in 5 mL of DMF, DIPEA (0.088 mL, 0.504 mmol) was added at 0° C. Then AOP (923 mg, 2.04 mmol) was further added, the solution was reacted at 0° C. for 2 hours. The reaction was monitored via LC/MS until T2407-8 consumed completely. The reaction solution was purified by preparative chromatography to obtain 25.1 mg of 2407 as a white solid with a yield of 22.8%. LC/MS: [M]⁺: 1603.8.

TABLE 3
Structural Identification Data Of Some Compounds
In the Synthesis Route of Target Molecules

| Nos. | Structural identification data |
|---|---|
| T2401-1 | LC/MS: [M + H]⁺: 926.7.¹H NMR (500 MHz, CD₃OD) δ: 7.33-7.42 (m, |
| | 4H), 7.32 (d, J = 9.5 Hz, 1H), 7.26 (t, J = 11.0 Hz, 2H), 7.14 (d, J = 9.5 Hz, 2H), |
| | 7.11 (d, J = 9.0 Hz, 2H), 6.84 (d, J = 11.0 Hz, 2H), 5.49-5.87 (m, 1H), 4.94-5.05 |
| | (m, 2H), 4.72-4.80 (m, 2H), 4.50 (s, 1H), 3.98-4.00 (m, 2H), 3.77-3.83 (m, |
| | 1H), 3.60 (d, J = 14.50 Hz, 2H), 3.40-3.48 (m, 4H), 3.15-3.18 (m, 1H), 2.99- |
| | 3.06 (m, 1H), 2.73-2.82 (m, 3H), 1.99-2.08 (m, 2H), 1.71-1.89 (m, 5H), 1.57- |
| | 1.65 (m, 1H), 1.50 (s, 3H), 1.45 (s, 9H), 1.14 (s, 9H), 1.07 (d, J = 7.0 Hz, 3H). |
| T2401-2 | LC/MS: [M + H]⁺: 1146.6.¹H NMR (500 MHz, CD₃OD) δ: 7.22-7.40 |
| | (m, 12H), 7.15 (d, J = 10 Hz, 2H), 7.09 (d, J = 9.0 Hz, 2H), 6.84 (d, J = 10.5 Hz, |
| | 2H), 5.51-5.86 (m, 1H), 4.94-5.06 (m, 4H), 4.71-4.81 (m, 2H), 4.52-4.57 (m, |
| | 1H), 4.48 (s, 1H), 3.97-4.01 (m, 2H), 3.80-3.84 (m, 1H), 3.74 (s, 3H), 3.56- |
| | 3.63 (m, 3H), 3.13-3.16 (m, 1H), 3.01-3.04 (m, 1H), 2.77-2.83 (m, 3H), 1.99- |
| | 2.07 (m, 2H), 1.86-1.89 (m, 1H), 1.70-1.79 (m, 4H), 1.56-1.61 (m, 1H), 1.50 |
| | (s, 3H), 1.45 (s, 9H), 1.14 (s, 9H), 1.07 (d, J = 7.0 Hz, 3H). |
| T2401-3 | LC/MS: [M + H]⁺: 990.4.¹H NMR (500 MHz, CD₃OD) δ: 7.22-7.40 (m, |
| | 12H), 7.15 (d, J = 9.0 Hz, 2H), 7.09 (d, J = 9.0 Hz, 1H), 6.84 (d, J = 11.0 Hz, 2H), |
| | 5.51-5.86 (m, 1H), 4.94-5.04 (m, 4H), 4.77-4.81 (m, 2H), 4.71 (s, 1H), 4.47 |
| | (s, 1H), 4.33-4.36 (m, 1H), 3.76-3.89 (m, 2H), 3.74 (s, 3H), 3.56-3.62 (m, 3H), |
| | 3.39-3.50 (m, 5H), 3.13-3.15 (m, 2H), 3.00-3.04 (m, 1H), 2.76-2.82 (m, 3H), |
| | 2.03-2.07 (m, 2H), 1.88-1.94 (m, 1H), 1.68-1.81 (m, 4H), 1.55-1.58 (m, 1H), |
| | 1.51 (s, 3H), 1.11 (d, J = 8.0 Hz, 3H). |
| T2401-4 | LC/MS: [M + H]⁺: 1664.8.¹H NMR (500 MHz, CD₃OD) δ: 7.39 (d, J = 7.5 |
| | Hz, 1H), 7.30-7.36 (m, 9H), 7.19-7.28 (m, 5H), 7.15 (d, J = 6.5 Hz, 2H), 7.12 |
| | (d, J = 6.0 Hz, 2H), 7.08 (d, J = 9.0 Hz, 2H), 6.88 (dd, J₁ = 2.5 Hz, J₂ = 9.0 Hz, 1H), |
| | 6.85 (s, 1H), 6.80-6.83 (m, 2H), 5.93-6.01 (m, 1H), 5.52-5.83 (m, 1H), 5.11 |
| | (dq, J₁ = 1.5 Hz, J₂ = 10.5 Hz, 1H), 4.95-5.06 (m, 4H), 4.79-4.84 (m, 2H), 4.66- |
| | 4.72 (m, 5H), 4.53-4.57 (m, 1H), 4.47 (s, 1H), 4.40 (dd, J₁ = 3.0 Hz, J₂ = 9.5 Hz, |
| | 1H), 4.25 (d, J = 4.0 Hz, 1H), 4.09-4.18 (m, 3H), 4.04-4.09 (m, 1H), 4.01 (d, |
| | J = 15.0 Hz, 1H), 3.89 (q, J = 9.0 Hz, 1H), 3.78-3.83 (m, 1H), 3.71 (s, 3H), 3.57- |
| | 3.60 (m, 3H), 3.44-3.50 (m, 2H), 3.36-3.43 (m, 3H), 3.27-3.29 (m, 1H), 3.20 |
| | (dd, J₁ = 6.5 Hz, J₂ = 15.0 Hz, 1H), 3.14 (t, J = 7.5 Hz, 1H), 2.97-3.04 (m, 3H), |
| | 2.92 (dd, J₁ = 3.5 Hz, J₂ = 13.5 Hz, 1H), 2.72-2.82 (m, 3H), 1.97-2.08 (m, 4H), |
| | 1.84-1.88 (m, 1H), 1.68-1.78 (m, 4H), 1.55-1.60 (m, 1H), 1.46 (s, 9H), 1.49 |
| | (s, 3H), 1.46 (s, 9H), 1.29-1.34 (m, 1H). |
| T2401-6 | LC/MS: [M + H]⁺: 1546.6.¹H NMR (500 MHz, CD₃OD) δ: 7.25-7.37 |
| | (m, 10H), 7.16-7.23 (m, 5H), 7.09-7.13 (m, 5H), 6.98-7.05 (m, 2H), 6.83-6.91 |
| | (m, 3H), 5.82-5.89 (m, 1H), 5.52-5.82 (m, 1H), 4.94-5.10 (m, 5H), 4.68-4.82 |
| | (m, 5H), 4.58-4.61 (m, 4H), 4.39-4.47 (m, 2H), 4.28 (s, 1H), 4.11-4.23 (m, |
| | 4H), 3.98-4.00 (m, 1H), 3.72-3.84 (m, 5H), 3.64-3.67 (m, 2H), 3.56-3.60 (m, |
| | 2H), 3.42-3.52 (m, 4H), 3.18-3.24 (m, 2H), 2.85-3.02 (m, 6H), 2.71-2.79 (m, |
| | 2H), 2.00-2.08 (m, 3H), 1.62-1.68 (m, 3H), 1.47-1.52 (m, 3H), 1.23-1.35 (m, |
| | 4H), 1.08-1.12 (m, 3H). |
| 2401 | HRMS: [M]⁺: 1540.8070.¹H NMR(500 MHz, CD₃OD) δ: 7.70 (dd, |
| | J₁ = 4.5 Hz, J₂ = 8.0 Hz, 1H), 7.61 (d, J = 8.0 Hz, 2H), 7.40 (d, J = 7.5 Hz, 2H), |
| | 7.28 (dd, J₁ = 2.5 Hz, J₂ = 9.5 Hz, 1H), 7.20-7.26 (m, 4H), 7.16 (dd, J₁ = 1.5 Hz, |
| | J₂ = 7.5 Hz, 1H), 7.12 (d, J = 8.0 Hz, 2H), 7.00 (d, J = 7.5 Hz, 2H), 6.94-6.98 (m, |
| | 4H), 6.41 (brs, 1H), 5.57 (brs, 1H), 5.51 (d, J = 14.5Hz, 1H), 4.73 (dd, J₁ = 3.5 |
| | Hz, J₂ = 11.5 Hz, 1H), 4.63-4.70 (m, 3H), 4.45 (s, 1H), 4.36 (dd, J₁ = 4.5 Hz, |
| | J₂ = 10.5 Hz, 1H), 4.28-4.30 (m, 3H), 4.16-4.22 (m, 2H), 4.09-4.12 (m, 1H), |
| | 4.01-4.09 (m, 3H), 3.77-3.87 (m, 7H), 3.69-3.75 (m, 2H), 3.47-3.54 (m, 2H), |
| | 3.36-3.39 (m, 1H), 3.27 (dd, J₁ = 6.0 Hz, J₂ = 11.0 Hz, 2H), 3.19-3.24 (m, 1H), |
| | 3.00-3.10 (m, 10H), 2.94-2.96 (m, 2H), 2.84 (dt, J₁ = 4.5 Hz, J₂ = 14.5 Hz, 1H), |
| | 2.72-2.80 (m, 2H), 2.48-2.55 (m, 2H), 2.26-2.32 (m, 3H), 2.16-2.22 (m, 1H), |
| | 2.01-2.05 (m, 1H), 1.75-1.82 (m, 2H), 1.67-1.73 (m, 2H), 1.36-1.45 (m, 6H), |
| | 1.32-1.35 (m, 1H), 1.17-1.22 (m, 1H), 1.07 (d, J = 6.0 Hz, 3H), 0.97 (s, 3H), |
| | 0.77-0.81 (m, 2H), 0.41-0.57 (m, 4H) |
| 2450 | LC/MS: [M]⁺: 1587.8.¹H NMR (500 MHz, CD₃OD) δ: 7.60 (d, J = 7.5 |
| | Hz, 2H), 7.39 (d, J = 7.5 Hz, 2H), 7.23-7.30 (m, 4H), 7.12-7.21 (m, 4H), 7.00 |
| | (d, J = 6.5 Hz, 2H), 6.95-6.97 (m, 4H), 6.42-6.44 (m, 2H), 5.46-5.49 (m, 2H), |
| | 5.33-5.36 (m, 1H), 4.73 (dd, J₁ = 3.5 Hz, J₂ = 11.5 Hz, 1H), 4.64-4.67 (m, 2H), |
| | 4.59 (s, 3H), 4.47 (s, 1H), 4.41 (dd, J₁ = 4.5 Hz, J₂ = 10.0 Hz, 1H), 4.28 (dd, |
| | J₁ = 3.0 Hz, J₂ = 8.5 Hz, 2H), 4.17-4.22 (m, 2H), 4.11-4.13 (m, 1H), 4.01-4.06 |
| | (m, 3H), 3.79-3.89 (m, 6H), 3.69-3.78 (m, 4H), 3.62 (s, 3H), 3.44-3.57 (m, |
| | 5H), 3.35-3.38 (m, 2H), 3.15-3.21 (m, 5H), 3.00-3.10 (m, 3H), 2.93-2.987 (m, |
| | 2H), 2.75-2.86 (m, 3H), 2.47-2.53 (m, 3H), 2.28-2.32 (m, 2H), 2.18-2.21 (m, |
| | 2H), 2.01-2.05 (m, 2H), 1.59-1.63 (m, 2H), 1.41-1.46 (m, 2H), 1.16-1.20 (m, |
| | 2H), 1.07 (d, J = 6.0 Hz, 3H), 0.98 (s, 3H), 0.09 (t, J = 6.5 Hz, 2H), 0.52-0.59 |
| | (m, 3H), 0.41-0.45 (m, 2H). |
| 2451 | HRMS: [M]⁺: 1526.7901.¹H NMR (500 MHz, CD₃OD) δ: 7.67 (dd, |
| | J₁ = 4.5 Hz, J₂ = 7.5 Hz, 1H), 7.61 (d, J = 8.0 Hz, 2H), 7.40 (d, J = 7.5 Hz, 2H), |
| | 7.20-7.29 (m, 4H), 7.15-7.17 (m, 1H), 7.12 (d, J = 7.5 Hz, 2H), 6.98-7.00 (m, |
| | 3H), 6.91 -6.96 (m, 3H), 6.40-6.43 (m, 2H), 5.49-5.42 (m, 2H), 5.33-5.36 (m, |
| | 1H), 4.72-4.75 (m, 2H), 4.63-4.69 (m, 2H), 4.47 (s, 1H), 4.28-4.36 (m, 4H), |
| | 4.16-4.22 (m, 2H), 4.10-4.12 (m, 1H), 4.01-4.08 (m, 3H), 3.69-3.88 (m, 9H), |
| | 3.49-3.51 (m, 1H), 3.44-3.48 (m, 1H), 3.33-3.39 (m, 2H), 3.16-3.22 (m, 2H), |
| | 3.07-3.08 (m, 7H), 2.94-2.97 (m, 2H), 2.84 (dt, J₁ = 4.5 Hz, J₂ = 14.5 Hz, 1H), |
| | 2.73-2.80 (m, 2H), 2.48-2.53 (m, 2H), 2.27-2.34 (m, 3H), 2.18-2.21 (m, 2H), |
| | 2.01-2.05 (m, 1H), 1.78-1.85 (m, 2H), 1.64-1.70 (m, 2H), 1.60 (t, J = 2.5 Hz, |
| | 1H), 1.40-1.45 (m, 3H), 1.70-1.22 (m, 2H), 1.07 (d, J = 6.5 Hz, 3H), 0.97 (s, |
| | 3H), 0.77-0.81 (m, 2H), 0.52-0.56 (m, 3H), 0.39-0.44 (m, 2H). |
| 2455 | HRMS: [M]⁺: 1484.7686.¹H NMR (500 MHz, CD₃OD) δ: 7.64-7.67 |
| | (m, 3H), 7.44 (d, J = 8.0 Hz, 2H), 7.23-7.29 (m, 4H), 7.17-7.21 (m, 2H), 7.11 |
| | (d, J = 8.0 Hz, 2H), 6.96-7.00 (m, 5H), 6.91-6.96 (m, 3H), 6.89 (brs, 1H), 6.83- |
| | 6.85 (m, 1H), 6.15-6.18 (m, 1H), 5.57 (brs, 1H), 5.30 (d, J = 15.0 Hz, 1H), 4.72- |
| | 4.77 (m, 2H), 4.63 (t, J = 8.0 Hz, 1H), 4.39-4.42 (m, 2H), 4.25-4.29 (m, 3H), |
| | 4.22 (s, 1H), 4.09-4.18 (m, 2H), 3.97-4.06 (m, 3H), 3.77-3.93 (m, 7H), 3.63- |
| | 3.75 (m, 3H), 3.33-3.40 (m, 3H), 3.20-3.24 (m, 1H), 2.97-3.07 (m, 11H), 2.94- |
| | 3.11 (m, 3H), 2.83 (dt, J₁ = 4.5 Hz, J₂ = 14.5 Hz, 1H), 2.94-2.97 (m, 2H), 2.84 |
| | (dt, J₁ = 4.5 Hz, J₂ = 14.5 Hz, 1H), 2.78 (dd, J₁ = 4.5 Hz, J₂ = 12.5 Hz, 1H), 2.63- |
| | 2.68 (m, 1H), 2.48-2.57 (m, 2H), 2.25 (dd, J₁ = 7.0 Hz, J₂ = 13.5 Hz, 1H), 2.13- |
| | 2.19 (m, 1H), 1.43-1.48 (m, 4H), 1.23-1.32 (m, 2H), 1.05 (d, J = 6.5 Hz, 3H), |
| | 0.94 (s, 3H), 0.76-0.82 (m, 3H), 0.58-0.63 (m, 1H), 0.42-0.49 (m, 2H). |
| 2457 | HRMS: [M]⁺: 1512.7736.¹H NMR (500 MHz, CD₃OD) δ: 7.63 (d, J = 7.5 |
| | Hz, 2H), 7.41 (d, J = 8.0 Hz, 2H), 7.19-7.28 (m, 5H), 7.15-7.17 (m, 1H), 7.11 |
| | (d, J = 8.0 Hz, 2H), 6.87-7.00 (m, 7H), 6.35-6.39 (m, 1H), 5.52 (d, J = 15.0 Hz, |
| | 2H), 4.74 (dd, J₁ = 3.5 Hz, J₂ = 11.0 Hz, 1H), 4.68 (d, J = 14.0 Hz, 1H), 4.64 (t, |
| | J = 8.0 Hz, 1H), 4.49 (s, 1H), 4.36 (dd, J₁ = 4.0 Hz, J₂ = 11.0 Hz, 1H), 4.32 (t, |
| | J = 5.5 Hz, 1H), 4.28-4.30 (m, 2H), 4.22 (d, J = 15.5 Hz, 1H), 4.17 (t, J = 9.0 Hz, |
| | 1H), 4.09-4.13 (m, 1H), 4.01-4.08 (m, 3H), 3.76-3.86 (m, 7H), 3.68-3.74 (m, |
| | 2H), 3.52 (dd, J₁ = 4.5 Hz, J₂ = 14.0 Hz, 1H), 3.44-3.45 (m, 1H), 3.33-3.42 (m, |
| | 4H), 3.19-3.23 (m, 1H), 3.00-3.13 (m, 9H), 2.92-2.97 (m, 2H), 2.79-2.86 (m, |
| | 2H), 2.71 (t, J = 12.0 Hz, 1H), 2.47-2.54 (m, 2H), 2.34 (t, J = 7.0 Hz, 2H), 2.29 |
| | (dd, J₁ = 6.5 Hz, J₂ = 13.5 Hz, 1H), 2.01-2.21 (m, 4H), 1.39-1.46 (m, 4H), 1.29- |
| | 1.32 (m, 2H), 1.18-1.21 (m, 1H), 1.06 (d, J = 6.0 Hz, 3H), 0.95 (s, 3H), 0.75- |
| | 0.86 (m, 3H), 0.51-0.56 (m, 2H), 0.39-0.44 (m, 1H). |
| 2460 | HRMS: [M]⁺: 1554.8195.¹H NMR (400 MHz, CD₃OD) δ: 7.60-7.62 |
| | (m, 3H), 7.39 (d, J = 8.0 Hz, 2H), 7.19-7.30 (m, 6H), 7.15-7.19 (m, 1H), 7.12 |
| | (d, J = 8.0 Hz, 2H), 6.94-7.02 (m, 6H), 5.55 (brs, 1H), 5.50 (d, J = 14.4 Hz, 1H), |
| | 4.70-4.73 (m, 1H), 4.64-4.70 (m, 2H), 4.47 (s, 1H), 4.36-4.41 (m, 1H), 4.26 - |
| | 4.32 (m, 3H), 4.16-4.22 (m, 2H), 4.01-4.12 (m, 4H), 3.69-3.89 (m, 10H), 3.47- |
| | 3.51 (m, 2H), 3.35-3.39 (m, 1H), 3.25-3.29 (m, 2H), 3.18-3.23 (m, 1H), 3.02- |
| | 3.14 (m, 10H), 2.91-2.98 (m, 3H), 2.84 (dt, J₁ = 4.2 Hz, J₂ = 14.4 Hz, 1H), 2.72- |
| | 2.80 (m, 2H), 2.50-2.56 (m, 2H), 2.17-2.33 (m, 4H), 1.71-1.79 (m, 2H), 1.62- |
| | 1.69 (m, 2H), 1.33-1.42 (m, 8H), 1.16-1.23 (m, 1H), 1.07 (d, J = 6.4 Hz, 3H), |
| | 0.97 (s, 3H), 0.89-0.92 (m, 2H), 0.77-0.81 (m, 2H), 0.41-0.56 (m, 3H). |
| 2461 | HRMS: [M]⁺: 1666.9744.¹H NMR (500 MHz, CD₃OD) &: 7.67 (dd, |
| | J₁ = 4.5 Hz, J₂ = 7.5 Hz, 1H), 7.60 (d, J = 7.5 Hz, 2H), 7.39 (d, J = 8.0 Hz, 2H), |
| | 7.28 (dd, J₁ = 2.5 Hz, J₂ = 10.0 Hz, 1H), 7.21-7.27 (m, 3H), 7.18-7.21 (m, 1H), |
| | 7.15 (d, J = 7.0 Hz, 1H), 7.12 (d, J = 7.5 Hz, 2H), 6.94-7.01 (m, 7H), 6.41-6.44 |
| | (m, 1H), 5.49-5.53 (m, 2H), 4.70-4.75 (m, 2H), 4.63-4.68 (m, 2H), 4.46 (s, |
| | 1H), 4.39-4.42 (m, 1H), 4.27-4.30 (m, 3H), 4.16-4.22 (m, 2H), 4.08-4.12 (m, |
| | 2H), 4.01-4.06 (m, 2H), 3.86-3.88 (m, 1H), 3.81-3.84 (m, 4H), 3.75-3.79 (m, |
| | 2H), 3.70 (d, J = 14.5 Hz, 1H), 3.45-3.51 (m, 1H), 3.33-3.89 (m, 2H), 3.19-3.23 |
| | (m, 1H), 3.10-3.12 (m, 11H), 3.00-3.09 (m, 2H), 2.94-2.97 (m, 2H), 2.84 (dt, |
| | J₁ = 4.5 Hz, J₂ = 14.5 Hz, 1H), 2.72-2.78 (m, 2H), 2.47-2.55 (m, 2H), 2.28-2.34 |
| | (m, 2H), 2.21 (t, J = 7.5 Hz, 2H), 1.75-1.81 (m, 3H), 1.551.65 (m, 3H), 1.33- |
| | 1.40 (m, 15H), 1.26-1.30 (m, 4H), 1.22 (d, J = 6.5 Hz, 2H), 1.07 (d, J = 6.0 Hz, |
| | 3H), 0.96 (s, 3H), 0.88-0.91 (m, 2H), 0.77-0.82 (m, 2H), 0.41-0.58 (m, 4H) |
| 2462 | LC/MS: [M]⁺: 1608.7.¹H NMR (400 MHz, CD₃OD) δ: 7.83 (d, J = 7.6 |
| | Hz, 2H), 7.71-7.74 (m, 1H), 7.55 (d, J = 8.0 Hz, 2H), 7.19-7.31 (m, 6H), 7.03 |
| | (d, J = 8.4 Hz, 4H), 6.92 (d, J = 8.0 Hz, 2H), 6.73 (s, 1H), 6.53-6.56 (m, 1H), |
| | 5.74-5.76 (m, 1H), 5.55-5.58 (m, 1H), 5.13-5.16 (m, 1H), 4.57-4.61 (m, 2H), |
| | 4.18-4.42 (m, 8H), 4.01-4.12 (m, 5H), 3.90-3.93 (m, 1H), 3.87 (s, 3H), 3.70- |
| | 3.81 (m, 3H), 3.37-3.56 (m, 6H), 3.18-3.26 (m, 3H), 3.09-3.18 (m, 8H), 2.89- |
| | 2.99 (m, 4H), 2.80-2.85 (m, 2H), 2.17-2.37 (m, 6H), 2.01-2.05 (m, 1H), 1.78- |
| | 1.86 (m, 2H), 1.63-1.69 (m, 3H), 1.49-1.63 (m, 5H), 1.33-1.36 (m, 2H), 1.00 |
| | (d, J = 6.4 Hz, 3H), 0.71 (s, 3H), 0.48-0.51 (m, 1H), 0.33-0.36 (m, 2H). |
| 2463 | LC/MS: [M]⁺: 1626.7.¹H NMR (600 MHz, CD₃OD) δ: 7.81 (d, J = 6.6 |
| | Hz, 2H), 7.74 (d, J = 7.8 Hz, 1H), 7.54 (d, J = 7.8 Hz, 2H), 7.35 (d, J = 7.2 Hz, |
| | 1H), 7.25-7.29 (m, 3H), 7.23 (d, J = 7.8 Hz, 1H), 7.20 (d, J = 7.2 Hz, 1H), 7.02- |
| | 7.04 (m, 4H), 6.93 (d, J = 7.8 Hz, 2H), 6.72 (s, 1H), 6.64 (brs, 1H), 5.81 (brs, |
| | 1H), 5.57 (d, J = 15.0 Hz, 1H), 5.19 (brs, 1H), 4.90-4.92 (m, 1H), 4.58-4.61 (m, |
| | 2H), 4.37-4.40 (m, 2H), 4.21-4.33 (m, 5H), 4.03-4.11 (m, 5H), 3.88-3.92 (m, |
| | 2H), 3.86 (s, 3H), 3.79 (d, J = 15.0 Hz, 1H), 3.71 (d, J = 13.2 Hz, 1H), 3.55 (d, |
| | J = 13.8 Hz, 1H), 3.42-3.50 (m, 3H), 3.33-3.36 (m, 2H), 3.19-3.26 (m, 3H), |
| | 3.11-3.14 (m, 8H), 2.91-3.01 (m, 4H), 2.80-2.86 (m, 2H), 2.40 (t, J = 13.2 Hz, |
| | 1H), 2.29-2.33 (m, 4H), 2.18-2.24 (m, 1H), 1.79-1.84 (m, 2H), 1.63-1.70 (m, |
| | 3H), 1.50-1.59 (m, 4H), 1.29-1.33 (m, 3H), 0.99 (d, J = 7.8 Hz, 3H), 0.72 (s, |
| | 3H), 0.37-0.51 (m, 4H). |
| T2402-1 | LC/MS: [M + H]⁺: 940.5.¹H NMR (500 MHz, CD₃OD) δ: 7.44 (d, J = 8.0 |
| | Hz, 1H), 7.35-7.37 (m, 3H), 7.31 (d, J = 7.5 Hz, 1H), 7.26 (dd, J₁ = 8.0 Hz, |
| | J₂ = 13.0 Hz, 2H), 7.15 (d, J = 5.0 Hz, 2H), 7.10 (d, J = 7.5 Hz, 1H), 6.83-6.86 |
| | (m, 2H), 5.48-5.86 (m, 1H), 4.96-5.01 (m, 2H), 4.79-4.84 (m, 2H), 4.72 (s, |
| | 1H), 4.50 (s, 1H), 3.97-4.01 (m, 2H), 3.78-3.83 (m, 1H), 3.74 (s, 3H), 3.35- |
| | 3.48 (m, 4H), 3.14-3.19 (m, 1H), 2.99-3.06 (m, 1H), 2.74-2.85 (m, 3H), 1.98- |
| | 2.09 (m, 2H), 1.86-1.91 (m, 1H), 1.71-1.82 (m, 4H), 1.55-1.61 (m, 1H), 1.50 |
| | (s, 3H), 1.41-1.48 (m, 12H), 1.14 (s, 9H), 1.07 (d, J = 6.0 Hz, 3H). |
| T2402-2 | LC/MS: [M + H]⁺: 1160.6.¹H NMR (500 MHz, CD₃OD) δ: 7.44 (t, J = 7.5 |
| | Hz, 1H), 7.20-7.39 (m, 11H), 7.16 (d, J = 8.0 Hz, 2H), 7.08 (t, J = 8.0 Hz, 1H), |
| | 6.84 (d, J = 8.5 Hz, 2H), 5.49-5.85 (m, 1H), 5.00-5.07 (m, 2H), 4.94-4.96 (m, |
| | 1H), 4.79-4.84 (m, 2H), 4.71 (s, 1H), 4.45-4.54 (m, 2H), 3.98-4.02 (m, 2H), |
| | 3.78-3.83 (m, 1H), 3.72-3.76 (m, 4H), 3.45-3.54 (m, 2H), 3.36-3.43 (m, 3H), |
| | 3.11-3.16 (m, 1H), 3.01-3.05 (m, 1H), 2.72-2.83 (m, 3H), 1.98-2.08 (m, 2H), |
| | 1.86-1.89 (m, 1H), 1.69-1.77 (m, 4H), 1.54-1.60 (m, 1H), 1.50 (s, 3H), 1.41- |
| | 1.47 (m, 13H), 1.14 (s, 9H), 1.07 (d, J = 6.0 Hz, 3H). |
| T2402-3 | LC/MS: [M + H]⁺: 1004.5.¹H NMR (500 MHz, CD₃OD) δ: 7.20-7.44(m, |
| | 12H), 7.08-7.15 (m, 3H), 6.83 (dd, J₁ = 3.5 Hz, J₂ = 10.5 Hz, 2H), 5.46-5.80 (m, |
| | 1H), 4.99-5.05 (m, 3H), 4.70-4.75 (m, 2H), 4.45-4.46 (m, 1H), 4.28-4.35 (m, |
| | 1H), 3.80-3.89 (m, 2H), 3.74 (s, 3H), 3.71-3.74 (m, 1H), 3.35-3.48 (m, 6H), |
| | 3.18-3.22 (m, 1H), 3.10-3.13 (m, 1H), 2.97-3.03 (m, 1H), 2.74-2.81 (m, 3H), |
| | 2.03-2.07 (m, 2H), 1.88-1.92 (m, 1H), 1.70-1.78 (m, 4H), 1.53-1.57 (m, 1H), |
| | 1.51 (s, 3H), 1.28-1.31 (m, 1H), 1.15 (d, J = 8.0 Hz, 3H). |
| T2402-4 | LC/MS: [M + H]⁺: 1677.8.¹H NMR (500 MHz, CD₃OD) δ: 7.36-7.43 (m, |
| | 2H), 7.19-7.36 (m, 13H), 7.12-7.16 (m, 3H), 7.07 (t, J = 9.0 Hz, 3H), 6.85-6.90 |
| | (m, 2H), 6.81 (d, J = 7.5 Hz, 2H), 5.93-6.01 (m, 1H), 5.49-5.82 (m, 1H), 5.11 |
| | (d, J = 10.5 Hz, 1H), 5.03-5.05 (m, 1H), 4.95-5.00 (m, 2H), 4.79-4.82 (m, 2H), |
| | 4.64-4.72 (m, 6H), 4.38-4.45 (m, 2H), 4.32-4.36 (m, 1H), 4.25 (d, J = 4.5 Hz, |
| | 1H), 4.11-4.15 (m, 2H), 4.05-4.09 (m, 1H), 4.00 (d, J = 14.5 Hz, 1H), 3.86-3.91 |
| | (m, 1H), 3.78-3.83 (m, 1H), 3.71-3.74 (m, 4H), 3.56-3.63 (m, 1H), 3.46-3.50 |
| | (m, 2H), 3.36-3.40 (m, 3H), 3.21 (dd, J₁ = 6.5 Hz, J₂ = 15.0 Hz, 1H), 3.10-3.12 |
| | (m, 1H), 2.98-3.05 (m, 4H), 2.90-2.94 (m, 2H), 2.73-2.82 (m, 3H), 1.99-2.07 |
| | (m, 5H), 1.82-1.87 (m, 1H), 1.67-1.75 (m, 4H), 1.54-1.57 (m, 1H), 1.49 (s, |
| | 3H), 1.36-1.47 (m, 12H), 1.12 (d, J = 6.5 Hz, 3H). |
| T2402-6A | ¹H NMR (500 MHz, CD₃OD) δ: 7.28-7.37 (m, 6H), 7.22-7.28 (m, 6H), |
| | 7.10-7.19 (m, 6H), 7.03-7.06 (m, 3H), 6.87 (d, J = 9.0 Hz, 3H), 6.77 (t, J = 9.0 |
| | Hz, 3H), 5.82-5.89 (m, 1H), 5.49-5.81 (m, 1H), 4.91-5.11 (m, 5H), 4.73-4.80 |
| | (m, 3H), 4.61-4.70 (m, 4H), 4.50-4.61 (m, 2H), 4.41-4.50 (m, 1H), 4.25-4.35 |
| | (m, 2H), 4.18-4.22 (m, 2H), 4.10-4.14 (m, 1H), 3.96-4.03 (m, 2H), 3.71-3.77 |
| | (m, 4H), 3.55-3.66 (m, 3H), 3.49-3.52 (m, 1H), 3.41-3.46 (m, 3H), 3.23-3.28 |
| | (m, 2H), 3.16-3.23 (m, 1H), 2.99-3.06 (m, 4H), 2.86-2.94 (m, 2H), 2.71-2.77 |
| | (m, 2H), 2.02-2.07 (m, 3H), 1.65-1.74 (m, 4H), 1.40-1.52 (m, 6H), 1.32 (s, |
| | 3H), 1.12 (dd, J₁ = 6.0 Hz, J₂ = 12.0 Hz, 3H)). |
| T2402-6B | ¹H NMR (500 MHz, CD₃OD) δ: 7.30-7.37 (m, 6H), 7.23-7.30 (m, 6H), |
| | 7.10-7.19 (m, 6H), 7.02-7.07 (m, 3H), 6.87 (d, J = 9.0 Hz, 3H), 6.71-6.79 (m, |
| | 1H), 5.82-5.90 (m, 1H), 5.49-5.52 (m, 1H), 4.94-5.11 (m, 5H), 4.71-4.81 (m, |
| | 4H), 4.55-4.68 (m, 5H), 4.46 (q, J = 16.0 Hz, 1H), 4.30-4.36 (m, 2H), 4.19-4.25 |
| | (m, 2H), 4.10-4.14 (m, 1H), 3.92-4.05 (m, 2H), 3.71-3.77 (m, 4H), 3.55-3.70 |
| | (m, 3H), 3.42-3.49 (m, 4H), 3.22-3.28 (m, 2H), 2.97-3.08 (m, 5H), 2.86-2.94 |
| | (m, 2H), 2.70-2.78 (m, 2H), 2.03-2.07 (m, 3H), 1.68-1.74 (m, 4H), 1.44-1.52 |
| | (m, 6H), 1.32 (s, 3H), 1.12 (dd, J₁ = 6.0 Hz, J₂ = 13.0 Hz, 3H). |
| 2402A | LC/MS: [M]⁺: 1554.8.¹H NMR (500 MHz, CD₃OD) δ: 7.70-7.78 (m, |
| | 3H), 7.52-7.54 (m, 2H), 7.18-7.29 (m, 6H), 6.96-7.06 (m, 6H), 6.65-6.79 (m, |
| | 3H), 5.86 (brs, 1H), 5.62 (brs, 1H), 5.31-5.38 (m, 3H), 4.82-4.85 (m, 2H), |
| | 4.57-4.76 (m, 5H), 4.25-4.35 (m, 3H), 3.92-4.19 (m, 7H), 3.71-3.91 (m, 5H), |
| | 3.53-3.65 (m, 3H), 3.35-3.45 (m, 2H), 3.16-3.21 (m, 4H), 2.99-3.02(m, 3H), |
| | 2.90-2.96 (m, 2H), 2.81-2.84(m, 2H), 2.43-2.48 (m, 2H), 2.34-2.37 (m, 3H), |
| | 2.19 (t, J = 7.5 Hz, 2H), 2.03 (q, J = 6.5 Hz, 2H), 1.49-1.62 (m, 5H), 1.29-1.35 |
| | (m, 10H), 1.02 (d, J = 6.5 Hz, 2H), 0.90 (t, J = 7.0 Hz, 3H), 0.53-0.69 (m, 4H), |
| | 0.34 (brs, 1H). |
| 2402B | LC/MS: [M]⁺: 1554.8.¹H NMR (500 MHz, CD₃OD) δ: 7.59 (d, J = 8.0 |
| | Hz, 2H), 7.36 (dd, J₁ = 2.5 Hz, J₂ = 9.5 Hz, 2H), 7.24-7.30 (m, 4H), 7.13-7.24 |
| | (m, 6H), 6.97 (d, J = 8.5 Hz, 2H), 6.91 (d, J = 7.5 Hz, 2H), 6.76 (t, J = 9.0 Hz, |
| | 2H), 5.50 (brs, 2H), 5.31-5.38 (m, 3H), 4.69-4.74 (m, 2H), 4.64 (s, 3H), 4.61 |
| | (s, 1H), 4.54 (dd, J₁ = 2.5 Hz, J₂ = 11.5 Hz, 1H), 4.49 (d, J = 14.0 Hz, 1H), 4.33 |
| | (d, J = 3.0 Hz, 1H), 4.25-4.29 (m, 2H), 4.13-4.17 (m, 1H), 4.09-4.13 (m, 2H), |
| | 4.05 (t, J = 5.5 Hz, 1H), 4.00-4.03 (m, 1H), 3.89-3.93 (m, 1H), 3.85-3.89 (m, |
| | 1H), 3.79 (s, 2H), 3.74 (s, 1H), 3.69-3.72 (m, 1H), 3.60-3.62 (m, 1H), 3.54- |
| | 3.60 (m, 2H), 3.50 (brs, 1H), 3.47 (brs, 1H), 3.45 (quintet, J = 1.5 Hz, 1H), |
| | 3.33-3.35 (m, 1H), 3.24-3.26 (m, 1H), 3.17 (quintet, J = 2.0 Hz, 1H), 3.02-3.10 |
| | (m, 5H), 2.96-3.00 (m, 2H), 2.84 (dt, J₁ = 5.0 Hz, J₂ = 14.5 Hz, 1H), 2.67-2.70 |
| | (m, 1H), 2.51-2.55 (m, 1H), 2.34-2.38 (m, 1H), 2.28 (t, J = 7.5 Hz, 2H), 2.19 |
| | (t, J = 7.5 Hz, 2H), 2.03 (q, J = 6.5 Hz, 2H), 1.75-1.80 (m, 1H), 1.66-1.71 (m, |
| | 2H), 3.17 (quintet, J = 7.0 Hz, 2H), 1.32-1.41 (m, 10H), 1.12 (d, J = 6.5 Hz, 3H), |
| | 1.01 (s, 2H), 0.90 (t, J = 7.0 Hz, 3H), 0.73 (brs, 1H), 0.46 (quintet, J = 8.0 Hz, |
| | 2H). |
| 204B | LC/MS: [M]⁺: 1526.8.¹H NMR (500 MHz, CD₃OD) δ: 7.60 (d, J = 7.5 |
| | Hz, 2H), 7.35 (dd, J₁ = 2.5 Hz, J₂ = 10.0 Hz, 1H), 7.23-7.30 (m, 4H), 7.13-7.25 |
| | (m, 7H), 6.97 (d, J = 8.0 Hz, 2H), 6.91 (d, J = 8.0 Hz, 2H), 6.75 (t, J = 9.0 Hz, |
| | 1H), 5.48-5.50 (m, 2H), 5.34 (t, J = 5.0 Hz, 1H), 4.69-4.74 (m, 2H), 4.49-4.57 |
| | (m, 2H), 4.26-4.34 (m, 3H), 4.06-4.17 (m, 5H), 4.01 (d, J = 14.5 Hz, 1H), 3.87- |
| | 3.93 (m, 2H), 3.79 (s, 3H), 3.69-3.74 (m, 1H), 3.60-3.66 (m, 2H), 3.52-3.58 |
| | (m, 1H), 3.45-3.52 (m, 2H), 3.35-3.40 (m, 2H), 3.21-3.25 (m, 1H), 3.08-3.14 |
| | (m, 9H), 2.99-3.08 (m, 2H), 2.96-2.99 (m, 2H), 2.84 (dt, J₁ = 5.0 Hz, J₂ = 14.5 |
| | Hz, 1H), 2.72 (d, J = 13.5 Hz, 1H), 2.64-2.67 (m, 1H), 2.50-2.56 (m, 1H), 2.37 |
| | (t, J = 7.0 Hz, 3H), 2.17-2.23 (m, 2H), 2.01-2.10 (m, 3H), 1.69 (d, J = 7.0 Hz, |
| | 3H), 1.31-1.45 (m, 4H), 1.12 (d, J = 6.5 Hz, 3H), 1.06-1.10 (m, 2H), 1.00 (s, |
| | 3H), 0.87-0.92 (m, 2H), 0.71 (brs, 1H), 0.43-0.49 (m, 2H). |
| 205B | LC/MS: [M]⁺: 1540.8.¹H NMR (500 MHz, CD₃OD) δ: 7.60 (d, J = 7.5 |
| | Hz, 2H), 7.34 (dd, J₁ = 2.5 Hz, J₂ = 10.0 Hz, 1H), 7.25-7.30 (m, 5H), 7.12-7.23 |
| | (m, 7H), 6.97 (d, J = 8.0 Hz, 2H), 6.90 (d, J = 7.5 Hz, 2H), 6.78 (t, J = 9.0 Hz, |
| | 1H), 5.58-5.63 (m, 1H), 5.47-5.52 (m, 2H), 4.67-4.74 (m, 3H), 4.47-4.55 (m, |
| | 3H), 4.34 (d, J = 3.5 Hz, 1H), 4.28-4.31 (m, 2H), 4.10-4.18 (m, 4H), 4.05-4.07 |
| | (m, 1H), 4.01 (d, J = 14.5 Hz, 1H), 3.88-3.96 (m, 2H), 3.79 (s, 3H), 3.68-3.74 |
| | (m, 2H), 3.59-3.64 (m, 3H), 3.90 (d, J = 15.5 Hz, 1H), 3.33-3.34 (m, 1H), 3.21- |
| | 3.25 (m, 2H), 3.00-3.17 (m, 9H), 2.94-3.00 (m, 3H), 2.84 (dt, J₁ = 5.0 Hz, |
| | J₂ = 14.5 Hz, 2H), 2.63-2.69 (m, 2H), 2.50-2.56 (m, 1H), 2.31-2.39 (m, 3H), |
| | 2.17-2.24 (m, 2H), 1.83 (brs, 3H), 1.68 (d, J = 7.0 Hz, 3H), 1.31-1.45 (m, 3H), |
| | 1.12(d, J = 6.0 Hz, 3H), 1.06-1.10 (m, 2H), 1.00 (s, 3H), 0.87-0.92 (m, 2H), |
| | 0.70 (brs, 1H), 0.43-0.49 (m, 2H). |
| 205A | HRMS: [M]⁺: 1540.8021. ¹H NMR (500 MHz, CD₃OD) δ: 7.78 (d, J = 8.0 |
| | Hz, 2H), 7.52 (d, J = 8.0 Hz, 2H), 7.22-7.27 (m, 4H), 7.14-7.22 (m, 3H), 7.05 |
| | (d, J = 7.5Hz, 2H), 6.99-7.02 (m, 2H), 6.96 (d, J = 7.5 Hz, 2H), 6.92 (s, 1H), 6.78 |
| | (s, 1H), 6.61-6.63 (m, 1H), 5.85 (brs, 1H), 5.52 (d, J = 15.0 Hz, 1H), 4.83 (d, |
| | J = 13.5 Hz, 1H), 4.73 (dd, J₁ = 4.0 Hz, J₂ = 11.5 Hz, 1H), 4.58 (dd, J₁ = 7.0 Hz, |
| | J₂ = 9.5 Hz, 1H), 4.36 (s, 1H), 4.27-4.30 (m, 4H), 4.14-4.23 (m, 2H), 3.97-4.11 |
| | (m, 5H), 3.79-3.92 (m, 6H), 3.64 (d, J = 13.0 Hz, 1H), 3.56-3.61 (m, 1H), 3.48 |
| | (dd, J₁ = 4.0 Hz, J₂ = 13.5 Hz, 1H), 3.33-3.36 (m, 4H), 3.26-3.28 (m, 1H), 3.18- |
| | 3.22 (m, 1H), 3.12-3.13 (m, 9H), 2.90-3.03 (m, 5H), 2.81-2.85 (m, 2H), 2.41- |
| | 2.49 (m, 2H), 2.30-2.34 (m, 3H), 2.25-2.29 (m, 1H), 2.17-2.21 (m, 1H), 1.79- |
| | 1.85 (m, 2H), 1.63-1.69 (m, 2H), 1.62 (d, J = 7.0 Hz, 3H), 1.47-1.51 (m, 2H), |
| | 1.36-1.51 (m, 2H), 1.02(d, J = 6.5 Hz, 4H), 0.89 (s, 3H), 0.53-0.61 (m, 4H), |
| | 0.32-0.35 (m, 2H). |
| 211B | LC/MS: [M]⁺: 1556.9.¹H NMR (500 MHz, CD₃OD) δ: 7.59 (d, J = 8.0 |
| | Hz, 2H), 7.35 (dd, J₁ = 2.5 Hz, J₂ = 9.5 Hz, 1H), 7.29 (d, J = 6.0 Hz, 3H), 7.25 (d, |
| | J = 8.5 Hz, 2H), 7.13-7.22 (m, 7H), 6.97 (d, J = 8.5 Hz, 2H), 6.91 (d, J = 7.5 Hz, |
| | 2H), 6.76 (t, J = 9.0 Hz, 1H), 5.62-5.66 (m, 2H), 5.43-5.50 (m, 1H), 4.69-4.74, |
| | (m, 2H), 4.56-4.62 (m, 2H), 4.56-4.62 (m, 3H), 4.48-4.52 (m, 1H), 4.33 (d, |
| | J = 3.0 Hz, 1H), 4.25-4.29 (m, 2H), 4.14-4.18 (m, 1H), 4.00-4.14 (m, 6H), 3.88- |
| | 3.92 (m, 2H), 3.84-3.87 (m, 2H), 3.79 (s, 3H), 3.77 (t, J = 6.0 Hz, 3H), 3.70- |
| | 3.72 (m, 1H), 3.60-3.66 (m, 1H), 3.57 (d, J = 4.0 Hz, 1H), 3.50-3.54 (m, 2H), |
| | 3.45-3.47 (m, 1H), 3.20-3.25 (m, 1H), 3.13 (s, 9H), 3.06 (d, J = 7.5 Hz, 1H), |
| | 3.01-3.03 (m, 1H), 2.95-3.00 (m, 2H), 2.81-2.86 (m, 1H), 2.63-2.70 (m, 2H), |
| | 2.50-2.57 (m, 3H), 2.33-2.37 (m, 1H), 2.17-2.23 (m, 1H), 1.67 (d, J = 7.0 Hz, |
| | 3H), 1.26-1.47 (m, 5H), 1.12(d, J = 6.5 Hz, 3H), 1.06-1.09 (m, 2H), 1.01 (s, |
| | 3H), 0.84-0.92 (m, 2H), 0.73 (brs, 1H), 0.44-0.50 (m, 2H). |
| 212B | LC/MS: [M]⁺: 1600.9.¹H NMR (500 MHz, CD₃OD) δ: 7.60 (d, J = 8.0 |
| | Hz, 2H), 7.35 (dd, J₁ = 2.5 Hz, J₂ = 9.5 Hz, 2H), 7.26-7.28 (m, 4H), 7.12-7.24 |
| | (m, 7H), 6.96 (d, J = 7.5 Hz, 2H), 6.91 (d, J = 7.5 Hz, 2H), 6.79 (t, J = 8.5 Hz, |
| | 1H), 5.45-5.49 (m, 1H), 5.34 (t, J = 5.0 Hz, 1H), 5.21 (s, 1H), 4.65-4.76 (m, |
| | 7H), 4.47-4.50 (m, 2H), 4.34 (d, J = 3.0 Hz, 1H), 4.29-4.31 (m, 2H), 4.11-4.17 |
| | (m, 4H), 4.06 (t, J = 6.0 Hz, 1H), 4.01 (d, J = 3.0 Hz, 1H), 3.87-3.96 (m, 4H), |
| | 3.78 (s, 3H), 3.72-3.76 (m, 2H), 3.58-3.62 (m, 6H), 3.44-3.51 (m, 2H), 3.08- |
| | 3.19 (m, 9H), 3.03-3.06 (m, 1H), 2.97-2.99 (m, 1H), 2.84 (dt, J₁ = 5.0 Hz, |
| | J₂ = 14.5 Hz, 1H), 2.65-2.67 (m, 2H), 2.52-2.55 (m, 3H), 2.35-2.37 (m, 1H), |
| | 2.20 (t, J = 7.5Hz, 2H), 2.01-2.04 (m, 1H), 1.67 (d, J = 7.0 Hz, 3H), 1.31-1.34 |
| | (m, 5H), 1.13 (d, J = 6.5 Hz, 3H), 1.06-1.09 (m, 2H), 1.02 (s, 3H), 0.85-0.91 |
| | (m, 2H), 0.72 (brs, 1H), 0.45-0.481 (m, 2H). |
| 212A | LC/MS: [M]⁺: 1600.9.¹H NMR (500 MHz, CD₃OD) δ: 7.77 (d, J = 8.0 |
| | Hz, 2H), 7.51 (d, J = 8.0 Hz, 2H), 7.17-7.32 (m, 7H), 6.92-7.06 (m, 7H), 6.78 |
| | (s, 1H), 6.64 (brs, 1H), 5.50 (d, J = 15.0 Hz, 2H), 5.34 (t, J = 5.0 Hz, 1H), 5.21 |
| | (s, 1H), 4.82 (d, J = 14.0 Hz, 2H), 4.70-4.74 (m, 2H), 4.57-4.59 (m, 1H), 4.36 |
| | (s, 1H), 4.29 (s, 1H), 4.21-4.27 (m, 3H), 4.13-4.18 (m, 1H), 4.07-4.11 (m, 1H), |
| | 3.98-4.07 (m, 3H), 3.94-3.98 (m, 1H), 3.88-3.92 (m, 2H), 3.86 (s, 3H), 3.83 |
| | (s, 1H), 3.72-3.78 (m, 2H), 3.61-3.66 (m, 4H), 3.58-3.60 (m, 1H), 3.54-3.56 |
| | (m, 1H), 3.49 (dd, J₁ = 5.0 Hz, J₂ = 14.0 Hz, 1H), 3.27 (d, J = 4.0 Hz, 1H), 3.17 |
| | (s, 9H), 2.92-3.03 (m, 4H), 2.81-2.85 (m, 2H), 2.59 (qd, J₁ = 1.5 Hz, J₂ = 5.0 Hz, |
| | 1H), 2.47 (t, J = 6.5 Hz, 3H), 2.33-2.43 (m, 2H), 2.26 (dd, J₁ = 6.5 Hz, J₂ = 13.5 |
| | Hz, 1H), 2.17-2.21 (m, 1H), 2.08-2.12 (m, 1H), 2.01-2.05 (m, 1H), 1.74-1.80 |
| | (m, 1H), 1.61 (d, J = 7.0 Hz, 3H), 1.47-1.53 (m, 2H), 1.29-1.35 (m, 2H), 1.20- |
| | 1.25 (m, 1H), 1.07-1.12 (m, 1H), 1.02 (d, J = 6.5 Hz, 3H), 0.90 (s, 3H), 0.62 |
| | (brs, 2H), 0.54 (brs, 2H), 0.32-0.37 (m, 2H). |
| 213B | LC/MS: [M]⁺: 1644.5.¹H NMR (500 MHz, CD₃OD) δ: 7.59 (d, J = 8.0 |
| | Hz, 2H), 7.35 (dd, J₁ = 2.5 Hz, J₂ = 9.5 Hz, 1H), 7.24-7.29 (m, 5H), 7.12-7.24 |
| | (m, 7H), 6.96 (d, J = 7.5 Hz, 2H), 6.91 (d, J = 8.0 Hz, 2H), 6.78 (t, J = 4.0 Hz, |
| | 1H), 5.62-5.66 (m, 1H), 5.43-5.50 (m, 2H), 4.64-4.74 (m, 2H), 4.58-4.60 (m, |
| | 2H), 4.48 (d, J = 14.5 Hz, 1H), 4.27-4.34 (m, 3H), 4.07-4.18 (m, 5H), 4.03 (t, |
| | J = 13.5 Hz, 2H), 3.87-3.93 (m, 4H), 3.79 (s, 3H), 3.74 (t, J = 6.0 Hz, 3H), 3.61- |
| | 3.66 (m, 3H), 3.54-3.61 (m, 8H), 3.44-3.54 (m, 3H), 3.08-3.24 (m, 11H), 2.94- |
| | 3.06 (m, 4H), 2.84 (dt, J₁ = 5.0 Hz, J₂ = 15.0 Hz, 1H), 2.63-2.68 (m, 2H), 2.49- |
| | 2.57 (m, 3H), 2.35-2.37 (m, 1H), 2.17-2.24 (m, 1H), 1.67 (d, J = 7.5 Hz, 3H), |
| | 1.32-1.46 (m, 4H), 1.13(d, J = 6.5 Hz, 3H), 1.06-1.09 (m, 2H), 1.02 (s, 3H), |
| | 0.83-0.88 (m, 3H), 0.73 (s, 1H), 0.46-0.51 (m, 2H). |
| T2403-1 | LC/MS: [M + H]⁺: 954.5.¹H NMR (500 MHz, CD₃OD) δ: 7.50 (d, J = 8.0 |
| | Hz, 1H), 7.43 (d, J = 8.0 Hz, 1H), 7.38 (t, J = 8.0 Hz, 2H), 7.31-7.33 (m, 1H), |
| | 7.26 (dd, J₁ = 8.0 Hz, J₂ = 12.5 Hz, 2H), 7.15 (t, J = 7.0 Hz, 2H), 7.10 (d, J = 8.0 |
| | Hz, 1H), 6.84 (d, J = 8.5 Hz, 2H), 5.47-5.85 (m, 1H), 4.94-5.05 (m, 2H), 4.78- |
| | 4.84 (m, 2H), 4.72 (s, 1H), 4.50 (s, 1H), 3.97-4.01 (m, 2H), 3.79-3.83 (m, 1H), |
| | 3.74 (s, 3H), 3.35-3.48 (m, 4H), 3.14-3.17 (m, 1H), 2.99-3.06 (m, 1H), 2.73- |
| | 2.82 (m, 3H), 2.05-2.09 (m, 1H), 1.98-2.03 (m, 1H), 1.85-1.88 (m, 1H), 1.70- |
| | 1.81 (m, 4H), 1.56 (d, J = 17.5 Hz, 6H), 1.51 (s, 3H), 1.45 (s, 9H), 1.38 (s, 9H), |
| | 1.07 (d, J = 6.0 Hz, 3H). |
| T2403-2 | LC/MS: [M + H]⁺: 1174.6.¹H NMR (500 MHz, CD₃OD) δ: 7.47 (d, J = 8.0 |
| | Hz, 1H), 7.17-7.41 (m, 11H), 7.14-7.17 (m, 2H), 7.09 (d, J = 7.5 Hz, 1H), 6.84 |
| | (d, J = 8.5 Hz, 2H), 5.51-5.84 (m, 1H), 4.94-5.04 (m, 3H), 4.81-4.84 (m, 2H), |
| | 4.71 (s, 1H), 4.44-4.49 (m, 2H), 3.96-4.01 (m, 2H), 3.79-3.83 (m, 1H), 3.76 |
| | (s, 3H), 3.43-3.58 (m, 3H), 3.35-3.43 (m, 3H), 3.13-3.16 (m, 1H), 3.01-3.05 |
| | (m, 1H), 2.71-2.83 (m, 3H), 1.99-2.08 (m, 2H), 1.87-1.91 (m, 1H), 1.68-1.81 |
| | (m, 4H), 1.49-1.60 (m, 10H), 1.45 (s, 9H), 1.14 (s, 9H), 1.07 (d, J = 6.0 Hz, |
| | 3H). |
| T2403-3 | LC/MS: [M + H]⁺: 1018.5.¹H NMR (500 MHz, CD₃OD) δ: 7.48 (d, J = 8.0 |
| | Hz, 1H), 7.41 (d, J = 8.0 Hz, 1H), 7.31-7.35 (m, 7H), 7.23-7.28 (m, 3H), 7.16 |
| | (d, J = 7.0 Hz, 2H), 7.10 (d, J = 8.0 Hz, 1H), 6.84 (d, J = 8.5 Hz, 2H), 5.52-5.83 |
| | (m, 1H), 4.94-5.08 (m, 3H), 4.79 (dd, J₁ = 6.5 Hz, J₂ = 8.0 Hz, 2H), 4.71 (s, 1H), |
| | 4.48 (s, 1H), 4.26-4.31 (m, 1H), 3.85-3.89 (m, 1H), 3.78-3.83 (m, 1H), 3.74 |
| | (s, 3H), 3.58-3.65 (m, 1H), 3.48-3.52 (m, 1H), 3.35-3.43 (m, 4H), 3.13-3.16 |
| | (m, 1H), 3.12 (d, J = 5.0 Hz, 1H), 3.03 (dd, J₁ = 6.0 Hz, J₂ = 14.0 Hz, 1H), 2.71- |
| | 2.82 (m, 3H), 1.99-2.08 (m, 2H), 1.89-1.95 (m, 1H), 1.74-1.81 (m, 3H), 1.68- |
| | 1.74 (m, 1H), 1.49-1.57 (m, 10H), 1.10 (d, J = 6.5 Hz, 3H). |
| T2403-4 | LC/MS: [M + H]⁺: 1691.8.¹H NMR (500 MHz, CD₃OD) δ: 7.47 (d, J = 8.0 |
| | Hz, 1H), 7.40 (d, J = 8.5 Hz, 1H), 7.29-7.37 (m, 8H), 7.20-7.29 (m, 5H), 7.14- |
| | 7.17 (m, 3H), 7.09-7.17 (m, 3H), 6.87-6.91 (m, 2H), 5.94-6.01 (m, 1H), 5.51- |
| | 5.85 (m, 2H), 5.12 (dq, J₁ = 1.5 Hz, J₂ = 10.5 Hz, 1H), 4.98-5.04 (m, 3H), 4.94- |
| | 4.96 (m, 2H), 4.79-4.82 (m, 2H), 4.70-4.73 (m, 3H), 4.64 (s, 1H), 4.43-4.49 |
| | (m, 2H), 4.38 (dd, J₁ = 1.5 Hz, J₂ = 10.5 Hz, 1H), 4.24 (d, J = 4.5 Hz, 1H), 4.09- |
| | 4.17 (m, 3H), 4.04-4.09 (m, 1H), 4.00 (d, J = 14.5 Hz, 1H), 3.86-3.91 (m, 1H), |
| | 3.79-3.84 (m, 1H), 3.72 (s, 1H), 3.47-3.57 (m, 2H), 3.37-3.45 (m, 4H), 3.27- |
| | 3.33 (m, 1H), 3.20 (dd, J₁ = 6.5 Hz, J₂ = 14.5 Hz, 1H), 3.13-3.18 (m, 1H), 2.91- |
| | 3.06 (m, 5H), 2.74-2.83 (m, 3H), 1.98-2.08 (m, 5H), 1.85-1.90 (m, 2H), 1.68- |
| | 1.81 (m, 4H), 1.52-1.54 (m, 3H), 1.48-1.50 (m, 5H), 1.46 (s, 9H), 1.13 (d, |
| | J = 6.5 Hz, 3H). |
| T2403-6 | LC/MS: [M + H]⁺: 1573.7.¹H NMR (500 MHz, CD₃OD) δ: 7.42 (d, J = 8.0 |
| | Hz, 1H), 7.30-7.35 (m, 5H), 7.16-7.29 (m, 10H), 7.11-7.15 (m, 3H), 6.97-7.07 |
| | (m, 3H), 6.75-6.89 (m, 4H), 5.88-5.95 (m, 1H), 5.46-5.81 (m, 1H), 5.05-5.09 |
| | (m, 3H), 4.94-5.02 (m, 3H), 4.78-4.86 (m, 4H), 4.66-4.73 (m, 4H), 4.43- |
| | 4.62 (m, 4H), 4.31-4.38 (m, 1H), 4.11-4.26 (m, 5H), 3.91-4.00 (m, 1H), 3.72- |
| | 3.77 (m, 4H), 3.53-3.60 (m, 2H), 3.40-3.47 (m, 2H), 3.19-3.24 (m, 2H), 3.04- |
| | 3.08 (m, 3H), 2.90-3.04 (m, 4H), 2.75-2.82 (m, 2H), 1.95-2.03 (m, 4H), 1.64- |
| | 1.78 (m, 5H), 1.49 (d, J = 18.0 Hz, 4H), 1.42-1.44 (m, 2H), 1.39 (s, 2H), 1.36 |
| | (s, 1H), 1.08-1.14 (m, 3H). |
| 2403 | HRMS: [M]⁺: 1568.8485.¹H NMR (500 MHz, CD₃OD) δ: 7.60-7.63 |
| | (m, 1H), 7.39 (d, J = 8.0 Hz, 2H), 7.23-7.30 (m, 4H), 7.15 (t, J = 7.5 Hz, 4H), |
| | 6.95-7.06 (m, 7H), 6.33 (brs, 1H), 5.01-5.35 (m, 3H), 4.66-4.79 (m, 4H), |
| | 4.56(s, 1H), 4.45-4.47 (m, 1H), 4.22-4.07 (m, 2H), 4.09-4.22 (m, 4H), 4.03- |
| | 4.10 (m, 2H), 3.95-3.98 (m, 1H), 3.90 (d, J = 13.5 Hz, 2H), 3.66-3.84 (m, 5H), |
| | 3.55-3.60 (m, 1H), 3.43-3.48 (m, 1H), 3.33-3.35 (m, 2H), 3.29-3.31 (m, 1H), |
| | 3.22-3.26 (m, 1H), 3.05-3.12 (m, 9H), 2.94-2.99 (m, 3H), 2.79-2.86(m, 3H), |
| | 2.55-2.63 (m, 2H), 2.84 (t, J = 7.5 Hz, 2H), 2.02-2.22 (m, 3H), 1.72-1.83 (m, |
| | 5H), 1.67-1.72 (m, 4H), 1.50-1.54 (m, 2H), 1.24-1.36 (m, 2H), 1.07-1.09 (m, |
| | 9H), 0.73-0.81 (m, 2H), 0.51-0.57(m, 2H). |
| 2430 | LC/MS: [M]⁺: 1455.7.¹H NMR (500 MHz, CD₃OD) δ: 7.50-7.56 (m, |
| | 2H), 7.36 (s, 4H), 7.13-7.27 (m, 9H), 7.02 (s, 2H), 6.90 (d, J = 8.0 Hz, 2H), |
| | 6.65-6.687 (m, 1H), 5.34 (t, J = 5.0 Hz, 1H), 4.99-5.07 (m, 2H), 4.63-4.70 (m, |
| | 4H), 4.04-4.23 (m, 9H), 3.74-3.86 (m, 4H), 3.54-3.69 (m, 4H), 3.42-3.45 (m, |
| | 1H), 3.33-3.35 (m, 2H), 3.15-3.21 (m, 9H), 2.91-3.07 (m, 6H), 2.81-2.84 (m, |
| | 2H), 2.62-2.65 (m, 1H), 2.27-2.31 (m, 1H), 2.20 (t, J = 7.5 Hz, 1H), 1.94-2.06 |
| | (m, 3H), 1.81-1.83 (m, 2H), 1.52-1.63 (m, 7H), 1.32-1.41 (m, 3H), 1.18-1.21 |
| | (m, 3H), 1.40 (d, J = 6.0 Hz, 3H), 0.57-0.76 (m, 4H). |
| T2404-1 | LC/MS: [M + H]⁺: 952.6.¹H NMR (400 MHz, CD₃OD) δ: 7.47 (d, J = 8.0 |
| | Hz, 1H), 7.40 (d, J = 7.6 Hz, 1H), 7.31-7.37 (m, 3H), 7.26 (t, J = 8.4 Hz, 2H), |
| | 7.15 (d, J = 8.0 Hz, 2H), 7.11 (d, J = 6.8 Hz, 1H), 6.84 (d, J = 8.8 Hz, 2H), 5.53- |
| | 5.83 (m, 1H), 4.94-5.06 (m, 1H), 4.72-4.82 (m, 3H), 4.51(s, 1H), 3.98-4.00 |
| | (m, 2H), 3.77-3.80 (m, 1H), 3.74 (s, 3H), 3.40-3.48 (m, 4H), 3.15-3.19 (m, |
| | 1H), 3.01-3.05 (m, 1H), 2.74-2.83 (m, 3H), 2.01-2.08 (m, 2H), 1.71-1.89 (m, |
| | 5H), 158-1.60 (m, 3H), 1.51 (s, 3H), 1.45 (s, 9H), 1.20-1.22 (m, 2H), 1.14 (s, |
| | 9H), 1.07 (d, J = 5.6 Hz, 3H). |
| T2404-2 | LC/MS: [M + H]⁺: 1172.6.¹H NMR (500 MHz, CD₃OD) δ: 7.46 (d, J = 7.5 |
| | Hz, 1H), 7.31-7.41 (m, 6H), 7.27-7.30 (m, 3H), 7.23-7.26 (m, 2H), 7.14-7.17 |
| | (m, 2H), 7.11 (d, J = 8.0 Hz, 1H), 6.84 (d, J = 9.0 Hz, 2H), 5.54-5.84 (m, 1H), |
| | 4.91-5.04 (m, 3H), 4.83 (t, J = 7.5 Hz, 2H), 4.72 (s, 1H), 4.49-4.57 (m, 1H), |
| | 4.36-4.41 (m, 1H), 3.96-4.01 (m, 2H), 3.79-3.83 (m, 1H), 3.73 (s, 3H), 3.44- |
| | 3.54 (m, 2H), 3.34-3.42 (m, 4H), 3.16-3.21 (m, 1H), 3.02-3.06 (m, 1H), 2.70- |
| | 2.83 (m, 3H), 1.99-2.08 (m, 2H), 1.86-1.92 (m, 1H), 1.73-1.83 (m, 3H), 1.67- |
| | 1.72 (m, 1H), 1.58-1.62 (m, 1H), 1.48-1.54 (m, 5H), 1.45 (s, 9H), 1.14 (s, 9H), |
| | 1.05-1.11 (m, 5H). |
| T2404-3 | LC/MS: [M + H]⁺: 1016.5.¹H NMR (400 MHz, CD₃OD) δ: 7.48 (d, J = 7.6 |
| | Hz, 1H), 7.31-7.39 (m, 7H), 7.24-7.31 (m, 4H), 7.12-7.18 (m, 3H), 6.84 (d, |
| | J = 8.8 Hz, 2H), 5.54-5.85 (m, 1H), 4.94-5.05 (m, 3H), 4.77-4.81 (m, 2H), 4.72 |
| | (s, 1H), 4.52 (s, 1H), 4.21 (s, 1H), 3.79-3.92 (m, 2H), 3.74 (s, 3H), 3.47-3.60 |
| | (m, 2H), 3.37-3.41 (m, 3H), 3.28-3.30 (m, 1H), 3.18-3.21 (m, 1H), 3.14 (d, |
| | J = 4.2 Hz, 1H), 3.03 (dd, J₁ = 6.4 Hz, J₂ = 14.0 Hz, 1H), 2.76-2.83 (m, 3H), 2.03- |
| | 2.08 (m, 2H), 1.90-1.95 (m, 1H), 1.70-1.80 (m, 4H), 1.48-1.59 (m, 6H), 1.28- |
| | 1.31 (m, 1H), 1.11 (d, J = 6.4 Hz, 3H), 0.98-1.03 (m, 1H). |
| T2404-4 | LC/MS: [M + H]⁺: 1689.8.¹H NMR (500 MHz, CD₃OD) δ: 7.46 (d, J = 7.5 |
| | Hz, 1H), 7.20-7.39 (m, 14H), 7.09-7.18 (m, 6H), 6.85-6.91 (m, 2H), 6.82 (d, |
| | J = 8.5 Hz, 2H), 5.94-6.01 (m, 1H), 5.55-5.82 (m, 1H), 5.12 (dd, J₁ = 1.5 Hz, |
| | J₂ = 10.0 Hz, 1H), 4.94-5.03 (m, 5H), 4.80-4.82 (m, 2H), 4.70-4.73 (m, 3H), |
| | 4.64-4.65 (m, 1H), 4.52 (d, J = 6.0 Hz, 2H), 4.36-4.40 (m, 2H), 4.24 (d, J = 4.0 |
| | Hz, 1H), 4.13-4.17 (m, 3H), 4.05-4.09 (m, 1H), 4.00 (d, J = 14.5 Hz, 1H), 3.86- |
| | 3.91 (m, 1H), 3.79-3.84 (m, 1H), 3.72 (s, 3H), 3.46-3.53 (m, 2H), 3.35-3.41 |
| | (m, 4H), 3.27-3.29 (m, 1H), 3.16-3.23 (m, 2H), 2.96-3.06 (m, 3H), 2.91-2.96 |
| | (m, 1H), 2.73-2.84 (m, 3H), 2.00-2.06 (m, 5H), 1.68-1.88 (m, 6H), 1.58-1.61 |
| | (m, 2H), 1.52-1.54 (m, 3H), 1.46 (s, 9H), 1.13 (d, J = 6.0 Hz, 3H), 1.04-1.10 |
| | (m, 2H). |
| T2404-6 | LC/MS: [M + H]⁺: 1571.7.¹H NMR (500 MHz, CD₃OD) δ: 7.40 (d, J = 8.0 |
| | Hz, 1H), 7.25-7.35 (m, 9H), 7.22-7.25 (m, 4H), 7.18 (d, J = 8.5 Hz, 2H), 7.11- |
| | 7.13 (m, 2H), 7.04-7.08 (m, 3H), 6.83-6.94 (m, 4H), 5.89-6.01 (m, 1H), 5.49- |
| | 5.81 (m, 1H), 4.96-5.14 (m, 4H), 4.77-4.84 (m, 2H), 4.72 (d, J = 5.5 Hz, 1H), |
| | 4.61-4.69 (m, 3H), 4.48-4.54 (m, 2H), 4.40-4.43 (m, 1H), 4.37 (s, 1H), 4.27 |
| | (t, J = 14.5 Hz, 1H), 4.17-4.21 (m, 2H), 4.07-4.14 (m, 3H), 3.92 (q, J = 9.5 Hz, |
| | 1H), 3.77-3.81 (m, 1H), 3.71-3.77 (m, 3H), 3.58-3.63 (m, 1H), 3.51-3.58 (m, |
| | 1H), 3.44-3.47 (m, 1H), 3.39 (dd, J₁ = 4.0 Hz, J₂ = 14.5 Hz, 1H), 3.19-3.27 (m, |
| | 2H), 3.03-3.18 (m, 4H), 2.91-3.00 (m, 3H), 2.72-2.78 (m, 3H), 1.99-2.12 (m, |
| | 4H), 1.75-1.93 (m, 3H), 1.63-1.69 (m, 3H), 1.51-1.55 (m, 1H), 1.45 (s, 3H), |
| | 1.29-1.36 (m, 2H), 1.67-1.21 (m, 1H), 1.13 (d, J = 6.0 Hz, 3H), 0.96-1.03 (m, |
| | 1H), 0.85-0.89 (m, 1H). |
| 2404 | HRMS: [M]⁺: 1552.8.¹H NMR (500 MHz, CD₃OD)) 8: 7.43-7.51 (m, |
| | 2H), 7.23-7.33 (m, 7H), 7.18 (d, J = 8.0 Hz, 2H), 7.11-7.14 (m, 3H), 6.99-7.04 |
| | (m, 2H), 6.94 (d, J = 9.0 Hz, 2H), 6.85-6.88 (m, 1H), 6.78-6.82 (m, 1H), 5.34- |
| | 5.36 (m, 1H), 4.70-4.77 (m, 4H), 4.60-4.61 (m, 1H), 4.39-4.42 (m, 2H), 4.08- |
| | 4.30 (m, 6H), 3.88-4.03 (m, 4H), 3.62-3.82 (m, 7H), 3.32-3.46 (m, 5H), 3.08- |
| | 3.18 (m, 10H), 3.02-3.03 (m, 2H), 2.74-2.96 (m, 7H), 2.18-2.32 (m, 4H), 2.01- |
| | 2.05 (m, 2H), 1.78-1.83 (m, 3H), 1.55-1.73 (m, 7H), 1.41-1.47 (m, 3H), 1.19- |
| | 1.23 (m, 2H), 1.15 (d, J = 6.0 Hz, 3H), 1.01-1.06 (m, 1H), 0.89-0.92 (m, 1H), |
| | 0.69 (brs, 3H). |
| T2416-1 | LC/MS: [M + H]⁺: 962.5.¹H NMR (600 MHz, CD₃OD) δ: 7.72 (d, J = 8.4 |
| | Hz, 1H), 7.65 (d, J = 7.8 Hz, 1H), 7.53 (d, J = 7.8 Hz, 2H), 7.33 (d, J = 7.8 Hz, |
| | 1H), 7.28 (d, J = 7.2 Hz, 1H), 7.25 (d, J = 7.8 Hz, 1H), 7.15 (t, J = 7.8 Hz, 2H), |
| | 7.09 (d, J = 7.8 Hz, 1H), 6.84 (d, J = 8.4 Hz, 2H), 5.46-5.85 (m, 1H), 4.96-5.06 |
| | (m, 1H), 4.77-4.84 (m, 2H), 4.74 (s, 1H), 4.46 (s, 1H), 3.98-4.01 (m, 2H), |
| | 3.79-3.83 (m, 1H), 3.74 (s, 3H), 3.35-3.49 (m, 4H), 3.12 (t, J = 7.8 Hz, 1H), |
| | 3.02 (td, J₁ = 6.6 Hz, J₂ = 13.8 Hz, 1H), 2.72-2.85 (m, 3H), 2.09 (q, J = 7.2 Hz, |
| | 1H), 1.97-2.05 (m, 1H), 1.87-1.91 (m, 1H), 1.73-1.81 (m, 4H), 1.56-1.61 (m, |
| | 1H), 1.50 (d, J = 7.2 Hz, 3H), 1.45 (s, 9H), 1.14 (s, 9H), 1.07 (d, J = 6.0 Hz, 3H). |
| T2416-2 | LC/MS: [M + H]⁺: 1196.6.¹H NMR (500 MHz, CD₃OD) δ: 7.74 (d, J = 8.0 |
| | Hz, 1H), 7.67 (d, J = 8.0 Hz, 1H), 7.49 (d, J = 8.0 Hz, 1H), 7.27-7.34 (m, 6H), |
| | 7.24 (d, J = 8.0 Hz, 1H), 7.14-7.17 (m, 3H), 7.08 (d, J = 8.0 Hz, 1H), 6.84 (d, |
| | J = 8.5 Hz, 2H), 5.48-5.85 (m, 1H), 4.98-5.11 (m, 3H), 4.79-4.84 (m, 2H), 4.73 |
| | (s, 1H), 4.58-4.62 (m, 1H), 4.43 (s, 1H), 3.97-4.01 (m, 2H), 3.79-3.83 (m, 1H), |
| | 3.74 (s, 3H), 3.61-3.68 (m, 4H), 3.48-3.56 (m, 2H), 3.35-3.44 (m, 3H), 3.10 |
| | (t, J = 8.0 Hz, 1H), 2.99-3.03 (m, 1H), 2.72-2.82 (m, 3H), 2.08 (q, J = 7.0 Hz, |
| | 1H), 1.96-2.04 (m, 1H), 1.86-1.91 (m, 1H), 1.70-1.79 (m, 4H), 1.57 (quintet, |
| | J = 7.5 Hz, 1H), 1.50 (d, J = 5.0 Hz, 3H), 1.45 (s, 9H), 1.14 (s, 9H), 1.07 (d, |
| | J = 6.0 Hz, 3H). |
| T2416-3 | LC/MS: [M + H]⁺: 1026.4.¹H NMR (400 MHz, CD₃OD) δ: 7.75 (d, J = 8.0 |
| | Hz, 1H), 7.69 (d, J = 8.0 Hz, 1H), 7.46-7.49 (m, 2H), 7.22-7.33 (m, 8H), 7.18 |
| | (t, J = 8.0 Hz, 2H), 7.07 (d, J = 7.6 Hz, 1H), 6.85 (d, J = 8.4 Hz, 2H), 5.46-5.87 |
| | (m, 1H), 4.95-5.07 (m, 3H), 4.73-4.81 (m, 2H), 453-4.58 (m, 1H), 4.42 (s, |
| | 1H), 3.99 (quintet, J = 6.4 Hz, 1H), 3.85-3.91 (m, 1H), 3.75 (s, 3H), 3.63-3.70 |
| | (m, 2H), 3.47-3.56 (m, 2H), 3.38-3.43 (m, 3H), 3.02-3.12 (m, 2H), 2.76-2.85 |
| | (m, 3H), 1.99-2.08 (m, 2H), 1.71-1.94 (m, 5H), 1.53-1.58 (m, 1H), 1.50 (d, |
| | J = 4.0 Hz, 1H), 1.36-1.38 (m, 1H), 1.28 (d, J = 6.4 Hz, 3H). |
| T2416-4 | LC/MS: [M + H]⁺: 1699.8.¹H NMR (600 MHz, CD₃OD) δ: 7.74 (d, J = 7.8 |
| | Hz, 1H), 7.68 (d, J = 7.8 Hz, 1H), 7.48 (t, J = 8.4 Hz, 2H), 7.38 (d, J = 8.4 Hz, |
| | 1H), 7.28-7.33 (m, 7H), 7.25-7.28 (m, 2H), 7.22 (t, J = 7.8 Hz, 2H), 7.12-7.18 |
| | (m, 3H), 7.08 (t, J = 7.8 Hz, 2H), 6.86-6.90 (m, 2H), 6.82 (d, J = 9.0 Hz, 2H), |
| | 5.94-6.01 (m, 1H), 5.48-5.85 (m, 1H), 5.10-5.13 (m, 1H), 4.99-5.08 (m, 3H), |
| | 4.94-4.97 (m, 2H), 4.79-4.82 (m, 2H), 4.67-4.73 (m, 4H), 4.58-4.65 (m, 2H), |
| | 4.42 (s, 1H), 4.37 (dd, J₁ = 3.0 Hz, J₂ = 9.6 Hz, 1H), 4.24 (d, J = 4.2 Hz, 1H), |
| | 4.10-4.16 (m, 3H), 4.05-4.09 (m, 1H), 4.01 (d, J = 14.4 Hz, 1H), 3.88 (q, J = 9.0 |
| | Hz, 1H), 3.79-3.83 (m, 1H), 3.72 (s, 3H), 3.64-3.69 (m, 1H), 3.48-3.56 (m, |
| | 2H), 3.35-3.44 (m, 3H), 3.28-3.29 (m, 1H), 3.20 (dd, J₁ = 6.6 Hz, J₂ = 14.4 Hz, |
| | 1H), 3.10 (t, J = 7.8 Hz, 1H), 2.98-3.05 (m, 2H), 2.92-2.97 (m, 1H), 2.73-2.83 |
| | (m, 3H), 1.98-2.09 (m, 4H), 1.84-1.88 (m, 1H), 1.72-1.79 (m, 4H), 1.54-1.59 |
| | (m, 1H), 1.50 (d, J = 6.6 Hz, 3H), 1.46 (s, 9H), 1.29-1.33 (m, 1H), 1.13 (d, |
| | J = 6.0 Hz, 3H). |
| T2416-6 | LC/MS: [M + H]⁺: 1581.6.¹H NMR (600 MHz, CD₃OD) δ: 7.61-7.71 |
| | (m, 3H), 7.42-7.51 (m, 2H), 7.28-7.39 (m, 5H), 7.22-7.28 (m, 2H), 7.10-7.19 |
| | (m, 6H), 6.97-7.06 (m, 3H), 6.77-6.90 (m, 3H), 6.70-6.71 (m, 1H), 5.84-5.92 |
| | (m, 1H), 5.44-5.81 (m, 1H), 5.01-5.15 (m, 4H), 4.93-4.98 (m, 1H), 4.70-4.79 |
| | (m, 4H), 4.54-4.65 (m, 5H), 4.27-4.33 (m, 2H), 4.09-4.23 (m, 4H), 3.92-4.00 |
| | (m, 2H), 3.71-3.81 (m, 5H), 3.52-3.64 (m, 4H), 3.34-3.43 (m, 2H), 3.18-3.29 |
| | (m, 2H), 2.86-2.99 (m, 5H), 2.71-2.83 (m, 2H), 2.01-2.08 (m, 3H), 1.61-1.70 |
| | (m, 3H), 1.42-1.54 (m, 3H), 1.25-1.34 (m, 5H), 1.08-1.12 (m, 3H). |
| 2416 | HRMS: [M]⁺: 1562.7685.¹H NMR (500 MHz, CD₃OD) δ: 7.93 (d, J = 8.5 |
| | Hz, 2H), 7.60 (d, J = 8.0 Hz, 2H), 7.54 (d, J = 9.0 Hz, 1H), 7.22-7.29 (m, 5H), |
| | 7.16-7.19 (m, 2H), 7.13 (d, J = 8.0 Hz, 2H), 6.97-7.00 (m, 5H), 6.93 (s, 1H), |
| | 6.86-6.89 (m, 1H), 6.23-6.27 (m, 1H), 5.53 (d, J = 15.0 Hz, 2H), 4.70-4.74 (m, |
| | 2H), 4.63 (t, J = 8.0 Hz, 1H), 4.41-4.45 (m, 2H), 4.35-4.37 (m, 1H), 4.28-4.30 |
| | (m, 2H), 4.23 (d, J = 15.5 Hz, 1H), 4.16-4.20 (m, 1H), 4.09-4.13 (m, 1H), 4.01- |
| | 4.07 (m, 3H), 3.78-3.86 (m, 6H), 3.68-3.75 (m, 2H), 3.65 (dd, J₁ = 4.0 Hz, |
| | J₂ = 14.5 Hz, 1H), 3.48 (dd, J₁ = 6.5Hz, J₂ = 14.0 Hz, 1H), 3.33-3.38 (m, 3H), |
| | 3.20-3.24 (m, 1H), 3.07-3.17 (m, 10H), 2.91-2.98 (m, 3H), 2.79-2.87 (m, 2H), |
| | 2.71-2.76 (m, 1H), 2.49-2.54 (m, 2H), 2.35 (t, J = 6.5 Hz, 2H), 2.27-2.31 (m, |
| | 1H), 2.17-2.19 (m, 1H), 1.81-1.89 (m, 2H), 1.65-1.76 (m, 2H), 1.37-1.46 (m, |
| | 4H), 1.16-1.20 (m, 1H), 1.06 (d, J = 6.5 Hz, 3H), 0.96 (s, 3H), 0.89-0.92 (m, |
| | 2H), 0.77-0.82 (m, 2H), 0.51-0.56 (m, 2H), 0.35-0.38 (m, 1H). |
| T2410-1 | LC/MS: [M + H]⁺: 940.6.¹H NMR (500 MHz, CD₃OD) δ: 7.31-7.41 (m, |
| | 3H), 7.21-7.28 (m, 4H), 7.13-7.16 (m, 2H), 7.09 (d, J = 8.0 Hz, 1H), 6.84 (d, |
| | J = 8.5 Hz, 2H), 5.51-5.86 (m, 1H), 4.95-5.05 (m, 2H), 4.72 (s, 1H), 4.47 (s, |
| | 1H), 3.97-4.01 (m, 2H), 3.79-3.83 (m, 1H), 3.74 (s, 3H), 3.38-3.42 (m, 4H), |
| | 3.13-3.16 (m, 1H), 3.00-3.05 (m, 1H), 2.96 (t, J = 7.5 Hz, 1H), 2.91 (t, J = 7.5 |
| | Hz, 1H), 2.76-2.82 (m, 3H), 2.63 (t, J = 7.5 Hz, 1H), 2.55 (t, J = 7.5 Hz, 1H), |
| | 2.00-2.10 (m, 2H), 1.86-1.92 (m, 1H), 1.70-1.80 (m, 4H), 1.55-1.61 (m, 1H), |
| | 1.51 (s, 3H), 1.45 (s, 9H), 1.29-1.33 (m, 1H), 1.14 (s, 9H), 1.07 (d, J = 5.5 Hz, |
| | 2H). |
| T2410-2 | LC/MS: [M + H]⁺: 1160.6.¹H NMR (500 MHz, CD₃OD) δ: 7.19-7.37 |
| | (m, 12H), 7.16 (d, J = 7.0 Hz, 1H), 7.09 (d, J = 7.5 Hz, 1H), 6.84 (d, J = 8.0 Hz, |
| | 2H), 5.50-5.85 (m, 1H), 5.01-5.10 (m, 3H), 4.96 (d, J = 10 Hz, 1H), 4.80-4.83 |
| | (m, 1H), 4.71 (s, 1H), 4.49-4.55 (m, 1H), 4.62 (s, 1H), 3.97-4.01 (m, 2H), |
| | 3.78-3.83 (m, 1H), 3.73 (s, 3H), 3.52-3.58 (m, 1H), 3.47-3.52 (m, 1H), 3.35- |
| | 3.45 (m, 4H), 3.14 (t, J = 7.5 Hz, 1H), 3.01-3.06 (m, 1H), 2.94 (t, J = 7.5 Hz, |
| | 1H), 2.89 (t, J = 7.5 Hz, 1H), 2.73-2.82 (m, 3H), 2.53 (t, J = 8.0 Hz, 1H), 2.46 |
| | (t, J = 8.0 Hz, 1H), 2.00-2.09 (m, 2H), 1.86-1.90 (m, 1H), 1.70-1.81 (m, 4H), |
| | 1.55-1.60 (m, 1H), 1.51 (s, 3H), 1.45 (s, 9H), 1.14 (s, 9H), 1.07 (d, J = 6.0 Hz, |
| | 3H). |
| T2410-3 | LC/MS: [M + H]⁺: 1004.5.¹H NMR (500 MHz, CD₃OD) δ: 7.23-7.37 |
| | (m, 10H), 7.19-7.20 (m, 2H), 7.15 (d, J = 8.5 Hz, 2H), 7.10 (d, J = 7.5 Hz, 1H), |
| | 6.83 (d, J = 9.0 Hz, 2H), 5.51-5.84 (m, 1H), 5.01-5.10 (m, 3H), 4.96 (d, J = 10.5 |
| | Hz, 1H), 4.77-4.80 (m, 1H), 4.72 (s, 1H), 4.47 (s, 1H), 4.33-4.37 (m, 1H), |
| | 3.84-3.89 (m, 1H), 3.79-3.83 (m, 1H), 3.74 (s, 3H), 3.54-3.60 (m, 1H), 3.47- |
| | 3.52 (m, 1H), 3.37-3.42 (m, 3H), 3.12-3.17 (m, 2H), 3.03 (dd, J₁ = 6.5 Hz, |
| | J₂ = 14.5 Hz, 1H), 2.93 (t, J = 8.0 Hz, 1H), 2.88 (t, J = 8.0 Hz, 1H), 2.76-2.82 (m, |
| | 3H), 2.49 (t, J = 8.5 Hz, 1H), 2.42 (t, J = 8.5 Hz, 1H), 2.01-2.10 (m, 2H), 1.90- |
| | 1.93 (m, 1H), 1.71-1.79 (m, 4H), 1.54-1.58 (m, 1H), 1.51 (s, 3H), 1.28-1.32 |
| | (m, 1H), 1.11 (d, J = 6.5 Hz, 3H). |
| T2410-4 | LC/MS: [M + H]⁺: 1678.8.¹H NMR (500 MHz, CD₃OD) δ: 7.30-7.37 (m, |
| | 7H), 7.19-7.29 (m, 8H), 7.16 (d, J = 6.5 Hz, 2H), 7.12 (d, J = 8.0 Hz, 2H), 7.09 |
| | (d, J = 7.5 Hz, 2H), 6.86-6.90 (m, 2H), 6.81 (d, J = 8.5 Hz, 2H), 5.94-6.01 (m, |
| | 1H), 5.50-5.84 (m, 1H), 5.12 (dq, J₁ = 1.5 Hz, J₂ = 10.0 Hz, 1H), 4.94-5.09 (m, |
| | 5H), 4.79-4.83 (m, 2H), 4.69-4.73 (m, 3H), 4.66 (d, J = 9.5 Hz, 1H), 4.49-4.55 |
| | (m, 1H), 4.46 (s, 1H), 4.39 (dd, J₁ = 3.0 Hz, J₂ = 9.0 Hz, 1H), 4.25 (d, J = 4.0 Hz, |
| | 1H), 4.09-4.18 (m, 3H), 4.05-4.09 (m, 1H), 4.00 (d, J = 15.0 Hz, 1H), 3.88 (q, |
| | J = 9.0 Hz, 1H), 3.79-3.84 (m, 1H), 3.71 (s, 3H), 3.48-3.57 (m, 2H), 3.35-3.45 |
| | (m, 4H), 3.27-3.29 (m, 1H), 3.20 (dd, J₁ = 6.5 Hz, J₂ = 15.0 Hz, 1H), 3.13 (t, |
| | J = 8.0 Hz, 1H), 2.98-3.05 (m, 3H), 2.87-2.96 (m, 3H), 2.73-2.82 (m, 3H), 2.52 |
| | (t, J = 8.0 Hz, 1H), 2.45 (t, J = 8.0 Hz, 1H), 1.99-2.09 (m, 4H), 1.84-1.91 (m, |
| | 1H), 1.70-1.79 (m, 4H), 1.57 (t, J = 8.0 Hz, 1H), 1.50 (d, J = 4.0 Hz, 3H), 1.46 |
| | (s, 9H), 1.29-1.33 (m, 1H), 1.13 (d, J = 6.0 Hz, 3H). |
| T2410-6 | LC/MS: [M + H]⁺: 1560.7.¹H NMR (500 MHz, CD₃OD) δ: 7.26-7.37 (m, |
| | 8H), 7.14-7.25 (m, 9H), 7.06 (t, J = 9.0 Hz, 2H), 6.99 (t, J = 8.5 Hz, 2H), 6.81- |
| | 6.90 (m, 4H), 5.93-5.98 (m, 1H), 5.50-5.84 (m, 1H), 5.03-5.11 (m, 3H), 4.94- |
| | 5.00 (m, 2H), 4.80-4.84 (m, 2H), 4.67-4.75 (m, 4H), 4.41.59 (m, 4H), 4.25- |
| | 4.28 (m, 2H), 4.15-4.17 (m, 1H), 4.06 (t, J = 14.0 Hz, 1H), 3.95-4.00 (m, 2H), |
| | 3.88-3.92 (m, 1H), 3.79-3.84 (m, 2H), 3.68 (d, J = 5.5 Hz, 3H), 3.50-3.60 (m, |
| | 3H), 3.38-3.43 (m, 2H), 3.11-3.28 (m, 4H), 3.01-3.07 (m, 4H), 2.89-2.97 (m, |
| | 2H), 2.77-2.80 (m, 1H), 2.68-2.73 (m, 3H), 2.33-2.35 (m, 2H), 2.01-2.07 (m, |
| | 4H), 1.79-1.85 (m, 1H), 1.68-1.71 (m, 2H), 1.58-1.62 (m, 1H), 1.45 (s, 3H), |
| | 1.30-1.33 (m, 1H), 1.15 (dd, J₁ = 6.5 Hz, J₂ = 11.5 Hz, 3H). |
| 2410 | LC/MS: [M]⁺: 1554.8.¹H NMR (500 MHz, CD₃OD) δ: 7.41-7.46 (m, |
| | 3H), 7.17-7.27 (m, 7H), 7.13 (d, J = 8.0 Hz, 1H), 7.01-7.08 (m, 4H), 6.92 (s, |
| | 1H), 6.87 (d, J = 8.0 Hz, 2H), 6.78 (td, J₁ = 2.5 Hz, J₂ = 9.0 Hz, 1H), 6.06-6.08 |
| | (m, 1H), 5.30-5.36 (m, 3H), 4.70 (t, J = 7.5 Hz, 1H), 4.56-4.59 (m, 3H), 4.52- |
| | 4.55 (m, 1H), 4.48 (s, 1H), 4.30 (d, J = 3.5 Hz, 1H), 4.22 (d, J = 3.5 Hz, 1H), |
| | 4.08-4.13 (m, 2H), 3.99-4.05 (m, 3H), 3.91-3.95 (m, 1H), 3.84-3.88 (m, 1H), |
| | 3.78 (s, 3H), 3.67-3.74 (m, 2H), 3.54-3.57 (m, 2H), 3.44-3.49 (m, 2H), 3.33- |
| | 3.37 (m, 2H), 3.16-3.20 (m, 2H), 3.03-3.09 (m, 9H), 2.93-3.03 (m, 4H), 2.83- |
| | 2.91(m, 3H), 2.77-2.81 (m, 1H), 2.60-2.62 (m, 3H), 2.28-2.32 (m, 2H), 2.19 |
| | (t, J = 7.5 Hz, 1H), 2.10-2.14 (m, 1H), 2.01-2.05 (m, 2H), 1.71-1.78 (m, 5H), |
| | 1.59-1.62 (m, 1H), 1.33-1.46 (m, 6H), 1.22 (s, 3H), 1.15-1.17 (m, 1H), 1.09 |
| | (d, J = 6.5 Hz, 3H), 1.04-1.11 (m, 1H), 0.90 (t, J = 6.0 Hz, 1H), 0.54-0.60(m, |
| | 2H). |
| 2452 | LC/MS: [M]⁺: 1540.8.¹H NMR (500 MHz, CD₃OD) δ: 7.35-7.44 (m, |
| | 4H), 7.22-7.27 (m, 3H), 7.16-7.21 (m, 3H), 7.30 (d, J = 8.0 Hz, 2H), 7.08 (s, |
| | 1H), 7.04 (d, J = 6.5 Hz, 1H), 7.01 (d, J = 8.0 Hz, 2H), 6.91 (s, 1H), 6.87 (d, |
| | J = 8.0 Hz, 2H), 6.78 (td, J₁ = 2.5 Hz, J₂ = 9.5 Hz, 1H), 5.30-5.35 (m, 2H), 5.20(s, |
| | 1H), 4.78-4.82 (m, 1H), 4.68-4.71 (m, 2H), 4.52-4.57 (m, 2H), 4.49 (s, 1H), |
| | 4.30 (d, J = 3.5 Hz, 1H), 4.22 (d, J = 3.5 Hz, 1H), 4.01-4.13 (m, 4H), 3.83-3.99 |
| | (m, 4H), 3.66-3.83 (m, 5H), 3.56-3.63 (m, 2H), 3.50-3.53 (m, 1H), 3.44-3.45 |
| | (m, 1H), 3.33-3.38 (m, 3H), 3.13-3.20 (m, 2H), 3.10 (s, 6H), 2.80-3.01 (m, |
| | 9H), 2.57-2.65 (m, 3H), 2.36 (t, J = 7.0 Hz, 2H), 2.20 (t, J = 2.5 Hz, 1H), 2.10- |
| | 2.15 (m, 1H), 2.01-2.06 (m, 2H), 1.79-1.82 (m, 2H), 1.69 (quintet, J = 2.5 Hz, |
| | 1H), 1.58-1.62 (m, 1H), 1.41-1.47 (m, 4H), 1.21 (s, 3H), 1.13-1.18 (m, 1H), |
| | 1.09 (d, J = 6.5 Hz, 3H), 0.95-0.98 (m, 1H), 0.90 (t, J = 7.0 Hz, 2H), 0.53-0.64 |
| | (m, 2H). |
| T2412-1 | LC/MS: [M + H]⁺: 940.5.¹H NMR (500 MHz, CD₃OD) δ: 7.79-7.94 (m, |
| | 2H), 7.34-7.49 (m, 2H), 7.20 (d, J = 7.8 Hz, 1H), 7.14-7.18 (m, 3H), 7.05 (d, |
| | J = 7.8 Hz, 1H), 7.00 (d, J = 8.0 Hz, 1H), 7.05 (dd, J₁ = 3.5 Hz, J₂ = 8.5 Hz, 2H), |
| | 5.67-5.80 (m, 1H), 4.91-5.00 (m, 4H), 4.52 (s, 1H), 4.46 (s, 1H), 3.98-4.01 |
| | (m, 2H), 3.78-3.82 (m, 1H), 3.73 (d, J = 2.0 Hz, 3H), 3.44-3.49 (m, 1H), 3.34- |
| | 3.43 (m, 2H), 3.27-3.31 (m, 1H), 3.13 (t, J = 8.0 Hz, 1H), 3.05 (t, J = 7.0 Hz, |
| | 1H), 2.93-3.03 (m, 2H), 2.69-2.83 (m, 4H), 2.64 (t, J = 7.0 Hz, 1H), 1.86-2.04 |
| | (m, 4H), 1.71-1.79 (m, 2H), 1.57 (quintet, J = 7.5 Hz, 1H), 1.50 (d, J = 8.0 Hz, |
| | 3H), 1.44 (s, 9H). |
| T2412-2 | LC/MS: [M + H]⁺: 1160.6.¹H NMR (500 MHz, CD₃OD) δ: 7.65-7.93 |
| | (m, 3H), 7.34-7.48 (m, 2H), 7.23-7.34 (m, 4H), 7.13-7.20 (m, 4H), 7.05 (d, |
| | J = 5.0 Hz, 1H), 7.01 (d, J = 8.0 Hz, 1H), 6.82-6.85 (m, 2H), 5.67-5.80 (m, 1H), |
| | 5.02-5.10 (m, 2H), 4.91-4.99 (m, 3H), 4.79-4.83 (m, 1H), 4.71-4.75 (m, 1H), |
| | 4.52 (s, 1H), 4.46 (s, 1H), 3.91-4.01 (m, 2H), 3.78-3.81 (m, 1H), 3.73 (d, J = 2.0 |
| | Hz, 3H), 3.60-3.65 (m, 1H), 3.41-3.49 (m, 1H), 3.34-3.41 (m, 2H), 3.26-3.31 |
| | (m, 1H), 3.14 (t, J = 8.0 Hz, 1H), 2.96-3.05 (m, 2H), 2.94 (t, J = 7.50 Hz, 1H), |
| | 2.71-2.82 (m, 4H), 2.65 (t, J = 7.5 Hz, 1H), 1.91-2.03 (m, 3H), 1.83-1.88 (m, |
| | 1H), 1.70-1.78 (m, 2H), 1.52-1.60 (m, 2H), 1.49 (d, J = 11 Hz, 3H), 1.45 (s, |
| | 9H), 1.14 (s, 9H), 1.06-1.07 (m, 2H). |
| T2412-3 | LC/MS: [M + H]⁺: 1004.5.¹H NMR (500 MHz, CD₃OD) δ: 7.65-7.78 |
| | (m, 2H), 7.33-7.43 (m, 2H), 7.08-7.26 (m, 10H), 7.03 (d, J = 8.0 Hz, 1H), 6.80- |
| | 6.85 (m, 2H), 5.66-5.80 (m, 1H), 4.99-5.05 (m, 2H), 4.91-4.96 (m, 1H), 4.74- |
| | 4.80 (m, 1H), 4.54-4.59 (m, 2H), 4.52 (s, 1H), 4.46 (s, 1H), 3.80-3.90 (m, 2H), |
| | 3.73 (d, J = 1.5 Hz, 3H), 3.66-3.71 (m, 1H), 3.55-3.60 (m, 1H), 3.46-3.52 (m, |
| | 1H), 3.37-3.42 (m, 2H), 3.26-3.29 (m, 1H), 3.18 (dd, J₁ = 3.0 Hz, J₂ = 5.5 Hz, |
| | 1H), 3.13 (t, J = 7.5 Hz, 1H), 2.92-3.05 (m, 3H), 2.71-2.82 (m, 4H), 2.64 (t, |
| | J = 7.5 Hz, 1H), 1.91-2.05 (m, 3H), 1.69-1.80 (m, 2H), 1.51-1.60 (m, 2H), 1.50 |
| | (d, J = 12.5 Hz, 3H), 1.28-1.32 (m, 1H), 1.12 (dd, J₁ = 1.6 Hz, J₂ = 6.5 Hz, 1H). |
| T2412-4 | LC/MS: [M + H]⁺: 1677.8.¹H NMR (500 MHz, CD₃OD) δ: 7.65-7.76 |
| | (m, 2H), 7.31-7.45 (m, 4H), 7.20-7.28 (m, 6H), 7.10-7.20 (m, 6H), 7.04-7.08 |
| | (m, 2H), 7.01 (d, J = 8.0 Hz, 1H), 6.86-6.90 (m, 2H), 6.80-6.82 (m, 2H), 5.94- |
| | 6.01 (m, 1H), 5.67-5.80 (m, 1H), 5.12 (dq, J₁ = 1.5 Hz, J₂ = 10.0 Hz, 1H), 5.01- |
| | 5.09 (m, 2H), 4.93-4.99 (m, 3H), 4.77-4.82 (m, 1H), 4.70-4.77 (m, 3H), 4.65- |
| | 4.70 (m, 2H), 4.52 (s, 1H), 4.46 (s, 1H), 4.38 (dd, J₁ = 3.0 Hz, J₂ = 9.5 Hz, 1H), |
| | 4.24 (dd, J₁ = 3.0 Hz, J₂ = 4.5 Hz, 1H), 4.09-4.17 (m, 3H), 4.05-4.08 (m, 1H), |
| | 4.00 (d, J = 14.5 Hz, 1H), 3.88 (q, J = 9.0 Hz, 1H), 3.76-3.83 (m, 1H), 3.66-3.73 |
| | (m, 4H), 3.59-3.66 (m, 1H), 3.47-3.52 (m, 1H), 3.35-3.43 (m, 2H), 3.27-3.30 |
| | (m, 1H), 3.20 (dd, J₁ = 6.5 Hz, J₂ = 14.5 Hz, 1H), 3.13 (t, J = 8.0 Hz, 1H), 2.96- |
| | 3.05 (m, 4H), 2.91-2.95 (m, 2H), 2.70-2.81 (m, 4H), 2.64 (t, J = 7.5 Hz, 1H), |
| | 2.01-2.06 (m, 2H), 1.91-2.00 (m, 3H), 1.82-1.90 (m, 1H), 1.68-1.81 (m, 3H), |
| | 1.49-1.59 (m, 4H), 1.45 (s, 9H), 1.29-1.33 (m, 1H), 1.12 (dd, J₁ = 2.5 Hz, J₂ = 6.5 |
| | Hz, 1H), 0.99 (t, J = 7.0 Hz, 1H). |
| T2412-6 | LC/MS: [M + H]⁺: 1559.9.¹H NMR (500 MHz, CD₃OD) δ: 7.70 (d, J = 8.0 |
| | Hz, 0.5H), 7.64 (d, J = 8.0 Hz, 0.5H), 7.61 (s, 0.5H), 7.57 (s, 0.5H), 7.50 (d, |
| | J = 8.0 Hz, 0.5H), 7.41-7.45 (m, 1H), 7.34-7.37 (m, 1.5H), 7.19-7.26 (m, 8H), |
| | 7.08-7.16 (m, 4H), 7.03 (d, J = 8.0 Hz, 1H), 6.91-6.94 (m, 3H), 6.83-6.87 (m, |
| | 2H), 6.77-6.83 (m, 2H), 5.89-5.97 (m, 1H), 5.68-5.80 (m, 1H), 5.06-5.10 (m, |
| | 2H), 4.95-5.04 (m, 3H), 4.84-4.88 (m, 1H), 4.74-4.81 (m, 2H), 4.65-4.71 (m, |
| | 4H), 4.55-4.59 (m, 1H), 4.48-4.52 (m, 1H), 4.38 (d, J = 15.0 Hz, 1H), 4.22-4.26 |
| | (m, 2H), 4.18 (dd, J₁ = 3.0 Hz, J₂ = 6.0 Hz, 1H), 4.03 (dd, J₁ = 6.5 Hz, J₂ = 15.0 |
| | Hz, 1H), 3.85-3.96 (m, 3H), 3.77-3.83 (m, 1H), 3.71 (d, J = 10.0 Hz, 1H), 3.58- |
| | 3.62 (m, 2H), 3.47-3.55 (m, 2H), 3.12-3.27 (m, 5H), 2.89-3.06 (m, 7H), 2.65- |
| | 2.79 (m, 4H), 1.93-2.04 (m, 5H), 1.82-1.86 (m, 1H), 1.73-1.77 (m, 1H), 1.64 |
| | (quintet, J = 6.0 Hz, 1H), 1.57 (quintet, J = 7.5 Hz, 2H), 1.42 (s, 3H), 1.30-1.32 |
| | (m, 1H), 1.13 (dd, J₁ = 2.5 Hz, J₂ = 3.5 Hz, 3H). |
| 2412 | LC/MS: [M]⁺: 1554.8.¹H NMR (500 MHz, CD₃OD) δ: 7.45-7.52 (m, |
| | 3H), 7.40 (dd, J₁ = 2.5 Hz, J₂ = 9.5 Hz, 1H), 7.23-7.28 (m, 3H), 7.14-7.19 (m, |
| | 4H), 7.04-7.06 (m, 3H), 6.94-6.99 (m, 2H), 6.85-6.90 (m, 2H), 6.78 (td, J₁ = 2.5 |
| | Hz, J₂ = 9.0 Hz, 1H), 5.33-5.36 (m, 1H), 5.25 (d, J = 14.5 Hz, 2H), 4.64-4.69 (m, |
| | 2H), 4.59-4.61 (m, 4H), 4.53-4.56 (m, 1H), 4.49 (d, J = 1.5 Hz, 1H), 4.27 (d, |
| | J = 3.5 Hz, 1H), 4.24 (d, J = 4.0 Hz, 1H), 4.08-4.18 (m, 4H), 3.94-4.04 (m, 3H), |
| | 3.72-3.78 (m, 4H), 3.62-3.69 (m, 3H), 3.45 (quintet, J = 1.5 Hz, 1H), 3.33-3.38 |
| | (m, 2H), 3.20-3.24 (m, 2H), 3.16-3.18 (m, 1H), 3.02- 3.08 (m, 7H), 2.92-2.99 |
| | (m, 2H), 2.85-2.90 (m, 2H), 2.78-2.83 (m, 2H), 2.71-2.76 (m, 1H), 2.63-2.71 |
| | (m, 2H), 2.54-2.59 (m, 2H), 2.25-2.32 (m, 3H), 2.18-2.21 (m, 2H), 2.08-2.13 |
| | (m, 2H), 2.01-2.05 (m, 2H), 1.61-1.72 (m, 8H), 1.44-1.45 (m, 1H), 1.32-1.34 |
| | (m, 2H), 1.16 (s, 3H), 1.09 (d, J = 6.5 Hz, 3H), 1.04-1.11 (m, 1H), 0.90 (t, J = 6.5 |
| | Hz, 2H). |
| 2453 | HRMS: [M]⁺: 1540.8051.¹H NMR (500 MHz, CD₃OD) δ: 7.52 (d, J = 7.5 |
| | Hz, 1H), 7.48 (t, J = 7.5 Hz, 1H), 7.37-7.46 (m, 2H), 7.30 (s, 1H), 7.27 (dd, |
| | J₁ = 4.5 Hz, J₂ = 9.0 Hz, 1H), 7.21-7.24 (m, 2H), 7.14-7.19 (m, 3H), 7.04-7.07 |
| | (m, 3H), 6.93-6.99 (m, 2H), 6.85-6.90 (m, 2H), 6.78 (td, J₁ = 2.5 Hz, J₂ = 9.0 Hz, |
| | 1H), 6.00-6.02 (m, 1H), 5.33-5.35 (m, 1H), 5.27 (d, J = 16.0 Hz, 2H), 4.65-4.70 |
| | (m, 2H), 4.55-4.62 (m, 2H), 4.51 (s, 1H), 4.28 (d, J = 3.5 Hz, 1H), 4.25 (d, |
| | J = 4.0 Hz, 1H), 4.08-4.20 (m, 4H), 3.95-4.06 (m, 3H), 3.77 (s, 3H), 3.63-3.75 |
| | (m, 5H), 3.36-3.39 (m, 1H), 3.33-3.35 (m, 1H), 3.19-3.24 (m, 1H), 3.10-3.18 |
| | (m, 3H), 3.03-3.08 (m, 9H), 2.93-2.99 (m, 3H), 2.86-2.91 (m, 2H), 2.78-2.86 |
| | (m, 2H), 2.63-2.77 (m, 3H), 2.53-2.58 (m, 1H), 2.31-2.38 (m, 2H), 2.19-2.23 |
| | (m, 1H), 2.08-2.18 (m, 2H), 1.99-2.05 (m, 1H), 1.75-1.84 (m, 3H), 1.58-1.72 |
| | (m, 5H), 1.32-1.34 (m, 2H), 1.19-1.23 (m, 1H), 1.15 (s, 3H), 1.04-1.12 (m, |
| | 5H), 0.94-0.99 (m, 2H). |
| 2454 | LC/MS: [M]⁺: 1601.8.¹H NMR (500 MHz, CD₃OD) δ: 7.42-7.52 (m, |
| | 3H), 7.39 (dd, J₁ = 2.5 Hz, J₂ = 9.5 Hz, 1H), 7.13-7.28 (m, 6H), 7.05 (t, J = 7.5 |
| | Hz, 3H), 6.94-7.00 (m, 2H), 6.77-6.84 (m, 2H), 6.07-6.09 (m, 1H), 5.34 (t, |
| | J = 5.0 Hz, 1H), 5.23 (d, J = 15.0 Hz, 1H), 4.66-4.70 (m, 3H), 4.57-4.60 (m, 2H), |
| | 4.49 (s, 1H), 4.23-4.28 (m, 2H), 4.09-4.18 (m, 3H), 3.97-4.03 (m, 3H), 3.76 |
| | (s, 3H), 3.58-3.75 (m, 7H), 3.44-3.45 (m, 1H), 3.32-3.35 (m, 11H), 3.04-3.22 |
| | (m, 9H), 2.93-3.01 (m, 3H), 2.66-2.89 (m, 6H), 2.46-2.59 (m, 3H), 2.09-2.21 |
| | (m, 3H), 2.02-2.05 (m, 2H), 1.55-1.65 (m, 4H), 1.45-1.48 (m, 1H), 1.31-1.41 |
| | (m, 3H), 1.16 (s, 3H), 1.03-1.11 (m, 4H), 0.90 (t, J = 7.0 Hz, 2H). |
| T2421-1 | LC/MS: [M + H]⁺: 961.50.¹H NMR (500 MHz, CD₃OD) δ: 7.31 (d, J = 8.0 |
| | Hz, 1H), 7.21-7.28 (m, 3H), 7.13-7.19 (m, 3H), 7.10 (d, J = 8.0 Hz, 1H), 6.84 |
| | (dd, J₁ = 2.5 Hz, J₂ = 9.0 Hz, 2H), 5.50-5.85 (m, 1H), 5.01-5.05 (m, 1H), 4.95- |
| | 4.97 (m, 1H), 4.81-4.84 (m, 2H), 4.43 (s, 1H), 3.97-4.01 (m, 2H), 3.79-3.83 |
| | (m, 1H), 3.74 (s, 3H), 3.71 (d, J = 14.0 Hz, 2H), 3.35-3.51 (m, 4H), 3.11-3.142 |
| | (m, 1H), 2.98-3.06 (m, 1H), 2.71-2.85 (m, 3H), 2.05-2.09 (m, 1H), 1.98-2.04 |
| | (m, 1H), 1.86-1.91 (m, 1H), 1.67-1.84 (m, 4H), 1.54-1.60 (m, 1H), 1.50 (d, |
| | J = 6.5 Hz, 3H), 1.45 (s, 9H), 1.14 (s, 9H), 1.07 (d, J = 6.5 Hz, 3H). |
| T2421-2 | LC/MS: [M + H]⁺: 1182.2.¹H NMR (500 MHz, CD₃OD) δ: 7.31-7.35 |
| | (m, 4H), 7.25-7.29 (m, 3H), 7.21-7.24 (m, 1H), 7.11-7.17 (m, 3H), 7.09 (d, |
| | J = 8.0 Hz, 1H), 6.84 (dd, J₁ = 2.5 Hz, J₂ = 9.0 Hz, 2H), 5.51-5.84 (m, 1H), 5.01- |
| | 5.08 (m, 2H), 4.94-4.91 (m, 1H), 4.80-4.86 (m, 3H), 4.74 (brs, 1H), 4.53-4.57 |
| | (m, 1H), 4.42 (s, 1H), 3.96-4.01 (m, 2H), 3.80-3.83 (m, 1H), 3.74 (s, 3H), |
| | 3.59-3.66 (m, 3H), 3.44-3.51 (m, 2H), 3.36-3.43 (m, 2H), 3.12 (t, J = 7.5 Hz, |
| | 1H), 3.02 (qd, J₁ = 6.5 Hz, J₂ = 17.5 Hz, 1H), 2.70-2.85 (m, 3H), 2.06 (q, J = 6.5 |
| | Hz, 1H), 2.00 (dd, J₁ = 6.0 Hz, J₂ = 12.5 Hz, 1H), 1.86-1.91 (m, 1H), 1.74-1.83 |
| | (m, 3H), 1.69 (quintet, J = 7.5 Hz, 1H), 1.56 (quintet, J = 7.5 Hz, 1H), 1.50 (d, |
| | J = 6.0 Hz, 3H), 1.45 (s, 9H), 1.14 (s, 9H), 1.07 (d, J = 6.0 Hz, 3H). |
| T2421-3 | LC/MS: [M + H]⁺: 1026.2.¹H NMR (500 MHz, CD₃OD) δ: 7.21-7.40 |
| | (m, 9H), 7.14-7.18 (m, 2H), 7.09-7.13 (m, 2H), 6.82-6.86 (m, 2H), 5.51-5.59 |
| | (m, 1H), 5.02-5.05 (m, 1H), 4.94-4.97 (m, 1H), 4.77-4.82 (m, 1H), 4.42 (s, |
| | 1H), 4.34-4.38 (m, 1H), 3.84-3.90 (m, 1H), 3.78-3.83 (m, 1H), 3.74 (s, 3H), |
| | 3.58-3.64 (m, 3H), 3.47-3.55 (m, 1H), 3.34-3.46 (m, 4H), 3.10-3.14 (m, 2H), |
| | 2.98-3.06 (m, 1H), 2.71-2.85 (m, 3H), 1.98-2.08 (m, 2H), 1.87-1.95 (m, 1H), |
| | 1.74-1.83 (m, 3H), 1.69 (quintet, J = 7.5 Hz, 1H), 1.53-1.59 (m, 1H), 1.51 (d, |
| | J = 6.0 Hz, 3H), 1.29 (s, 3H), 1.10 (d, J = 6.5 Hz, 3H). |
| T2421-4 | LC/MS: [M + H]⁺: 1699.8.¹H NMR (500 MHz, CD₃OD) δ: 7.18-7.33 |
| | (m, 12H), 7.12-7.18 (m, 5H), 7.08 (d, J = 7.5 Hz, 2H), 6.86-6.90 (m, 2H), 6.81- |
| | 6.84 (m, 2H), 5.93-6.01 (m, 1H), 5.51-5.83 (m, 1H), 5.10-5.13 (m, 1H), 5.04 |
| | (d, J = 14.5 Hz, 2H), 4.94-5.00 (m, 4H), 4.78-4.85 (m, 4H), 4.66-4.72(m, 5H), |
| | 4.50-4.52 (m, 1H), 4.38-4.41 (m, 2H), 4.25 (d, J = 4.5 Hz, 1H), 4.09-4.18 (m, |
| | 3H), 4.04-4.09 (m, 1H), 4.00 (d, J = 14.5 Hz, 1H), 3.89 (d, J = 9.0 Hz, 1H), 3.78- |
| | 3.83 (m, 1H), 3.72 (s, 3H), 3.60-3.65 (m, 2H), 3.44-3.51 (m, 2H), 3.37-3.44 |
| | (m, 2H), 3.21 (dd, J₁ = 6.5 Hz, J₂ = 14.5 Hz, 1H), 3.11 (t, J = 8.0 Hz, 1H), 2.97- |
| | 3.06 (m, 3H), 2.90-2.94 (m, 1H), 2.70-2.84 (m, 3H), 1.96-2.12 (m, 4H), 1.65- |
| | 1.90 (m, 5H), 1.54-1.58 (m, 1H), 1.49 (d, J = 7.0 Hz, 3H), 1.45 (s, 9H), 1.12 (d, |
| | J = 6.5 Hz, 3H). |
| T2421-6 | LC/MS: [M + H]⁺: 1581.7.¹H NMR (500 MHz, CD₃OD) δ: 7.37-7.45 |
| | (m, 1H), 7.29-7.37 (m, 4H), 6.98-7.29 (m, 13H), 6.82-6.93 (m, 3H), 6.66-6.80 |
| | (m, 2H), 5.84-5.91 (m, 1H), 5.46-5.80 (m, 1H), 4.92-5.14 (m, 5H), 4.71-4.81 |
| | (m, 4H), 4.56-4.69 (m, 4H), 4.39-4.51 (m, 2H), 4.30 (d, J = 3.0 Hz, 3H), 4.22- |
| | 4.32 (m, 3H), 4.09-4.20 (m, 2H), 3.84(d, J = 14.5 Hz, 1H), 3.76-3.78 (m, 3H), |
| | 3.59-3.73 (m, 4H), 3.51-3.55 (m, 2H), 3.40-3.45 (m, 1H), 3.33-3.37 (m, 1H), |
| | 3.23 (dd, J₁ = 6.0 Hz, J₂ = 14.5 Hz, 1H), 3.12-3.18 (m, 1H), 2.91-3.01 (m, |
| | 6H),2.67-2.81 (m, 2H), 1.97-2.04 (m, 3H), 1.67-1.70 (m, 2H), 1.60-1.66 (m, |
| | 1H), 1.42-1.47 (m, 3H), 1.27-1.36 (m, 4H), 1.10 (dd, J₁ = 6.0 Hz, J₂ = |
| | 17.0 Hz, 3H). |
| 2421 | HRMS: [M]⁺: 1562.7756.¹H NMR (500 MHz, CD₃OD) δ: 7.70-7.76 |
| | (m, 1H), 7.40 (dd, J₁ = 6.0 Hz, J₂ = 9.0 Hz, 1H), 7.35 (d, J = 9.0 Hz, 1H), 7.20- |
| | 7.25 (m, 5H), 7.13-7.17 (m, 5H), 6.97 (d, J = 8.5 Hz, 2H), 6.90 (d, J = 7.5 Hz, |
| | 2H), 6.75-6.80 (m, 1H), 5.65-5.67 (m, 1H), 5.52-5.55 (m, 2H), 4.67-4.73 (m, |
| | 2H), 4.53-4.57 (m, 2H), 4.47-4.83 (m, 1H), 4.38-4.40 (m, 1H), 4.33 (d, J = 3.0 |
| | Hz, 1H), 4.30 (d, J = 3.5 Hz, 1H), 4.06-4.20 (m, 5H), 4.00-4.04 (m, 2H), 3.80- |
| | 3.91 (m, 1H), 3.70-3.83 (m, 6H), 3.60-3.64 (m, 1H), 3.53 (dd, J₁ = 5.0 Hz, |
| | J₂ = 14.0 Hz, 1H), 3.44-3.47 (m, 1H), 3.34-3.37 (m, 2H), 3.25-3.28 (m, 1H), |
| | 3.17-3.21 (m, 1H), 3.07-3.12 (m, 9H), 2.96 (d, J = 8.0 Hz, 3H), 2.90-2.93 (m, |
| | 1H), 2.82-2.88 (m, 1H), 2.75 (d, J = 13.0 Hz, 1H), 2.60-2.63 (m, 1H), 2.50-2.55 |
| | (m, 1H), 2.30-2.36 (m, 3H), 2.16-2.23 (m, 1H), 1.77-1.85 (m, 2H), 1.65-1.71 |
| | (m, 2H), 1.50-1.54 (m, 1H), 1.38-1.45 (m, 2H), 1.27-1.33 (m, 2H), 1.10 (d, |
| | J = 6.5 Hz, 3H), 0.89-1.04 (m, 5H), 0.73-0.78 (m, 2H), 0.49-0.56 (m, 2H). |
| 2427 | LC/MS: [M]⁺: 1598.7.¹H NMR (500 MHz, CD₃OD) δ: 7.66 (brs, 2H), |
| | 7.27-7.31 (m, 3H), 7.19-7.24 (m, 4H), 7.13 (d, J = 7.5 Hz, 2H), 6.96-7.06 (m, |
| | 5H), 5.43-5.47 (m, 3H), 4.52 (s, 2H), 4.43 (d, J = 14.0 Hz, 2H), 4.27-4.33 (m, |
| | 2H), 4.16-4.21 (m, 2H), 4.10-4.13 (m, 1H), 4.04 (d, J = 15.0 Hz, 2H), 3.96 (d, |
| | J = 15.0 Hz, 2H), 3.88-3.90 (m, 1H), 3.86 (s, 3H), 3.70-3.76 (m, 2H), 3.58-3.67 |
| | (m, 4H), 3.56 (t, J = 5.0 Hz, 1H), 3.47-3.50 (m, 3H), 3.15-3.25 (m, 3H), 2.93- |
| | 3.11 (m, 11H), 2.79 (d, J = 13.0 Hz, 1H), 2.67-2.74 (m, 1H), 2.57-2.63(m, 2H), |
| | 2.36 (t, J = 6.5 Hz, 2H), 2.18-2.23 (m, 1H), 1.99-2.10 (m, 2H), 1.75-1.82 (m, |
| | 2H), 1.66-1.74 (m, 2H), 1.52-1.59 (m, 2H), 1.37-1.43 (m, 2H), 1.25-1.30 (m, |
| | 2H), 1.12-1.18 (m, 2H), 1.07 (d, J = 6.5 Hz, 3H), 0.94 (t, J = 7.5 Hz, 2H), 0.80- |
| | 0.83 (m, 2H), 0.40-0.49 (m, 2H), 0.30-0.40 (m, 2H). |
| 2428 | LC/MS: [M]⁺: 1644.7.¹H NMR (500 MHz, CD₃OD) δ: 7.20-7.37 (m, |
| | 10H), 7.03 (dd, J₁ = 8.5 Hz, J₂ = 12.0 Hz, 4H), 6.95 (d, J = 8.0 Hz, 2H), 6.72-6.75 |
| | (m, 1H), 5.59 (d, J = 13.0 Hz, 2H), 5.40 (dd, J₁ = 4.0 Hz, J₂ = 8.0 Hz, 1H), 5.34 |
| | (t, J = 4.5 Hz, 1H), 5.21 (brs, 1H), 5.11-5.15 (m, 1H), 5.52-5.55 (brs, 2H), 4.26- |
| | 4.33 (m, 3H), 4.11-4.14 (m, 3H), 4.03-4.06 (m, 2H), 3.86 (s, 3H), 3.79-3.83 |
| | (m, 2H), 3.66 (t, J = 5.0 Hz, 1H), 3.62-3.64 (m, 1H), 3.58-3.61 (m, 1H), 3.53- |
| | 3.58 (m, 2H), 3.49 (t, J = 6.5 Hz, 2H), 3.44-3.45 (m, 1H), 3.34-3.37 (m, 1H), |
| | 3.10-3.22 (m, 9H), 2.93-2.97 (m, 3H), 2.81-2.85 (m, 3H), 2.41-2.45(m, 2H), |
| | 2.26-2.37 (m, 5H), 2.18-2.25 (m, 2H), 2.01-2.05 (m, 1H), 1.79-1.86 (m, 3H), |
| | 1.62-1.69 (m, 3H), 1.54-1.60 (m, 2H), 1.44-1.46 (m, 1H), 1.37-1.43 (m, 2H), |
| | 1.29 (s, 3H), 1.01 (d, J = 6.5 Hz, 3H), 0.94 (t, J = 7.5 Hz, 1H), 0.71 (brs, 3H). |
| T2407-1 | LC/MS: [M + H]⁺: 988.5.¹H NMR (400 MHz, CD₃OD) δ: 8.00-8.21 (m, |
| | 2H), 7.63-7.74(m, 1H), 7.50-7.60(m, 2H), 7.41-7.51(m, 3H), 7.18 (dd, J₁ = 8.0 |
| | Hz, J₂ = 4.0 Hz, 1H), 7.10-7.15 (m, 2H), 7.07 (d, J = 7.6 Hz, 1H), 6.81-6.86 (m, |
| | 4H), 5.37-5.49 (m, 1H), 4.95-4.99 (m, 2H), 4.74-4.81 (m, 2H), 4.11-4.25 (m, |
| | 1H),3.92-4.00 (m, 2H), 3.77-3.85 (m, 1H), 3.74 (d, J = 4.0 Hz, 3H), 3.42-3.66 |
| | (m, 2H), 3.34-3.39 (m, 1H), 2.96-3.06 (m, 1H), 2.74-2.93 (m, 3H), 2.52-2.74 |
| | (m, 2H), 1.98-2.03 (m, 1H), 1.98-2.03 (m, 1H), 1.86-1.92 (m, 1H), 1.64-1.80 |
| | (m, 4H), 1.50 (d, J = 9.6 Hz, 3H), 1.45 (s, 9H), 1.26-1.36 (m, 2H), 1.14 (s, 9H), |
| | 1.07 (d, J = 5.6 Hz, 3H). |
| T2407-2 | LC/MS: [M + H]⁺: 1208.6.¹H NMR (400 MHz, CD₃OD) δ: 7.90-8.02 |
| | (m, 2H), 7.43-7.69 (m, 6H), 7.19-7.42 (m, 5H), 7.07-7.19 (m, 4H), 6.82-6.88 |
| | (m, 4H), 5.39-5.69 (m, 1H), 4.98-5.07 (m, 4H), 4.71-4.81 (m, 3H), 4.13-4.28 |
| | (m, 1H), 3.95-4.00 (m, 2H), 3.74-3.83 (m, 2H), 3.73 (d, J = 3.6 Hz, 3H), 3.60- |
| | 3.67 (m, 1H), 3.33-3.60 (m, 4H), 2.98-3.06 (m, 1H), 2.76-2.92 (m, 2H), 2.54- |
| | 2.74 (m, 2H), 1.85-2.03 (m, 2H), 1.70-1.77 (m, 3H), 1.60-1.68 (m, 1H), 1.50 |
| | (d, J = 8.0 Hz, 3H), 1.45 (s, 9H), 1.26-1.34 (m, 2H), 1.13 (s, 9H), 1.07 (d, J = 6.0 |
| | Hz, 3H). |
| T2407-3 | LC/MS: [M + H]⁺: 1052.5.¹H NMR (400 MHz, CD₃OD) δ: 7.98-8.01 |
| | (m, 1H), 7.92 (d, J = 8.0 Hz, 1H), 7.49-7.66 (m, 4H), 7.38-7.45 (m, 2H), 7.20- |
| | 7.24 (m, 5H), 7.09-7.18 (m, 4H), 6.81-6.90 (m, 4H), 5.36-5.69 (m, 1H), 4.93- |
| | 5.02 (m, 3H), 4.73-4.81 (m, 2H), 4.57-4.60 (m, 2H), 4.17-4.27 (m, 1H), 3.78- |
| | 3.90 (m, 2H), 3.74 (d, J = 4.0 Hz, 3H), 3.68-3.73 (m, 1H), 3.44-3.67 (m, 3H), |
| | 3.33-3.40 (m, 2H), 3.15 (d, J = 5.6 Hz, 2H), 2.97-3.07 (m, 1H), 2.76-2.93 (m, |
| | 2H), 2.54-2.74 (m, 2H), 1.90-2.07 (m, 2H), 1.69-1.83 (m, 3H), 1.60-1.65 (m, |
| | 1H), 1.51 (d, J = 10.4 Hz, 3H), 1.24-1.36 (m, 2H), 1.11 (d, J = 6.4 Hz, 3H). |

### Experimental Example 1 Activity Test

### 1. In Vitro Enzymatic Activity Test

PCSK9-LDLR HTRF assay was used to test the enzymatic activity of the synthesized new compounds in this experiment.

### 1) the Experimental Steps are as Follows:

i) the test compound was diluted according to the reference concentration, and using the diluted solution as the first point and then serially diluted 3-fold for a total of 10 points.
ii) the diluted compound was transferred onto a 384 analytical plate, with 2 duplicate wells set for each concentration. The 384-well plate was centrifuged at 1000 r/min.
iii) 2.5 μL of PCSK9 solution was added to each well and then centrifuged at 1000 r/min for 1 minute in a centrifuge.
iv) The plate was incubated at 25° C. for 10 minutes.
v) 2.5 μL of LDLR solution was added to each well and then centrifuged at 1000 r/min for 1 minute in a centrifuge.
vi) 5 μL of streptavidin-TB& PAB ANTI human IGG-XL665 working solution was added to each well, and centrifuged at 1000 r/min for 1 minute in a centrifuge.
vii) The plate was incubated at 25° C. for 60 minutes.
viii) BMG microplate reader was used to detect the HTRF ratio (665/620) for data analysis.

### 2) the Enzymatic Activity Results of the New Compounds are as Shown in Table4:

TABLE 4
Enzymatic Activity Test Results of New Compounds

| Compound | | Compound | | Compound | | Compound | |
|---|---|---|---|---|---|---|---|
| Nos. | Ki | Nos. | Ki | Nos. | Ki | Nos. | Ki |
| MK-0616 | 0.06 nm | 2407 | 1.88 nm | 2401 | 0.05 nm | 2450 | 0.05 nm |
| 2451 | 0.05 nm | 2455 | 0.05 nm | 2457 | 0.02 nm | 2460 | 0.04 nm |
| 2461 | 0.02 nm | 2462 | 0.03 nm | 2463 | 0.04 nm | 2421 | 0.05 nm |
| 2427 | 14.01 nm | 2428 | 0.05 nm | 2403 | 0.11 nm | 2404 | 0.05 nm |
| 2416 | 0.05 nm | 2402A | 0.02 nm | 2402B | 0.03 nm | 205A | 0.04 nm |
| 212A | 0.05 nm | 204B | 0.06 nm | 205B | 0.08 nm | 211B | 0.06 nm |
| 212B | 0.06 nm | 213B | 0.06 nm | 2430 | 0.21 nm | 2410 | 0.10 nm |
| 2412 | 0.07 nm | 2452 | 0.12 nm | 2453 | 0.06 nm | 2454 | 0.18 nm |

### 2. Pharmacodynamic Test of Intraperitoneal Injection in Mice

8-week-old male C₅₇ mice were divided into 7 groups, with (mice in each group. The control group received daily injection of Control (2.5% DMSO in PBS), while the experimental group received daily intraperitoneal injection of the test compound (1 mg/kg/day). Blood was collected from the orbital sinus before administration and on day 7 after administration to measure the concentration of LDL-C in mouse serum, The results are shown in Tables 5 and 6 below:

TABLE 5
Summary of Two Measured Values of Blood
Lipids Before and After administration

| | Low-density lipoprotein cholesterol (LDL-C) (mmol/L) | | |
|---|---|---|---|
| Compounds | Before administration | D7 | Vs control (%) |
| Control | 2.66 ± 0.09 | 2.64 ± 0.12 | NA |
| MK-0616 | 2.65 ± 0.10 | 2.07 ± 0.10 | −21.33 |
| 2421 | 2.63 ± 0.09 | 1.71 ± 0.12 | −35.03 |
| 2451 | 2.62 ± 0.08 | 1.78 ± 0.04 | −32.63 |
| 2453 | 2.65 ± 0.08 | 1.70 ± 0.06 | −35.56 |
| 205A | 2.65 ± 0.09 | 1.78 ± 0.08 | −32.63 |

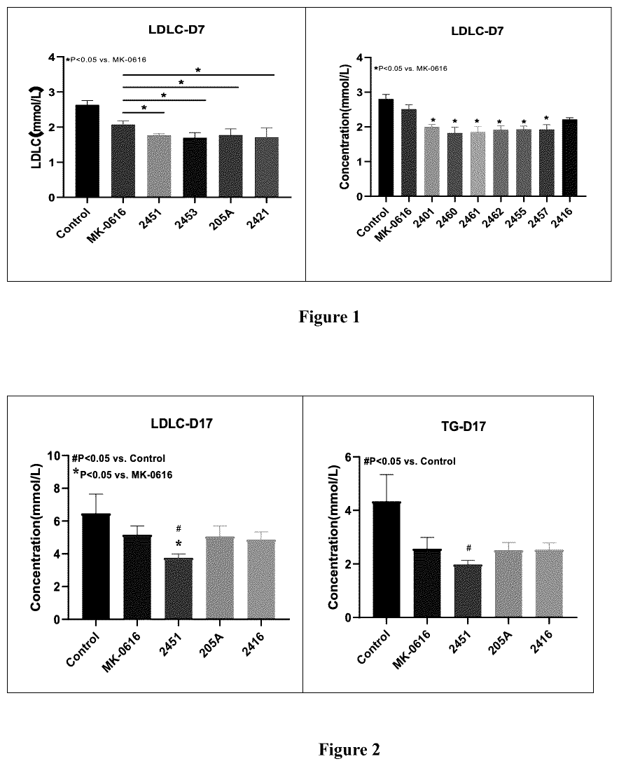

From Table 5 and FIG. 1, it can be observed that under the present experimental conditions, after 7 days of intraperitoneal injection, the LDL-C levels of groups 2451, 2453, 205A, and 2421 decreased by 32.63%, 35.56%, 32.63%, and 35.03%, respectively, compared to the control group, and these reductions showed significant differences when compared to the MK-0616 group.

TABLE 6
Summary of Two Measured Values of Blood
Lipids Before and After administration

| | Low-density lipoprotein cholesterol (LDL-C) (mmol/L) | | |
|---|---|---|---|
| | Before | | Vs |
| Compounds | administration | D7 | control (%) |
| Control | 3.46 ± 0.15 | 2.80 ± 0.13 | NA |
| MK-0616 | 3.42 ± 0.14 | 2.51 ± 0.12 | −10.39 |
| 2416 | 3.44 ± 0.14 | 2.22 ± 0.04 | −20.83 |
| 2460 | 3.42 ± 0.15 | 1.82 ± 0.16 | −34.94 |
| 2401 | 3.46 ± 0.14 | 2.01 ± 0.07 | −28.44 |
| 2462 | 3.47 ± 0.14 | 1.92 ± 0.11 | −31.56 |
| 2455 | 3.44 ± 0.12 | 1.93 ± 0.09 | −31.09 |
| 2461 | 3.42 ± 0.17 | 1.86 ± 0.15 | −33.76 |
| 2457 | 3.44 ± 0.15 | 1.92 ± 0.14 | −31.39 |

From Table 6 and FIG. 1, it can be observed that under the present experimental conditions, after 7 days of intraperitoneal injection, the LDL-C levels of groups 2401, 2455, 2457, 2460, 2461 and 2462 decreased by 28.44%, 31.09%, 31.39%, 34.94%, 33.76% and 31.56%, respectively, compared to the control group, and these reductions showed significant differences when compared to the MK-0616 group.

### Experimental Example 2 Oral Administration Experiment

Objective: Hyperlipidemic model in golden Syrian hamsters was induced by feeding them with high-fat diet, and the therapeutic effect of the test compounds on the hyperlipidemic model of golden Syrian hamsters was evaluated by detecting and monitoring animal blood lipids LDL-C and TG.

Method: golden Syrian hamsters were fed with high-fat diet for 6 weeks to establish a hyperlipidemic model. At the end of week 6, blood samples were collected from the orbital sinus of all golden Syrian hamsters to monitor the levels of LDL-C and TG in their serum, afterwards, the golden Syrian hamsters fed with high-fat diet were evenly divided into 5 groups with 8 in each group based on their body weights and the four levels of blood lipids monitored before grouping. Group 1 was the vehicle control group (360 mg/kg of sodium decanoate solution dissolved in 2% DMSO+98% physiological saline), while groups 2 to 5 were the MK-0616 treatment group, 2451 treatment group, 205A treatment group, and 2416 treatment group, respectively, which were orally administered at a dose of 20 mg/kg once a day for 14 consecutive days. The results are shown in Table 7 below:

TABLE 7
Statistical Table of Blood Lipids in Each Group

| | | Low-density | | | | | |
|---|---|---|---|---|---|---|---|
| | | lipoprotein (LDL-C) | | | Triglyceride (TG) | | |
| | | (mmol/L, Mean) | | | (mmol/L, Mean) | | |
| | Vehicle/Test | Before | | VS | Before | | VS |
| Groups | samples | administration | D 17 | Con. | administration | D 17 | Con. |
| 1 | Vehicle control | 8.018 | 6.468 | NA | 4.051 | 4.344 | NA |
| | (Con.) | | | | | | |
| 2 | MK-0616 | 8.806 | 5.159 | −20.2% | 4.601 | 2.571 | −40.8% |
| 3 | 2451 | 8.889 | 3.750 | −42.0% | 4.594 | 1.981 | −54.4% |
| 4 | 205A | 8.613 | 5.066 | −21.7% | 4.665 | 2.516 | −42.1% |
| 5 | 2416 | 8.387 | 4.869 | −24.7% | 4.947 | 2.536 | −41.6% |

Results: In the hyperlipidemic model of golden Syrian hamsters, on the 17th day after the initial administration, compared with the vehicle control group, the MK-0616 treatment group, 2451 treatment group, 205A treatment group, and 2416 treatment group exhibited reductions in low-density lipoprotein (LDL-C) by 20.2%, 42.0%, 21.7%, and 24.7%, respectively; and triglyceride (TG) levels were reduced by 40.8%, 54.4%, 42.1%, and 41.6%, respectively.

Compared with the vehicle control group, the LDL-C and TG of animals in the 2451 treatment group were both significantly reduced (P<0.05), the other treatment groups also demonstrated a trend Toward reduced LDLC-C and TG levels, but no significant difference was observed. Compared with the MK-0616 treatment group, the 2451 treatment group exhibited significantly greater reductions in LDL-C (P<0.05) (see FIG. 2).

Conclusions: In a hyperlipidemic Golden Syrian hamster model, all treatment groups showed reductions in LDL-C and triglyceride (TG) levels compared to the vehicle control group. Among these, the 2451 treatment group demonstrated the most significant decrease in both parameters, with its LDL-C lowering effect being statistically superior not only to the vehicle control group but also to the MK-0616 treatment group.

1. A compound as shown in formula I or a pharmaceutically acceptable salt thereof: 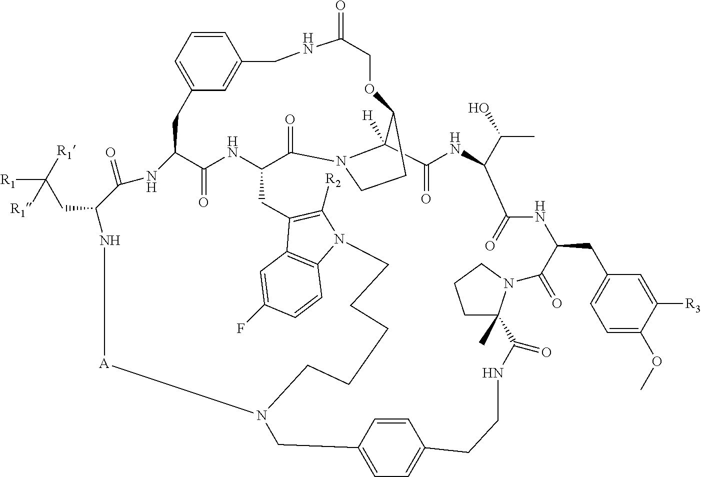
wherein,
R₁ is selected from —CH₃, —C(O)—(CH₂)n₁-N⁺(CH₃)₃, or —C(O)—(CH₂CH₂O)n₂-CH₂CH₂N⁺(CH₃)₃, where n₁ is selected from an integer of 1 to 14, and n₂ is selected from an integer of 1 to 5;
R₁′ and R₁″ are each independently selected from absence, hydrogen or —CH₃;
R₂ is selected from hydrogen, halogen, —SCH₃, —SCH₂X, —SCHX₂, or —SCX₃;
R₃ is selected from hydrogen, or halogen;
A is selected from: 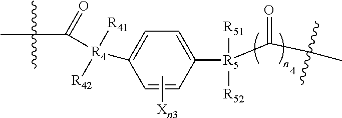
wherein R₄ and R₅ are each independently selected from a bond, a linear C₁-C₅ alkyl, phenyl, provided that R₄ and R₅ are not a bond or phenyl at the same time;
R₄₁ and R₄₂ are each independently selected from absence, hydrogen, deuterium, substituted or unsubstituted C₁-C₃ alkyl, halogen, or, R₄₁ and R₄₂ together with one carbon atom to which they are connected, form a C₃-C₅ cycloalkyl;
R₅₁ and R₅₂ are each independently selected from absence, hydrogen, deuterium, substituted or unsubstituted C₁-C₃ alkyl, halogen, or, R₅₁ and R₅₂ together with one carbon atom to which they are connected, form a C₃-C₅ cycloalkyl;
n₃ is selected from an integer of 0 or 1-4; n₄ is selected from 0 or 1;
among the substituted C₁-C₃ alkyl, the substituent is selected from deuterium, or halogen.

2. The compound or a pharmaceutically acceptable salt thereof according to claim 1, wherein, when R₁ is not —CH₃, either of R₁′ and R₁″ is selected from absence, the other one is selected from hydrogen; or, when R₁ is —CH₃, R₁′ and R₁″ are —CH₃ at the same time.

3. The compound or a pharmaceutically acceptable salt thereof according to claim 1, wherein, R₁ is selected from —C(O)—(CH₂)n₁-N⁺(CH₃)₃ or —C(O)—(CH₂CH₂O)n₂-CH₂CH₂N⁺(CH₃)₃.

4. The compound or a pharmaceutically acceptable salt thereof according to claim 1, wherein, R₂ is selected from hydrogen, —SCHX₂, or —SCX₃.

5. The compound or a pharmaceutically acceptable salt thereof according to claim 1, wherein, R₃ is selected from hydrogen.

6. The compound or a pharmaceutically acceptable salt thereof according to claim 1, wherein, when R₄ is a bond, R₄₁ and R₄₂ are absent; or, when R₅ is a bond, R₅₁ and R₅₂ are absent;
or, when R₄ is a linear alkyl, R₄₁ and R₄₂ are hydrogen, deuterium, substituted or unsubstituted C₁-C₃ alkyl, halogen, or R₄₁ and R₄₂ are connected to the same carbon atom, and together with the carbon atom to which they are connected form a C₃-C₅ cycloalkyl; or, when R₅ is a linear alkyl, R₅₁ and R₅₂ are hydrogen, deuterium, substituted or unsubstituted C₁-C₃ alkyl, halogen, or R₅₁ and R₅₂ are connected to the same carbon atom, and together with the carbon atom to which they are connected form a C₃-C₅ cycloalkyl;
or, when R₄ is phenyl, R₄₁ and R₄₂ are hydrogen, deuterium, substituted or unsubstituted C₁-C₃ alkyl, or halogen; or, when R₅ is phenyl, R₅₁ and R₅₂ are hydrogen, deuterium, substituted or unsubstituted C₁-C₃ alkyl, or halogen.

7. The compound or a pharmaceutically acceptable salt thereof according to claim 1, wherein, R₄ and R₅ are each independently selected from a bond, a linear C₁-C₂ alkyl, or phenyl; R₄ and R₅ are not a bond or phenyl at the same time.

8. The compound or a pharmaceutically acceptable salt thereof according to claim 1, wherein, R₄₁ and R₄₂ are each independently selected from absence, H, D, —CH₃, halogen, —CH₂X, —CHX₂, —CX₃, —CH₂D, —CHD₂, or —CD₃;
R₅₁ and R₅₂ are each independently selected from absence, H, D, —CH₃, halogen, —CH₂X, —CHX₂, —CX₃, —CH₂D, —CHD₂, or —CD₃.

9. The compound or a pharmaceutically acceptable salt thereof according to claim 1, wherein, A is selected from the following structures:

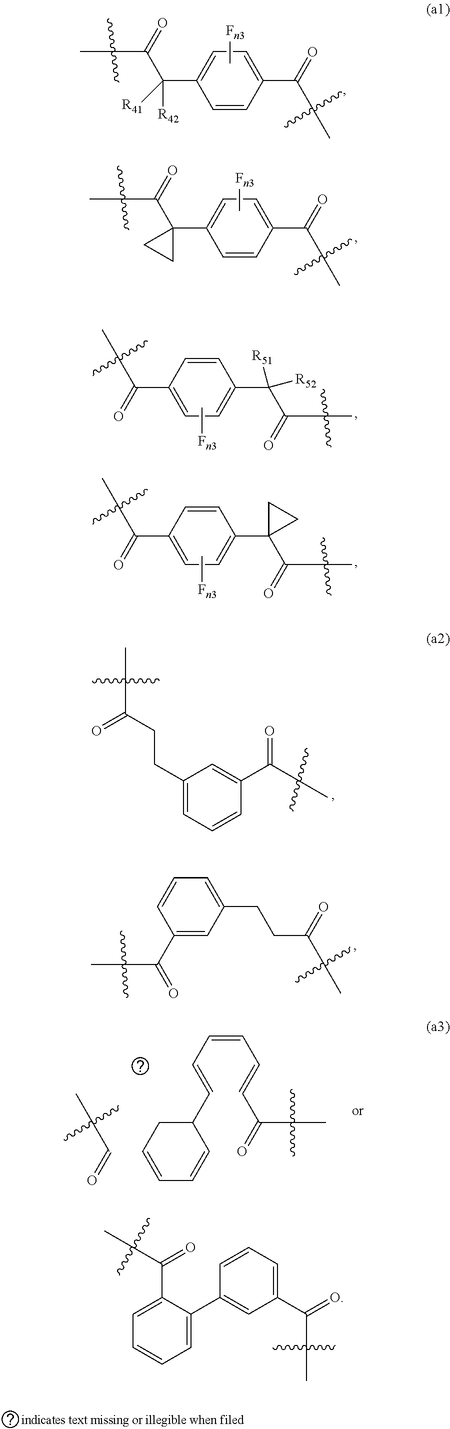

10. The compound or a pharmaceutically acceptable salt thereof according to claim 1, wherein, R₄₁ and R₄₂ are each independently selected from hydrogen, methyl or halogen; R₅₁ and R₅₂ are each independently selected from hydrogen.

11. The compound or a pharmaceutically acceptable salt thereof according to claim 1, wherein, the halogen is selected from F.

12. The compound or a pharmaceutically acceptable salt thereof according to claim 1, wherein, the n₃ is selected from 0, 1, or 2.

13. The compound or a pharmaceutically acceptable salt thereof according to claim 1, wherein the structural formula of the compound is selected from the following:

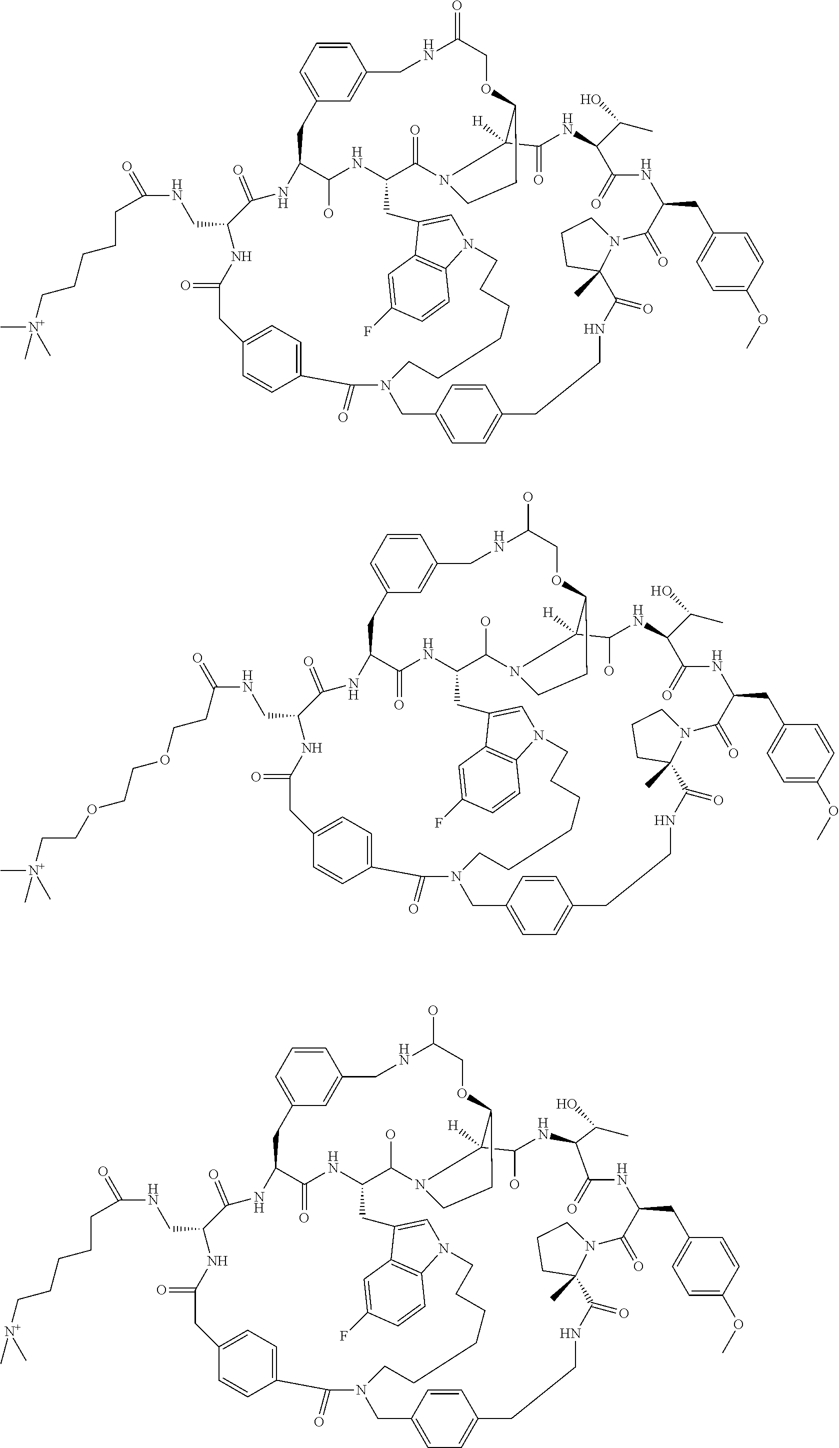

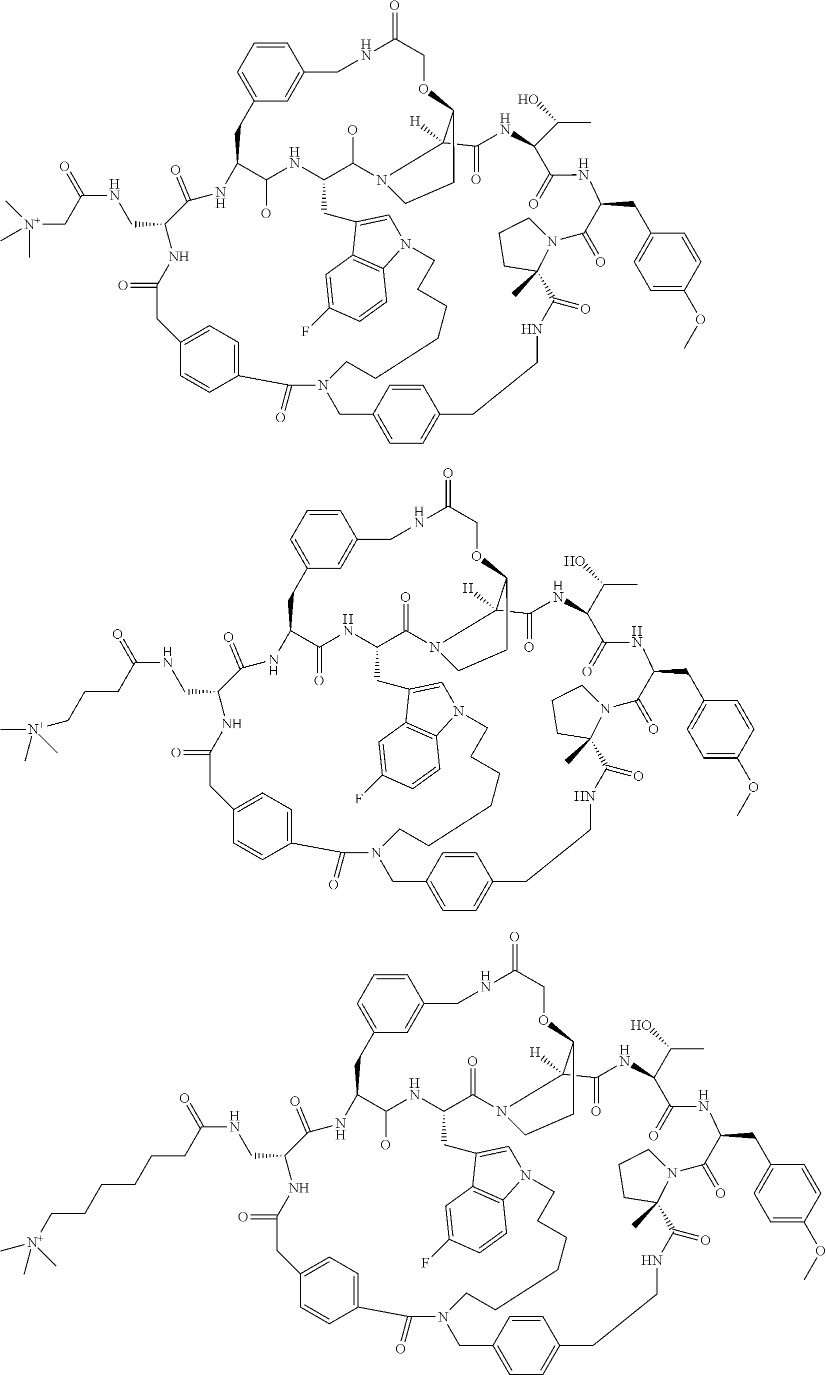

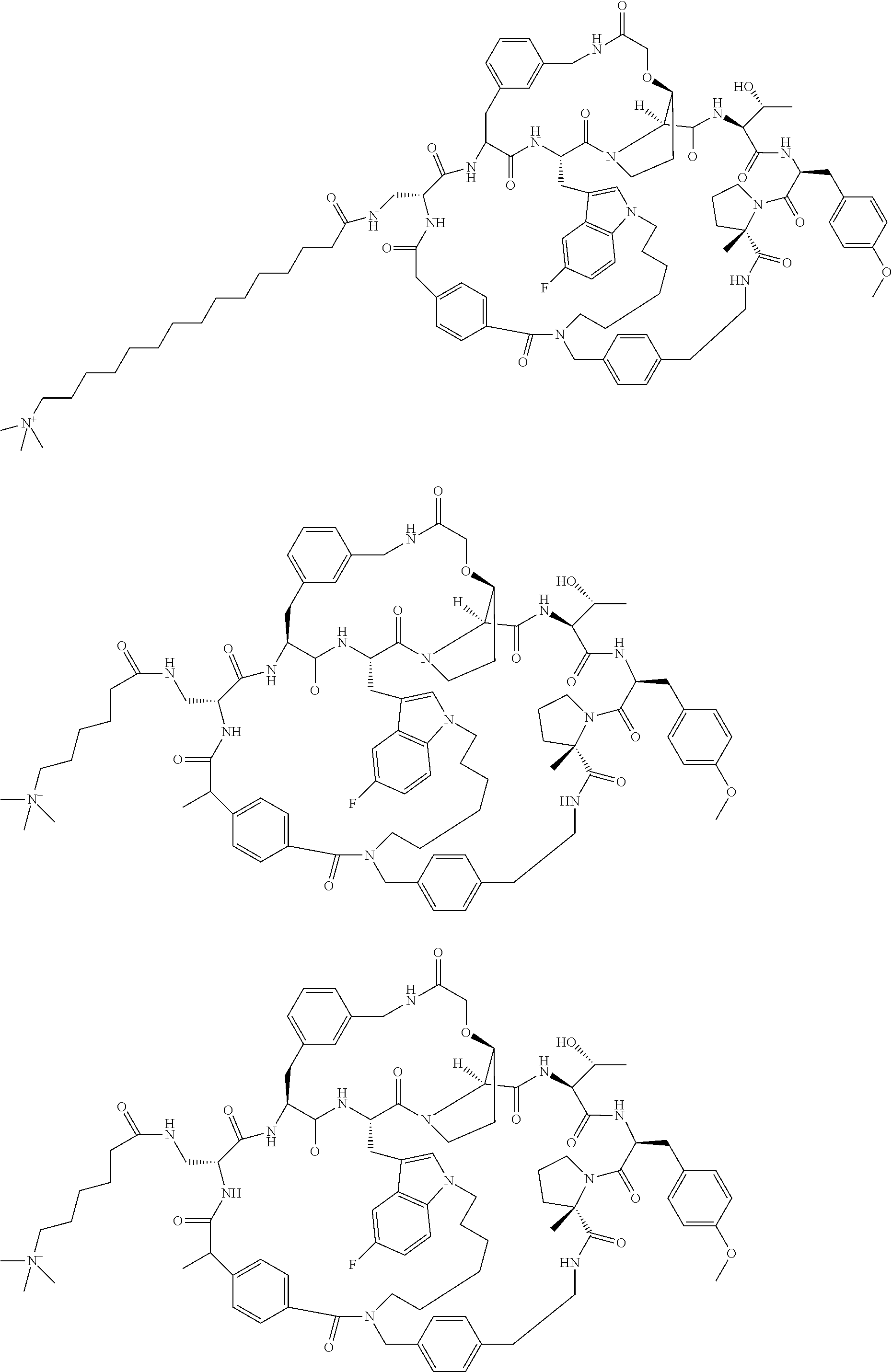

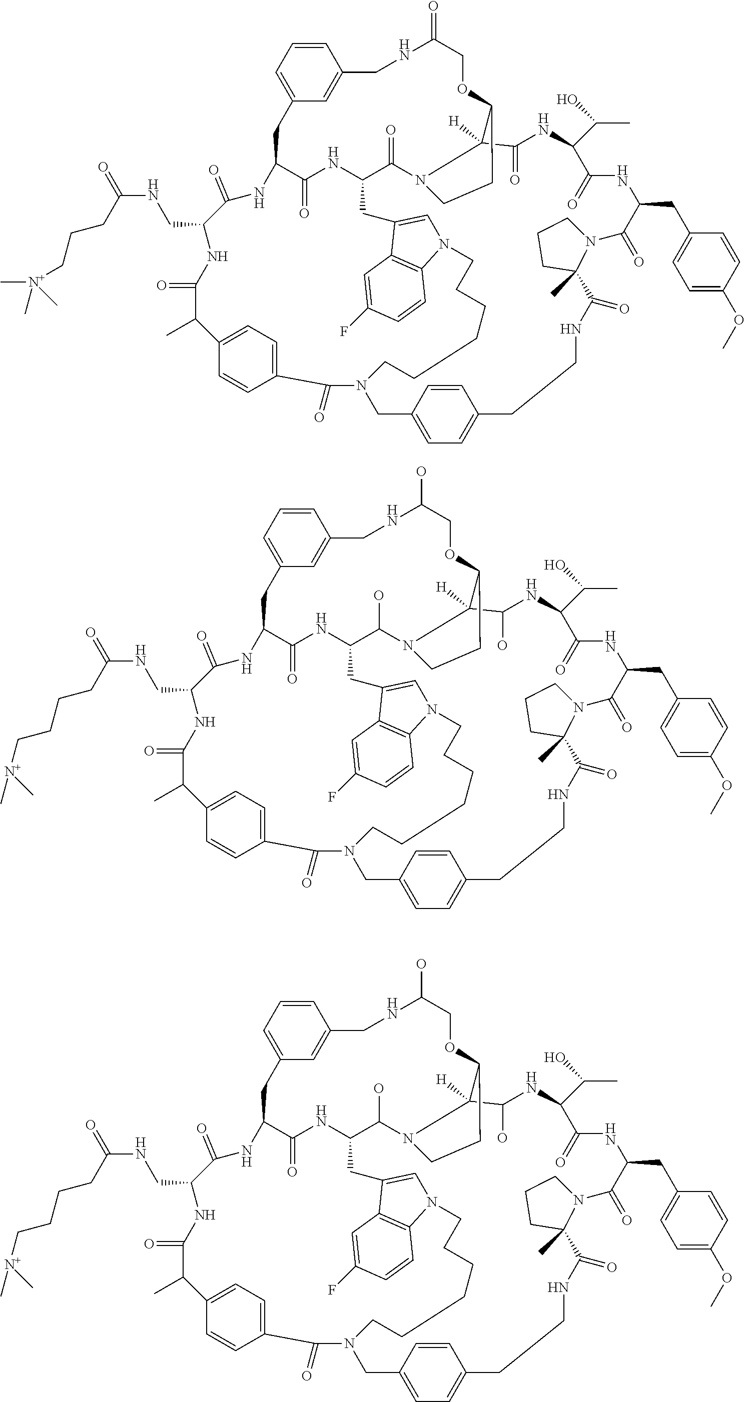

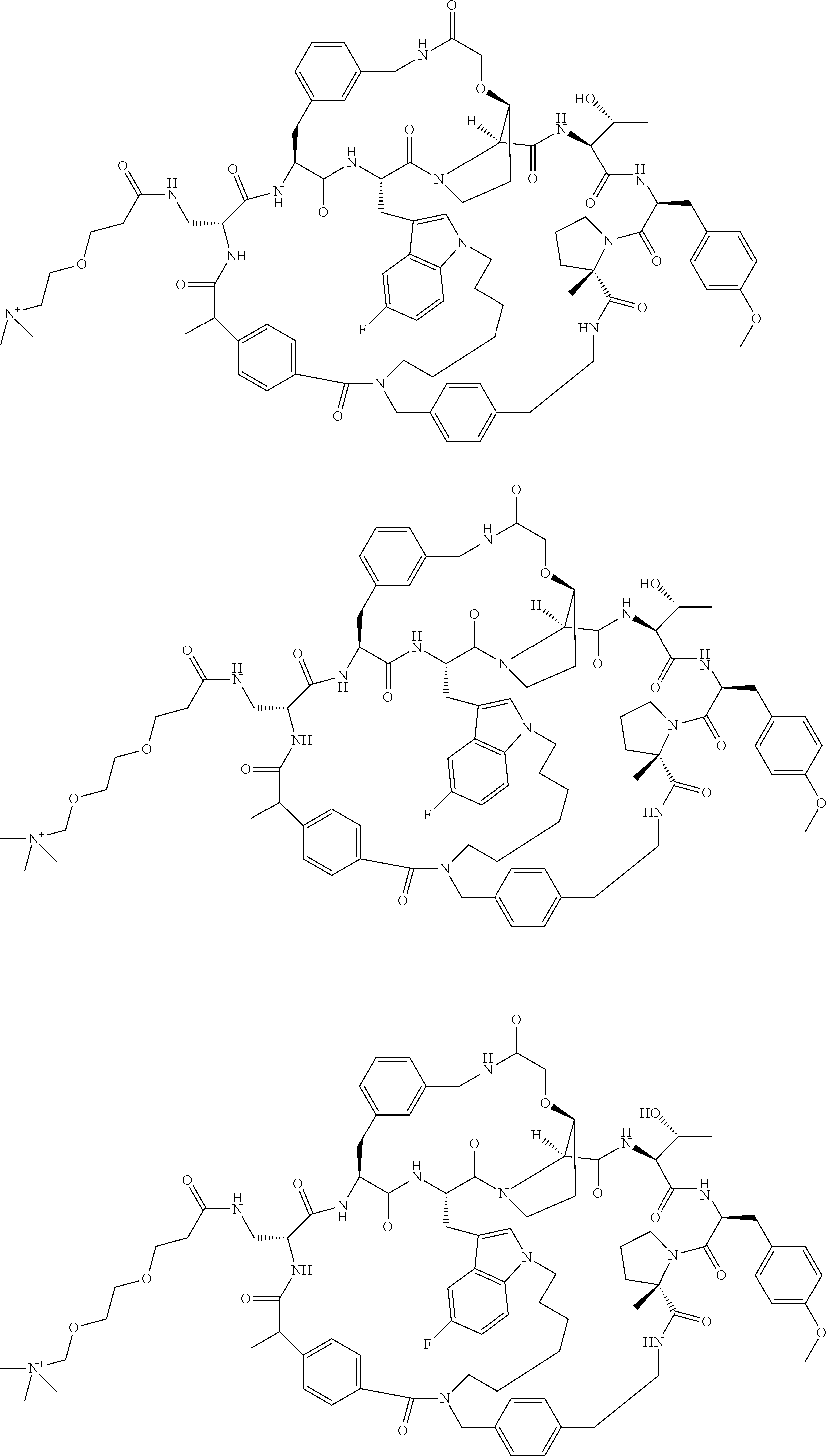

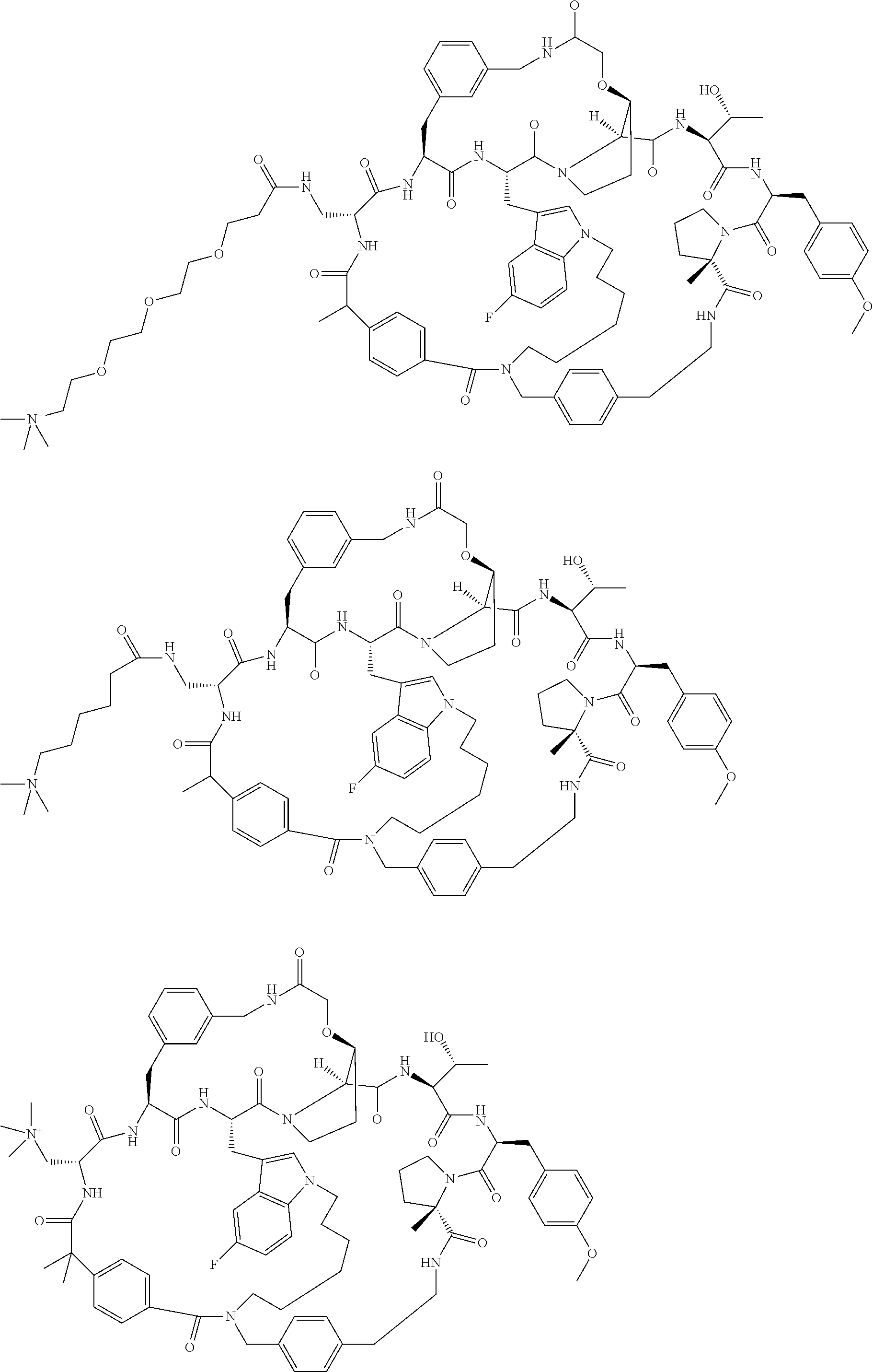

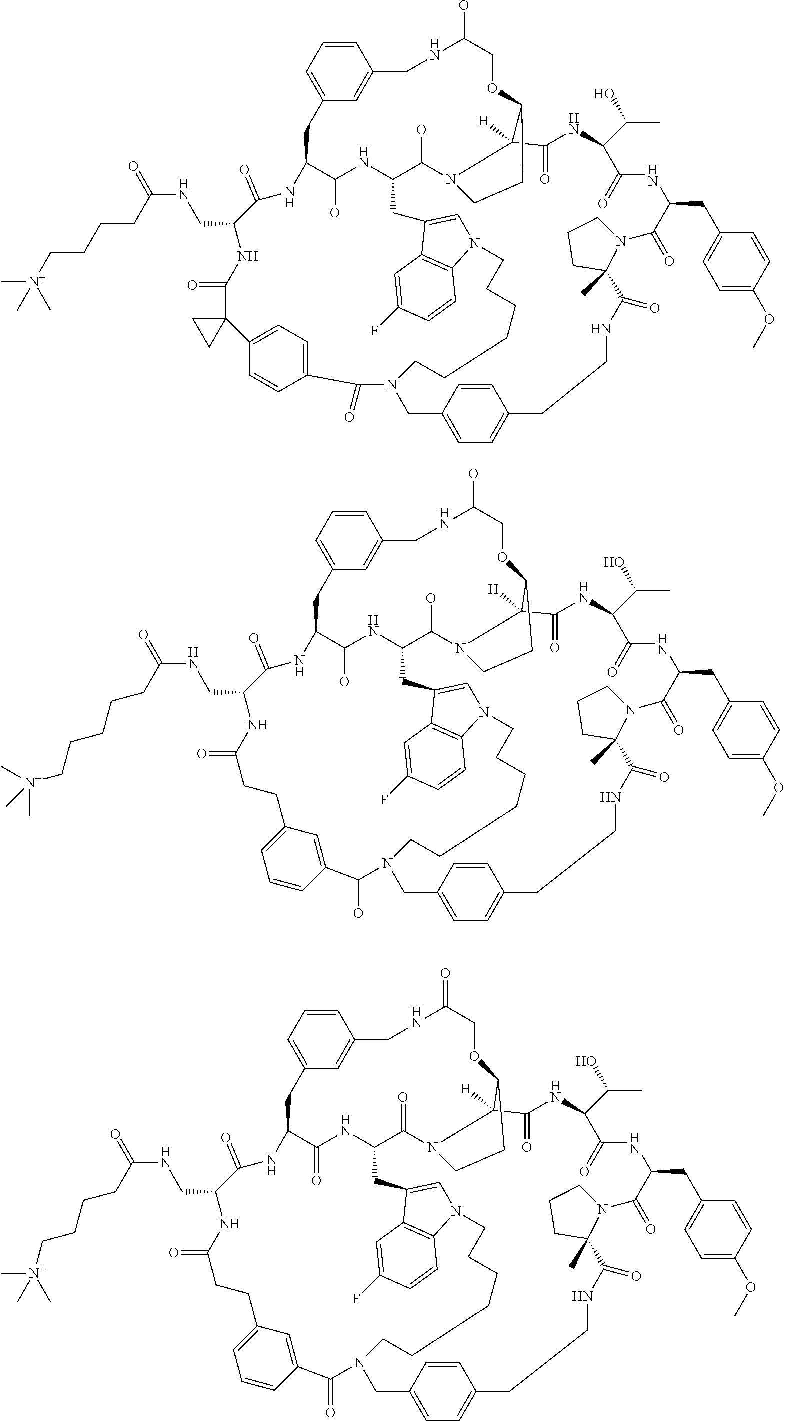

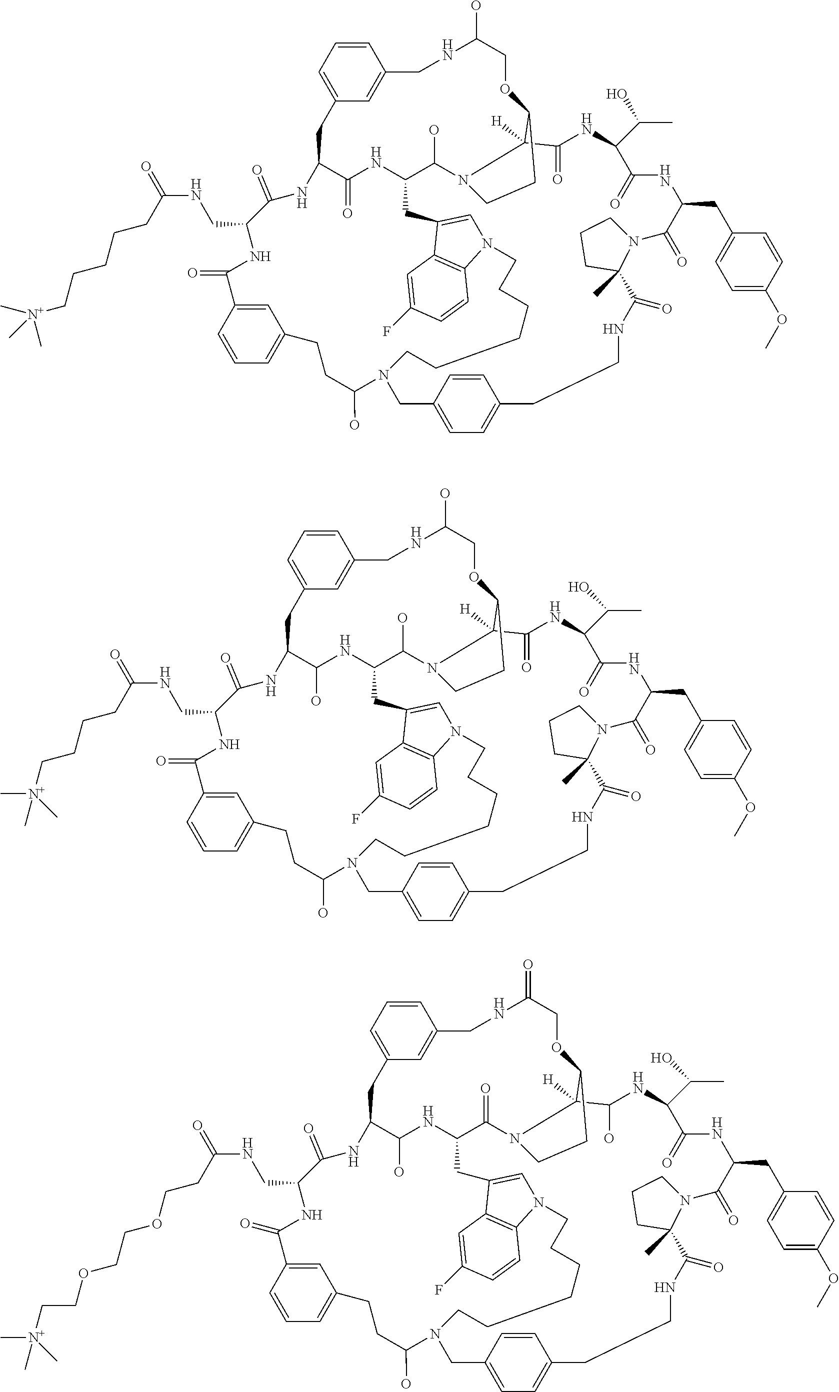

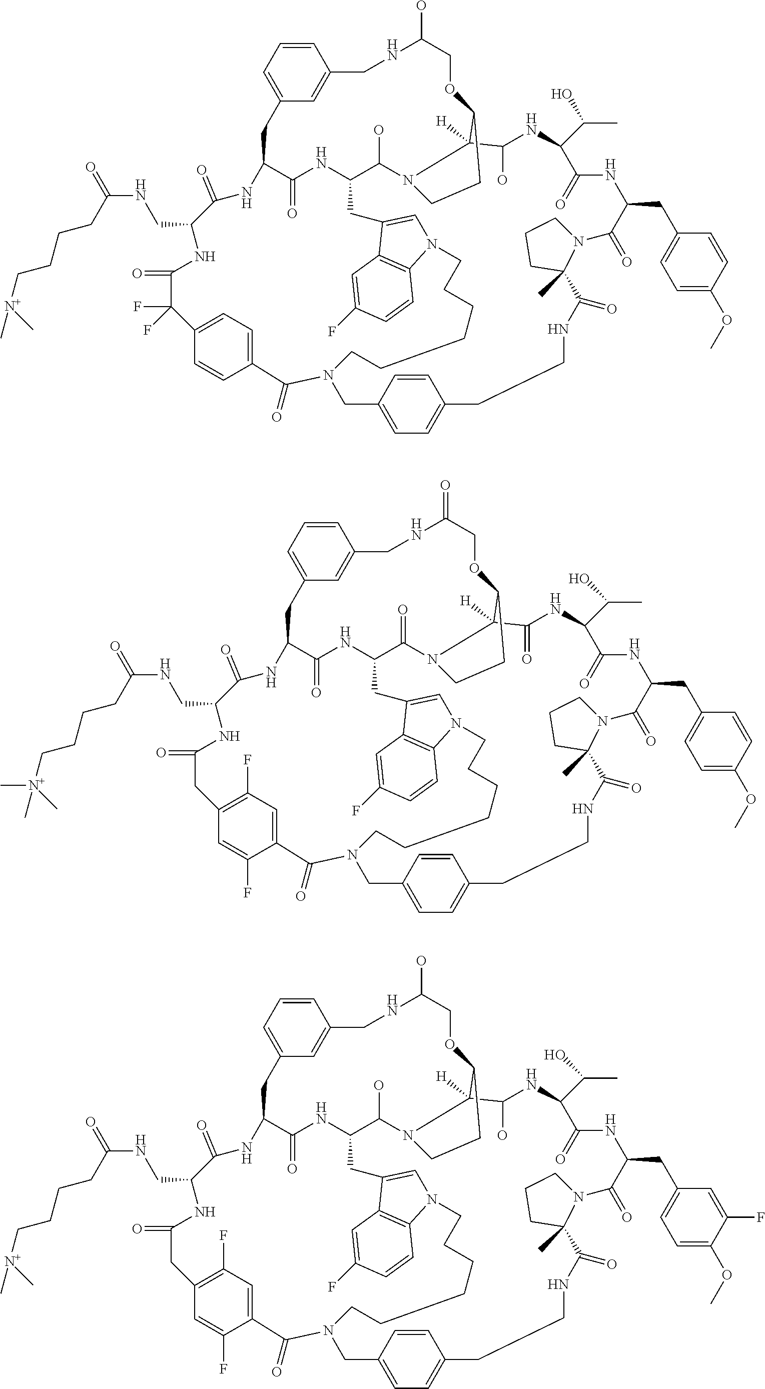

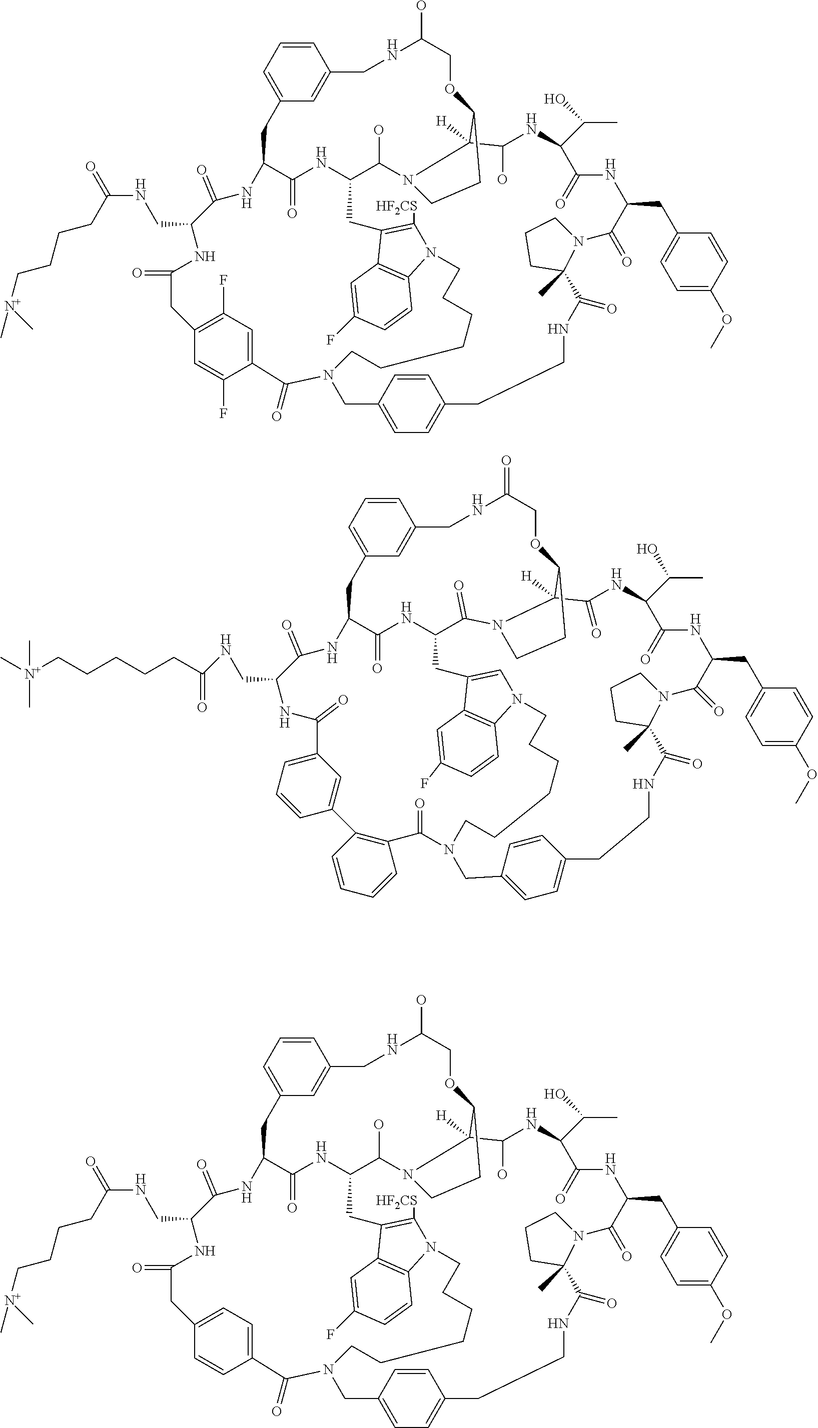

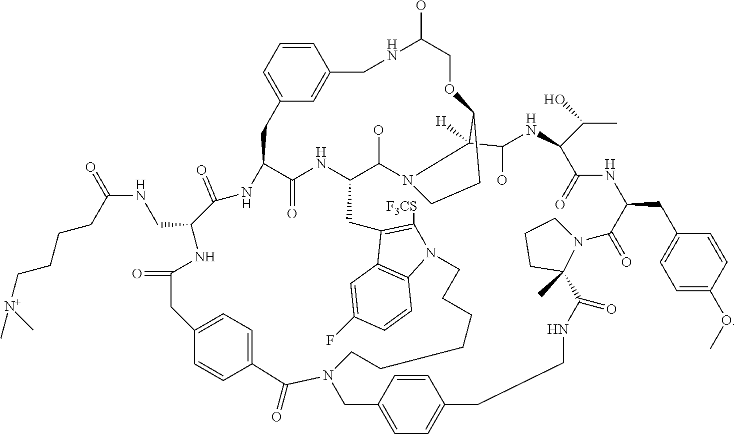

14. The compound or a pharmaceutically acceptable salt thereof according to claim 1, wherein the structural formula of the compound is selected from the following:

| Nos. | Structure |
|---|---|
| 2401 | 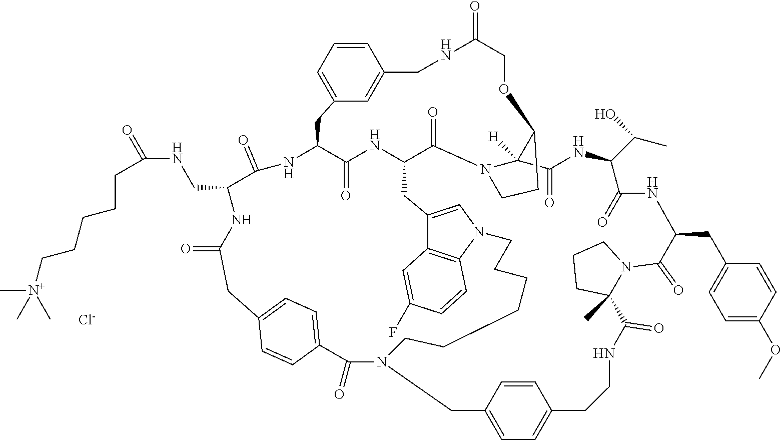 [SMILES: COc1ccc(C[C@@H]2NC(=O)[C@H]([C@@H](C)O)NC(=O)[C@@H]3[C@@H]4CCN3C(=O)[C@@H]3Cc5cn(c6ccc(F)cc56)CCCCCCN(Cc5ccc(cc5)CCNC(=O)[C@]5(C)CCCN5C2=O)C(=O)c2ccc(cc2)CC(=O)N[C@H](CNC(=O)CCCCC[N+](C)(C)C)C(=O)N[C@@H](Cc2cccc(c2)CNC(=O)CO4)C(=O)N3)cc1 ; [Cl-]] |
| 2450 | 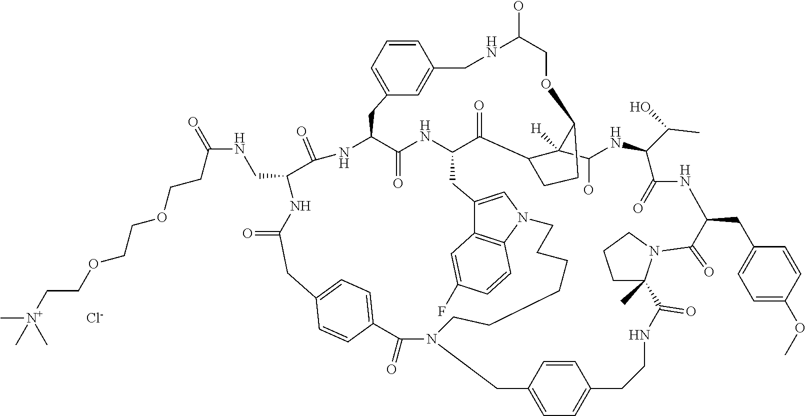 [SMILES: COc1ccc(C[C@@H]2NC(=O)[C@H]([C@@H](C)O)NC(O)[C@H]3C4CC[C@@H]3OCC(O)NCc3cccc(c3)C[C@@H]3NC(=O)[C@@H](CNC(=O)CCOCCOCC[N+](C)(C)C)NC(=O)Cc5ccc(cc5)C(=O)N(CCCCCCn5cc(c6cc(F)ccc65)C[C@H](NC3=O)C4=O)Cc3ccc(cc3)CCNC(=O)[C@]3(C)CCCN3C2=O)cc1 ; [Cl-]] |
| 2451 | 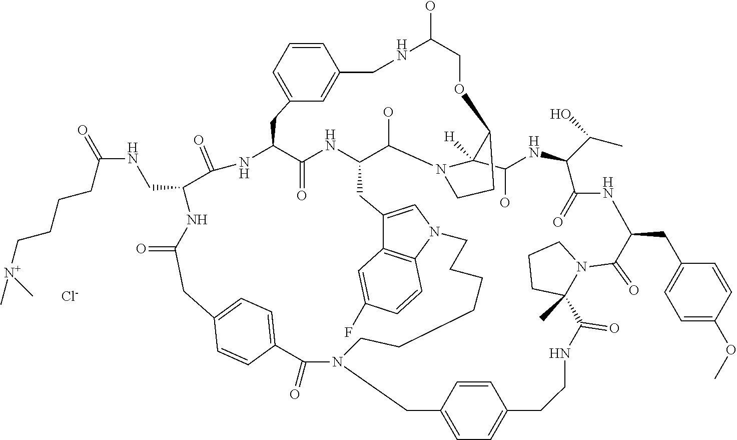 [SMILES: COc1ccc(C[C@@H]2NC(=O)[C@H]([C@@H](C)O)NC(O)[C@@H]3[C@@H]4CCN3C(O)[C@@H]3Cc5cn(c6ccc(F)cc56)CCCCCCN(Cc5ccc(cc5)CCNC(=O)[C@]5(C)CCCN5C2=O)C(=O)c2ccc(cc2)CC(=O)N[C@H](CNC(=O)CCCC[NH+](C)C)C(=O)N[C@@H](Cc2cccc(c2)CNC(O)CO4)C(=O)N3)cc1 ; [Cl-]] |
| 2455 | 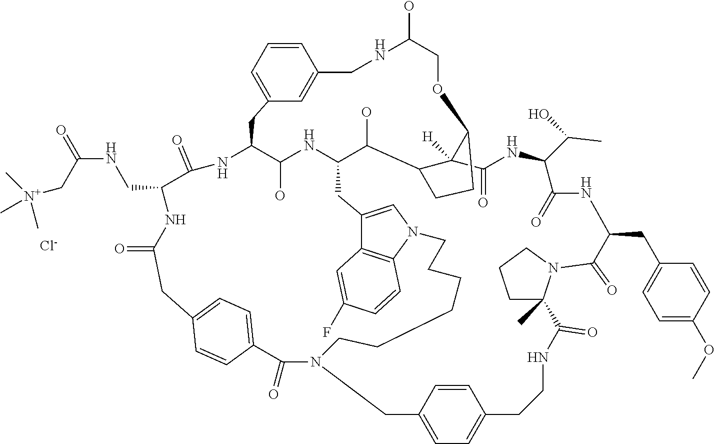 [SMILES: COc1ccc(C[C@@H]2NC(=O)[C@H]([C@@H](C)O)NC(=O)[C@H]3C4CC[C@@H]3OCC(O)NCc3cccc(c3)C[C@@H]3NC(=O)[C@@H](CNC(=O)C[N+](C)(C)C)NC(=O)Cc5ccc(cc5)C(=O)N(CCCCCCn5cc(c6cc(F)ccc65)C[C@H](NC3O)C4O)Cc3ccc(cc3)CCNC(=O)[C@]3(C)CCCN3C2=O)cc1 ; [Cl-]] |
| 2457 | 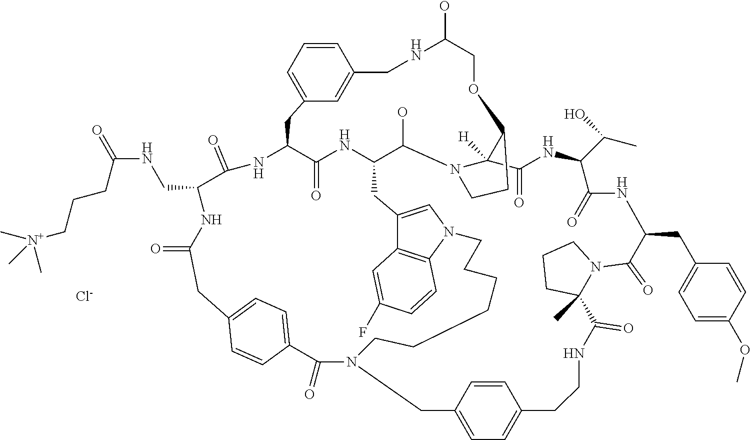 |
| 2460 | 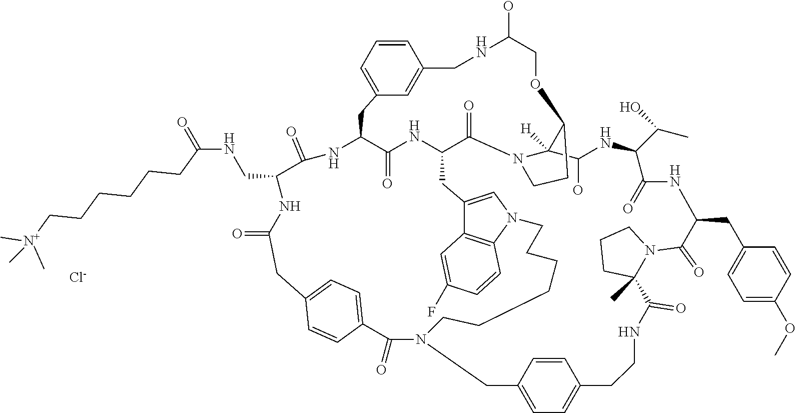 [SMILES: COc1ccc(C[C@@H]2NC(=O)[C@H]([C@@H](C)O)NC(O)[C@@H]3[C@@H]4CCN3C(=O)[C@@H]3Cc5cn(c6ccc(F)cc56)CCCCCCN(Cc5ccc(cc5)CCNC(=O)[C@]5(C)CCCN5C2=O)C(=O)c2ccc(cc2)CC(=O)N[C@H](CNC(=O)CCCCCC[N+](C)(C)C)C(=O)N[C@@H](Cc2cccc(c2)CNC(O)CO4)C(=O)N3)cc1 ; [Cl-]] |
| 2461 | 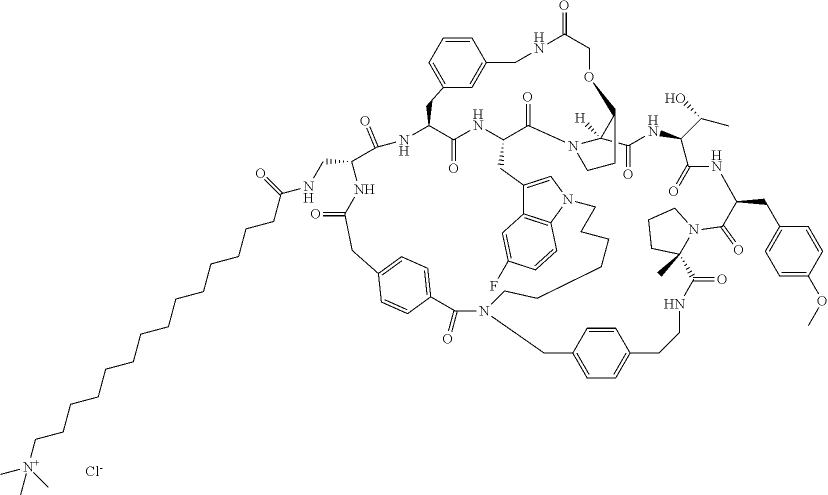 |
| 2402A | 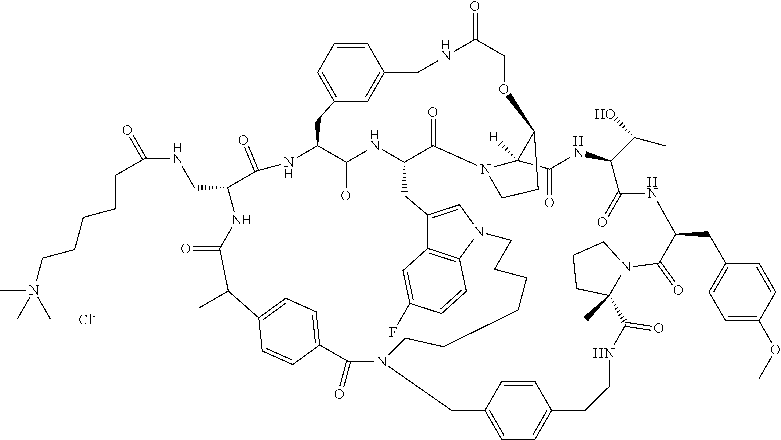 [SMILES: COc1ccc(C[C@@H]2NC(=O)[C@H]([C@@H](C)O)NC(=O)[C@@H]3[C@@H]4CCN3C(=O)[C@@H]3Cc5cn(c6ccc(F)cc56)CCCCCCN(Cc5ccc(cc5)CCNC(=O)[C@]5(C)CCCN5C2=O)C(=O)c2ccc(cc2)C(C)C(=O)N[C@H](CNC(=O)CCCCC[N+](C)(C)C)C(=O)N[C@@H](Cc2cccc(c2)CNC(=O)CO4)C(O)N3)cc1 ; [Cl-]] |
| 2402B | 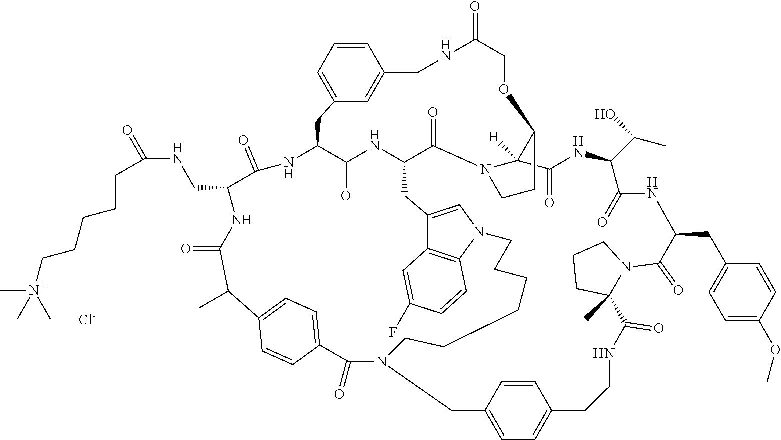 [SMILES: COc1ccc(C[C@@H]2NC(=O)[C@H]([C@@H](C)O)NC(=O)[C@@H]3[C@@H]4CCN3C(=O)[C@@H]3Cc5cn(c6ccc(F)cc56)CCCCCCN(Cc5ccc(cc5)CCNC(=O)[C@]5(C)CCCN5C2=O)C(=O)c2ccc(cc2)C(C)C(=O)N[C@H](CNC(=O)CCCCC[N+](C)(C)C)C(=O)N[C@@H](Cc2cccc(c2)CNC(=O)CO4)C(O)N3)cc1 ; [Cl-]] |
| 204B | 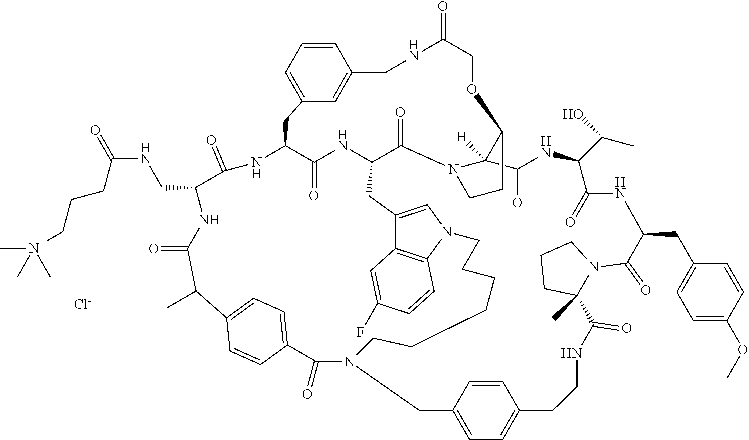 [SMILES: COc1ccc(C[C@@H]2NC(=O)[C@H]([C@@H](C)O)NC(O)[C@@H]3[C@@H]4CCN3C(=O)[C@@H]3Cc5cn(c6ccc(F)cc56)CCCCCCN(Cc5ccc(cc5)CCNC(=O)[C@]5(C)CCCN5C2=O)C(=O)c2ccc(cc2)C(C)C(=O)N[C@H](CNC(=O)CCC[N+](C)(C)C)C(=O)N[C@@H](Cc2cccc(c2)CNC(=O)CO4)C(=O)N3)cc1 ; [Cl-]] |
| 205A | 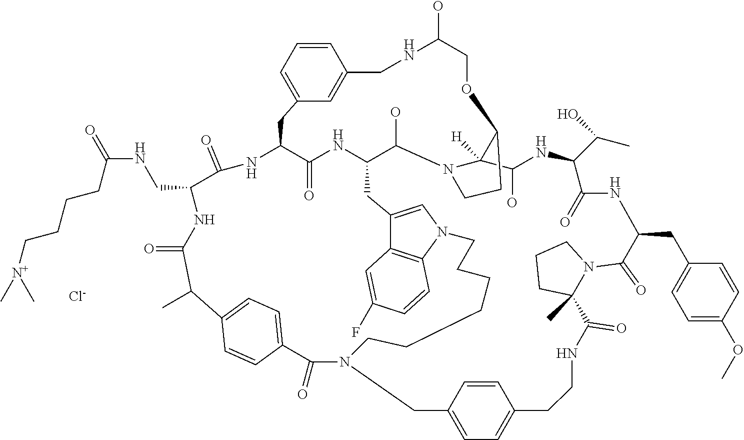 [SMILES: COc1ccc(C[C@@H]2NC(=O)[C@H]([C@@H](C)O)NC(O)[C@@H]3[C@@H]4CCN3C(O)[C@@H]3Cc5cn(c6ccc(F)cc56)CCCCCCN(Cc5ccc(cc5)CCNC(=O)[C@]5(C)CCCN5C2=O)C(=O)c2ccc(cc2)C(C)C(=O)N[C@H](CNC(=O)CCCC[NH+](C)C)C(=O)N[C@@H](Cc2cccc(c2)CNC(O)CO4)C(=O)N3)cc1 ; [Cl-]] |
| 205B | 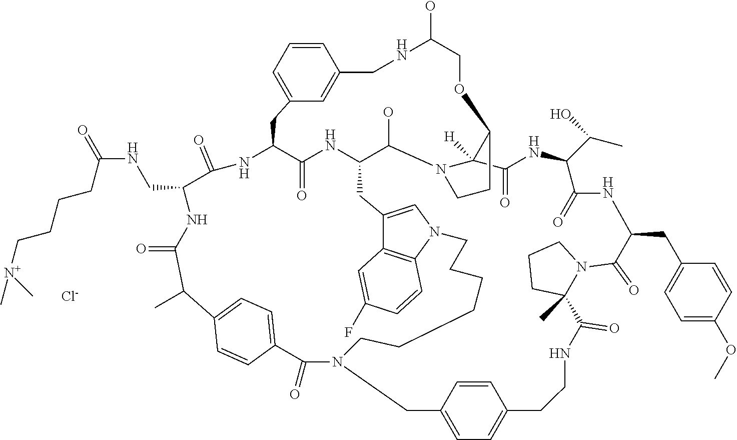 [SMILES: COc1ccc(C[C@@H]2NC(=O)[C@H]([C@@H](C)O)NC(=O)[C@@H]3[C@@H]4CCN3C(O)[C@@H]3Cc5cn(c6ccc(F)cc56)CCCCCCN(Cc5ccc(cc5)CCNC(=O)[C@]5(C)CCCN5C2=O)C(=O)c2ccc(cc2)C(C)C(=O)N[C@H](CNC(=O)CCCC[NH+](C)C)C(=O)N[C@@H](Cc2cccc(c2)CNC(O)CO4)C(=O)N3)cc1 ; [Cl-]] |
| 211B | 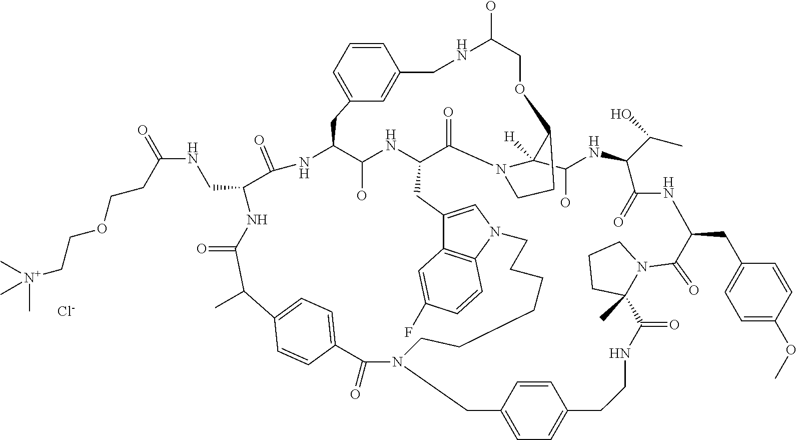 |
| 212A | 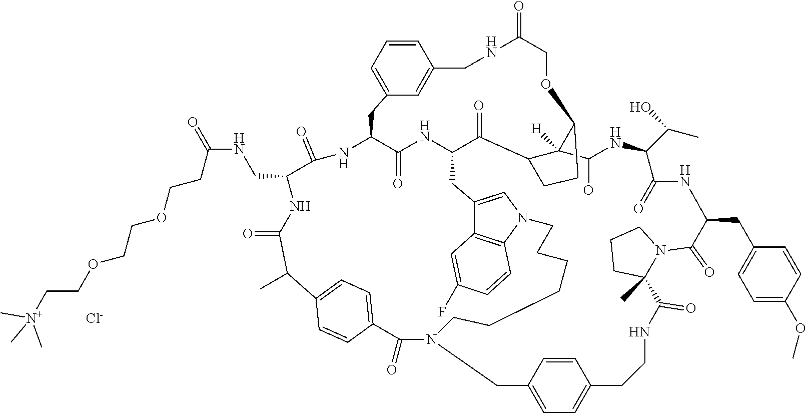 [SMILES: COc1ccc(C[C@@H]2NC(=O)[C@H]([C@@H](C)O)NC(O)[C@H]3C4CC[C@@H]3OCC(=O)NCc3cccc(c3)C[C@@H]3NC(=O)[C@@H](CNC(=O)CCOCCOCC[N+](C)(C)C)NC(=O)C(C)c5ccc(cc5)C(=O)N(CCCCCCn5cc(c6cc(F)ccc65)C[C@H](NC3=O)C4=O)Cc3ccc(cc3)CCNC(=O)[C@]3(C)CCCN3C2=O)cc1 ; [Cl-]] |
| 212B | 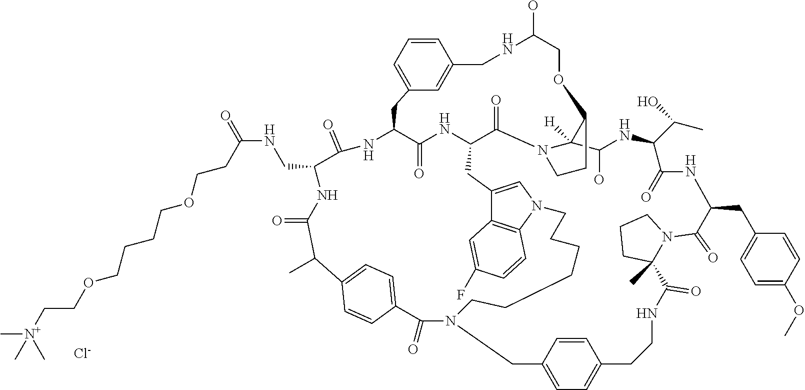 |
| 213B | 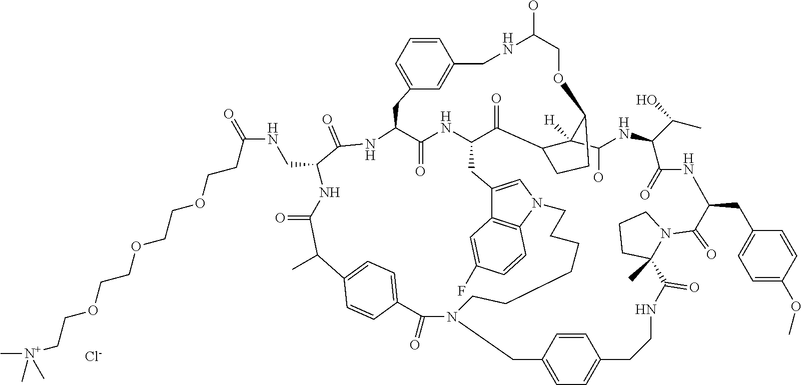 [SMILES: COc1ccc(C[C@@H]2NC(=O)[C@H]([C@@H](C)O)NC(O)[C@H]3C4CC[C@@H]3OCC(O)NCc3cccc(c3)C[C@@H]3NC(=O)[C@@H](CNC(=O)CCOCCOCCOCC[N+](C)(C)C)NC(=O)C(C)c5ccc(cc5)C(=O)N(CCCCCCn5cc(c6cc(F)ccc65)C[C@H](NC3=O)C4=O)Cc3ccc(cc3)CCNC(=O)[C@]3(C)CCCN3C2=O)cc1 ; [Cl-]] |
| 2403 | 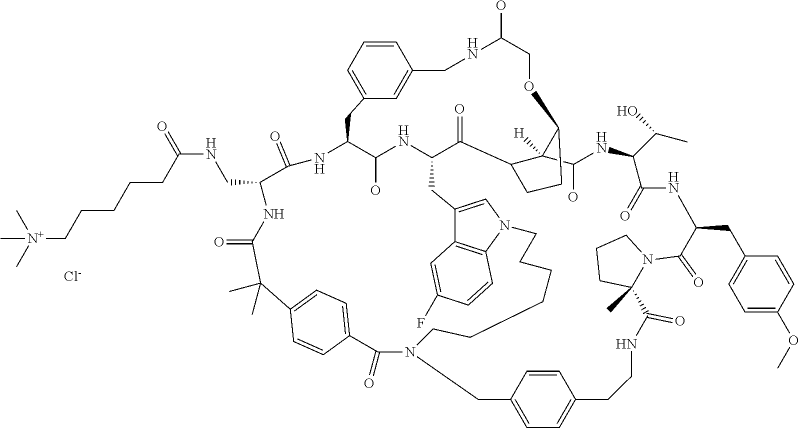 [SMILES: COc1ccc(C[C@@H]2NC(=O)[C@H]([C@@H](C)O)NC(O)[C@H]3C4CC[C@@H]3OCC(O)NCc3cccc(c3)C[C@@H]3NC(=O)[C@@H](CNC(=O)CCCCC[N+](C)(C)C)NC(=O)C(C)(C)c5ccc(cc5)C(=O)N(CCCCCCn5cc(c6cc(F)ccc65)C[C@H](NC3O)C4=O)Cc3ccc(cc3)CCNC(=O)[C@]3(C)CCCN3C2=O)cc1 ; [Cl-]] |
| 2430 | 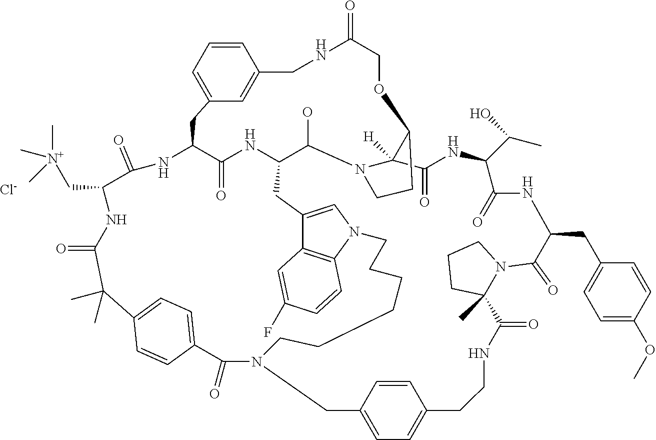 [SMILES: [Cl-] ; COc1ccc(C[C@@H]2NC(=O)[C@H]([C@@H](C)O)NC(=O)[C@@H]3[C@@H]4CCN3C(O)[C@@H]3Cc5cn(c6ccc(F)cc56)CCCCCCN(Cc5ccc(cc5)CCNC(=O)[C@]5(C)CCCN5C2=O)C(=O)c2ccc(cc2)C(C)(C)C(=O)N[C@H](C[N+](C)(C)C)C(=O)N[C@@H](Cc2cccc(c2)CNC(=O)CO4)C(=O)N3)cc1] |
| 2404 | 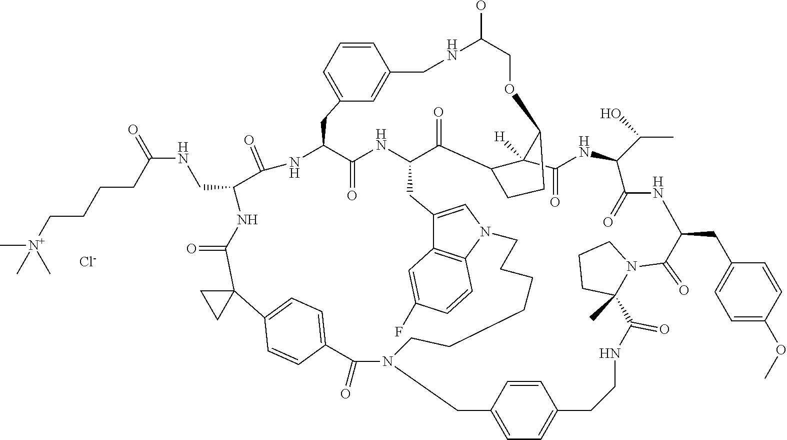 [SMILES: COc1ccc(C[C@@H]2NC(=O)[C@H]([C@@H](C)O)NC(=O)[C@H]3C4CC[C@@H]3OCC(O)NCc3cccc(c3)C[C@@H]3NC(=O)[C@@H](CNC(=O)CCCC[N+](C)(C)C)NC(=O)C5(CC5)c5ccc(cc5)C(=O)N(CCCCCCn5cc(c6cc(F)ccc65)C[C@H](NC3=O)C4=O)Cc3ccc(cc3)CCNC(=O)[C@]3(C)CCCN3C2=O)cc1 ; [Cl-]] |
| 2410 | 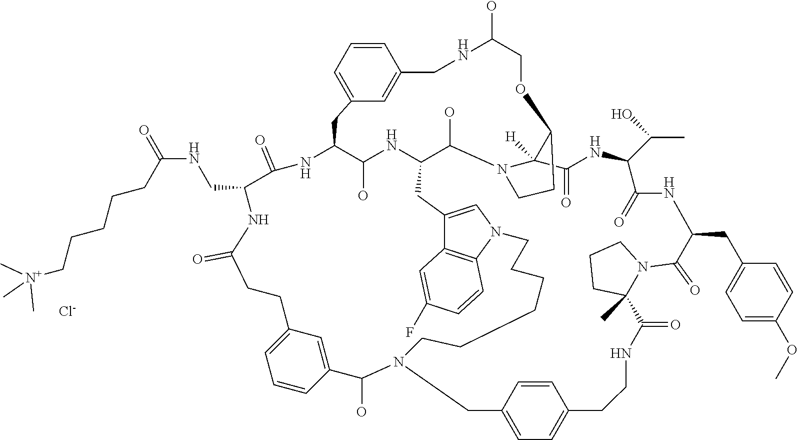 [SMILES: COc1ccc(C[C@@H]2NC(=O)[C@H]([C@@H](C)O)NC(=O)[C@@H]3[C@@H]4CCN3C(O)[C@@H]3Cc5cn(c6ccc(F)cc56)CCCCCCN(Cc5ccc(cc5)CCNC(=O)[C@]5(C)CCCN5C2=O)C(O)c2cccc(c2)CCC(=O)N[C@H](CNC(=O)CCCCC[N+](C)(C)C)C(=O)N[C@@H](Cc2cccc(c2)CNC(O)CO4)C(O)N3)cc1 ; [Cl-]] |
| 2452 | 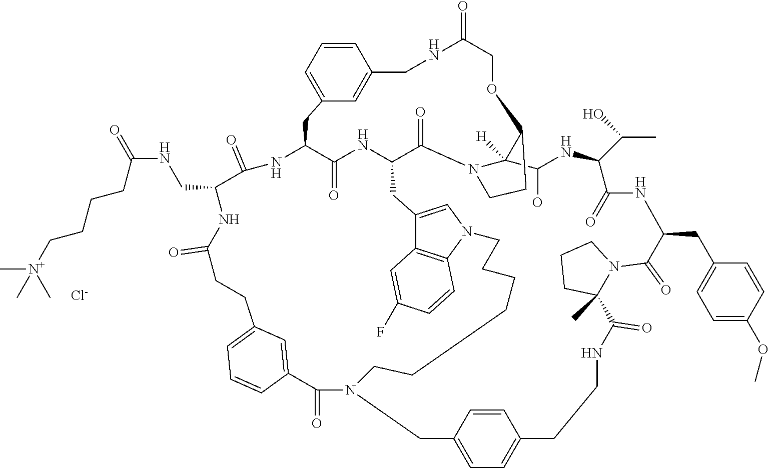 |
| 2412 | 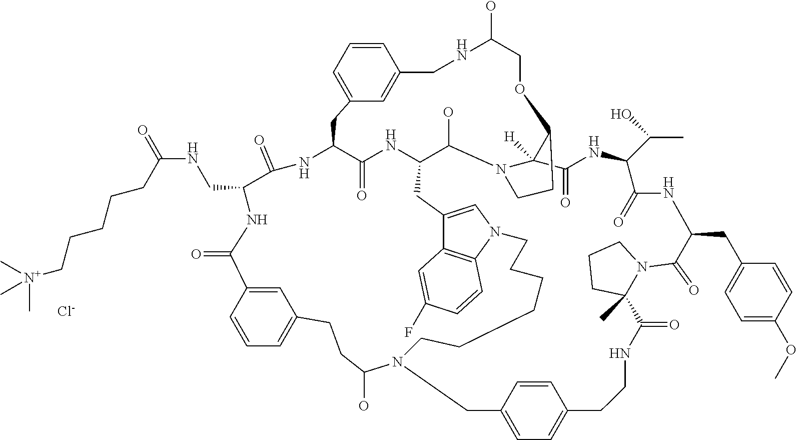 |
| 2453 | 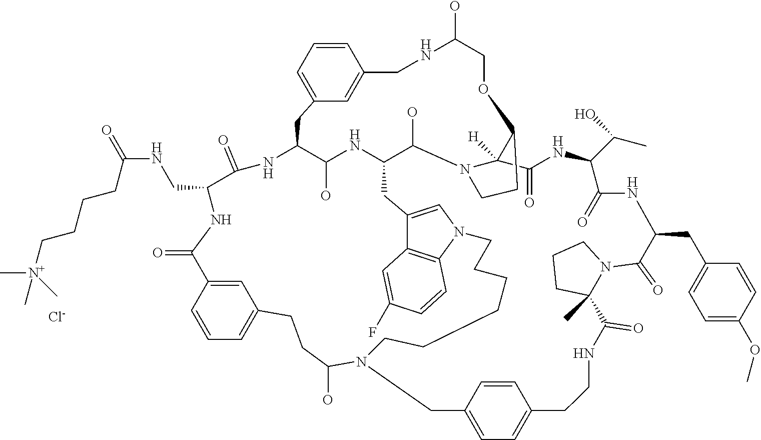 |
| 2454 | 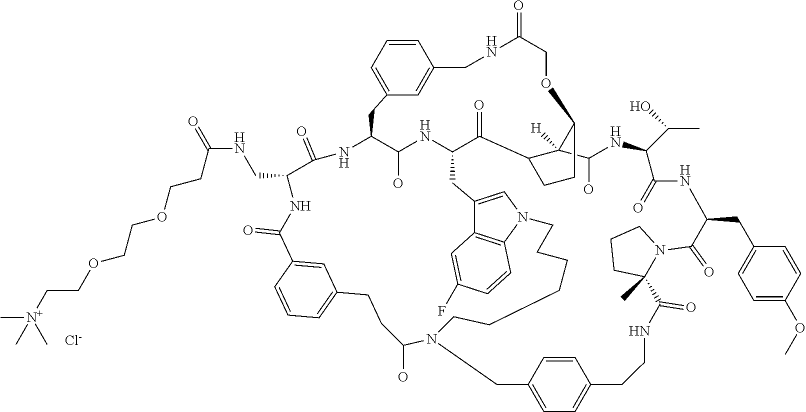 [SMILES: COc1ccc(C[C@@H]2NC(=O)[C@H]([C@@H](C)O)NC(O)[C@H]3C4CC[C@@H]3OCC(=O)NCc3cccc(c3)C[C@@H]3NC(=O)[C@@H](CNC(=O)CCOCCOCC[N+](C)(C)C)NC(=O)c5cccc(c5)CCC(O)N(CCCCCCn5cc(c6cc(F)ccc65)C[C@H](NC3O)C4=O)Cc3ccc(cc3)CCNC(=O)[C@]3(C)CCCN3C2=O)cc1 ; [Cl-]] |
| 2416 | 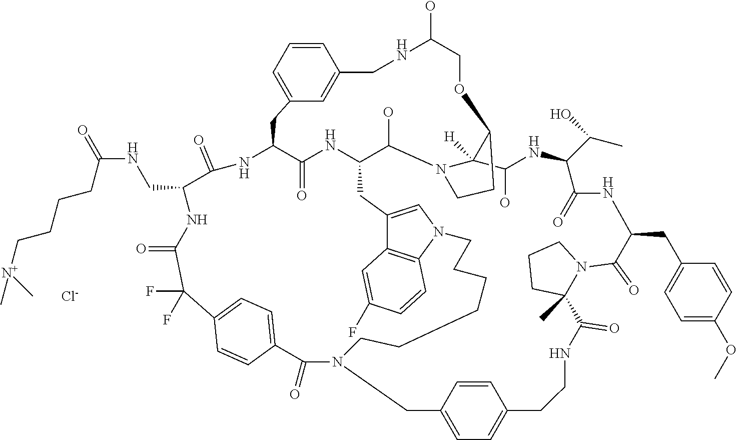 [SMILES: COc1ccc(C[C@@H]2NC(=O)[C@H]([C@@H](C)O)NC(O)[C@@H]3[C@@H]4CCN3C(O)[C@@H]3Cc5cn(c6ccc(F)cc56)CCCCCCN(Cc5ccc(cc5)CCNC(=O)[C@]5(C)CCCN5C2=O)C(=O)c2ccc(cc2)C(F)(F)C(=O)N[C@H](CNC(=O)CCCC[NH+](C)C)C(=O)N[C@@H](Cc2cccc(c2)CNC(O)CO4)C(=O)N3)cc1 ; [Cl-]] |
| 2421 | 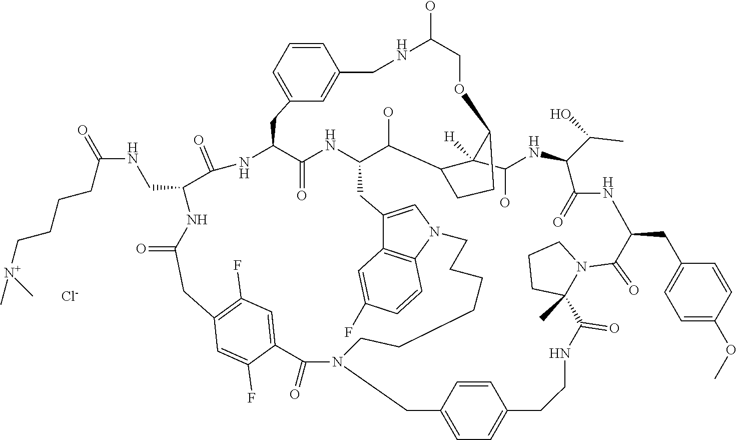 [SMILES: COc1ccc(C[C@@H]2NC(=O)[C@H]([C@@H](C)O)NC(O)[C@H]3C4CC[C@@H]3OCC(O)NCc3cccc(c3)C[C@@H]3NC(=O)[C@@H](CNC(=O)CCCC[NH+](C)C)NC(=O)Cc5cc(F)c(cc5F)C(=O)N(CCCCCCn5cc(c6cc(F)ccc65)C[C@H](NC3=O)C4O)Cc3ccc(cc3)CCNC(=O)[C@]3(C)CCCN3C2=O)cc1 ; [Cl-]] |
| 2427 | 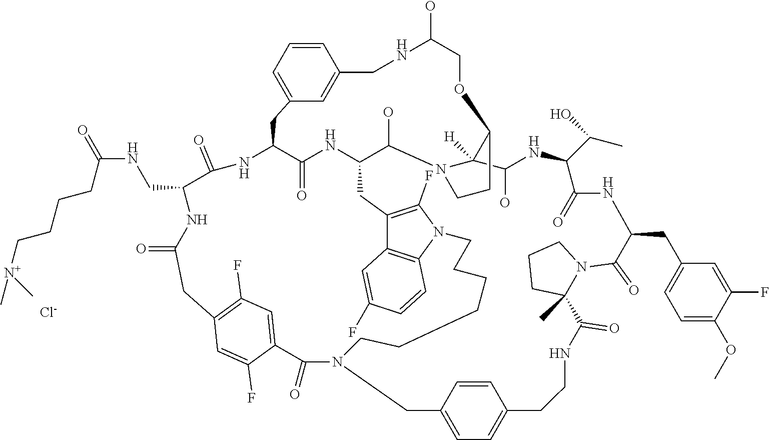 [SMILES: COc1ccc(C[C@@H]2NC(=O)[C@H]([C@@H](C)O)NC(O)[C@@H]3[C@@H]4CCN3C(O)[C@@H]3Cc5c(F)n(c6ccc(F)cc56)CCCCCCN(Cc5ccc(cc5)CCNC(=O)[C@]5(C)CCCN5C2=O)C(=O)c2cc(F)c(cc2F)CC(=O)N[C@H](CNC(=O)CCCC[NH+](C)C)C(=O)N[C@@H](Cc2cccc(c2)CNC(O)CO4)C(=O)N3)cc1F ; [Cl-]] |
| 2428 | 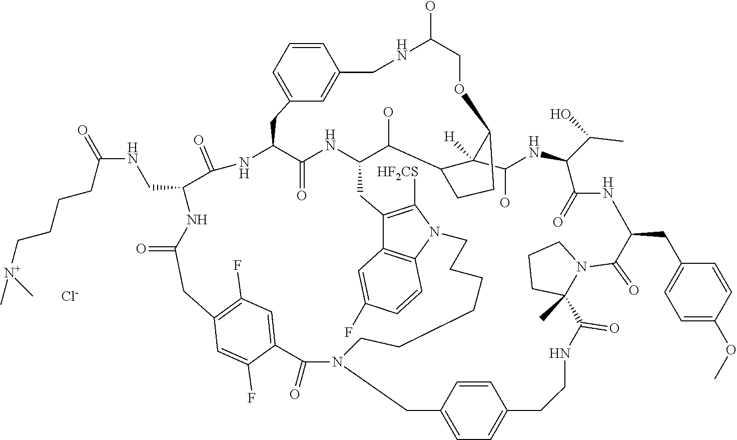 |
| 2407 | 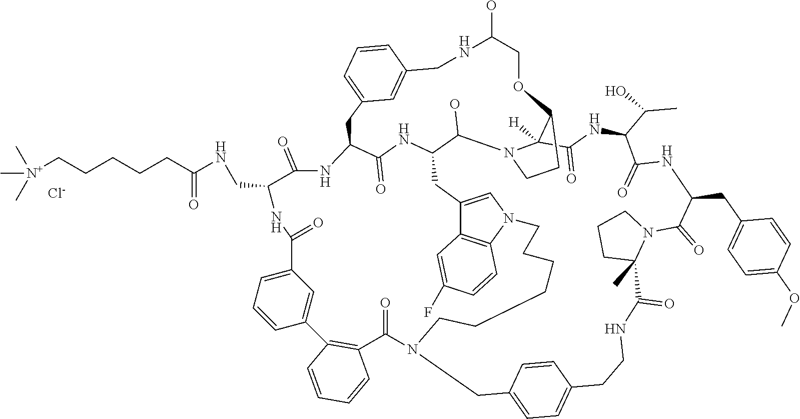 |
| 2462 | 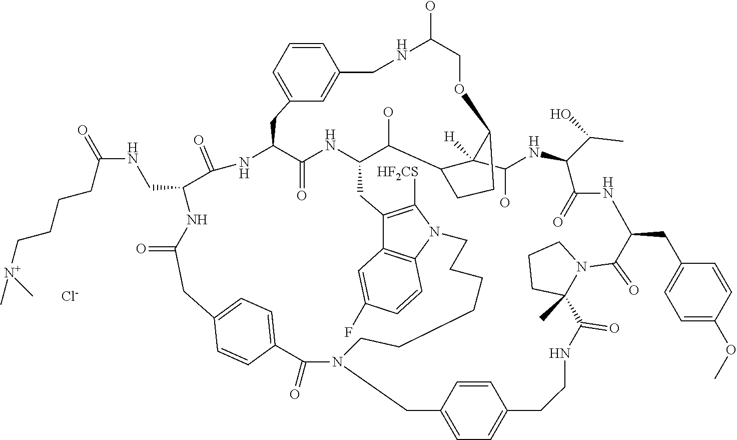 |
| 2463 | 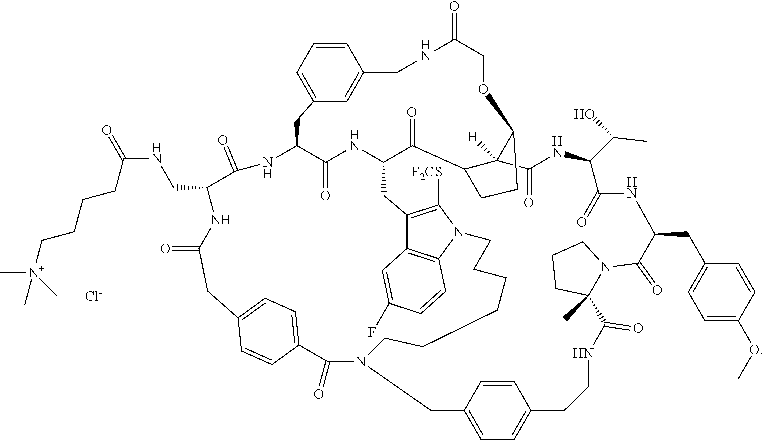 |

15. A pharmaceutical composition, wherein, it comprises a compound or a pharmaceutically acceptable salt thereof of claim 1.

16. The pharmaceutical composition according to claim 15, wherein, the pharmaceutical composition is selected from an oral preparation or an injectable preparation.

17. A method for inhibiting PCSK9, comprising administering an effective dose of the compound or a pharmaceutically acceptable salt thereof of claim 1 to a patient.

18. The method according to claim 17, wherein, the patient has hypercholesterolemia, and/or atherosclerosis, and/or coronary heart disease, and/or metabolic syndrome, and/or acute coronary syndrome.

19. A method for treating the following diseases, comprising administering an effective dose of the compound or a pharmaceutically acceptable salt thereof claim 1 to a patient; the disease is selected from atherosclerosis, and/or hypercholesterolemia, and/or coronary heart disease, and/or metabolic syndrome, and/or acute coronary syndrome.
